(19) United States
(12) Patent Application Publication
(10) Pub. No.: US 2023/0377709 A1
(43) Pub. Date: Nov. 23, 2023

(54) METHOD OF CONTROLLING AUTONOMOUS OPERATIONS IN A SURGICAL SYSTEM

(71) Applicant: Cilag GmbH International, Zug (CH)

(72) Inventors: Frederick E. Shelton, IV, Hillsboro, OH (US); Kevin L. Houser, Springboro, OH (US); Taylor W. Aronhalt, Loveland, OH (US); Sarah A. Worthington, Cincinnati, OH (US); Kevin M. Fiebig, Cincinnati, OH (US); Curtis Anthony Maples, Cincinnati, OH (US); Nicholas James Ross, Franklin, OH (US); Shane R. Adams, Lebanon, OH (US); Matthew David Cowperthwait, Cincinnati, OH (US); Raymond Edward Parfett, Loveland, OH (US); Jacqueline Corrigan Aronhalt, Loveland, OH (US); Charles J. Scheib, Loveland, OH (US); Jason L. Harris, Lebanon, OH (US); Aaron Chow, Cincinnati, OH (US)

(21) Appl. No.: 17/747,806

(22) Filed: May 18, 2022

(51) Int. Cl. G16H 20/30

(52) U.S. Cl. CPC G16H 20/30

(57) ABSTRACT

Examples described herein may include a surgical computing system that determines an autonomous operation parameter and generates a control signal for an autonomous operation based on the autonomous operation parameter. The surgical computing system may obtain surgical data and determine the autonomous operation parameter based on the surgical data. The surgical computing system may obtain surgical data and determine the autonomous operation parameter based on the surgical data. The surgical computing system may send the control signal for the autonomous operation, for example, to one or more smart surgical devices.

### CROSS-REFERENCE TO RELATED APPLICATIONS

This application is related to the following, filed contemporaneously, the contents of each of which are incorporated by reference herein:
U.S. patent application, entitled DYNAMICALLY DETERMINING SURGICAL AUTONOMY LEVEL, with attorney docket number END9430USNP2;
U.S. patent application, entitled DETECTING FAILURE MITIGATION ASSOCIATED WITH AUTONOMOUS SURGICAL TASK, with attorney docket number END9430USNP3;
U.S. patent application, entitled ADAPTED AUTONOMY FUNCTIONS AND SYSTEM INTERCONNECTIONS, with attorney docket number END9430USNP4;
U.S. patent application, entitled AUTONOMOUS ADAPTATION OF SURGICAL DEVICE CONTROL ALGORITHM, with attorney docket number END9430USNP5;
U.S. patent application, entitled AUTONOMOUS INTRA-INSTRUMENT SURGICAL SYSTEM ACTUATION, with attorney docket number END9430USNP6;
U.S. patent application, entitled AUTONOMOUS SURGICAL SYSTEM INSTRUMENT ACTUATION, with attorney docket number END9430USNP7;
U.S. patent application, entitled AUTOMATIC COMPILATION, ANNOTATION, AND DISSEMINATION OF SURGICAL DATA TO SYSTEMS TO ANTICIPATE RELATED AUTOMATED OPERATIONS, with attorney docket number END9430USNP8;
U.S. patent application, entitled AGGREGATION OF PATIENT, PROCEDURE, SURGEON, AND FACILITY PRE-SURGICAL DATA AND POPULATION AND ADAPTATION OF A STARTING PROCEDURE PLAN TEMPLATE, with attorney docket number END9430USNP9; and
U.S. patent application, entitled IDENTIFICATION OF IMAGES SHAPES BASED ON SITUATIONAL AWARENESS OF A SURGICAL IMAGE AND ANNOTATION OF SHAPES OR PIXELS, with attorney docket number END9430USNP10.

### BACKGROUND

Surgical procedures are typically performed in surgical operating theaters or rooms in a healthcare facility such as, for example, a hospital. Various surgical devices and systems are utilized in performance of a surgical procedure. In the digital and information age, medical systems and facilities are often slower to implement systems or procedures utilizing newer and improved technologies due to patient safety and a general desire for maintaining traditional practices.

### SUMMARY

A surgical task may be automated. A device may receive an indication of a surgical task to be performed with a surgical instrument. Capabilities of the surgical instrument may be associated with levels of automation. The device may monitor a performance of the surgical task with the surgical instrument operating at a first level of automation associated with the levels of automation. The device may detect a trigger event associated with the performance of the surgical task and switch operation of the surgical instrument from the first level of automation to a second level of automation associated with levels of automation, for example, based on the trigger event. The capabilities of the surgical instrument may include a set of surgical instrument tasks and the level of automation may be associated with automating one or more surgical instrument tasks from the set of surgical instrument tasks. The performance of the surgical task may be based on real-time surgical data. The real-time surgical data may include one or more of the following: user data, surgical environment data, surgical instrument data, task data, historical data, or the like. In example, the device may detect the trigger event by comparing the performance of the surgical task with a trigger event threshold and may switch operation of the surgical instrument from the first level of automation to the second level of automation based on the trigger event. The second level of automation may be associated with automating less surgical instrument tasks when compared to the first level of automation. Monitoring the performance of the surgical task may include comparing one or more of the real-time surgical data to respective ideal surgical data.

In examples, the device may operate at a first level of automation of the levels of automation associated with the surgical task. The device may obtain an indication to switch to a second level of automation of the levels of automation associated with the surgical task. The indication may be based on detecting a trigger. The device may operate at the second level of automation of the levels of automation associated with the surgical task based on the indication. The indication may be based on monitoring a performance of the first level of automation. The performance of the first level of automation may be based on real-time surgical data.

Systems, methods, and instrumentalities are described herein for detecting failure mitigation associated with a surgical task. A device may perform a first autonomous function associated with a surgical task. Performing the first autonomous function may be associated with control feedback. The first autonomous function may be monitored by tracking one or more outputs associated with the control feedback. If the one or more outputs cross a failure threshold, the device may switch from performing the first autonomous function to performing a second autonomous function associated with the surgical task. The second autonomous function may be associated with failure mitigation feedback. The device may generate a failure magnitude based on the one or more outputs that crossed the failure threshold. The device may adjust one or more parameters associated with the failure mitigation feedback based on the failure magnitude. In examples, the failure magnitude may be generated based on one or more of the following: surgical context, type of the first autonomous function, type of the second autonomous function, historical data associated with the first autonomous function, a number of outputs that crossed failure threshold, or a degree of the one or more outputs crossing the failure threshold.

Systems, methods, and instrumentalities are described herein for adapted autonomy functions and system interconnections. A surgical hub may detect a first surgical device in the surgical environment. The first surgical device may include a first plurality of features. The surgical hub may include a plurality of resources. The surgical hub may detect a second surgical device in the surgical environment. The second surgical device may include a second plurality of features. The surgical hub may determine one or more features from the first plurality of features for the first surgical device to perform and one or more features from the second plurality of features for the second surgical device to perform based on the plurality of resources.

Systems, methods, and instrumentalities are described herein for autonomous adaptation of surgical device control algorithm. A computing system for autonomous surgical device control algorithm adaptation may include a processor. The processor may be configured to perform one or more actions. The processor may be configured to receive first operation data associated with a first surgical procedure and second operation data associated with a second surgical procedure. In some examples, the first operation data is associated with a first aspect of a control algorithm of a first surgical device. In some examples, the second operation data is associated with a first aspect of a control algorithm of a second surgical device, and the first surgical device and second surgical device are of a first surgical device type. The processor is configured to receive first outcome data associated with the first surgical procedure and second outcome data associated with the second surgical procedure. The processor is configured to determine that each of the control algorithm of the first surgical device and the control algorithm of the second surgical device is an up-to-date control algorithm associated with the first surgical device type. The processor is configured to generate first aggregation data based on at least the first operation data, the second operation data, the first outcome data, and the second outcome data. Based on at least the first aggregation data, the processor is configured to determine a correlation between a first aspect of the up-to-date control algorithm and outcome data. Based on the determined correlation, the processor is configured to generate an updated up-to-date control algorithm.

Systems, methods, and instrumentalities are described herein for autonomous operation of a surgical device. For example, the surgical device may be a surgical cutting device or a surgical energy device. A first discrete signal associated with clamping control may be received by the surgical device. The first discrete signal may be associated with initiating closure of a clamping jaw. The first discrete signal may be triggered by a healthcare professional or autonomously activated. The surgical device, in response to the first discrete signal, may generate a first continuous signal to cause a continuous application of force based on a first autonomous control algorithm. For example, the continuous application of force may be adjusted autonomously based on at least a first measurement (e.g., a measurement associated with a tissue).

A second discrete signal associated with clamping control may be received by the surgical device. The second discrete signal may be associated with initiating a firing sequence. The second discrete signal may be triggered by a healthcare professional or autonomously activated. The deployment operation may be advancing of a cutting member and retracting of the cutting member. The surgical device, in response to the second discrete signal, may generate a second continuous signal to cause the deployment operation based on a second autonomous control algorithm. The second measurement may be a ratio of collagen to elastin in the tissue. The deployment operation may be adjusted autonomously based on at least the second measurement.

Systems, methods, and instrumentalities are described herein for autonomous operation of a surgical device within a predefined boundary. For example, the surgical device may be a smart grasper, a smart surgical stapler, or a smart energy device. The predefined boundary may be a virtual movement boundary associated with a surgical task. The predefined boundary may be a field of view defined by a scope device.

The surgical device, for example, a smart gasper, based at least on a condition tit a tissue tension measurement associated with the smart grasper being equal to or greater than a maximum tissue tension may determine a safety adjustment to the operation of the smart grasper. The safety adjustment may be a reduction of grasping force. The surgical device, for example, a smart stapler, based at least on a condition that an inrush current measurement being below a lowest threshold may determine a safety adjustment to the operation of the smart stapler. The safety adjustment may be stopping a firing sequence. The surgical device, for example, a smart energy device, based at least on a condition that a distance between the smart energy device and the smart grasper is below a threshold may determine a safety adjustment to movement of the smart energy device based on one or more location data and orientation data.

Systems, methods, and instrumentalities are disclosed for automatic compilation, annotation, and dissemination of surgical data to systems to anticipate related automated operations. Surgical procedure data may be selectively sent to surgical systems, for example, to perform autonomous tasks (e.g., without being requested for the data). A surgical computing system may obtain the surgical procedure data from surgical systems. The surgical computing system may annotate the surgical procedure data with surgical context data. The surgical computing system may determine a data need associated with a subsequent target system task associated with a target system. The surgical computing system may determine the target system, for example, based on the annotated surgical procedure data. The surgical computing system may generate a data package (e.g., selectively discriminated data) associated with the data needs, which may comprise a portion of the annotated surgical procedure data. The surgical computing system may send the data package to the target system.

The surgical computing system may be configured to determine surgical context data, for example, based on the surgical procedure data. The surgical computing system may be configured to send an indication to the target system indicating the current surgical procedure step.

The surgical computing system may be configured to determine a risk level based on the surgical procedure data. The data needs may be determined, for example, based on the risk level.

The surgical computing system may be configured to perform redaction on at least a portion of the surgical procedure data and/or annotated surgical procedure data. For example, the surgical computing system may determine a classification associated with surgical procedure data. The classification may indicate private and/or confidential information (e.g., with respect to HIPAA). The surgical computing system may determine that a target system may not receive confidential information, for example, based on the type of target device and/or geographic location associated with the target device (e.g., outside the HIPAA boundary). The surgical computing system may perform redaction on the portion of surgical procedure data, for example, before sending the data package to the target system.

Systems, methods, and instrumentalities are disclosed for aggregating patient, procedure, surgeon, and/or facility pre-surgical data and population and adaptation of a starting procedure plan template. Pre-surgical data may be used to populate a patient specific surgical procedure plan. A surgical system (e.g., surgical hub) may be configured to obtain patient specific pre-surgical data for a patient specific surgical procedure. The surgical system may obtain a surgical procedure template comprising surgical procedure steps. The surgical system may determine surgical task options for the surgical procedure steps, for example, based on the patient specific pre-surgical data. The surgical system may determine characterizations (e.g., predicted outcomes, risk levels, efficiency) for the surgical task options. A populated patient specific procedure plan may be generated, for example, which may include the determined surgical procedure steps and the respective characterizations.

The surgical system may select a set of surgical tasks from the surgical task options (e.g., pre-select a set), for example, based on the respective characterizations. The surgical task options selected may include the tasks with the best outcome success. The surgical system may generate a pre-selected populated procedure plan comprising the selected set of surgical tasks. The pre-selected populated procedure plan may be used as a recommendation for the surgical procedure.

The surgical system may obtain information separate from pre-surgical data, for example, such as facility information. The facility information may include information associated with surgical tools, surgical equipment, health care professional (HCP) availability, facility room availability, and/or the like. The patient specific procedure plan may be generated based on the facility information.

The surgical system may determine that surgical task options were determined using information that is incomplete and/or conflicting. The surgical system may indicate (e.g., in the populated surgical procedure plan) that the surgical task option may be determined using the incomplete and/or conflicting information. The surgical system may indicate to a user to input information that may be used to clarify the incomplete and/or conflicting data. The surgical system may redetermine the surgical task option and/or characterization associated with the surgical task option by considering the input information.

Systems, methods, and instrumentalities are disclosed for identification of image shapes based on situational awareness of a surgical image and annotation of shapes or pixels. A surgical video associated with a surgical procedure may be obtained. Surgical context data for the surgical procedure may be obtained. Elements in the video frames may be identified based on the surgical context data using image processing. Annotation data may be determined and generated for the video frames, for example, based on the surgical context data and the identified element(s).

The video frames in the surgical video may comprise pixels. The identified element(s) may correspond to a respective group of pixels. The identified element(s) may be separated into sub-elements that comprise the respective element. The sub-elements may be comprised of sub-groups of pixels associated with each sub-element.

The annotation data may be inserted into the video frames. For example, the annotation data may be inserted on a pixel level (e.g., respective annotation data be associated with each pixel and/or group of pixels). For example, the annotation data may include one or more of the following: element identification information, element grouping information, element subgrouping information, element type information, element description information, element condition information, surgical step information, surgical task information, surgical event information, surgical instrument information, surgical equipment information, and/or the like.

The surgical context data may be refined and/or updated based on the surgical video. For example, the surgical context data may be refined and/or updated based on the identified element(s) in a video frame. Element(s) in another video frame may be identified, for example, using the refined and/or updated surgical context data (e.g., using image processing). Annotation data for the second video frame may be determined using the refined surgical context data and the element(s) identified in the second video frame.

Tracking information associated with a surgical video may be determined, for example, by analyzing multiple video frames. Elements identified in a first video frame and elements identified in a second video frame may be used, for example, to determine tracking data. The tracking data may be associated with element behavior, element movement, and/or outcome information (e.g., associated with the elements). The annotation data may include the determined tracking data.

The tracking data and/or information may be verified, for example, using anticipated tracking information. The anticipated tracking information may be obtained, for example, based on the surgical context data and/or a surgical procedure plan. The annotation data may include an indication that indicates whether the elements and/or tracking data is verified. For example, the annotation data may indicate that a subset of the identified elements have been verified. The annotation data may indicate that a subset of the identified elements have not been verified.

Jobs, outcomes, and/or constraints may be determined, for example, for a surgical procedure based on the surgical video. The jobs, outcomes, and/or constraints may be determined based on the surgical context data. Surgical task information associated with a first video frame may be determined using the surgical context data and identified elements in the first video frame. Complication information may be determined associated with the determined surgical task, for example, using the surgical context data, the surgical task information, and the identified elements in the first surgical video frame. Outcome information associated with the surgical task may be determined, for example, based on the surgical context data, the surgical task information, and the complication information.

Change and/or control parameters may be determined for a surgical procedure by analyzing the surgical video. Change and/or control parameters may be determined, for example, based on the surgical task information, complication information, and/or the outcome information. A determination to change parameters associated with a surgical procedure may be performed. Parameter may include parameters associated with operating a surgical instrument, surgical equipment, and/or the like. Based on a determination to change parameters associated with a surgical procedure, change and/or control parameters may be determined. A control signal may be generated. The control signal may include an indication that indicates the determined control parameters. The control signal may be sent to a surgical control system, for example, that send parameter information to surgical systems (e.g., surgical instruments, surgical equipment, and/or the like). The change and/or control parameters may be used to perform the surgical procedure.

### BRIEF DESCRIPTION OF THE DRAWINGS

Examples described herein may include a Brief Description of the Drawings.

FIG. 1 is a block diagram of a computer-implemented surgical system.

FIG. 2 shows an example surgical system in a surgical operating room.

FIG. 3 illustrates an example surgical hub paired with various systems.

FIG. 4 illustrates a surgical data network having a set of communication surgical hubs configured to connect with a set of sensing systems, an environmental sensing system, and a set of devices, etc.

FIG. 5 illustrates a logic diagram of a control system of a surgical instrument.

FIG. 6 shows an example surgical system that includes a handle having a controller and a motor, an adapter releasably coupled to the handle, and a loading unit releasably coupled to the adapter.

FIG. 7 shows an example situationally aware surgical system.

FIG. 8 shows an example of a surgical autonomous system.

FIG. 9 shows an example of an autonomy module associated with a surgical instrument and surgical instrument capabilities.

FIG. 10 shows an example of computer-implemented autonomous surgical system.

FIG. 11 shows an example of monitoring the performance of surgical tasks associated with autonomy levels.

FIG. 12 shows an example of the relationship between error magnitude and autonomy levels.

FIG. 13 shows an example of the relationship between error magnitude and autonomy levels.

FIG. 14 shows an example of the relationship between autonomy level, machine learning, and the surgical task.

FIG. 15 shows an example determining the autonomy levels at which the surgical instrument performs a surgical task.

FIG. 16 shows an example flow chart for automating surgical tasks associated with autonomy levels.

FIG. 17 shows an example of a surgical autonomous system.

FIG. 18 shows the relationship between control feedback and failure mitigation feedback.

FIG. 19 shows an example of performing an autonomous function with an ideal model and a failure model.

FIG. 20 shows an example of performing an autonomous function with control feedback and failure mitigation feedback.

FIG. 21 shows an example overview of the surgical instrument with control feedback and the failure mitigation feedback and the surgical hub.

FIG. 22 shows an example of a surgical autonomous system.

FIG. 23 shows an example overview of an adapting interconnected surgical system.

FIG. 24 shows an example of the relationship between feature sets, surgical hub, and resource management.

FIG. 25 shows an example of a matrix generator and feature determination.

FIG. 26 shows a flow chart of an adapted interconnected surgical system.

FIG. 27 shows an example of an overview of a rules engine.

FIG. 28 illustrates an example process of autonomous update 1 surgical device control algorithm.

FIG. 29 illustrates a computer-implemented adaptive surgical system.

FIG. 30 illustrates an illustrative analytics system updating a surgical instrument control program.

FIG. 31 illustrates in example process of autonomous update of surgical device control algorithm.

FIG. 32 illustrates an example process of autonomous update of surgical device control algorithm.

FIG. 33 is a flow chart of an example process of autonomous update of surgical device control algorithm.

FIG. 34 illustrates an example autonomous operation of a surgical instrument.

FIG. 35 illustrates an example autonomous operation of a surgical instrument.

FIG. 36 is a flow chart of an example autonomous operation of a surgical instrument.

FIG. 37A through FIG. 37B illustrate example trocar placements.

FIG. 38 illustrates example trocar placements in a laparoscopic surgical procedure.

FIG. 39A through FIG. 39C illustrate an example surgical step autonomously controlled by a computing device.

FIG. 40 is a flow chart of an example autonomous operation.

FIG. 41 illustrates an example flow diagram of a surgical computing system automatically performing selective dissemination of annotated surgical procedure data to surgical systems.

FIG. 42 illustrates an example of generating and sending data packages to target systems.

FIG. 43 illustrates an example of selectively redacting of data in a data set.

FIG. 44 illustrates an example aggregation of pre-surgical data and generation of a patient specific procedure plan.

FIG. 45 illustrates an example of an image of a lung generated from multiple sources.

FIG. 46 illustrates an example image sourced by a laparoscopic camera or endobronchial ultrasound bronchoscopy (EBUS) to fill in missing portions of a full 3D view.

FIG. 47 illustrates an example of a patient specific procedure plan report.

FIG. 48 illustrates an initial access port location identification.

FIG. 49 illustrates an example overlay of patient data and imaging on a procedure plan.

FIG. 50 illustrates an example of annotating surgical video using situational awareness.

FIG. 51 illustrates example annotations associated with a surgical procedure.

FIG. 52 illustrates an example of determining element tracking information and verification for elements in a surgical video.

FIG. 53 illustrates an example of generating annotation data into a surgical video using surgical context data and situational awareness.

### DETAILED DESCRIPTION

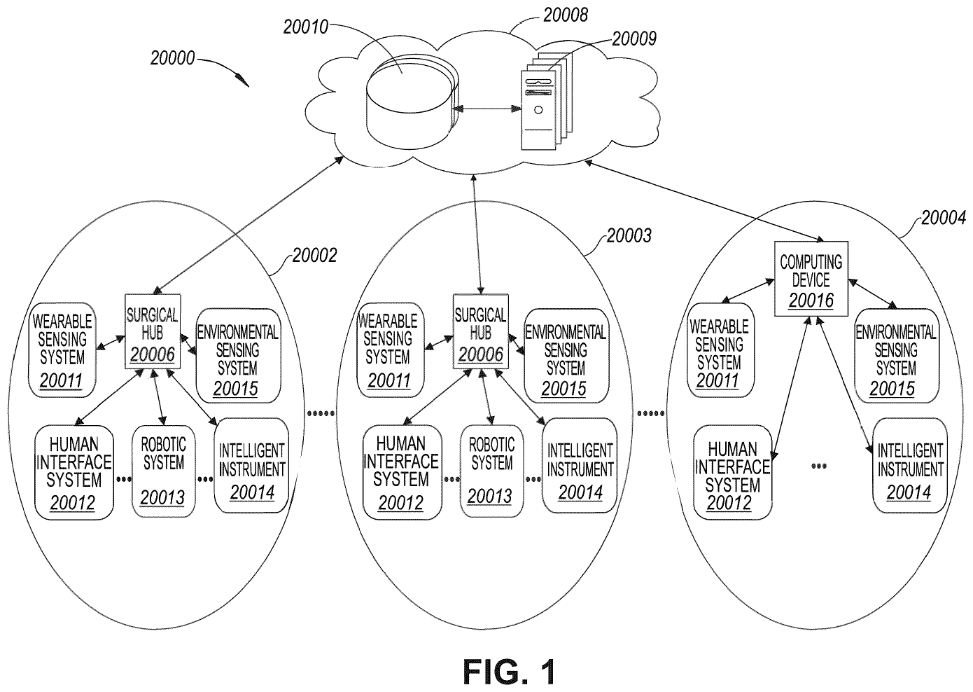

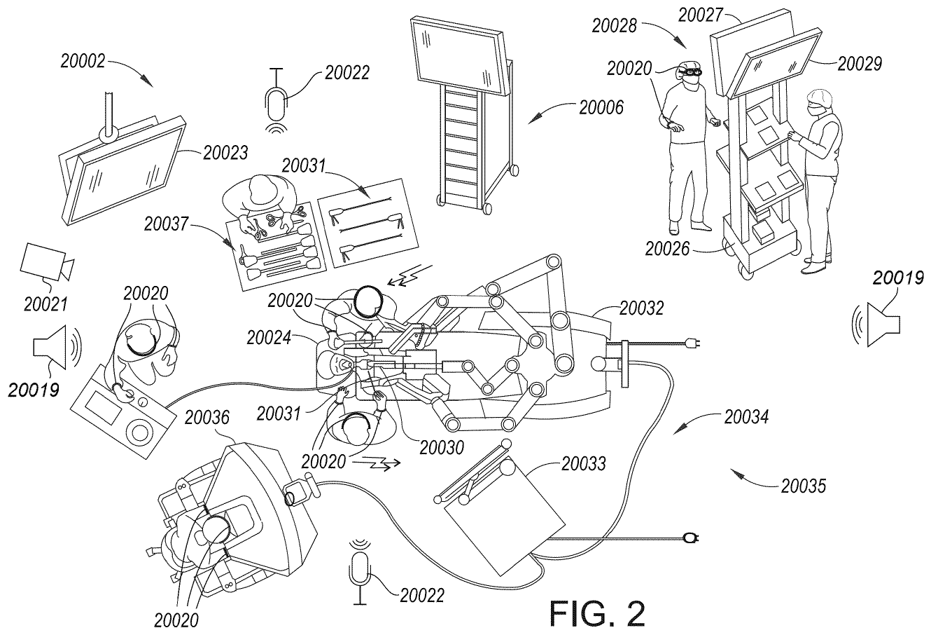

FIG. 1 is a block diagram of a computer-implemented surgical system 20000. An example surgical system such as the surgical system 20000 may include one or more surgical systems (e.g., surgical sub-systems) 20002, 20003 and 20004. For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include a wearable sensing system 20011, an environmental sensing system 20015, a robotic system 20013, one or more intelligent instruments 20014, human interface system 20012, etc. The human interface system is also referred herein as the human interface device. The wearable sensing system 20011 may include one or more HCP sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G.

A surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the one or more sensing systems 20011 and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems 20001 may include the wearable sensing system 20011 (which may include one or more HCP sensing systems and one or more patient sensing systems) and the environmental sensing system 20015 as discussed in FIG. 1. The one or more sensing systems 20001 may measure data relating to various biomarkers. The one or more sensing systems 20001 may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The one or more sensors may measure the biomarkers as described herein using one of more of the following sensing technologies: photoplethysmography, electrocardiography, electroencephalography, colorimetry, impedimentary, potentiometry, amperometry, etc.

The biomarkers measured by the one or more sensing systems 20001 may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example. The one or more sensing systems 20001, biomarkers 20005, and physiological systems are described in more detail in U.S. application Ser. No. 17/156,287 (attorney docket number END9290USNP1), tided METHOD OF ADJUSTING A SURGICAL PARAMETER BASED ON BIOMARKER MEASUREMENTS, filed Jan. 22, 2021, the disclosure of which is herein incorporated by reference in its entirety.

FIG. 2 shows an example of a surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

In one aspect, the surgical hub 20006 may be configured to route a diagnostic input or feedback entered by a non-sterile operator at the visualization tower 20026 to the primary display 20023 within the sterile field, where it can be viewed by a sterile operator at the operating table. In one example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument 20031. For example, in U.S. Patent Application Publication No. US 2019-0200844 A1 (U.S. patent application Ser. No. 16/209,385), tided METHOD OF HUB COMMUNICATION, PROCESSING, STORAGE AND DISPLAY, filed Dec. 4, 2018, the disclosure of which is herein incorporated by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

FIG. 2 illustrates an example of a surgical system 20002 being used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), tided METHOD OF CLOUD BASED DATA ANALYTICS FOR USE WITH THE HUB, filed Dec. 4, 2018, the disclosure of which is herein incorporated by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The one or more illumination sources may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (i.e., can be detected by) the human eye and may be referred to as visible light or simply light. A typical human eye will respond to wavelengths in air that range from about 380 nm to about 750 nm.

The invisible spectrum (e.g., the non-luminous spectrum) is the portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” i.e., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1 may include one or more sensing systems, for example, HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, a sensing system 20020 may measure a set of biomarkers to monitor the heart rate of an HCP. In an example, a sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing. One or more environmental sensing devices may send environmental information to the surgical hub 20006. For example, the environmental sensing devices may include a camera 20021 for detecting hand/body position of an HCP. The environmental sensing devices may include microphones 20022 for measuring the ambient noise in the surgical theater. Other environmental sensing devices may include devices, for example, a thermometer to measure temperature and a hygrometer to measure humidity of the surroundings in the surgical theater, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors. In an example, the HCP sensing systems 20020 may measure one or more surgeon biomarkers associated with an HCP and send the measurement data associated with the surgeon biomarkers to the surgical hub 20006. The HCP sensing systems 20020 may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

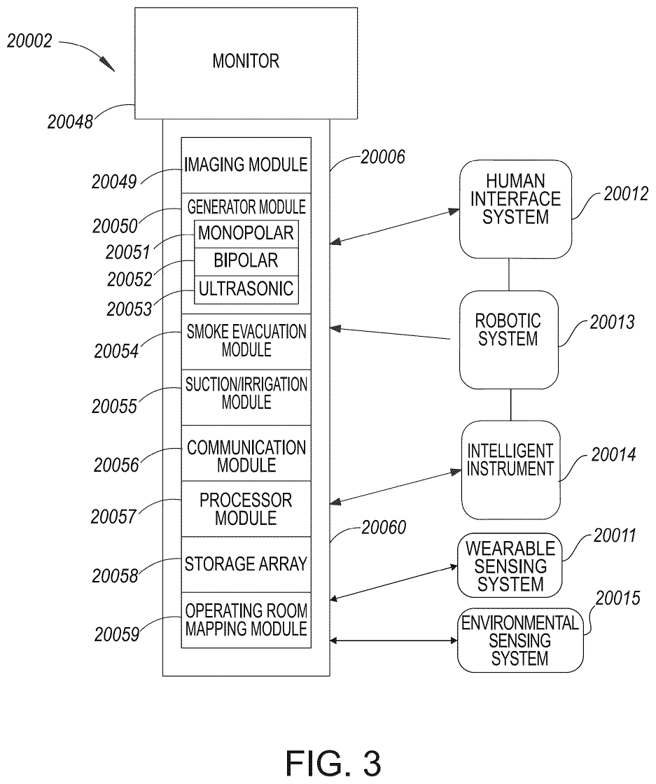

FIG. 3 shows an example surgical system 20002 with a surgical hub 20006. The surgical hub 20006 may be paired with, via a modular control, a wearable sensing system 20011, an environmental sensing system 20015, a human interface system 20012, a robotic system 20013, and an intelligent instrument 20014. The hub 20006 includes a display 20048, an imaging module 20049, a generator module 20050, a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. The various modules and systems may be connected to the modular control either directly via a router or via the communication module 20056. The operating theater devices may be coupled to cloud computing resources and data storage via the modular control. The human interface system 20012 may include a display sub-system and a notification sub-system.

The modular control may be coupled to non-contact sensor module. The non-contact sensor module may measure the dimensions of the operating theater and generate a map of the surgical theater using, ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, tided INTERACTIVE SURGICAL PLATFORM, filed Dec. 28, 2017, which is herein incorporated by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

During a surgical procedure, energy application to tissue, for sealing and/or cutting, is generally associated with smoke evacuation, suction of excess fluid, and/or irrigation of the tissue. Fluid, power, and/or data lines from different sources are often entangled during the surgical procedure. Valuable time can be lost addressing this issue during a surgical procedure. Detangling the lines may necessitate disconnecting the lines from their respective modules, which may require resetting the modules. The hub modular enclosure 20060 offers a unified environment for managing the power, data, and fluid lines, which reduces the frequency of entanglement between such lines. Aspects of the present disclosure present a surgical hub 20006 for use in a surgical procedure that involves energy application to tissue at a surgical site. The surgical hub 20006 includes a hub enclosure 20060 and a combo generator module slidably receivable in a docking station of the hub enclosure 20060. The docking station includes data and power contacts. The combo generator module includes two or more of an ultrasonic energy generator component, a bipolar RF energy generator component, and a monopolar RF energy generator component that are housed in a single unit. In one aspect, the combo generator module also includes a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. In one aspect, the fluid line may be a first fluid line, and a second fluid line may extend from the remote surgical site to a suction and irrigation module 20055 slidably received in the hub enclosure 20060. In one aspect, the hub enclosure 20060 may include a fluid interface. Certain surgical procedures may require the application of more than one energy type to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. One of the advantages of the hub modular enclosure 20060 is enabling the quick removal and/or replacement of various modules. Aspects of the present disclosure present a modular surgical enclosure for use in a surgical procedure that involves energy application to tissue. The modular surgical enclosure includes a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. Further to the above, the modular surgical enclosure also includes a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module. Referring to FIG. 3, aspects of the present disclosure are presented for a hub modular enclosure 20060 that allows the modular integration of a generator module 20050, a smoke evacuation module 20054, and a suction/irrigation module 20055. The hub modular enclosure 20060 further facilitates interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 can be configured to connect to a monopolar device 20051, a bipolar device 20052, and an ultrasonic device 20053. Alternatively, the generator module 20050 may comprise a series of monopolar, bipolar, and/or ultrasonic generator modules that interact through the hub modular enclosure 20060. The hub modular enclosure 20060 can be configured to facilitate the insertion of multiple generators and interactive communication between the generators docked into the hub modular enclosure 20060 so that the generators would act as a single generator.

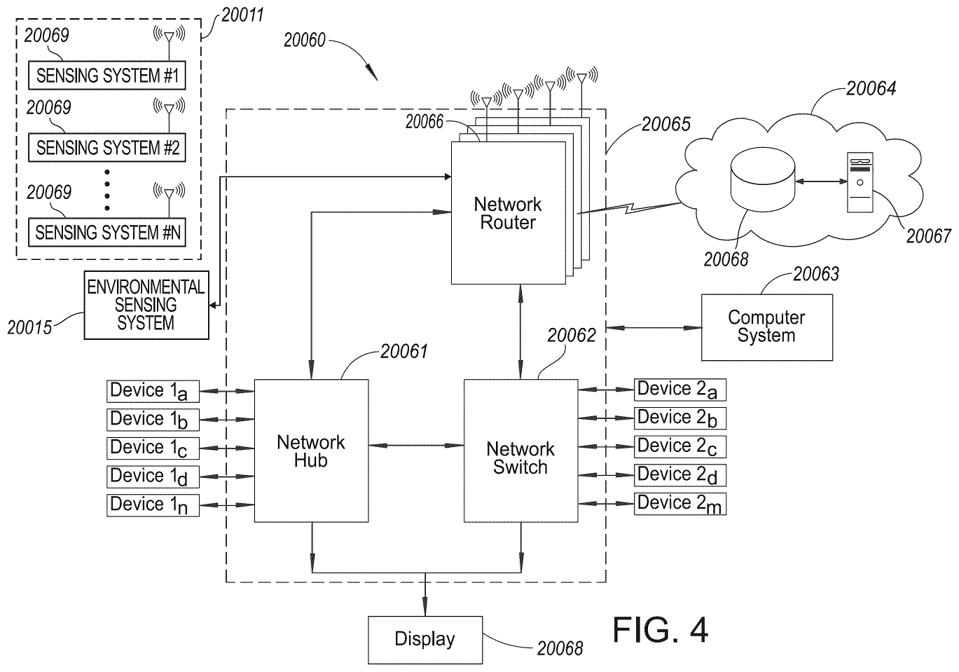

FIG. 4 illustrates a surgical data network having a set of communication hubs configured to connect a set of sensing systems, environment sensing system(s), and a set of other modular devices located in one or more operating theaters of a healthcare facility, a patient recovery room, or a room in a healthcare facility specially equipped for surgical operations, to the cloud, in accordance with at least one aspect of the present disclosure.

As illustrated in FIG. 4, a surgical hub system 20060 may include a modular communication hub 20065 that is configured to connect modular devices located in a healthcare facility to a cloud-based system (e.g., a cloud computing system 20064 that may include a remote server 20067 coupled to a remote storage 20068). The modular communication hub 20065 and the devices may be connected in a room in a healthcare facility specially equipped for surgical operations. In one aspect, the modular communication hub 20065 may include a network hub 20061 and/or a network switch 20062 in communication with a network router 20066. The modular communication hub 20065 may be coupled to a local computer system 20063 to provide local computer processing and data manipulation.

The computer system 20063 may comprise a processor and a network interface 20100. The processor may be coupled to a communication module, storage, memory, non-volatile memory, and input/output (I/O) interface via a system bus. The system bus can be any of several types of bus structure(s) including the memory bus or memory controller, a peripheral bus or external bus, and/or a local bus using any variety of available bus architectures including, but not limited to, 9-bit bus, Industrial Standard Architecture (ISA), Micro-Charmel Architecture (MSA), Extended ISA (EISA), Intelligent Drive Electronics (IDE), VESA Local Bus (VLB), Peripheral Component Interconnect (PCI), USB, Advanced Graphics Port (AGP), Personal Computer Memory Card International Association bus (PCMCIA), Small Computer Systems Interface (SCSI), or any other proprietary bus.

The processor may be any single-core or multicore processor such as those known under the trade name ARM Cortex by Texas Instruments. In one aspect, the processor may be an LM4F230H5QR ARM Cortex-M4F Processor Core, available from Texas Instruments, for example, comprising an on-chip memory of 256 KB single-cycle flash memory, or other non-volatile memory, up to 40 MHz, a prefetch buffer to improve performance above 40 MHz, a 32 KB single-cycle serial random access memory (SRAM), an internal read-only memory (ROM) loaded with StellarisWare® software, a 2 KB electrically erasable programmable read-only memory (EEPROM), and/or one or more pulse width modulation (PWM) modules, one or more quadrature encoder inputs (QEI) analogs, one or more 12-bit analog-to-digital converters (ADCs) with 12 analog input channels, details of which are available for the product datasheet.

In an example, the processor may comprise a safety controller comprising two controller-based families such as TMS570 and RM4x, known under the trade name Hercules ARM Cortex R4, also by Texas Instruments. The safety controller may be configured specifically for IEC 61508 and ISO 26262 safety critical applications, among others, to provide advanced integrated safety features while delivering scalable performance, connectivity, and memory options.

It is to be appreciated that the computer system 20063 may include software that acts as an intermediary between users and the basic computer resources described in a suitable operating environment. Such software may include an operating system. The operating system, which can be stored on the disk storage, may act to control and allocate resources of the computer system. System applications may take advantage of the management of resources by the operating system through program modules and program data stored either in the system memory or on the disk storage. It is to be appreciated that various components described herein can be implemented with various operating systems or combinations of operating systems.

A user may enter commands or information into the computer system 20063 through input device(s) coupled to the I/O interface. The input devices may include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processor 20102 through the system bus via interface port(s). The interface port(s) include, for example, a serial port, a parallel port, a game port, and a USB. The output device(s) use some of the same types of ports as input device(s). Thus, for example, a USB port may be used to provide input to the computer system 20063 and to output information from the computer system 20063 to an output device. An output adapter may be provided to illustrate that there can be some output devices like monitors, displays, speakers, and printers, among other output devices that may require special adapters. The output adapters may include, by way of illustration and not limitation, video and sound cards that provide a means of connection between the output device and the system bus. It should be noted that other devices and/or systems of devices, such as remote computer(s), may provide both input and output capabilities.

The computer system 20063 can operate in a networked environment using logical connections to one or more remote computers, such as cloud computer(s), or local computers. The remote cloud computer(s) can be a personal computer, server, router, network PC, workstation, microprocessor-based appliance, peer device, or other common network node, and the like, and typically includes many or all of the elements described relative to the computer system. For purposes of brevity, only a memory storage device is illustrated with the remote computer(s). The remote computer(s) may be logically connected to the computer system through a network interface and then physically connected via a communication connection. The network interface may encompass communication networks such as local area networks (LANs) and wide area networks (WANs). LAN technologies may include Fiber Distributed Data Interface (FDDI), Copper Distributed Data Interface (CDDI), Ethernet/IEEE 802.3, Token Ring/IEEE 802.5, and the like. WAN technologies may include, but are not limited to, point-to-point links, circuit-switching networks like Integrated Services Digital Networks (ISDN) and variations thereon, packet-switching networks, and Digital Subscriber Lines (DSL).

In various examples, the computer system 20063 may comprise an image processor, image-processing engine, media processor, or any specialized digital signal processor (DSP) used for the processing of digital images. The image processor may employ parallel computing with single instruction, multiple data (SIMD) or multiple instruction, multiple data (MIMD) technologies to increase speed and efficiency. The digital image-processing engine can perform a range of tasks. The image processor may be a system on a chip with multicore processor architecture.

The communication connection(s) may refer to the hardware/software employed to connect the network interface to the bus. While the communication connection is shown for illustrative clarity inside the computer system 20063, it can also be external to the computer system 20063. The hardware/software necessary for connection to the network interface may include, for illustrative purposes only, internal and external technologies such as modems, including regular telephone-grade modems, cable modems, optical fiber modems, and DSL modems, ISDN adapters, and Ethernet cards. In some examples, the network interface may also be provided using an RF interface.

Surgical data network associated with the surgical hub system 20060 may be configured as passive, intelligent, or switching. A passive surgical data network serves as a conduit for the data, enabling it to go from one device (or segment) to another and to the cloud computing resources. An intelligent surgical data network includes additional features to enable the traffic passing through the surgical data network to be monitored and to configure each port in the network hub 20061 or network switch 20062. An intelligent surgical data network may be referred to as a manageable hub or switch. A switching hub reads the destination address of each packet and then forwards the packet to the correct port.

Modular devices 1a-1n located in the operating theater may be coupled to the modular communication hub 20065. The network hub 20061 and/or the network switch 20062 may be coupled to a network router 20066 to connect the devices 1a-1n to the cloud computing system 20064 or the local computer system 20063. Data associated with the devices 1a-1n may be transferred to cloud-based computers via the router for remote data processing and manipulation. Data associated with the devices 1a-1n may also be transferred to the local computer system 20063 for local data processing and manipulation. Modular devices 2a-2m located in the same operating theater also may be coupled to a network switch 20062. The network switch 20062 may be coupled to the network hub 20061 and/or the network router 20066 to connect the devices 2a-2m to the cloud 20064. Data associated with the devices 2a-2m may be transferred to the cloud computing system 20064 via the network router 20066 for data processing and manipulation. Data associated with the devices 2a-2m may also be transferred to the local computer system 20063 for local data processing and manipulation.

The wearable sensing system 20011 may include one or more sensing systems 20069. The sensing systems 20069 may include an HCP sensing system and/or a patient sensing system. The one or more sensing systems 20069 may be in communication with the computer system 20063 of a surgical hub system 20060 or the cloud server 20067 directly via one of the network routers 20066 or via a network hub 20061 or network switching 20062 that is in communication with the network routers 20066.

The sensing systems 20069 may be coupled to the network router 20066 to connect to the sensing systems 20069 to the local computer system 20063 and/or the cloud computing system 20064. Data associated with the sensing systems 20069 may be transferred to the cloud computing system 20064 via the network router 20066 for data processing and manipulation. Data associated with the sensing systems 20069 may also be transferred to the local computer system 20063 for local data processing and manipulation.

As illustrated in FIG. 4, the surgical hub system 20060 may be expanded by interconnecting multiple network hubs 20061 and/or multiple network switches 20062 with multiple network routers 20066. The modular communication hub 20065 may be contained in a modular control tower configured to receive multiple devices 1a-1n/2a-2m. The local computer system 20063 also may be contained in a modular control tower. The modular communication hub 20065 may be connected to a display 20068 to display images obtained by some of the devices 1a-1n/2a-2m, for example during surgical procedures. In various aspects, the devices 1a-1n/2a-2m may include, for example, various modules such as an imaging module coupled to an endoscope, a generator module coupled to an energy-based surgical device, a smoke evacuation module, a suction/irrigation module, a communication module, a processor module, a storage array, a surgical device coupled to a display, and/or a non-contact sensor module, among other modular devices that may be connected to the modular communication hub 20065 of the surgical data network.

In one aspect, the surgical hub system 20060 illustrated in FIG. 4 may comprise a combination of network hub(s), network switch(es), and network router(s) connecting the devices 1a-1n/2a-2m or the sensing systems 20069 to the cloud-base system 20064. One or more of the devices 1a-1n/2a-2m or the sensing systems 20069 coupled to the network hub 20061 or network switch 20062 may collect data in real-time and transfer the data to cloud computers for data processing and manipulation. It will be appreciated that cloud computing relies on sharing computing resources rather than having local servers or personal devices to handle software applications. The word “cloud” may be used as a metaphor for “the Internet,” although the term is not limited as such. Accordingly, the term “cloud computing” may be used herein to refer to “a type of Internet-based computing,” where different services-such as servers, storage, and applications—are delivered to the modular communication hub 20065 and/or computer system 20063 located in the surgical theater (e.g., a fixed, mobile, temporary, or field operating room or space) and to devices connected to the modular communication hub 20065 and/or computer system 20063 through the Internet. The cloud infrastructure may be maintained by a cloud service provider. In this context, the cloud service provider may be the entity that coordinates the usage and control of the devices 1a-1n/2a-2m located in one or more operating theaters. The cloud computing services can perform a large number of calculations based on the data gathered by smart surgical instruments, robots, sensing systems, and other computerized devices located in the operating theater. The hub hardware enables multiple devices, sensing systems, and/or connections to be connected to a computer that communicates with the cloud computing resources and storage.

Applying cloud computer data processing techniques on the data collected by the devices 1a-1n/2a-2m, the surgical data network can provide improved surgical outcomes, reduced costs, and improved patient satisfaction. At least some of the devices 1a-1n/2a-2m may be employed to view tissue states to assess leaks or perfusion of sealed tissue after a tissue sealing and cutting procedure. At least some of the devices 1a-1n/2a-2m may be employed to identify pathology, such as the effects of diseases, using the cloud-based computing to examine data including images of samples of body tissue for diagnostic purposes. This may include localization and margin confirmation of tissue and phenotypes. At least some of the devices 1a-1n/2a-2m may be employed to identify anatomical structures of the body using a variety of sensors integrated with imaging devices and techniques such as overlaying images captured by multiple imaging devices. The data gathered by the devices 1a-1n/2a-2m, including image data, may be transferred to the cloud computing system 20064 or the local computer system 20063 or both for data processing and manipulation including image processing and manipulation. The data may be analyzed to improve surgical procedure outcomes by determining if further treatment, such as the application of endoscopic intervention, emerging technologies, a targeted radiation, targeted intervention, and precise robotics to tissue-specific sites and conditions, may be pursued. Such data analysis may further employ outcome analytics processing and using standardized approaches may provide beneficial feedback to either confirm surgical treatments and the behavior of the surgeon or suggest modifications to surgical treatments and the behavior of the surgeon.

Applying cloud computer data processing techniques on the measurement data collected by the sensing systems 20069, the surgical data network can provide improved surgical outcomes, improved recovery outcomes, reduced costs, and improved patient satisfaction. At least some of the sensing systems 20069 may be employed to assess physiological conditions of a surgeon operating on a patient or a patient being prepared for a surgical procedure or a patient recovering after a surgical procedure. The cloud-based computing system 20064 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The operating theater devices 1a-1n may be connected to the modular communication hub 20065 over a wired channel or a wireless channel depending on the configuration of the devices 1a-1n to a network hub 20061. The network hub 20061 may be implemented, in one aspect, as a local network broadcast device that works on the physical layer of the Open System Interconnection (OSI) model. The network hub may provide connectivity to the devices 1a-1n located in the same operating theater network. The network hub 20061 may collect data in the form of packets and sends them to the router in half duplex mode. The network hub 20061 may not store any media access control/Internet Protocol (VIAC/IP) to transfer the device data. Only one of the devices 1a-1n can send data at a time through the network hub 20061. The network hub 20061 may not have routing tables or intelligence regarding where to send information and broadcasts all network data across each connection and to a remote server 20067 of the cloud computing system 20064. The network hub 20061 can detect basic network errors such as collisions but having all information broadcast to multiple ports can be a security risk and cause bottlenecks.

The operating theater devices 2a-2m may be connected to a network switch 20062 over a wired channel or a wireless channel. The network switch 20062 works in the data link layer of the OSI model. The network switch 20062 may be a multicast device for connecting the devices 2a-2m located in the same operating theater to the network. The network switch 20062 may send data in the form of frames to the network router 20066 and may work in full duplex mode. Multiple devices 2a-2m can send data at the same time through the network switch 20062. The network switch 20062 stores and uses MAC addresses of the devices 2a-2m to transfer data.

The network hub 20061 and/or the network switch 20062 may be coupled to the network router 20066 for connection to the cloud computing system 20064. The network router 20066 works in the network layer of the OSI model. The network router 20066 creates a route for transmitting data packets received from the network hub 20061 and/or network switch 20062 to cloud-based computer resources for further processing and manipulation of the data collected by any one of or all the devices 1a-1n/2a-2m and wearable sensing system 20011. The network router 20066 may be employed to connect two or more different networks located in different locations, such as, for example, different operating theaters of the same healthcare facility or different networks located in different operating theaters of different healthcare facilities. The network router 20066 may send data in the form of packets to the cloud computing system 20064 and works in full duplex mode. Multiple devices can send data at the same time. The network router 20066 may use IP addresses to transfer data.

In an example, the network hub 20061 may be implemented as a USB hub, which allows multiple USB devices to be connected to a host computer. The USB hub may expand a single USB port into several tiers so that there are more ports available to connect devices to the host system computer. The network hub 20061 may include wired or wireless capabilities to receive information over a wired channel or a wireless channel. In one aspect, a wireless USB short-range, high-bandwidth wireless radio communication protocol may be employed for communication between the devices 1a-1n and devices 2a-2m located in the operating theater.

In examples, the operating theater devices 1a-1n/2a-2m and/or the sensing systems 20069 may communicate to the modular communication hub 20065 via Bluetooth wireless technology standard for exchanging data over short distances (using short-wavelength UHF radio waves in the ISM band from 2.4 to 2.485 GHz) from fixed and mobile devices and building personal area networks (PANs). The operating theater devices 1a-1n/2a-2m and/or the sensing systems 20069 may communicate to the modular communication hub 20065 via a number of wireless or wired communication standards or protocols, including but not limited to Bluetooth, Low-Energy Bluetooth, near-field communication (NFC), Wi-Fi (IEEE 802.11 family), WiMAX (IEEE 802.16 family), IEEE 802.20, new radio (NR), long-term evolution (LTE), and Ev-DO, HSPA+, HSDPA+, HSUPA+, EDGE, GSM, GPRS, CDMA, TDMA, DECT, and Ethernet derivatives thereof, as well as any other wireless and wired protocols that are designated as 3G, 4G, 5G, and beyond. The computing module may include a plurality of communication modules. For instance, a first communication module may be dedicated to shorter-range wireless communications such as Wi-Fi and Bluetooth Low-Energy Bluetooth, Bluetooth Smart, and a second communication module may be dedicated to longer-range wireless communications such as GPS, EDGE, GPRS, CDMA, WiMAX, LTE, Ev-DO, HSPA+, HSDPA+, HSUPA+, EDGE, GSM, GPRS, CDMA, TDMA, and others.

The modular communication hub 20065 may serve as a central connection for one or more of the operating theater devices 1a-1n/2a-2m and/or the sensing systems 20069 and may handle a data type known as frames. Frames may carry the data generated by the devices 1a-1n/2a-2m and/or the sensing systems 20069. When a frame is received by the modular communication hub 20065, it may be amplified and/or sent to the network router 20066, which may transfer the data to the cloud computing system 20064 or the local computer system 20063 by using a number of wireless or wired communication standards or protocols, as described herein.

The modular communication hub 20065 can be used as a standalone device or be connected to compatible network hubs 20061 and network switches 20062 to form a larger network. The modular communication hub 20065 can be generally easy to install, configure, and maintain, making it a good option for networking the operating theater devices 1a-1n/2a-2m.

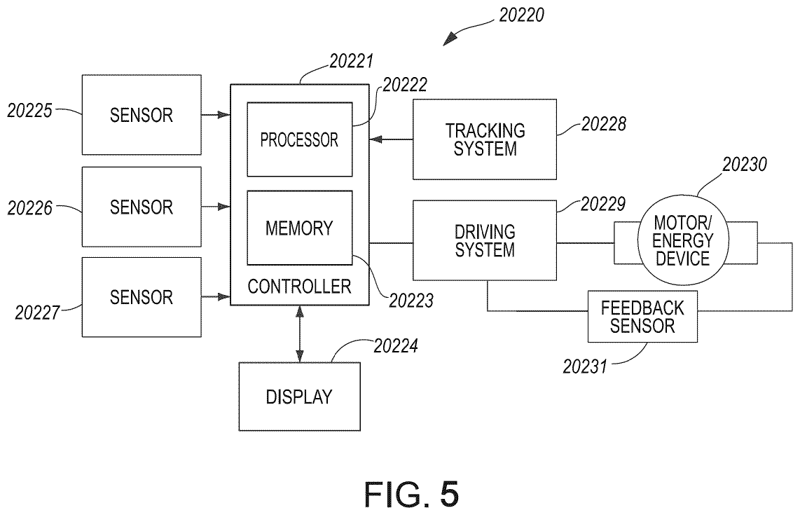

FIG. 5 illustrates a logical diagram of a control system 20220 of a surgical instrument or a surgical tool in accordance with one or more aspects of the present disclosure. The surgical instrument or the surgical tool may be configurable. The surgical instrument may include surgical fixtures specific to the procedure at-hand, such as imaging devices, surgical staplers, energy devices, endocutter devices, or the like. For example, the surgical instrument may include any of a powered stapler, a powered stapler generator, an energy device, an advanced energy device, an advanced energy jaw device, an endocutter clamp, an energy device generator, an in-operating-room imaging system, a smoke evacuator, a suction-irrigation device, an insufflation system, or the like. The system 20220 may comprise a control circuit. The control circuit may include a microcontroller 20221 comprising a processor 20222 and a memory 20223. One or more of sensors 20225, 20226, 20227, for example, provide real-time feedback to the processor 20222. A motor 20230, driven by a motor driver 20229, operably couples a longitudinally movable displacement member to drive the I-beam knife element. A tracking system 20228 may be configured to determine the position of the longitudinally movable displacement member. The position information may be provided to the processor 20222, which can be programmed or configured to determine the position of the longitudinally movable drive member as well as the position of a firing member, firing bar, and I-beam knife element. Additional motors may be provided at the tool driver interface to control I-beam firing, closure tube travel, shaft rotation, and articulation. A display 20224 may display a variety of operating conditions of the instruments and may include touch screen functionality for data input. Information displayed on the display 20224 may be overlaid with images acquired via endoscopic imaging modules.

The microcontroller 20221 may be any single-core or multicore processor such as those known under the trade name ARM Cortex by Texas Instruments. In one aspect, the main microcontroller 20221 may be an LM4F230H5QR ARM Cortex-M4F Processor Core, available from Texas Instruments, for example, comprising an on-chip memory of 256 KB single-cycle flash memory, or other non-volatile memory, up to 40 MHz, a prefetch buffer to improve performance above 40 MHz, a 32 KB single-cycle SRAM, and internal ROM loaded with StellarisWare® software, a 2 KB EEPROM, one or more PWM modules, one or more QEI analogs, and/or one or more 12-bit ADCs with 12 analog input channels, details of which are available for the product datasheet.

The microcontroller 20221 may comprise a safety controller comprising two controller-based families such as TMS570 and RM4x, known under the trade name Hercules ARM Cortex R4, also by Texas Instruments. The safety controller may be configured specifically for IEC 61508 and ISO 26262 safety critical applications, among others, to provide advanced integrated safety features while delivering scalable performance, connectivity, and memory options.

The microcontroller 20221 may be programmed to perform various functions such as precise control over the speed and position of the knife and articulation systems. In one aspect, the microcontroller 20221 may include a processor 20222 and a memory 20223. The electric motor 20230 may be a brushed direct current (DC) motor with a gearbox and mechanical links to an articulation or knife system. In one aspect, a motor driver 20229 may be an A3941 available from Allegro Microsystems, Inc. Other motor drivers may be readily substituted for use in the tracking system 20228 comprising an absolute positioning system. A detailed description of an absolute positioning system is described in U.S. Patent Application Publication No. 2017/0296213, titled SYSTEMS AND METHODS FOR CONTROLLING A SURGICAL STAPLING AND CUTTING INSTRUMENT, which published on Oct. 19, 2017, which is herein incorporated by reference in its entirety.

The microcontroller 20221 may be programmed to provide precise control over the speed and position of displacement members and articulation systems. The microcontroller 20221 may be configured to compute a response in the software of the microcontroller 20221. The computed response may be compared to a measured response of the actual system to obtain an “observed” response, which is used for actual feedback decisions. The observed response may be a favorable, tuned value that balances the smooth, continuous nature of the simulated response with the measured response, which can detect outside influences on the system.

The motor 20230 may be controlled by the motor driver 20229 and can be employed by the firing system of the surgical instrument or tool. In various forms, the motor 20230 may be a brushed DC driving motor having a maximum rotational speed of approximately 25,000 RPM. In some examples, the motor 20230 may include a brushless motor, a cordless motor, a synchronous motor, a stepper motor, or any other suitable electric motor. The motor driver 20229 may comprise an H-bridge driver comprising field-effect transistors (FETs), for example. The motor 20230 can be powered by a power assembly releasably mounted to the handle assembly or tool housing for supplying control power to the surgical instrument or tool. The power assembly may comprise a battery which may include a number of battery cells connected in series that can be used as the power source to power the surgical instrument or tool. In certain circumstances, the battery cells of the power assembly may be replaceable and/or rechargeable. In at least one example, the battery cells can be lithium-ion batteries which can be couplable to and separable from the power assembly.

The motor driver 20229 may be an A3941 available from Allegro Microsystems, Inc. A3941 may be a full-bridge controller for use with external N-channel power metal-oxide semiconductor field-effect transistors (MOSFETs) specifically designed for inductive loads, such as brush DC motors. The driver 20229 may comprise a unique charge pump regulator that can provide full (>10 V) gate drive for battery voltages down to 7 V and can allow the A3941 to operate with a reduced gate drive, down to 5.5 V. A bootstrap capacitor may be employed to provide the above battery supply voltage required for N-channel MOSFETs. An internal charge pump for the high-side drive may allow DC (100% duty cycle) operation. The full bridge can be driven in fast or slow decay modes using diode or synchronous rectification. In the slow decay mode, current recirculation can be through the high-side or the low-side FETs. The power FETs may be protected from shoot-through by resistor-adjustable dead time. Integrated diagnostics provide indications of undervoltage, overtemperature, and power bridge faults and can be configured to protect the power MOSFETs under most short circuit conditions. Other motor drivers may be readily substituted for use in the tracking system 20228 comprising an absolute positioning system.

The tracking system 20228 may comprise a controlled motor drive circuit arrangement comprising a position sensor 20225 according to one aspect of this disclosure. The position sensor 20225 for an absolute positioning system may provide a unique position signal corresponding to the location of a displacement member. In some examples, the displacement member may represent a longitudinally movable drive member comprising a rack of drive teeth for meshing engagement with a corresponding drive gear of a gear reducer assembly. In some examples, the displacement member may represent the firing member, which could be adapted and configured to include a rack of drive teeth. In some examples, the displacement member may represent a firing bar or the I-beam, each of which can be adapted and configured to include a rack of drive teeth. Accordingly, as used herein, the term displacement member can be used generically to refer to any movable member of the surgical instrument or tool such as the drive member, the firing member, the firing bar, the I-beam, or any element that can be displaced. In one aspect, the longitudinally movable drive member can be coupled to the firing member, the firing bar, and the I-beam. Accordingly, the absolute positioning system can, in effect, track the linear displacement of the I-beam by tracking the linear displacement of the longitudinally movable drive member. In various aspects, the displacement member may be coupled to any position sensor 20225 suitable for measuring linear displacement. Thus, the longitudinally movable drive member, the firing member, the firing bar, or the I-beam, or combinations thereof, may be coupled to any suitable linear displacement sensor. Linear displacement sensors may include contact or non-contact displacement sensors. Linear displacement sensors may comprise linear variable differential transformers (LVDT), differential variable reluctance transducers (DVRT), a slide potentiometer, a magnetic sensing system comprising a movable magnet and a series of linearly arranged Hall effect sensors, a magnetic sensing system comprising a fixed magnet and a series of movable, linearly arranged Hall effect sensors, an optical sensing system comprising a movable light source and a series of linearly arranged photo diodes or photo detectors, an optical sensing system comprising a fixed light source and a series of movable linearly, arranged photodiodes or photodetectors, or any combination thereof.

The electric motor 20230 can include a rotatable shaft that operably interfaces with a gear assembly that is mounted in meshing engagement with a set, or rack, of drive teeth on the displacement member. A sensor element may be operably coupled to a gear assembly such that a single revolution of the position sensor 20225 element corresponds to some linear longitudinal translation of the displacement member. An arrangement of gearing and sensors can be connected to the linear actuator, via a rack and pinion arrangement, or a rotary actuator, via a spur gear or other connection. A power source may supply power to the absolute positioning system and an output indicator may display the output of the absolute positioning system. The displacement member may represent the longitudinally movable drive member comprising a rack of drive teeth formed thereon for meshing engagement with a corresponding drive gear of the gear reducer assembly. The displacement member may represent the longitudinally movable firing member, firing bar, I-beam, or combinations thereof.

A single revolution of the sensor element associated with the position sensor 20225 may be equivalent to a longitudinal linear displacement d1 of the displacement member, where d1 is the longitudinal linear distance that the displacement member moves from point “a” to point “b” after a single revolution of the sensor element coupled to the displacement member. The sensor arrangement may be connected via a gear reduction that results in the position sensor 20225 completing one or more revolutions for the full stroke of the displacement member. The position sensor 20225 may complete multiple revolutions for the full stroke of the displacement member.

A series of switches, where n is an integer greater than one, may be employed alone or in combination with a gear reduction to provide a unique position signal for more than one revolution of the position sensor 20225. The state of the switches may be fed back to the microcontroller 20221 that applies logic to determine a unique position signal corresponding to the longitudinal linear displacement d1+d2+ . . . dn of the displacement member. The output of the position sensor 20225 is provided to the microcontroller 20221. The position sensor 20225 of the sensor arrangement may comprise a magnetic sensor, an analog rotary sensor like a potentiometer, or an array of analog Hall-effect elements, which output a unique combination of position signals or values.

The position sensor 20225 may comprise any number of magnetic sensing elements, such as, for example, magnetic sensors classified according to whether they measure the total magnetic field or the vector components of the magnetic field. The techniques used to produce both types of magnetic sensors may encompass many aspects of physics and electronics. The technologies used for magnetic field sensing may include search coil, fluxgate, optically pumped, nuclear precession, SQUID, Hall-effect, anisotropic magnetoresistance, giant magnetoresistance, magnetic tunnel junctions, giant magnetoimpedance, magnetostrictive/piezoelectric composites, magnetodiode, magnetotransistor, fiber-optic, magneto-optic, and microelectromechanical systems-based magnetic sensors, among others.

The position sensor 20225 for the tracking system 20228 comprising an absolute positioning system may comprise a magnetic rotary absolute positioning system. The position sensor 20225 may be implemented as an AS5055EQFT single-chip magnetic rotary position sensor available from Austria Microsystems, AG. The position sensor 20225 is interfaced with the microcontroller 20221 to provide an absolute positioning system. The position sensor 20225 may be a low-voltage and low-power component and may include four Hall-effect elements in an area of the position sensor 20225 that may be located above a magnet. A high-resolution ADC and a smart power management controller may also be provided on the chip. A coordinate rotation digital computer (CORDIC) processor, also known as the digit-by-digit method and Volder's algorithm, may be provided to implement a simple and efficient algorithm to calculate hyperbolic and trigonometric functions that require only addition, subtraction, bit-shift, and table lookup operations. The angle position, alarm bits, and magnetic field information may be transmitted over a standard serial communication interface, such as a serial peripheral interface (SPI) interface, to the microcontroller 20221. The position sensor 20225 may provide 12 or 14 bits of resolution. The position sensor 20225 may be an AS5055 chip provided in a small QFN 16-pin 4×4×0.85 mm package.

The tracking system 20228 comprising an absolute positioning system may comprise and/or be programmed to implement a feedback controller, such as a PID, state feedback, and adaptive controller. A power source converts the signal from the feedback controller into a physical input to the system: in this case the voltage. Other examples include a PWM of the voltage, current, and force. Other sensor(s) may be provided to measure physical parameters of the physical system in addition to the position measured by the position sensor 20225. In some aspects, the other sensor(s) can include sensor arrangements such as those described in U.S. Pat. No. 9,345,481, tided STAPLE CARTRIDGE TISSUE THICKNESS SENSOR SYSTEM, which issued on May 24, 2016, which is herein incorporated by reference in its entirety; U.S. Patent Application Publication No. 2014/0263552, titled STAPLE CARTRIDGE TISSUE THICKNESS SENSOR SYSTEM, which published on Sep. 18, 2014, which is herein incorporated by reference in its entirety; and U.S. patent application Ser. No. 15/628,175, titled TECHNIQUES FOR ADAPTIVE CONTROL OF MOTOR VELOCITY OF A SURGICAL STAPLING AND CUTTING INSTRUMENT, filed Jun. 20, 2017, which is herein incorporated by reference in its entirety. In a digital signal processing system, an absolute positioning system is coupled to a digital data acquisition system where the output of the absolute positioning system will have a finite resolution and sampling frequency. The absolute positioning system may comprise a compare-and-combine circuit to combine a computed response with a measured response using algorithms, such as a weighted average and a theoretical control loop, that drive the computed response towards the measured response. The computed response of the physical system may take into account properties like mass, inertia, viscous friction, inductance resistance, etc., to predict what the states and outputs of the physical system will be by knowing the input.

The absolute positioning system may provide an absolute position of the displacement member upon power-up of the instrument, without retracting or advancing the displacement member to a reset (zero or home) position as may be required with conventional rotary encoders that merely count the number of steps forwards or backwards that the motor 20230 has taken to infer the position of a device actuator, drive bar, knife, or the like.

A sensor 20226, such as, for example, a strain gauge or a micro-strain gauge, may be configured to measure one or more parameters of the end effector, such as, for example, the amplitude of the strain exerted on the anvil during a clamping operation, which can be indicative of the closure forces applied to the anvil. The measured strain may be converted to a digital signal and provided to the processor 20222. Alternatively, or in addition to the sensor 20226, a sensor 20227, such as, for example, a load sensor, can measure the closure force applied by the closure drive system to the anvil. The sensor 20227, such as, for example, a load sensor, can measure the firing force applied to an I-beam in a firing stroke of the surgical instrument or tool. The I-beam is configured to engage a wedge sled, which is configured to upwardly cam staple drivers to force out staples into deforming contact with an anvil. The I-beam also may include a sharpened cutting edge that can be used to sever tissue as the I-beam is advanced distally by the firing bar. Alternatively, a current sensor 20231 can be employed to measure the current drawn by the motor 20230. The force required to advance the firing member can correspond to the current drawn by the motor 20230, for example. The measured force may be converted to a digital signal and provided to the processor 20222.

For example, the strain gauge sensor 20226 can be used to measure the force applied to the tissue by the end effector. A strain gauge can be coupled to the end effector to measure the force on the tissue being treated by the end effector. A system for measuring forces applied to the tissue grasped by the end effector may comprise a strain gauge sensor 20226, such as, for example, a micro-strain gauge, that can be configured to measure one or more parameters of the end effector, for example. In one aspect, the strain gauge sensor 20226 can measure the amplitude or magnitude of the strain exerted on a jaw member of an end effector during a clamping operation, which can be indicative of the tissue compression. The measured strain can be converted to a digital signal and provided to a processor 20222 of the microcontroller 20221. A load sensor 20227 can measure the force used to operate the knife element, for example, to cut the tissue captured between the anvil and the staple cartridge. A magnetic field sensor can be employed to measure the thickness of the captured tissue. The measurement of the magnetic field sensor also may be converted to a digital signal and provided to the processor 20222.

The measurements of the tissue compression, the tissue thickness, and/or the force required to close the end effector on the tissue, as respectively measured by the sensors 20226, 20227, can be used by the microcontroller 20221 to characterize the selected position of the firing member and/or the corresponding value of the speed of the firing member. In one instance, a memory 20223 may store a technique, an equation, and/or a lookup table which can be employed by the microcontroller 20221 in the assessment.

The control system 20220 of the surgical instrument or tool also may comprise wired or wireless communication circuits to communicate with the surgical hub 20065 as shown in FIG. 4.

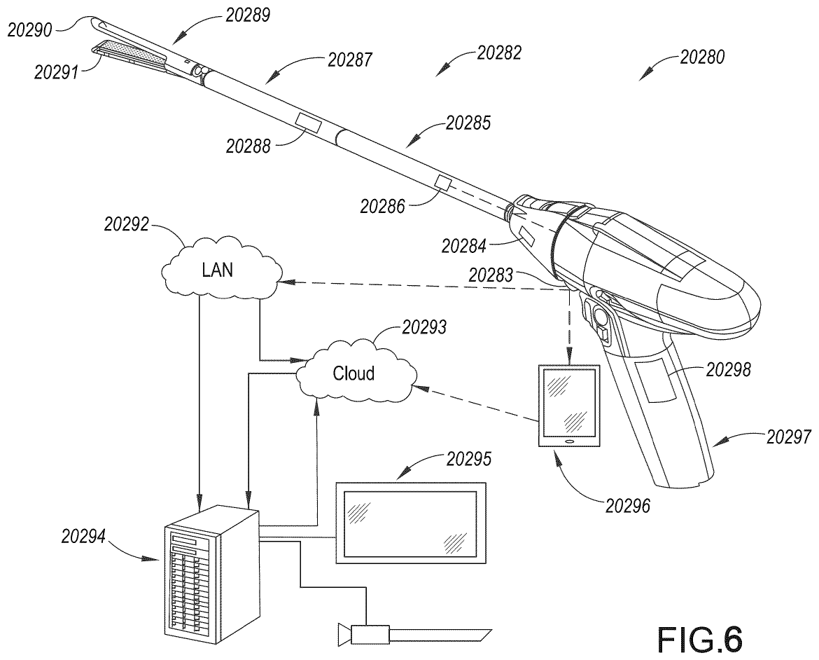

FIG. 6 illustrates an example surgical system 20280 in accordance with the present disclosure and may include a surgical instrument 20282 that can be in communication with a console 20294 or a portable device 20296 through a local area network 20292 and/or a cloud network 20293 via a wired and/or wireless connection. The console 20294 and the portable device 20296 may be any suitable computing device. The surgical instrument 20282 may include a handle 20297, an adapter 20285, and a loading unit 20287. The adapter 20285 releasably couples to the handle 20297 and the loading unit 20287 releasably couples to the adapter 20285 such that the adapter 20285 transmits a force from a drive shaft to the loading unit 20287. The adapter 20285 or the loading unit 20287 may include a force gauge (not explicitly shown) disposed therein to measure a force exerted on the loading unit 20287. The loading unit 20287 may include an end effector 20289 having a first jaw 20291 and a second jaw 20290. The loading unit 20287 may be an in-situ loaded or multi-firing loading unit (MFLU) that allows a clinician to fire a plurality of fasteners multiple times without requiring the loading unit 20287 to be removed from a surgical site to reload the loading unit 20287.

The first and second jaws 20291, 20290 may be configured to clamp tissue therebetween, fire fasteners through the clamped tissue, and sever the clamped tissue. The first jaw 20291 may be configured to fire at least one fastener a plurality of times or may be configured to include a replaceable multi-fire fastener cartridge including a plurality of fasteners (e.g., staples, clips, etc.) that may be fired more than one time prior to being replaced. The second jaw 20290 may include an anvil that deforms or otherwise secures the fasteners, as the fasteners are ejected from the multi-fire fastener cartridge.

The handle 20297 may include a motor that is coupled to the drive shaft to affect rotation of the drive shaft. The handle 20297 may include a control interface to selectively activate the motor. The control interface may include buttons, switches, levers, sliders, touchscreens, and any other suitable input mechanisms or user interfaces, which can be engaged by a clinician to activate the motor.

The control interface of the handle 20297 may be in communication with a controller 20298 of the handle 20297 to selectively activate the motor to affect rotation of the drive shafts. The controller 20298 may be disposed within the handle 20297 and may be configured to receive input from the control interface and adapter data from the adapter 20285 or loading unit data from the loading unit 20287. The controller 20298 may analyze the input from the control interface and the data received from the adapter 20285 and/or loading unit 20287 to selectively activate the motor. The handle 20297 may also include a display that is viewable by a clinician during use of the handle 20297. The display may be configured to display portions of the adapter or loading unit data before, during, or after firing of the instrument 20282.

The adapter 20285 may include an adapter identification device 20284 disposed therein and the loading unit 20287 may include a loading unit identification device 20288 disposed therein. The adapter identification device 20284 may be in communication with the controller 20298, and the loading unit identification device 20288 may be in communication with the controller 20298. It will be appreciated that the loading unit identification device 20288 may be in communication with the adapter identification device 20284, which relays or passes communication from the loading unit identification device 20288 to the controller 20298.

The adapter 20285 may also include a plurality of sensors 20286 (one shown) disposed thereabout to detect various conditions of the adapter 20285 or of the environment (e.g., if the adapter 20285 is connected to a loading unit, if the adapter 20285 is connected to a handle, if the drive shafts are rotating, the torque of the drive shafts, the strain of the drive shafts, the temperature within the adapter 20285, a number of firings of the adapter 20285, a peak force of the adapter 20285 during firing, a total amount of force applied to the adapter 20285, a peak retraction force of the adapter 20285, a number of pauses of the adapter 20285 during firing, etc.). The plurality of sensors 20286 may provide an input to the adapter identification device 20284 in the form of data signals. The data signals of the plurality of sensors 20286 may be stored within or be used to update the adapter data stored within the adapter identification device 20284. The data signals of the plurality of sensors 20286 may be analog or digital. The plurality of sensors 20286 may include a force gauge to measure a force exerted on the loading unit 20287 during firing.

The handle 20297 and the adapter 20285 can be configured to interconnect the adapter identification device 20284 and the loading unit identification device 20288 with the controller 20298 via an electrical interface. The electrical interface may be a direct electrical interface (i.e., include electrical contacts that engage one another to transmit energy and signals therebetween). Additionally, or alternatively, the electrical interface may be a non-contact electrical interface to wirelessly transmit energy and signals therebetween (e.g., inductively transfer). It is also contemplated that the adapter identification device 20284 and the controller 20298 may be in wireless communication with one another via a wireless connection separate from the electrical interface.

The handle 20297 may include a transceiver 20283 that is configured to transmit instrument data from the controller 20298 to other components of the system 20280 (e.g., the LAN 20292, the cloud 20293, the console 20294, or the portable device 20296). The controller 20298 may also transmit instrument data and/or measurement data associated with one or more sensors 20286 to a surgical hub. The transceiver 20283 may receive data (e.g., cartridge data, loading unit data, adapter data, or other notifications) from the surgical hub 20270. The transceiver 20283 may receive data (e.g., cartridge data, loading unit data, or adapter data) from the other components of the system 20280. For example, the controller 20298 may transmit instrument data including a serial number of an attached adapter (e.g., adapter 20285) attached to the handle 20297, a serial number of a loading unit (e.g., loading unit 20287) attached to the adapter 20285, and a serial number of a multi-fire fastener cartridge loaded into the loading unit to the console 20294. Thereafter, the console 20294 may transmit data (e.g., cartridge data, loading unit data, or adapter data) associated with the attached cartridge, loading unit, and adapter, respectively, back to the controller 20298. The controller 20298 can display messages on the local instrument display or transmit the message, via transceiver 20283, to the console 20294 or the portable device 20296 to display the message on the display 20295 or portable device screen, respectively.

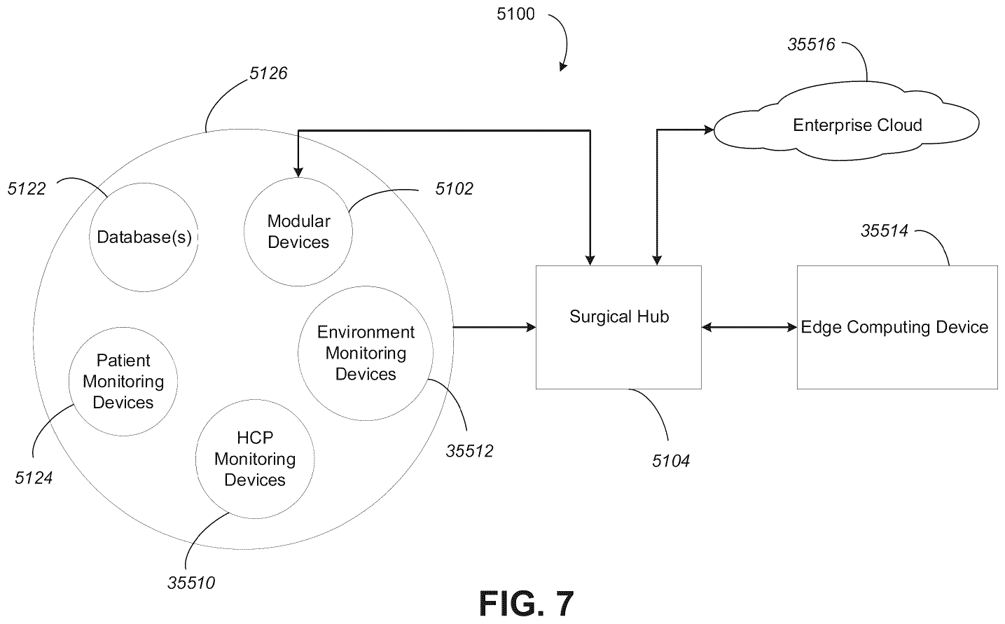

FIG. 7 illustrates a diagram of a situationally aware surgical system 5100, in accordance with at least one aspect of the present disclosure. The data sources 5126 may include, for example, the modular devices 5102 (which can include sensors configured to detect parameters associated with the patient, HCPs and environment and/or the modular device itself), databases 5122 (e.g., an EMR database containing patient records), patient monitoring devices 5124 (e.g., a blood pressure (BP) monitor and an electrocardiography (EKG) monitor), HCP monitoring devices 35510, and/or environment monitoring devices 35512. The surgical hub 5104 can be configured to derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which is the hardware and/or programming associated with the surgical hub 5104 that derives contextual information pertaining to the surgical procedure from the received data and/or a surgical plan information received from the edge computing system 35514 or an enterprise cloud server 35516.

The situational awareness system of the surgical hub 5104 can be configured to derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. A machine learning system can be trained to accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a further machine learning system, lookup table, or other such system, which generates or retrieves one or more control adjustments for one or more modular devices 5102 when provided the contextual information as input.

A surgical hub 5104 incorporating a situational awareness system can provide a number of benefits for the surgical system 5100. One benefit may include improving the interpretation of sensed and collected data, which would in turn improve the processing accuracy and/or the usage of the data during the course of a surgical procedure. To return to a previous example, a situationally aware surgical hub 5104 could determine what type of tissue was being operated on; therefore, when an unexpectedly high force to close the surgical instrument's end effector is detected, the situationally aware surgical hub 5104 could correctly ramp up or ramp down the motor of the surgical instrument for the type of tissue.

The type of tissue being operated can affect the adjustments that are made to the compression rate and load thresholds of a surgical stapling and cutting instrument for a particular tissue gap measurement. A situationally aware surgical hub 5104 could infer whether a surgical procedure being performed is a thoracic or an abdominal procedure, allowing the surgical hub 5104 to determine whether the tissue clamped by an end effector of the surgical stapling and cutting instrument is lung (for a thoracic procedure) or stomach (for an abdominal procedure) tissue. The surgical hub 5104 could then adjust the compression rate and load thresholds of the surgical stapling and cutting instrument appropriately for the type of tissue.

The type of body cavity being operated in during an insufflation procedure can affect the function of a smoke evacuator. A situationally aware surgical hub 5104 could determine whether the surgical site is under pressure (by determining that the surgical procedure is utilizing insufflation) and determine the procedure type. As a procedure type can be generally performed in a specific body cavity, the surgical hub 5104 could then control the motor rate of the smoke evacuator appropriately for the body cavity being operated in. Thus, a situationally aware surgical hub 5104 could provide a consistent amount of smoke evacuation for both thoracic and abdominal procedures.

The type of procedure being performed can affect the optimal energy level for an ultrasonic surgical instrument or radio frequency (RF) electrosurgical instrument to operate at. Arthroscopic procedures, for example, may require higher energy levels because the end effector of the ultrasonic surgical instrument or RF electrosurgical instrument is immersed in fluid. A situationally aware surgical hub 5104 could determine whether the surgical procedure is an arthroscopic procedure. The surgical hub 5104 could then adjust the RF power level or the ultrasonic amplitude of the generator (e.g., “energy level”) to compensate for the fluid filled environment. Relatedly, the type of tissue being operated on can affect the optimal energy level for an ultrasonic surgical instrument or RF electrosurgical instrument to operate at. A situationally aware surgical hub 5104 could determine what type of surgical procedure is being performed and then customize the energy level for the ultrasonic surgical instrument or RF electrosurgical instrument, respectively, according to the expected tissue profile for the surgical procedure. Furthermore, a situationally aware surgical hub 5104 can be configured to adjust the energy level for the ultrasonic surgical instrument or RF electrosurgical instrument throughout the course of a surgical procedure, rather than just on a procedure-by-procedure basis. A situationally aware surgical hub 5104 could determine what step of the surgical procedure is being performed or will subsequently be performed and then update the control algorithms for the generator and/or ultrasonic surgical instrument or RF electrosurgical instrument to set the energy level at a value appropriate for the expected tissue type according to the surgical procedure step.

In examples, data can be drawn from additional data sources 5126 to improve the conclusions that the surgical hub 5104 draws from one data source 5126. A situationally aware surgical hub 5104 could augment data that it receives from the modular devices 5102 with contextual information that it has built up regarding the surgical procedure from other data sources 5126. For example, a situationally aware surgical hub 5104 can be configured to determine whether hemostasis has occurred (e.g., whether bleeding at a surgical site has stopped) according to video or image data received from a medical imaging device. The surgical hub 5104 can be further configured to compare a physiologic measurement (e.g., blood pressure sensed by a BP monitor communicably connected to the surgical hub 5104) with the visual or image data of hemostasis (e.g., from a medical imaging device communicably coupled to the surgical hub 5104) to make a determination on the integrity of the staple line or tissue weld. The situational awareness system of the surgical hub 5104 can consider the physiological measurement data to provide additional context in analyzing the visualization data. The additional context can be useful when the visualization data may be inconclusive or incomplete on its own.

For example, a situationally aware surgical hub 5104 could proactively activate the generator to which an RF electrosurgical instrument is connected if it determines that a subsequent step of the procedure requires the use of the instrument. Proactively activating the energy source can allow the instrument to be ready for use as soon as the preceding step of the procedure is completed.

The situationally aware surgical hub 5104 could determine whether the current or subsequent step of the surgical procedure requires a different view or degree of magnification on the display according to the feature(s) at the surgical site that the surgeon is expected to need to view. The surgical hub 5104 could proactively change the displayed view (supplied by, e.g., a medical imaging device for the visualization system) accordingly so that the display automatically adjusts throughout the surgical procedure.

The situationally aware surgical hub 5104 could determine which step of the surgical procedure is being performed or will subsequently be performed and whether particular data or comparisons between data will be required for that step of the surgical procedure. The surgical hub 5104 can be configured to automatically call up data screens based upon the step of the surgical procedure being performed, without waiting for the surgeon to ask for the particular information.

Errors may be checked during the setup of the surgical procedure or during the course of the surgical procedure. For example, the situationally aware surgical hub 5104 could determine whether the operating theater is setup properly or optimally for the surgical procedure to be performed. The surgical hub 5104 can be configured to determine the type of surgical procedure being performed, retrieve the corresponding checklists, product location, or setup needs (e.g., from a memory), and then compare the current operating theater layout to the standard layout for the type of surgical procedure that the surgical hub 5104 determines is being performed. In some exemplifications, the surgical hub 5104 can compare the list of items for the procedure and/or a list of devices paired with the surgical hub 5104 to a recommended or anticipated manifest of items and/or devices for the given surgical procedure. If there are any discontinuities between the lists, the surgical hub 5104 can provide an alert indicating that a particular modular device 5102, patient monitoring device 5124, HCP monitoring devices 35510, environment monitoring devices 35512, and/or other surgical item is missing. In some examples, the surgical hub 5104 can determine the relative distance or position of the modular devices 5102 and patient monitoring devices 5124 via proximity sensors, for example. The surgical hub 5104 can compare the relative positions of the devices to a recommended or anticipated layout for the particular surgical procedure. If there are any discontinuities between the layouts, the surgical hub 5104 can be configured to provide an alert indicating that the current layout for the surgical procedure deviates from the recommended layout.

The situationally aware surgical hub 5104 could determine whether the surgeon (or other HCP(s)) was making an error or otherwise deviating from the expected course of action during the course of a surgical procedure. For example, the surgical hub 5104 can be configured to determine the type of surgical procedure being performed, retrieve the corresponding list of steps or order of equipment usage (e.g., from a memory), and then compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

Surgical autonomous systems, devices, and methods may include aspects of integration with other medical equipment, data sources, processes, and institutions. Surgical autonomous systems, devices, and methods may include aspects of integration with a computer-implemented interactive surgical system and/or with one or more elements of a computer-implemented interactive surgical system, for example. Surgical system, surgical autonomous system, and autonomous surgical system may be interchangeably as described herein.

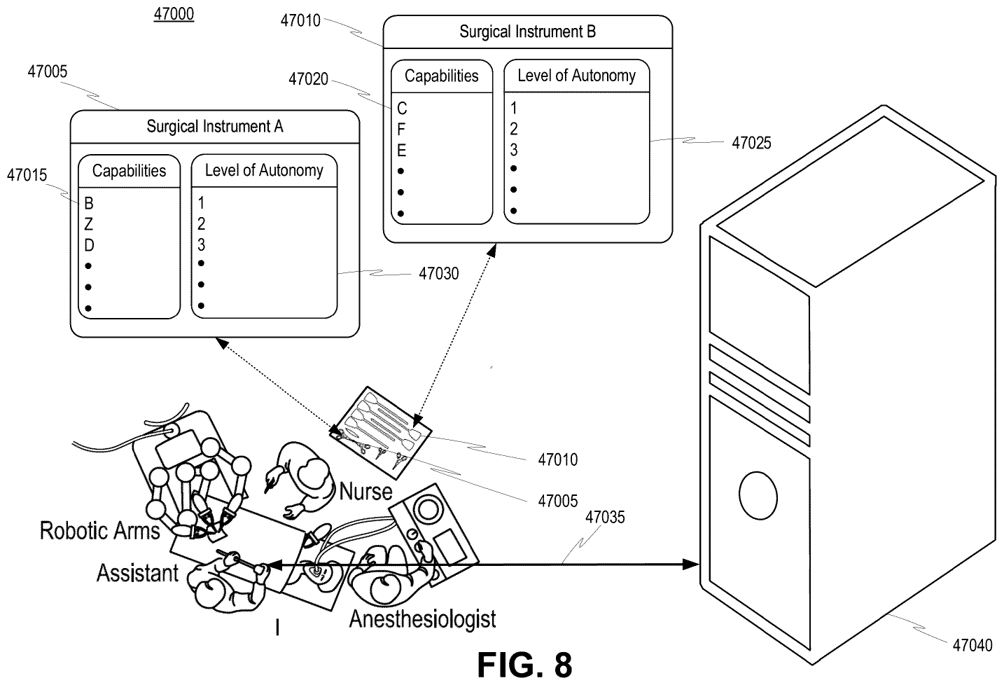

Referring to FIG. 8, an overview of the surgical autonomous system 47000 may be provided. Surgical instrument A 47005 and/or surgical instrument B 47010 may be used in a surgical procedure as part of the surgical system 47000. The surgical hub 47040 may be configured to coordinate information flow to a display of the surgical instrument. For example, the surgical hub may be described in U.S. Patent Application Publication No. US 2019-0200844 A1 (U.S. patent application Ser. No. 16/209,385), tided METHOD OF HUB COMMUNICATION, PROCESSING, STORAGE AND DISPLAY, filed Dec. 4, 2018, the disclosure of which is herein incorporated by reference in its entirety. Example surgical instruments that are suitable for use with the surgical system 47000 are described under the heading “Surgical Instrument Hardware” and in U.S. Patent Application Publication No. US 2019-0200844 A1 (U.S. patent application Ser. No. 16/209,385), tided METHOD OF HUB COMMUNICATION, PROCESSING, STORAGE AND DISPLAY, filed Dec. 4, 2018, the disclosure of which is herein incorporated by reference in its entirety, for example.

FIG. 8 shows an example of a surgical autonomous system 47000. The system 47000 may be used to perform a surgical procedure on a patient who is lying down on an operating table in a surgical operating room. A robotic system may be used in the surgical procedure as a part of the surgical system. For example, the robotic system may be described in U.S. Patent Application Publication No. US 2019-0200844 A1 (U.S. patent application Ser. No. 16/209,385), tided METHOD OF HUB COMMUNICATION, PROCESSING, STORAGE AND DISPLAY, filed Dec. 4, 2018, the disclosure of which is herein incorporated by reference in its entirety. The robotic hub may be used to process the images of the surgical site for subsequent display to the surgeon through the surgeon's console.

Other types of robotic systems may be readily adapted for use with the surgical system 47000. Various examples of robotic systems and surgical tools that are suitable for use with the present disclosure are described in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

Various examples of cloud-based analytics that are performed by the cloud, and are suitable for use with the present disclosure, are described in U.S. Patent Application Publication No. US 2019-0206569 A1 (U.S. patent application Ser. No. 16/209,403), tided METHOD OF CLOUD BASED DATA ANALYTICS FOR USE WITH THE HUB, filed Dec. 4, 2018, the disclosure of which is herein incorporated by reference in its entirety.

In various aspects, an imaging device may be used in the surgical system 47000 and may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The one or more illumination sources may be configured to radiate electromagnetic energy in the visible spectrum as well as the invisible spectrum. The visible spectrum, sometimes referred to as the optical spectrum or luminous spectrum, is that portion of the electromagnetic spectrum that is visible to (e.g., can be detected by) the human eye and may be referred to as visible light or simply light. A typical human eye will respond to wavelengths in air that are from about 380 nm to about 750 nm.

The invisible spectrum (e.g., the non-luminous spectrum) is that portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device may be configured for use in a minimally invasive procedure. Examples of imaging devices suitable for use with the present disclosure include, but not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), tided METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” i.e., an operating or treatment room, necessitate the highest possible sterility of all medical devices and equipment. Part of that sterilization process is the need to sterilize anything that comes in contact with the patient or penetrates the sterile field, including the imaging device and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

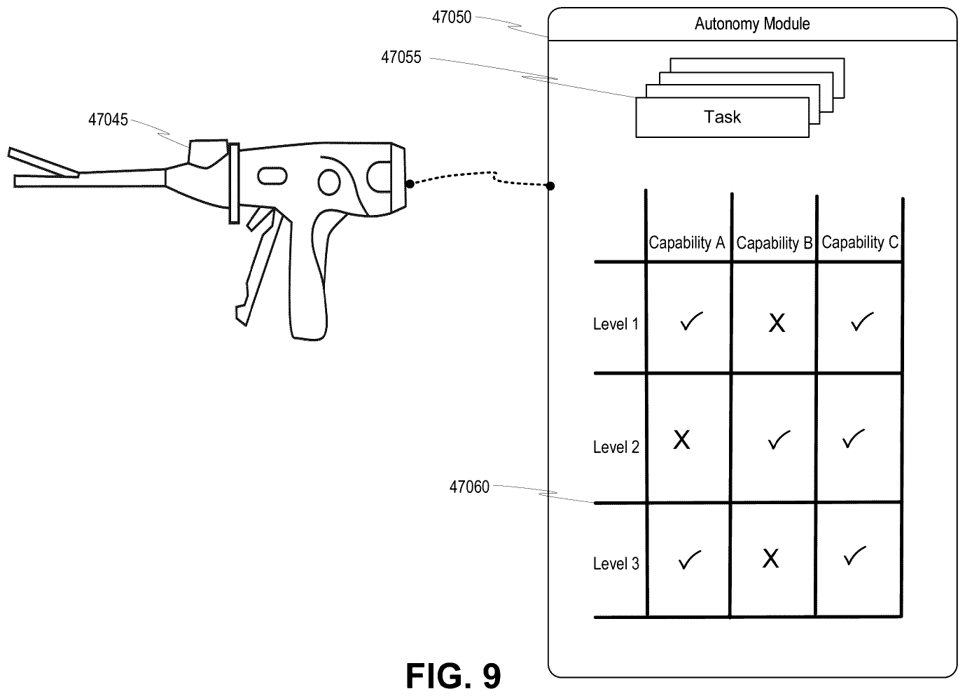

As described with respect to FIGS. 2, 3, 5, and 7, surgical instrument A 47005 and/or surgical instrument B 47010 may comprise one or more capabilities (e.g., capabilities 47015 associated with surgical instrument A 47005 which may comprise B, Z, and D and capabilities 47020 associated with surgical instrument B 470010 which may comprise C, F, and E). The capabilities may be associated with features that the surgical instrument is capable of performing. Examples of the features may be described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example. For example, if the surgical instrument is an endocutter, one of the capabilities may be resecting tissue (e.g., tissue surrounding a colon when the surgeon in performing a colorectomy). The capabilities may comprise surgical tasks as described with respect to FIG. 9. For example, the capability of resecting tissue may comprise controlling an energy source, cutting, stapling, knob orientation, body orientation, body position, anvil jaw force, reload alignment slot management, and/or the like.

The capabilities (e.g., each of the capabilities) of surgical instrument A 47005 and/or surgical instrument B 47010 may be associated with respective autonomy levels (e.g, capabilities 47015 associated with surgical instrument A 47005 may be associated with levels of autonomy 47030 and capabilities 47020 associated with surgical instrument B 470010 may be associated with autonomy levels 47025). For example, the autonomy levels may be categorized by numbers such as 1, 2, 3, etc. 1 may represent an autonomy level with the least manual input when compared to the other autonomy levels. For example, an autonomy level with the least manual input may result in the user of the surgical instrument having to perform less surgical tasks associated with the capability when compared to other levels of autonomy. If the surgical instrument is an endocutter and the capability is resecting tissue, autonomy level 1 may result in the surgeon having only to perform controlling the energy source whereas autonomy level 2 may result in the surgeon having to perform controlling an energy source, body orientation, and/or body position. The tasks not performed by the surgeon may be performed using an autonomous function.

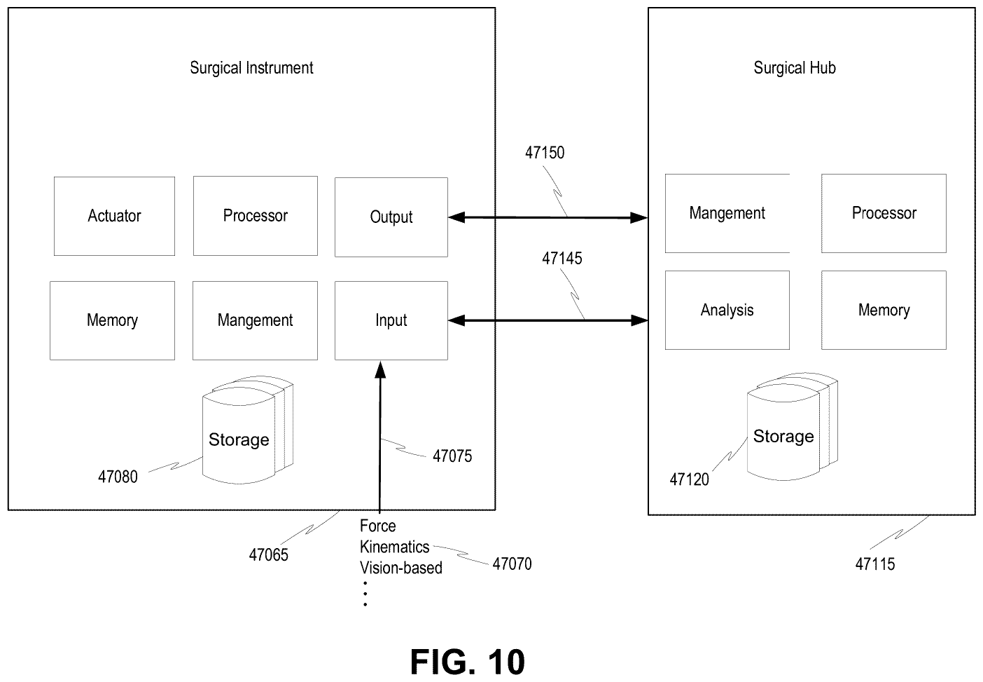

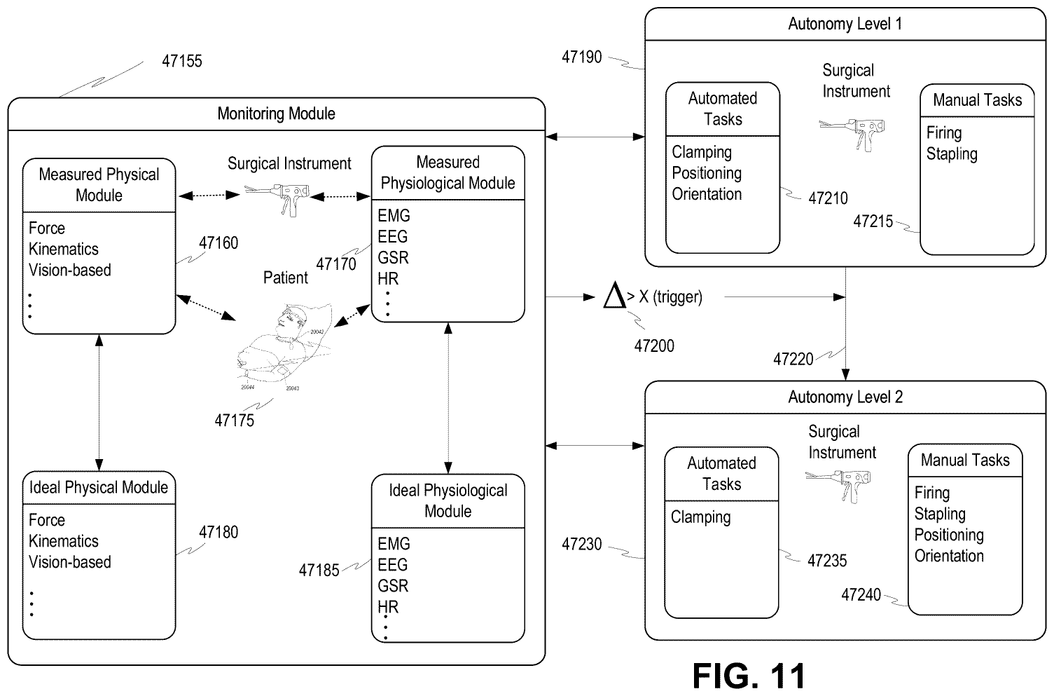

Data may be generated (e.g., by a monitoring module located at the surgical hub 47040 or locally by the surgical instrument as described with respect to FIG. 11) based on the performance of surgical instrument A 47005 and/or B 47010. The data may be relevant to how the current level of autonomy at which the surgical instrument is operating is doing in terms of performance. For example, the data may be associated with physical measurement physiological measurements, and/or the like as described with respect to FIGS. 10 and 11. The measurements are described in greater detail under the heading “Monitoring Of Adjusting A Surgical Parameter Based On Biomarker Measurements” in U.S. patent application Ser. No. 17/156,028, filed Nov. 10, 2021, the disclosure of which is herein incorporated by reference in its entirety.

An indication 47035 of the data may be transmitted to the surgical hub 47040, for example, where it may be evaluated. In examples, the indication 47035 may be transmitted to a cloud service (e.g., amazon web services) as described herein. The data may be used as input in an analysis module, for example, to check if the performance of the surgical instrument falls within a satisfactory range. The surgical instrument may comprise a capability associated with a current level of autonomy (e.g., autonomy level 1). If the data related to the performance falls outside the range (e.g., crosses a threshold as described with respect to FIGS. 11 and 9), the surgical hub 47040 may send an indication 47035 back to the surgical instrument (e.g., surgical instrument A 47005 and/or B 47010) to switch the level of autonomy associated with the capability. For example, resecting tissue may be switched from autonomy level 1 to autonomy level 2 if data related to performance crosses a threshold.

FIG. 9 shows an example of an autonomy module 47050 associated with a surgical instrument 47045 and surgical instrument capabilities 47060. The autonomy module 47050 may be associated with a surgical instrument 47045. In examples, the autonomy module 47045 may be part of the surgical instrument's software. In examples, the autonomy module 47045 may be part of the surgical hub as described with respect to FIG. 8. The autonomy module 47045 may keep track of the link between the surgical instrument's capabilities 47060 and the level of autonomy. For example, the autonomy module 47050 may reference, e.g., via a query, a database (e.g., structured query language (SQL) database) that maintains the link between capabilities 47060 and autonomy levels at a given time (e.g., maintains that, at the current time, capability A is running on autonomy level 1). The database may be updated based on the autonomy level associated with a capability changing. In examples, the database may link the one or more tasks 47055 to the autonomy level. In such a case, the capability 47060, autonomy level associated with the capability 47060, and the surgical tasks 47055 associated with the autonomy level may be linked and queried by the autonomy module.

In examples, the autonomy module 47050 may comprise all surgical tasks associated with the surgical instrument 47045. The autonomy module 47050 may append the surgical tasks into data structures (e.g., lists) that are associated with respective capabilities 47060. The module 47050 may designate one or more surgical tasks 47055 for the surgical instrument 47045 to perform autonomously based on the capability 47060 and the autonomy level as described herein. For example, the surgical instrument 47045 may be an endocutter. The endocutter's capability may be resecting a tissue and the autonomy level for this capability 47060 may be 1. The autonomy module 47050 may designate controlling the energy source, cutting, stapling, knob orientation, body orientation, body position, anvil jaw force, and reload alignment slot management to be performed autonomously. In such a case, if the capability 47060 switched to autonomy level 2, the autonomy module 47050 may designate less tasks 47055 to be performed autonomously. For example, for autonomy level 2, the autonomy module 47055 may designate body position, anvil jaw force, and reload alignment slot management to be performed autonomously. The other tasks 47055 may be performed manually by a surgeon.

A surgical instrument 47045 may comprise capabilities 47060 where each capability 47060 can only operate at certain autonomy levels. In examples, the autonomy module 47050 may reference a rules engine to check if the capability 47060 is permitted to operate at a given autonomy level. For example, capability A may be organ mobilization. Tissue mobilization may only allow the surgical instrument 47045 to operate at autonomy level 1 or autonomy level 3 (e.g., as shown in FIG. 9). Capability B may be anastomosis. Anastomosis may only allow the surgical instrument 47045 to operate at autonomy level 2 (e.g., as shown in FIG. 9.

FIG. 10 shows an example of computer-implemented autonomous surgical system. The system may comprise a processor associated with the surgical instrument 47065 and a processor associated with the surgical hub 47115. The surgical instrument processor may be coupled to a communication (e.g., communication module), storage 47080, memory (e.g., non-volatile memory), management module, actuator(s), and sensor(s), input interface (e.g., which may obtain 47075 measurements 47070 such as physical, physiological, and vision-based as described with respect to FIGS. 9 and 11, from an external source), and output interface via a system bus. The system bus may be any of several types of bus structure(s) including the memory bus or memory controller, a peripheral bus or external bus, and/or a local bus using any variety of available bus architectures including, but not limited to, 9-bit bus, Industrial Standard Architecture (ISA), Micro-Charmel Architecture (VISA), Extended ISA (EISA), Intelligent Drive Electronics (IDE), VESA Local Bus (VLB), Peripheral Component Interconnect (PCI), USB, Advanced Graphics Port (AGP), Personal Computer Memory Card International Association bus (PCMCIA), Small Computer Systems Interface (SCSI), or any other proprietary bus.

The surgical hub processor may be coupled to communication, storage 47080, memory (e.g., non-volatile memory), input/output interfaces, analysis module, and management module via a system bus.

Each processor may be any single-core or multicore processor such as those known under the trade name ARM Cortex by Texas Instruments. In one aspect, the processor may be an LM4F230H5QR ARM Cortex-M4F Processor Core, available from Texas Instruments, for example, comprising an on-chip memory of 256 KB single-cycle flash memory, or other non-volatile memory, up to 40 MHz, a prefetch buffer to improve performance above 40 MHz, a 32 KB single-cycle serial random access memory (SRAM), an internal read-only memory (ROM) loaded with Stellaris Ware® software, a 2 KB electrically erasable programmable read-only memory (EEPROM), and/or one or more pulse width modulation (PWM) modules, one or more quadrature encoder inputs (QEI) analogs, one or more 12-bit analog-to-digital converters (ADCs) with 12 analog input channels, details of which are available for the product datasheet.

In examples, each processor may comprise a safety controller comprising two controller-based families such as TMS570 and RM4x, known under the trade name Hercules ARM Cortex R4, also by Texas Instruments. The safety controller may be configured specifically for IEC 61508 and ISO 26262 safety critical applications, among others, to provide advanced integrated safety features while delivering scalable performance, connectivity, and memory options.

The system memory may include volatile memory and non-volatile memory. The basic input/output system (BIOS), containing the basic routines to transfer information between elements within the computer system, such as during start-up, is stored in non-volatile memory. For example, the non-volatile memory can include ROM, programmable ROM (PROM), electrically programmable ROM (EPROM), EEPROM, or flash memory. Volatile memory includes random-access memory (RAM), which acts as external cache memory. Moreover, RAM is available in many forms such as SRAM, dynamic RAM (DRAM), synchronous DRAM (SDRAM), double data rate SDRAM (DDR SDRAM), enhanced SDRAM (ESDRAM), Synchlink DRAM (SLDRAM), and direct Rambus RAM (DRRAM).

The system may include removable/non-removable, volatile/non-volatile computer storage media, such as for example disk storage. The disk storage can include, but is not limited to, devices like a magnetic disk drive, floppy disk drive, tape drive, Jaz drive, Zip drive, LS-60 drive, flash memory card, or memory stick. In addition, the disk storage can include storage media separately or in combination with other storage media including, but not limited to, an optical disc drive such as a compact disc ROM device (CD-ROM), compact disc recordable drive (CD-R Drive), compact disc rewritable drive (CD-RW Drive), or a digital versatile disc ROM drive (DVD-ROM). To facilitate the connection of the disk storage devices to the system bus, a removable or non-removable interface may be employed.

It is to be appreciated that the system may include software that acts as an intermediary between users and the basic computer resources described in a suitable operating environment. Such software may include an operating system. In examples, the operating system may be associated with the management modules for the surgical instrument 47065 and surgical hub 47115, respectively. The operating system, which can be stored on the disk storage, may act to control and allocate resources of the computer system. System applications may take advantage of the management of resources by the operating system through program modules and program data stored either in the system memory or on the disk storage. It is to be appreciated that various components described herein can be implemented with various operating systems or combinations of operating systems.

A user may enter commands or information into the system through input device(s) coupled to the I/O interface. These and other input devices connect to the processor through the system bus via interface port(s). The interface port(s) include, for example, a serial port, a parallel port, a game port, and a USB. The output device(s) use some of the same types of ports as input device(s). Thus, for example, a USB port may be used to provide input to the computer system and to output information from the computer system to an output device. The output device may be the surgical hub 47115. The output device may be the surgical instrument 47065. An output adapter may be provided to illustrate that there can be some output devices like monitors, displays, speakers, and printers, among other output devices that may require special adapters. The output adapters may include, by way of illustration and not limitation, video and sound cards that provide a means of connection between the output device and the system bus. It should be noted that other devices and/or systems of devices, such as remote computer(s), may provide both input and output capabilities.

The system may operate in a networked environment using logical connections to one or more remote computers, such as cloud computer(s) (e.g., as described with respect to FIG. 8), or local computers. The remote cloud computer(s) can be a personal computer, server, router, network PC, workstation, microprocessor-based appliance, peer device, or other common network node, and the like, and typically includes many or all of the elements described relative to the computer system. For purposes of brevity, only a memory storage device is illustrated with the remote computer(s). The remote computer(s) may be logically connected to the computer system through a network interface and then physically connected via a communication connection. The network interface may encompass communication networks such as local area networks (LANs) and wide area networks (WANs). LAN technologies may include Fiber Distributed Data Interface (FDDI), Copper Distributed Data Interface (CDDI), Ethernet/IEEE 802.3, Token Ring/IEEE 802.5 and the like. WAN technologies may include, but are not limited to, point-to-point links, circuit-switching networks like Integrated Services Digital Networks (ISDN) and variations thereon, packet-switching networks, and Digital Subscriber Lines (DSL).

In various aspects, the computer system may comprise an image processor, image-processing engine, media processor, or any specialized digital signal processor (DSP) used for the processing of digital images. The image processor may employ parallel computing with single instruction, multiple data (SIMD) or multiple instruction, multiple data (MIMD) technologies to increase speed and efficiency. The digital image-processing engine can perform a range of tasks. The image processor may be a system on a chip with multicore processor architecture.

The communication connection(s) may refer to the hardware/software employed to connect the network interface to the bus. While the communication connection is shown for illustrative clarity inside the computer system, it can also be external to the computer system. The hardware/software necessary for connection to the network interface may include, for illustrative purposes only, internal and external technologies such as modems, including regular telephone-grade modems, cable modems, and DSL modems, ISDN adapters, and Ethernet cards.

The surgical instrument 47065 may include one or more hardware components such as actuators, sensors, etc. As the surgical instrument is performing a surgical, data may be generated from the hardware components, communicated to the surgical instrument's memory, and archived in the surgical instrument's storage 47080. The data may relate to physical, physiological, vision-based, etc. conditions of the surgical instrument's performance. As described herein, data may be received 47075 by the surgical instrument from an outside source. Physical conditions may relate to the force that the surgical instrument is applying (e.g., such as a mechanical grasp) and/or the kinematics of the surgical instrument 47065 (e.g., position, speed, and/or orientation of the instrument).

The data (e.g., an indication 47150 of the data) may be transmitted via the communication, as described herein, to the surgical hub 47115. The surgical hub 47115 may use the data as input in an analysis module. The analysis module may assess whether the data crosses the threshold (e.g., is within the range of satisfactory outcome as described with respect to FIGS. 9 and 11). In examples, the analysis module may be a cloud service, such as Amazon Web Services's (TM) (AWS)'s lambda function. The surgical hub 47115 may archive the data in storage 47120 (e.g., data may be used for a subsequent surgery). The data may be analyzed (e.g., by the analysis module) and an indication 47145 of the analyzed data may be sent back to the surgical instrument 47065 as input.

FIG. 11 shows an example of monitoring the performance of surgical tasks associated with autonomy levels. A monitoring module 47155 may be part of the surgical instrument, surgical hub, or a remote device (e.g., a third-party cloud service). In examples, aspects of the monitoring module 48155 may be distributed among multiple devices (e.g., each device may be responsible for executing a certain set of instructions). The monitoring module 47155 may be the analysis module as described with respect to FIG. 10. The monitoring module 47155 may assess whether the performance of the surgical instrument capability, operating at an autonomy level, is within an acceptable (e.g., satisfactory) range.

The monitoring module 48155 may obtain data associated with the surgical instrument and/or patient 47175, as is shown in FIG. 11. The data may be generated from an external source (e.g., a wearable on the patient 47175 that measures biomarker data) and an indication of the data may be transmitted to the monitoring module 47155 via a message. The message may be in response to the monitoring module 47155 requesting the data via a request message. The data may be in raw form and may be transformed by the processor (e.g., the surgical hub processor or the surgical instrument processor as described with respect to FIG. 10) into a form suitable for analysis.

The data may relate to the performance of the surgery. For example, the data may relate to measured physiological conditions of the patient 47175, surgeon, and/or staff who are participating in the surgery. For example, the patient 47175 may be wearing a heart rate monitoring wearable. Monitoring wearables are described in greater detail under the heading “Method Of Adjusting A Surgical Parameter Based On Biomarker Measurements” in U.S. patent application Ser. No. 17/156,28, filed Nov. 10, 2021, the disclosure of which is herein incorporated by reference in its entirety. In such a case, data related to the patient's heart rate may be obtained and used by the monitoring module. The surgeon may be wearing a headband wearable that measures stress via sweat sensors (e.g., by deducing the level of cortisol that the surgeon is producing). The data related to the measured physiological conditions may be organized in a measured physiological module 47170, which may be a part of the monitoring module 47155.

The data may relate to measured physical conditions of the instrument, patient 47175, surgeon, and/or staff. For example, potentiometers and/or sensors may be placed on the surgical instrument and data related to the instrument's movement and/or orientation may be generated and sent to the monitoring module 47155. For example, the instrument, performing tasks autonomously based on its autonomy level as described with respect to FIG. 9, may move from position A to position B. The rate at which the instrument moved may be calculated (e.g., based on calculating readings from the potentiometers) and an indication of the rate may be transmitted to the monitoring module. In examples, the measured physical conditions may be obtained via data produced by a vision-based device. This data may be organized in the measured physical module 47160. In an example, physical and/or physiological conditions may be objects of operation. In an example, conditions other than physical and/or physiological may be objects of operation.

The monitoring module 47155 may be in communication with a surgical instrument. The surgical instrument may comprise a capability and the surgical instrument may be operating at a current autonomy level, for example, autonomy level 1 47190 as shown in FIG. 11, to perform the capability. Operating at autonomy level 1 47190 may result in the surgical instrument performing a number of tasks associated with the capability autonomously. For example, the automated tasks 47210 may be clamping, positioning, and orientation as shown in FIG. 11. This may leave the other tasks associated with the capabilities of firing and stapling to be performed manually (e.g., by the surgeon). Data related to the automated tasks 47210 and manual tasks 47215 may be generated and transmitted to the monitoring module 47155. The data may be organized in the measured physical module 47160 as described herein.

The monitoring module 47155 may include an ideal physical module 47180 and/or an ideal physiological module 47185. The module(s) may include the ideal values associated with a number of physical and physiological conditions. The ideal value may be dynamic and change based on the surgical context. For example, the ideal value associated with a surgeon's heart rate may increase if the surgeon is at a critical stage of the surgery. The ideal values may be generated based on historical data as described with respect to FIG. 9. The ideal values may be entered manually, for example, by the surgeon or other healthcare expert. In examples, the measured physical module 47160, measured physiological module 47170, ideal physical module 47180, and ideal physiological module 47185 may be organized in a different format.

The monitoring module 47155 may compare the measured physical module 47160 and measured physiological module 47170 to the ideal physical module 47180 and ideal physiological module 47185, respectively. In examples, an analysis module may be included in the monitoring module 47155 and may be responsible for assessing the comparison. The difference between the measured modules and ideal modules may be calculated and a delta output 47200 may be generated. In examples, each physiological condition may be assigned a weight and the weights may be considered when generating the delta output 47200. In examples, only a subset of the measured physical and/or measured physiological conditions may be compared to the ideal conditions, which may be determined based on surgical context as described with respect to FIG. 9.

The delta output 47200 may be compared to a trigger (e.g., trigger event), for example, to assess whether the trigger has been met. This trigger may represent whether the difference between the measured modules and ideal modules exceeds an acceptable value and/or range. In examples, a delta output 47200 may be generated for each physiological and/or physical conditions and each delta output 47200 may be compared to respective triggers.

Based on the trigger being met, the autonomy level associated with the surgical instrument capability may switch 47220 to another autonomy level. In examples, the autonomy level may only switch to autonomy levels that are allowed for the capability as described with respect to FIG. 9. As shown in FIG. 11, the trigger has been met and the autonomy level that the surgical instrument is operating at to perform its capability switched 47220 from autonomy level 1 47190 to autonomy level 2 47230. Autonomy level 2 47230 may result in less tasks being performed autonomously (e.g., automated tasks 47235) and more tasks being performed manually (e.g., manual tasks 47240). For example, as shown in FIG. 11, when the surgical instrument is in autonomy level 2 47230, it may perform clamping autonomously and may result in the surgeon manually performing firing, stapling, positioning, and orientation. The update to the level of autonomy may be sent to the databased as described with respect to FIG. 9.

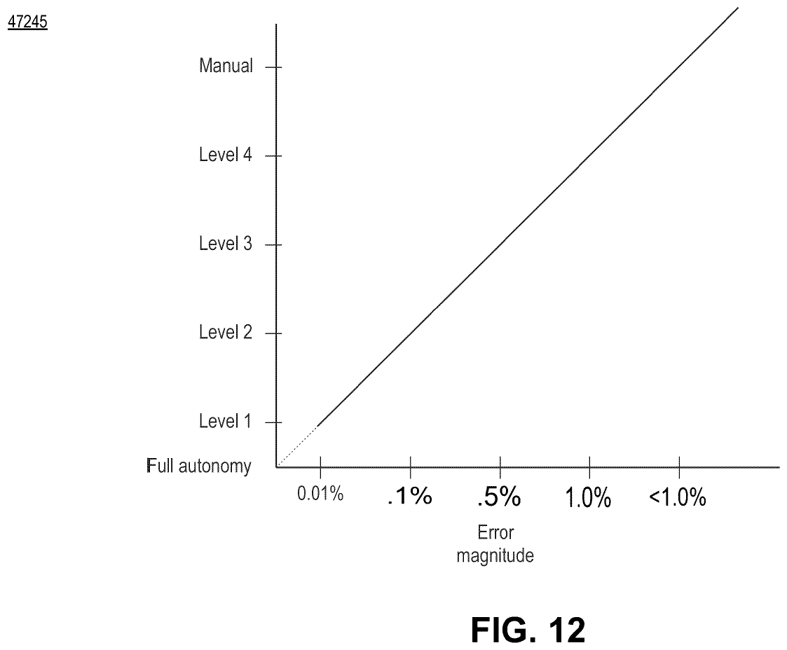

FIG. 12 shows an example of the relationship 47245 between error magnitude and autonomy levels. In examples, an error magnitude may be associated (e.g., linked) with respective autonomy levels. The error magnitude may be used as the threshold described with respect to FIG. 11. For example, the monitoring module, described with respect to FIG. 11, may produce an output (e.g., delta output) based on the difference between measured conditions and ideal conditions. The output may be compared to the error magnitude (e.g., used as the threshold) to determine whether the current autonomy level is leading to an acceptable outcome. For example, as shown in FIG. 12, autonomy level 1 may be associated with error magnitude 0.01%. If the output, e.g., which may be a numerical value, from the monitoring module exceeds 0.01%, an indication may be sent to the surgical instrument to switch the autonomy level, for example, to autonomy level 2.

As shown in FIG. 12, the error magnitude may increase as the autonomy levels increase, where an increased autonomy level has less tasks performed autonomously as described with respect to FIG. 9. If the autonomy level is switched, an indication may be sent to the monitoring module about the switch and the indication may include an updated error magnitude. For example, if the autonomy level is switched to autonomy level 2, an error magnitude of 0.1% may be sent to the monitoring module. The monitoring module may include a database, for example, the database described with respect to FIGS. 9 and 11. The monitoring module may send a message to the database to update both the current level of autonomy at which a surgical instrument is operating and the error magnitude value associated with the updated level of autonomy. The monitoring module may infer the level of autonomy based on the error magnitude.

The error magnitude associated an autonomy level may be determined. For example, the surgical hub may determine the error magnitude for an autonomy level based on historical data. The historical data may show a correlation between the likelihood of a poor outcome occurring and the error magnitude. The historical data may be based on an analysis of what happened when autonomous functions (e.g., associated with autonomy levels) were used to perform the capability in the past. The surgical hub may include an error magnitude module that calculates, based on the historical data, an error magnitude that minimizes the likelihood of a poor outcome occurring. In examples, the error magnitude module may determine the error magnitude based on weighing the benefits of performing a capability with an autonomous level versus performing the capability manually. For example, performing the capability manually may result in a likelihood of a poor outcome, which the error magnitude module may consider when determining the error magnitude. An indication of the error magnitudes may be sent to the monitoring module as described with respect to FIG. 11.

The error magnitudes associated with respective autonomy levels may be dynamic. For example, the surgical hub may send a range of acceptable error magnitudes for an autonomy level to the monitoring module. As the surgical instrument is performing the capability, surgical context data may be sent to the monitoring module, for example, from the surgical hub, the surgical instrument, or other devices associated with the surgery (e.g., wearables that the surgeon may be wearing). The monitoring module may send an indication to adjust the error magnitude based on the surgical context data. For example, the surgical context data may indicate that a critical step of the surgery is being performed. The monitoring module may obtain the data and send an indication to decrease the error magnitude (e.g., the decrease resulting in the threshold being crossed with fewer errors being made).

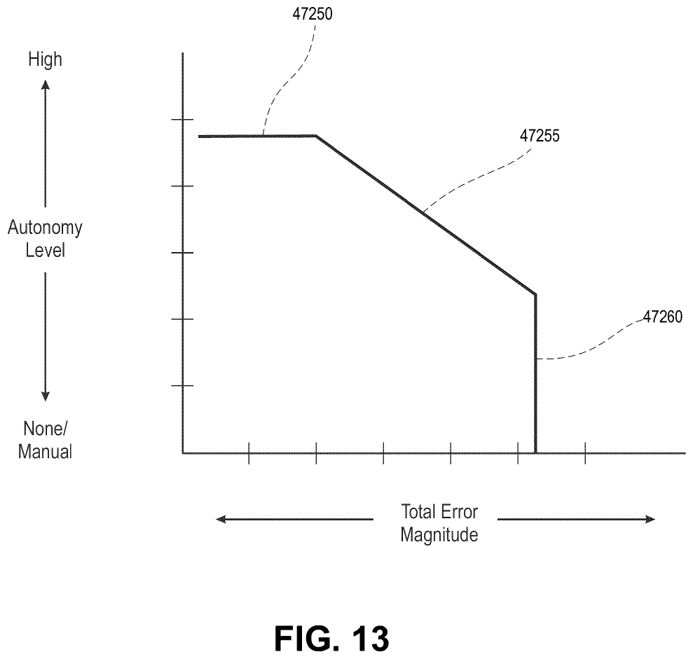

FIG. 13 shows an example of the relationship between error magnitude and autonomy levels.

As shown in FIG. 13 a high level of autonomy 47250 (e.g., which results in a high number of tasks associated with the surgical instrument capability being performed autonomously) may be associated with a low error magnitude (e.g., total error magnitude). In examples, a high level of autonomy 47250 may be represented as autonomy level 1 as described herein. A low level 47260 of autonomy (e.g., which results in a low number of tasks associated with the surgical instrument capability being performed autonomously) may be associated with a high error magnitude (e.g., total error magnitude). As described with respect to FIG. 12, the error magnitude may be related to the threshold, which is used to determined if the autonomy level at which the surgical instrument is operating is to be switched.

As described with respect to FIGS. 12 and 9, the error magnitude may be determined based on historical data, surgical context (e.g., which may include environment data such as number of staff members in the surgical operating room (OR), surgical instrument data as described with respect to FIG. 11, and/or the like). The error magnitude may be determined based on the surgical tasks being performed autonomously. For example, an endocutter's capability may be resecting tissue. It may be set to autonomy level 1 to perform this capability, which may result in the surgical tasks of controlling the energy source, cutting, stapling, knob orientation, body orientation, body position, anvil jaw force, and reload alignment slot management (e.g., which are the tasks associated with resecting tissue) being performed autonomously. Data related to one or more of these surgical tasks, for example, knob orientation, may be obtained by the surgical hub and the surgical hub may use this data when determining the error magnitude. For example, the data may indicate that knob orientation is a task that is likely to be performed successfully when done autonomously. In such a case, the surgical hub may determine a low error magnitude. In examples, the error magnitude may be sent to a monitoring module, as described with respect to FIG. 12, and the monitoring module may infer the autonomy level being using by the instrument. The data may indicate that stapling is a task that is unlikely to be performed successfully when done autonomously. In such a case, the surgical hub may determine a high error magnitude. Data related to multiple tasks may be used, e.g., by the surgical hub, to determine the error magnitude. For example, both data from the knob orientation and data from the stapling may be sent to the surgical hub. The surgical hub may weigh both sets of data and determine a medium level of autonomy 47255 to be used, e.g., since the knob orientation a task that is likely to be performed successfully when done autonomously and stapling is a task that is unlikely to be performed successfully when done autonomously. Error magnitude may be determined for each task associated with the capability to be performed. For example, a surgical hub may determine knob orientation error magnitude, which may be used when assessing whether the autonomy level associated with knob orientation is to be switched as described with the respect to FIG. 12 and stapling error magnitude, which may be used when assessing whether the autonomy level associated with stapling is to be switched.

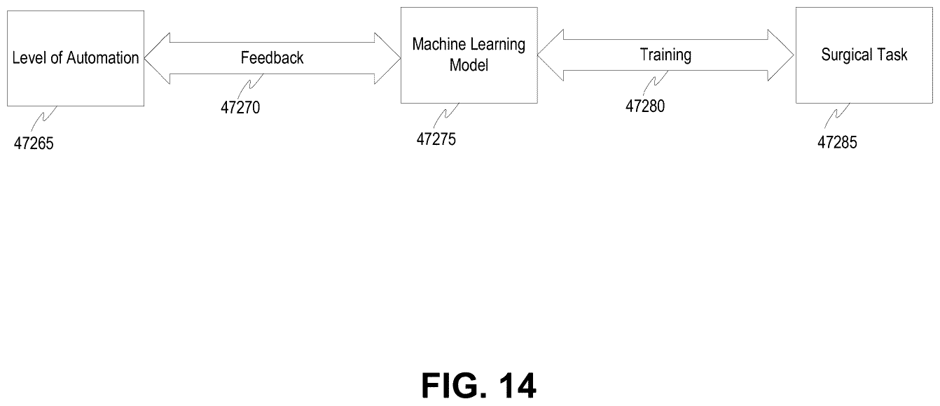

FIG. 14 shows an example of the relationship between autonomy level, machine learning, and the surgical task. Autonomy level and automation level may be used interchangeably herein.

As described herein, performing a surgical task, associated with a surgical instrument capability, autonomously may involve using a machine learning framework. Machine learning framework is described in greater detail under the heading “Method for Surgical Simulation” in U.S. patent application Ser. No. 17/332,593, filed May 27, 2021, the disclosure of which is herein incorporated by reference in its entirety. For example, inputs (e.g., parameters) associated with the surgical task may be sent to a machine learning model 47275 and the machine learning model may be trained 47280 to perform the surgical task 47285 autonomously. The machine learning model 47275 may be involved in determining the level of autonomy 47265 that the surgical task 47285 should be performed at (e.g., autonomy level 1). Data related to the level of autonomy 47265 may be generated and sent to the machine learning model 47275 in the form of feedback 47270, where the model 47275 may adjust the input(s) associated with the surgical task 47285 and/or change the level of autonomy 47265 at which the surgical instrument 47285 is operating. The machine learning model 47275 may be located locally (e.g., a module) on the surgical instrument or on a remote device.

Dynamic variables associated with performing the surgical task 47285 autonomously may be updated by a machine learning algorithm based on performance metrics as described with respect to FIG. 11. For example, the autonomy level 47265 associated with the surgical task 47285 may be updated based on an error magnitude (e.g., threshold) being crossed as described with respect to FIG. 12. The machine learning model 47275 may use real-world data sets of previous surgeries when determining whether the error magnitude has been crossed and/or when determining the autonomy level 47265 associated with the surgical task 47285. The real-world data sets of previous surgeries may be an aggregation of the procedures done by a surgeon, the surgeries from that facility and/or a compilation of surgeries using hubs within the same network.

The machine learning algorithm may be updated by data from a cloud and/or remote system, for example, which may be compiling best practices, regional data on surgeries, and/or worldwide outcomes and step-of-use from any number of other facilities worldwide.

The real-world information may be derived from procedure outcomes, for example, from the region, population etc. and/or may be interpolation and/or aggregation of sub-biomarker measures and outcomes.

For example, a machine learning model 47275 based on GANs (GANs model) may be trained using past surgical procedure data. The GANs model may model that data pattern that given a surgical step, a list surgical tasks 47285 may be performed autonomously. The GANs model may model the probability distribution of the surgical tasks 47285 present in the past surgical procedure data. That is, when the GANs model generates a surgical task 47285 from the list of possible surgical tasks 47285, the surgical task 47285 is generated at a probability according to the probability distribution.

Machine learning may be a part of a technology platform called cognitive computing (CC), which may constitute various disciplines such as computer science and cognitive science. CC systems may be capable of learning at scale, reasoning with purpose, and interacting with humans naturally. A CC system may be capable of performing surgical tasks autonomously.

The output of machine learning's training 47280 process may be a model 47275 for predicting outcome(s) on a new dataset. For example, a linear regression learning algorithm may be a cost function that may minimize the prediction errors of a linear prediction function during the training process by adjusting the coefficients and constants of the linear prediction function. When a minimal may be reached, the linear prediction function with adjusted coefficients may be deemed trained and constitute the model 47275 the training 47280 process has produced. For example, a neural network (NN) algorithm (e.g., multilayer perceptrons (MLP)) for classification may include a hypothesis function represented by a network of layers of nodes that are assigned with biases and interconnected with weight connections. The hypothesis function may be a non-linear function (e.g., a highly non-linear function) that may include linear functions and logistic functions nested together with the outermost layer consisting of one or more logistic functions. The NN algorithm may include a cost function to minimize classification errors by adjusting the biases and weights through a process of feedforward propagation and backward propagation. When a global minimum may be reached, the optimized hypothesis function with its layers of adjusted biases and weights may be deemed trained and constitute the model 47275 the training process 47280 has produced.

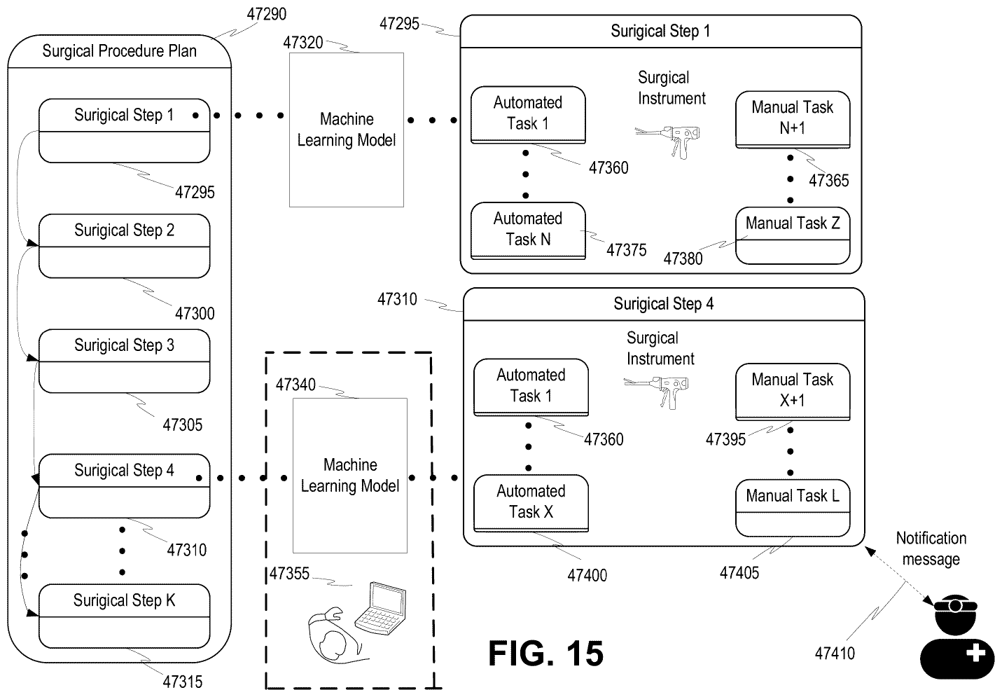

FIG. 15 shows an example determining the autonomy levels at which the surgical instrument performs a surgical task. As described with respect to FIG. 9, the surgical task(s) may be associated with a capability of the surgical instrument, for example, tissue resection. The surgical steps may be associated with the capabilities.

A surgery may follow a surgical procedure plan 47290 which may outline surgical steps to be performed (e.g., surgical step 1 47295, surgical step 2 47300, surgical step 3 47305, surgical step 4 47310, and surgical step K 47315). The surgical steps may be performed in sequence. In examples, the surgical steps may be performed in parallel. A surgical hub or other device may obtain the name of the surgery to be performed and generate the surgical procedure plan based on historical data related to the success of pervious surgeries or previous simulations of the surgery (e.g., performed at a facility). The surgical hub may consider data (e.g., additional data) when generating the surgical procedure plan such as the experience level of the surgeon.

One or more surgical steps (e.g., each surgical step) may include surgical task(s) to be performed by a surgical instrument. As described with respect to FIG. 11, one or more of the tasks may be performed autonomously and one or more may be performed manually, which may be determined based on the autonomy level associated with the capability (e.g., surgical step). In examples, all the surgical tasks may be performed autonomously, for example, if the capability is associated with the highest autonomy level (e.g., “full autonomy” level as shown in FIG. 12).

A machine learning model may be used in determining the autonomy level for a surgical step (e.g., machine learning model 47320 associated with surgical step 1 47295 and machine learning model 47340 associated with surgical step 4), as described with respect to FIG. 14. In examples, parameters for the machine learning model may be entered by a user 47355. The machine learning model 47320 may output that for surgical step 1 47295, tasks 1 47360 to N 47375 are to be performed autonomously and tasks N+1 47365 to Z 47380 are to be performed manually (e.g., by the surgeon or another surgical staff member). The machine learning framework may receive feedback about the performance of the surgical step. If the performance crosses a threshold, the machine learning model may obtain a message that the autonomy level is to be switched. In such a case, the model may determine an updated autonomy level based on the feedback about the performance of the surgical step, which may include one or more of real-time surgical data, user data, surgical environment data, surgical instrument data, task data, or historical data. The update may occur during the surgical step. The update may occur based on the transition from one surgical step to another surgical step (e.g., during the transition from surgical step 1 to surgical step 2). The updated autonomy level may be associated with a different set of surgical tasks to automate (e.g., perform autonomously). For example, based on the transition to surgical step 4 47310, the tasks 1 47360 to X 47400 may be performed autonomously and tasks X+1 47395 to L 47405 may be performed manually. A message 47410 (e.g., a simple notification service (SNS)) may be sent to the surgeon or other surgical staff member of the updated autonomy level. The surgical tasks to be performed autonomously and manually may be sent via the message.

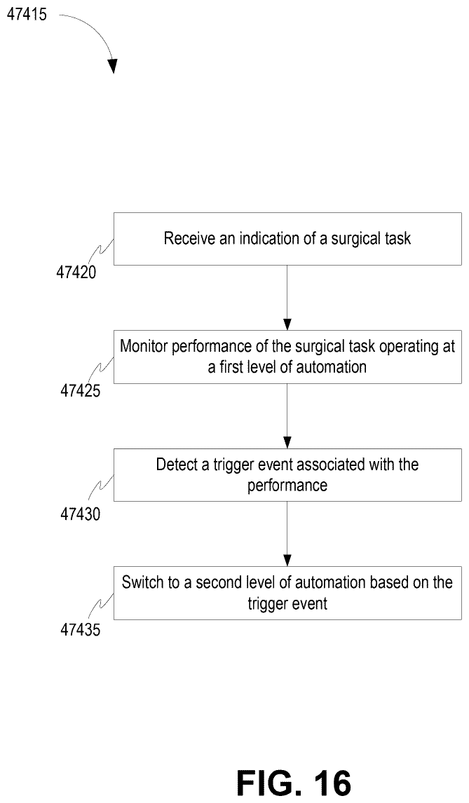

FIG. 16 shows an example flow chart 47415 for automating surgical tasks associated with autonomy levels. At 47420, the device may receive an indication of a surgical task to be performed with a surgical instrument. Capabilities of the surgical instrument may be associated with levels of automation.

At 47425, the device may monitor a performance of the surgical task with the surgical instrument operating at a first level of automation associated with the levels of automation. The capabilities of the surgical instrument may include a set of surgical instrument tasks and the level of automation may be associated with automating one or more surgical instrument tasks from the set of surgical instrument tasks.

At 47430, the device may detect a trigger event associated with the performance of the surgical task and switch operation of the surgical instrument from the first level of automation to a second level of automation associated with levels of automation, for example, based on the trigger event. The performance of the surgical task may be based on real-time surgical data. The real-time surgical data may include one or more of the following: user data, surgical environment data, surgical instrument data, task data, historical data, or the like.

At 47435, the device may detect the trigger event by comparing the performance of the surgical task with a trigger event threshold and may switch operation of the surgical instrument from the first level of automation to the second level of automation based on the trigger event. The second level of automation may be associated with automating less surgical instrument tasks when compared to the first level of automation. Monitoring the performance of the surgical task may include comparing one or more of the real-time surgical data to respective ideal surgical data.

In examples, the device may operate at a first level of automation of the levels of automation associated with the surgical task. The device may obtain an indication to switch to a second level of automation of the levels of automation associated with the surgical task. The indication may be based on detecting a trigger. The device may operate at the second level of automation of the levels of automation associated with the surgical task based on the indication. The indication may be based on monitoring a performance of the first level of automation. The performance of the first level of automation may be based on real-time surgical data.

Autonomous decision-making and assisting may be provided. Auto-determination of the level of autonomy (e.g., level of automation) from a predefined set of options may be based on identifying the situation (e.g., using situation awareness) of the surgical task.

Smart medical device determination of the level of automation may be based on the monitored situation of the procedure. For example, a powered adaptable medical device control algorithm for controlling a medical instrument function may include a variable magnitude of automation. Monitoring of the instrument, surgeon, and/or patient may control the magnitude or level of automation (e.g., the control algorithm) without direct user control. In examples, the level of automation may be based on one or more of the following: the capabilities of the device, the connections of the device to other devices, the presence of a number of personnel, or detection of an aspect of its status and/or configuration.

Automated task categorization (e.g., selectable set of choices that are available) may be requested by the surgical instrument (e.g., a request message may be sent to the surgical hub) and an assessment of the complexity may be used to automatically determine the level of appropriate automation for the surgical instrument, e.g., in order to perform the surgical task.

Features describe herein may result in more autonomy. For example, there may be a release from automated operation based on product in-servicing. The level of autonomy may be determined based on user (e.g., surgeon) skill level. For example, inexperienced users (e.g., resident surgeon) may trigger a lower level of automation to be used, e.g., to ensure proper operation of the surgical instrument. For example, an inexperienced user (e.g., resident surgeon) may have to pull an endocutter in an emergency situation of the surgical task. The surgical hub and/or surgical instrument may recognizes that the skill level of the user as inexperienced (e.g., novel) and may set automation to minimize choices and maximize easy-of-use with limited user control activations. An autonomy level may be associated with user selectable options and, if an inexperienced user may have less selectable options than an experienced user (e.g., the selectable options are locked-out and automatically grayed-out). More advanced users may be given an option to automate more (e.g., surgical tasks which results from using a higher level of automation) than inexperience users.

The level of autonomy may be based on a user field of view. For example, if the surgical move outside the field of view (e.g., laparoscopic field of view), an autonomous instrument control may be adjusted. For example, during surgery the surgeon may have multiple ports link to multiple different instruments to perform the intended task and may have one laparoscopic camera with a restricted/defined view. Throughout the surgery, the surgeon may switch between instruments and/or move instruments to increase access. In such a case, one or more of the instruments may no longer be in the field of view. The instrument may activate an autonomous mode in which the device function may not be activated until it is moved back into the field of view.

Device (e.g., surgical instrument) orientation may be autonomously controlled. For example, if the instrument is outside the field of view and the surgeon attempts to place the instrument back into the field of view, the device end effector may autonomously control itself in which it may rotate and/or orient itself to not contact other structures/tissue/organs until it was back into the field of view, which may prevent unintended actions.

Autonomous instrument control may be based on the surgeon's focus or viewing angle which may be fixated on a portion of the screen. For example, during surgery, the surgeon may be viewing from a laparoscopic camera. The surgeon may get disoriented and/or fixated on a certain task of function (e.g., while mobilization or creating access to the targeted site, his/her focus may be fixated on a portion of the screen). In such a case, the surgical instrument may switch to an autonomous mode that would limit certain functions until the surgeon's eyes were redirected to that instrument. In such case, instruments may determine an autonomous mode that may result in no contact to other structures. For example, the autonomous mode may allow the end effector to move or orient autonomously as the surgeon translates the device into the focus area of the screen.

The facility operators and/or surgeon may choose to limit autonomy customization based on risk level. The level of autonomy to assist in surgery may be set/controlled (e.g., preemptively). For example, this may be based on administrative approval, safety concerns and/or risk to patients. The facility operators may choose to use the default automation, which may enable more inexperienced surgeons to get more repeatable results. The facility operators may choose to disable or deactivate available levels of autonomous operation, e.g., until it validates the behavior aligns with their approach to surgical intervention and/or outcomes. This may prevent devices from being brought into the facility and causing poor outcomes because the autonomous function that was used was incompatible.

Adjusting the autonomy level may be based on task and/or patient risk. Laparoscopic ultrasonic devices may have multifunctional uses such as one or more of the following: coagulation, cutting, dissecting, or grasping. Based on the intended function, targeted zone, and/or surgical task, the autonomy level may be controlled by the intended function and/or patient risk, for example, to control the allowed operations by the autonomous system and/or the combination of human activation. In examples, the higher the risk to the patient may transfer the execution of a task from an autonomous state to a mixed state with the surgeon having to approve an action before it is performed since the action may result in a greater negative outcome to the patient. In such a case, the surgeon may be prepared to react if an unintended outcome occurs (e.g., computer-controlled systems may have more precise controls, however humans/surgeons may anticipate issues and adjust their response to complete the task). For example, the patient risk may be level 1. The surgical instrument may be used for grasping and tissue manipulation. Based on the level 1 risk, the surgical instrument may allow for full autonomy of the jaws opening and closing. For example, the patient risk may be level 2. The surgical instrument may be used for cutting and dissecting. Based on the level 2 risk, the surgical instrument may allow full autonomy of the jaws and energy activation when on non-vascular tissue but may not on fatty tissue. In examples, the patient risk level may be 3. The surgical instrument may be used for vessel sealing and/or coagulation. Based on the level 3 risk level, the surgical instrument may allow full autonomy of the jaws but energy activation may be controlled/applied by the surgeon. This may result in less automation and more discrete operation. The autonomy level may be based on procedure complexity and/or patient specific implications (e.g., co-morbidities). The autonomy level may be based on detected issues.

The facility may limit devices automation (e.g., level of automation) of processes, e.g., until the facility has validation of use (e.g., the device function are intended and are cost beneficial).

An autonomy level may be based (e.g., switched based on) safety risk, incorrect operation of surgical instrument, and/or detected issue with surgical instrument. For example, safety energy activation may be used. For ultrasonic and/or RF energy devices. keeping the jaws and active electrodes clean and free of debris throughout the procedure may prevent tissue build-up which may lead to unintended generator errors and/or reduction in performance. To complete a task, the surgeon may remove the device from the patient and the surgeon and/or scrub nurse may use a sponge to clean the jaws. As a safety precaution, the device may autonomously activate a safety mode when sensed that the jaws be cleaned to ensure energy activation may not be activated while cleaning. In examples, for harmonic devices, if the blade is accidentally activated while using hemostasis while cleaning the blade and/or the blade makes contact with something while cleaning and/or in motion back to the patient, this may lead to scratches, nicks and/or notches in the blade which may increase the potential for premature blade failures. Having the power autonomously deactivated while not in the patient may minimize these issues. For example, safety jaw closure may be used. For surgical procedures, devices with end effectors may be be closed while passing through the trocar. Autonomously, the devices may close the jaws prior to being removed or inserted into the trocar. Thermal damage may be used. Energy devices like the Harmonic Scalpel (e.g., ACE Family) may reach temperatures in excess of 200° C. while performing the intended task, or after deactivation of the energy button. These devices may take longer to cool. Autonomously, the system may control the allowable movement of the jaws and/or restrict movement until an acceptable temperature of the jaws is met to ensure adjacent tissue is not inadvertently impacted by thermal damage.

Having selectable levels of automation may result in more or less autonomy based on the setting and/or options available. Facility operators and/or user may use more advanced features (e.g., associated with higher level of autonomy) in a certain setting. For example, smart software modules, more comprehensive control programs, and/or capacity of the hardware may be used to determine the level of autonomy. The user of the more advanced functions may select a more constrained level of autonomy based on their needs (e.g., a teaching facility may use higher levels of autonomy to ensure safety of less experienced surgeons or a regional facility may deactivate an autonomous function because in that region it is seen as not benefiting the patient or it may cause more challenges than it solves).

A tiered approach may be used to assess risks associated with a surgical task, which may include one or more of the following: accept risk, avoid risk, transfer risk, or reduce risk.

A device may be identified within a larger digital ecosystem. The device may be detected by other systems that are capable of integration or intercommunicating with the device and/or system.

The autonomy level may be based on the presence of cooperative devices or systems. For example, a first smart device may detect the presence of a second smart device or surgical hub which may initiate automated communication interaction. Once the smart devices are communicating, the type and/or configuration of the second smart hub may enable the automated update or communication of operational parameters for the first device that may enable an updating of the system. Once the first device is operated, it may automatically update the second smart Hub with the information regarding its use which may be in-turn automatically compiled with other information from other devices and automatically parsed and disseminated to other systems that use some portion of that data. This distributed data may be used when determining the level of autonomy for the surgical instrument within the cooperative system. Product detections may be used as a trigger to perform the features described herein. For example, one or more of the following may be a trigger: the presence of a smart device in a smart hub proximity; the presence of a smart component within a smart device (e.g., RFID within a smart stapler); the presence or proximity of cooperative compatible devices (e.g., smart proximity sensing scope or scope add-on and the presence of a smart device with integrated fiducial markers); a first hub within range of a second hub, smart OR, network gateway, and/or communication backplane generator; or a first imaging system within range of a second imaging system.

The system's ability to measure and/or detect the information to operate at a level of autonomy may be determined.

HCPs within the room capable of interacting or operating with the device may be determined. For example, the adequate number of appropriate users may be determined, which may include one or more of the following: area of expertise and/or job function (e.g., surgeon, anesthesiologist, scrub tech, circulator nurses, etc.); experience level (e.g., overall time within medical field, time/quantity of work within given specialty, time/quantity of work within given a procedure, time/quantity of work with specific equipment, and/or certifications and training); skill level (e.g., detected by previous operations, device usage, outcomes, etc.); or manually inputted into the system by users/management. One or more of the following may be used to determine who is in the room: manual input; manual badge scanner; room badger scanner (e.g., auto-check and update who is present in room); facial recognition, or the like.

The autonomy level may be based on one or more of the following: are there insufficient number of people in the room which defaults to automated operation by a limited number of users. For example, if an insufficient number of staff are present, a level of automation may be determined (e.g., adjusted to complete the procedure. In examples, the procedure (e.g., tasks of the procedure) may be updated, which may result in greater efficiency as automation increases by the adjustments.

Notification of the reasoning why a certain automation level has been selected may be provided, which may be used to change selection criteria to be used when selection the automation level. Users may be informed of variables that are affecting the level of automation. For example, if there are insufficient personnel in the room, the surgical instrument may default to manual operation (e.g., or default to autonomous).

Determination of the triggers and/or the magnitude of the autonomy may be based on monitored and/or calculated data feeds. During the performance of a task being performed autonomously, there may be a no good autonomy level options. In such a case, it may be determined how to adjust its level of autonomy. For example, it may be determined to change from one autonomy to a lower level autonomously safely. In examples, it may be determined to finish the current task and stop (e.g., do not proceed the next task). In examples, it may be determined to terminate immediately.

The detected magnitude of error in the information may be used as a means to determine a level of autonomy. For example, as the level of error increases, the level of autonomy decreases. The magnitude of error may include a system-based cumulative total error and/or the criticality of a single error. In examples, a single error with a high degree of criticality may reduce the autonomy of the system more than multiple errors of small or insignificant magnitude. There may be errors which partially remove autonomy of the system. For example, a highly autonomous equipment system such as a surgical robot may have multiple arms connected to it. During a start up test sequence, one of the arms may be detected to have an error with it's feedback sensor based on output data and feedback. It may be determined that a feature of the arm is not operating correctly. The system may eliminate autonomy activity for that arm, while maintaining autonomous activities with other arms. There may be errors which completely remove autonomy of the system. For example, a highly autonomous equipment system such as a surgical robot may use a mainframe processing location. While redundancies exist with certain aspects (e.g., power supplies), there may be no redundancy for the central processor. If there is a failure of the processor, all autonomy for the system may be removed. There may be errors that have no impact on the level of autonomy of the system. For example, a highly autonomous system such as a surgical robot may rely on a GPS signal to calculate time and date, as well as country of location, for record keeping. That GPS signal may become lost and may create an error. There may a holdover period for 24 hours from which prior data is still deemed accurate. In such a case, although an error has occurred, there may be no impact to functionality or autonomy level.

In examples, true cumulative error may not be calculated. In such a case, specific functions and features related to autonomy may be removed dependent upon the associated failure mode. Each potential autonomous function of the device may be mapped to one or more corresponding physical/software functions of the device.

User controlled input actuator may be provided. An autonomy kill switch may include one or more of the following: button that removes all autonomy from the system, e.g., regardless of the current step or state the system; button that removes autonomy from the system, e.g., after it has completed its current activity; multiple state button (e.g., state one for the button allows autonomy to finish its current activities and state two for the button removes all autonomy immediately regardless of current activities); or configurable Button (e.g., the button may be configured for how it should operate such as removal of autonomy immediately or at completion of activities).

Sensed event related to the device may be used, which may include one or more of the following energy events: initial sensed force contact, rate of force increase, impendence thresholds, thermal spread/damage, or jaw temperature. For example, as the jaws are closed, the first timing that the current through the motor may be sensed and may be used to determine tissue height. If that was determined thick for the end-effector, the motor speed may be changed based on that threshold. Triggers may be used to cause alteration to autonomy. For example, a too thick condition may cause the device to stop automation and request user input.

Impedance may be used as a trigger. For example, when energy activated within the jaws, the generator may monitor the impedance of the tissue and/or vessel to determine when energy should be turned off. A product code may be indicated for a maximum vessel sealing size (e.g., typical 5 mm or 7 mm size). An energy algorithm may account for impedance over time. If the energy activation cycle was too short compared to a normal cycle, this may indicate and/or signal damaged and/or diseased tissue and indicate/notify the user to take over activation control. If the energy activation cycle was longer compared to a normal cycle, this may indicate and/or signal that tissue is fatty and/or is a larger vessel size than the product is indicated for and may transfer responsibility to the surgeon. Built-up tissue on the jaws may alter the impedance control. When this condition occurs based on calculated time verse actual, it may notify the user to clean the jaws. In examples, a harmonic 7 may be operated at a power level 3 only for vessels 5 mm in diameter and an advance hemostasis mode for vessel sizes 7 mm. In such a case, the system may autonomously select/alter the power level based on identified vessel size. When a vessel is above or between sizes, the system may request from the user which power level to proceed with.

Thermal spread may be used as a trigger. During energy activation and vessel sealing, criteria that may be monitored along with hemostasis is thermal damage and/or thermal spread. Temperature and/or lateral thermal damage may be considerations for surgeons when using energy-based technologies. Surgeons may be concerned about injury to nearby structures either by direct contact or by the visually unrecognizable transmission of energy. Surgeons may be concerned about the potential impact of tissue damage on the inflammatory response and overall recovery of the patient. Monitoring thermal spread during energy activation, along with the tissue type, thickness, power level, and/or clamp pressure, may lead to calculating the actual verse nominal, which may be used to notify user to and/or to autonomously adjust power settings and/or clamp pressure (e.g., Harmonic ACE+7 2.54+/−0.48 mm, which refers to mean & std. deviation). Preclinical comparisons of caprine vessel sealing may be used when autonomously adjusting. Thermal damage may result from the generation of heat by an advanced energy device and may be a critical component to vessel sealing and/or tissue transection. The temperature the instrument reaches may be dependent on multiple variables, including tissue type, tissue thickness, energy used, and/or power setting. An advanced energy devices may reach instrument temperatures of at least 100° C. during activation on tissue. There may be situations when the temperature of an ultrasonic device may reach beyond the 100° C. range due to tissue conditions. When jaws reach this temperature, the surgeon may unintentionally make contact with unintended tissue and cause trauma. When this occurs, this may trigger a wait time until the jaw temperature is reduced to an acceptable temperature. In such a case, if it made contact with tissue no trauma would occur or if the surgeon attempts to move the device, the autonomy control may take over the finite control (e.g., if the surgeon moved the device distal with a fast and/or larger motion than allowed, the autonomy may slow down and/or reduce the displacement to ensure the high temperature jaws may not make contact with the tissue and/or unintended treatment area).

Stapling device may be used as trigger. For example, an unanticipated articulation force may be used.

Automatic risk determination level may be provided. User choices may be compared to a benchmark on other facility users or a global user choice level to determine permission level associated with autonomy level. Active surgeon choices may be monitored control the autonomous choices. Setting uncertainty level or pre-selected user risk level may be used to determine acceptable risk level in its decisions. Previous operations or outcomes may be used to determine what the effective risk level may be.

Risk matrices for a system to use to determine appropriate risk may be provided. The matrices may include medical or patient (e.g., Claims History such as International Classification of Diseases, Tenth Revision, Clinical Modification (ICD010-CM), hierarchical condition category (HCC), electronic health records (‘HER-health information technology (HIT) database and/or the like).

The overall risk determination may be a combination of the risks associated with the patient, device and procedure.

Adjustment of the level of autonomous function may be based on historic user control, previous autonomous operation, and/or historic outcomes resulting from previous autonomous determination. Autonomous failures and/or user overrides/assistance may reduce the level of autonomy and/or the frequency of suggested autonomous operation. Undesirable outcomes may adjust the level of autonomous engagement (e.g., if user errors caused it, less autonomous engagement when compared to if autonomous operation caused it). Historic user interactions that resulted in confusion, misuse, or instructions-for-use issues may result in the system assisting with autonomous prompting and/or control to minimize future issues with use, for example, delay of procedure counting.

Aggregation of data may be utilized to adjust autonomy levels. For example, weighted comparison of deciding how to react to historic information may be used. Criticality of the failure, local frequency, uniqueness of the failure, etc. may be used. Uniqueness may include compete motor failure and/or complete reset of the system (e.g., RF interference with the system operation resulting in a complete restart to continue use).

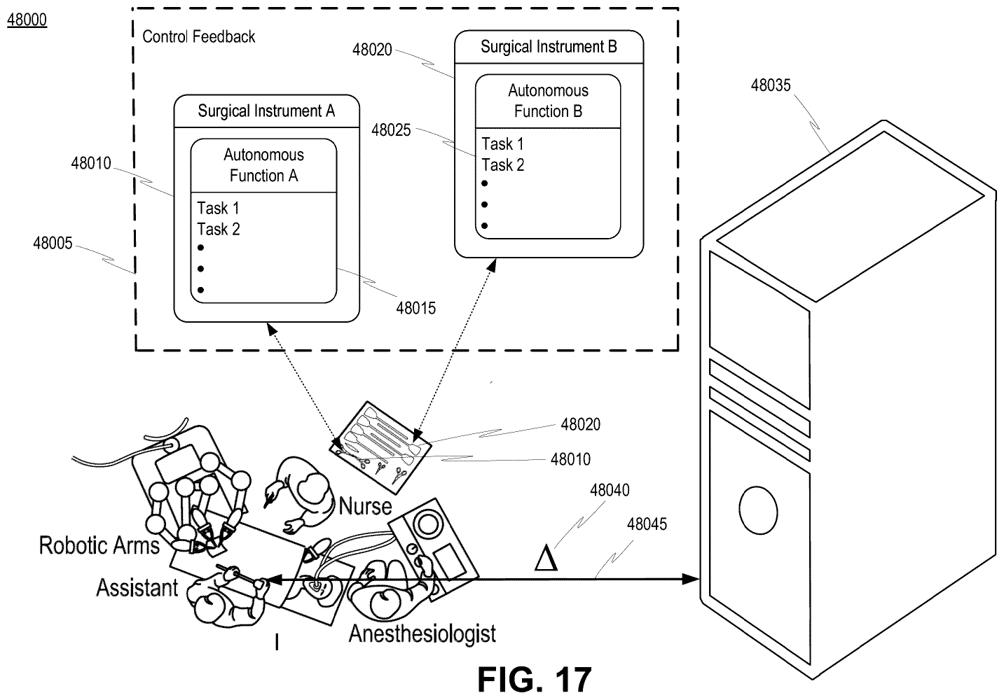

Referring to FIG. 17, an overview of the surgical autonomous system 48000 may be provided. Surgical instrument A 48010 and/or surgical instrument B 48020 may be used in a surgical procedure as part of the surgical system 48000. The surgical hub 48035 may be configured to coordinate information flow to a display of the surgical instrument. For example, the surgical hub may be described in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Example surgical instruments that are suitable for use with the surgical system 48000 are described under the heading “Surgical Instrument Hardware” and in U.S. Patent Application Publication No. US 2019-0200844 A1 (U.S. patent application Ser. No. 16/209,385), tided METHOD OF HUB COMMUNICATION, PROCESSING, STORAGE AND DISPLAY, filed Dec. 4, 2018, the disclosure of which is herein incorporated by reference in its entirety, for example.

FIG. 17 shows an example of a surgical autonomous system 48000. The system 48000 may be used to perform a surgical procedure on a patient who is lying down on an operating table in a surgical operating room. A robotic system may be used in the surgical procedure as a part of the surgical system. For example, the robotic system may be described in U.S. Patent Application Publication No. US 2019-0200844 A1 (U.S. patent application Ser. No. 16/209,385), tided METHOD OF HUB COMMUNICATION, PROCESSING, STORAGE AND DISPLAY, filed Dec. 4, 2018, the disclosure of which is herein incorporated by reference in its entirety. The robotic hub may be used to process the images of the surgical site for subsequent display to the surgeon through the surgeon's console.

Other types of robotic systems may be readily adapted for use with the surgical system 48000. Various examples of robotic systems and surgical tools that are suitable for use with the present disclosure are described in U.S. Patent Application Publication No. US 2019-0201137 A1 (U.S. patent application Ser. No. 16/209,407), tided METHOD OF ROBOTIC HUB COMMUNICATION, DETECTION, AND CONTROL, filed Dec. 4, 2018, the disclosure of which is herein incorporated by reference in its entirety.

Various examples of cloud-based analytics that are performed by the cloud, and are suitable for use with the present disclosure, are described in U.S. Patent Application Publication No. US 2019-0206569 A1 (U.S. patent application Ser. No. 16/209,403), tided METHOD OF CLOUD BASED DATA ANALYTICS FOR USE WITH THE HUB, filed Dec. 4, 2018, the disclosure of which is herein incorporated by reference in its entirety.

In various aspects, an imaging device may be used in the surgical system and may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The one or more illumination sources may be configured to radiate electromagnetic energy in the visible spectrum as well as the invisible spectrum. The visible spectrum, sometimes referred to as the optical spectrum or luminous spectrum, is that portion of the electromagnetic spectrum that is visible to (e.g., can be detected by) the human eye and may be referred to as visible light or simply light. A typical human eye will respond to wavelengths in air that are from about 380 nm to about 750 nm.

The invisible spectrum (e.g., the non-luminous spectrum) is that portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device may be configured for use in a minimally invasive procedure. Examples of imaging devices suitable for use with the present disclosure include, but not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), tided METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” i.e., an operating or treatment room, necessitate the highest possible sterility of all medical devices and equipment. Part of that sterilization process is the need to sterilize anything that comes in contact with the patient or penetrates the sterile field, including the imaging device and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

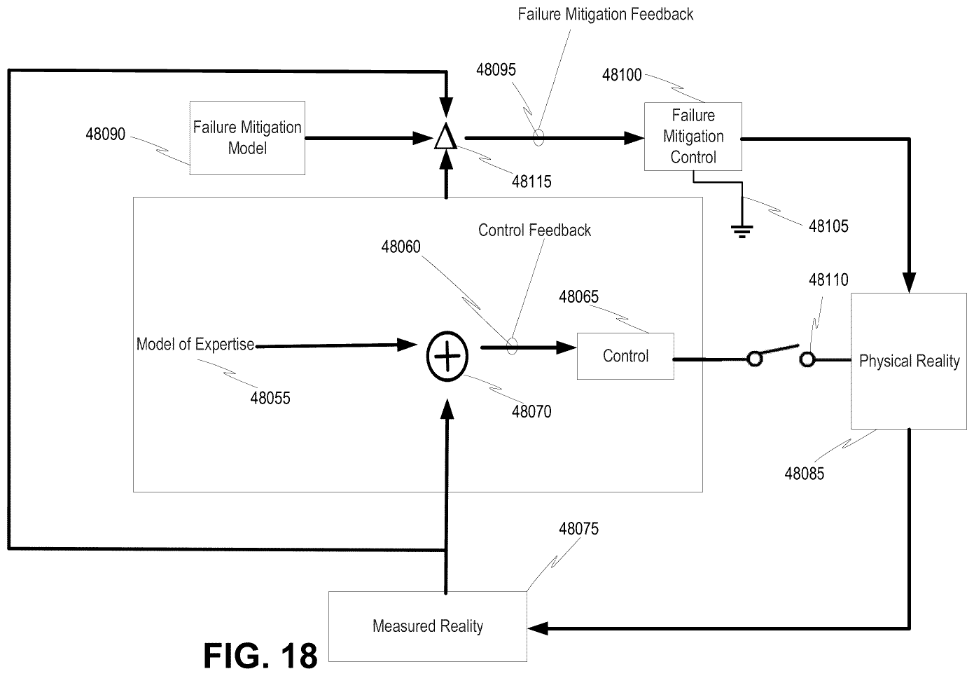

Surgical instrument A 48010 and/or surgical instrument B 48020 may comprise one or more capabilities. (e.g., surgical instrument A comprises capabilities B, Z, and D and surgical instrument comprises capabilities C, F, and E). The capabilities may be associated with features (e.g., autonomous function A 48015 associated with surgical instrument A and autonomous function B 48025 associated with surgical instrument B) that the surgical instrument is capable of performing. Examples of the features may be described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example. For example, if the surgical instrument is an endocutter, one of the capabilities may be resecting tissue (e.g., tissue surrounding a colon when the surgeon in performing a colorectomy). The capabilities may comprise surgical tasks as described with respect to FIG. 18. For example, the capability of resecting tissue may comprise controlling an energy source, cutting, stapling, knob orientation, body orientation, body position, anvil jaw force, reload alignment slot management, and/or the like.

Surgical instrument A and/or B may be associated with control feedback or failure mitigation feedback, as described herein. For example, an autonomy level with the least manual input may be associated with the control feedback. For example, an autonomy level with increased manual input may be associated with the failure mitigation feedback.

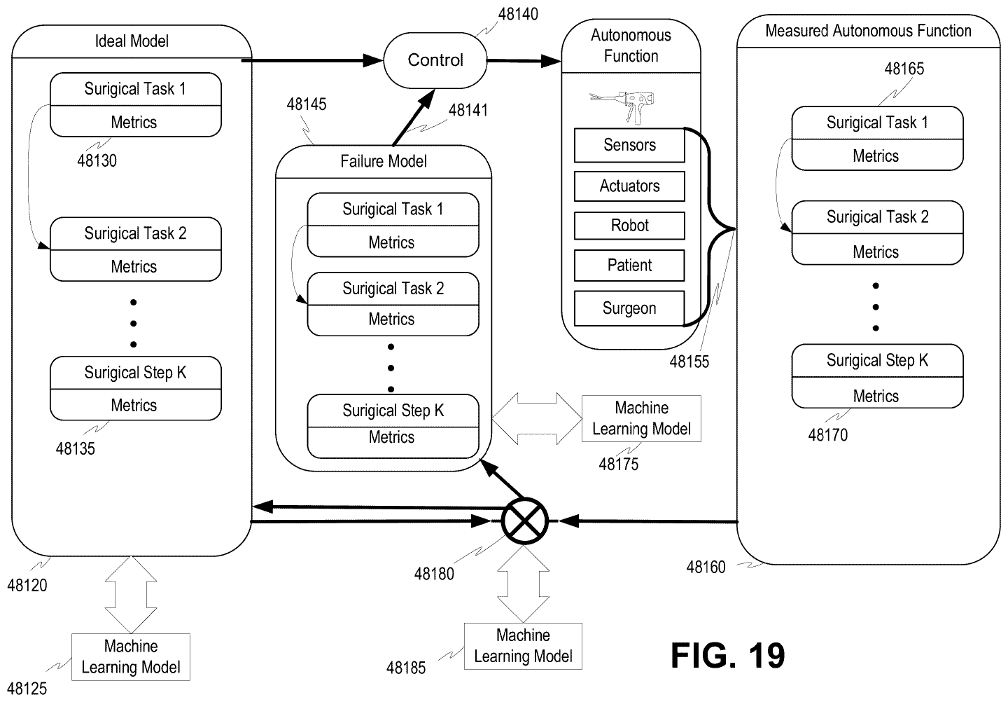

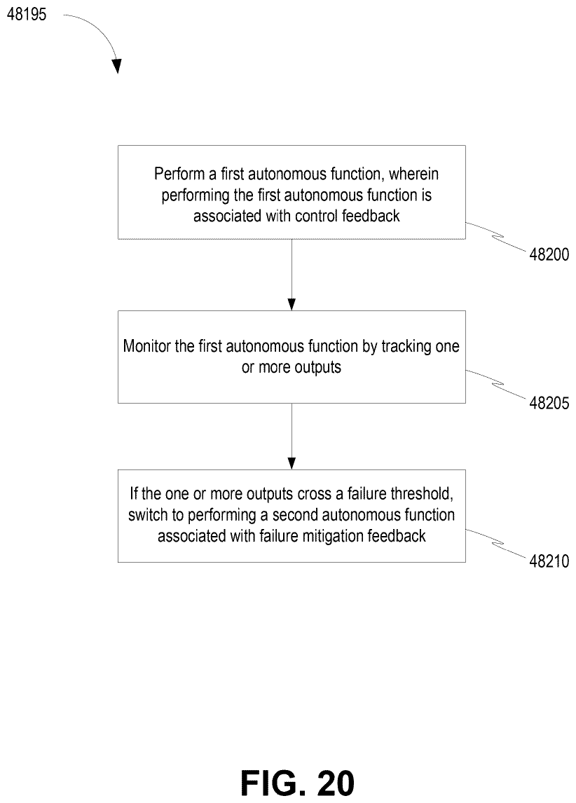

Data may be generated (e.g., by a monitoring module located at the surgical hub 48035 or locally by the surgical instrument as described with respect to FIG. 20) based on the performance of surgical instrument A and/or B. The data may be relevant to how the current level of autonomy at which the surgical instrument is operating is doing in terms of performance. For example, the data may be associated with physical measurement physiological measurements, and/or the like as described with respect to FIGS. 19 and 20. The measurements are described in greater detail under the heading “Monitoring Of Adjusting A Surgical Parameter Based On Biomarker Measurements” in U.S. patent application Ser. No. 17/156,28, filed Nov. 10, 2021, the disclosure of which is herein incorporated by reference in its entirety.

An indication 48045 of the data may be transmitted to the surgical hub 48035, for example, where it may be evaluated. In examples, the indication 48045 may be transmitted to a cloud service (e.g., Amazon Web Services) as described herein. The data may be used as input in an analysis module, for example, to check if the performance of the surgical instrument falls within a satisfactory range (e.g., crosses a failure threshold 48040). The indication may be associated with switching from using the control feedback 48005 to the failure mitigation feedback.

FIG. 18 shows the relationship between control feedback 48060 and failure mitigation feedback 48095. As shown in FIG. 18, the device may include a model of expertise 48055. The model of expertise 48055 may provide parameter(s) such that a surgical instrument may be operated autonomously. For example, the surgical instrument may perform (e.g., be capable of performing) one or more surgical tasks autonomously. For example, the model of expertise 48055 may be linked to a machine learning algorithm. The machine learning algorithm, as described herein, may include a neural network structure that may generate parameter(s) (e.g., weights of parameters) to be used, for example, on a script located internally in the surgical instrument. In examples, the script may be executed remotely, for example, at the surgical hub. The parameters outputted by the model of expertise 48055 and executed by the script may be dynamic. The script may be used to execute one or more actuators, as described with respect to FIG. 6, located on the surgical instrument. For example, the one or more actuators may be linear actuators and/or rotary actuators.

The actuators may be servomotors located on the surgical instrument. The script may be generated using a machine learning algorithm with adjustable parameters and may be used in connection with hardware, such as linear and rotary actuators, to allow the surgical instrument to perform a surgical task. For example, the script may produce control feedback 48060, which may be used as input to the actuators. The actuators may allow the surgical instrument to be controlled autonomously via the script. For example, the actuators may receive the adjustable parameters as input and use the parameters to perform the surgical task autonomously.

Physical reality measurement(s) 48085 may be obtained. For example, physical reality measurements 48085 may relate to one or more sensors placed on the surgical instrument performing the surgical task. In examples, physical reality measurements 48085 may be obtained and/or may be generated from a patient wearing wearable sensors that measure the patient's biomarkers. In examples, the physical reality measurements 48085 may be generated from a surgeon wearing wearable sensors that measure the surgeon's biomarkers and other health markers.

The physical reality measurements 48085 may be sent to a surgical hub and may be analyzed at the surgical hub. In examples, the physical reality measurements 48085 may be generated locally at the surgical instrument, for example, by the surgical instrument processor, as described herein. In examples, the surgical instrument and/or surgical hub may include an analysis module that may analyze the physical reality measurements 48085. If the physical reality measurements 48085 are generated by a remote source, such as a surgical hub, the physical reality measurements 48085 may be sent via a message to the surgical instrument. In examples, the message may pass through a surgical application program interface (API). The physical reality measurements 48085, or in other words, the data related to the physical reality measurements may be compared to model measurements (e.g., model data) as described herein.

The physical reality measurements 48085 may be translated into measured reality data 48075. For example, the physical reality measurements 48085 may be passed through an analysis module located at the surgical hub or locally at the surgical instrument. The output of the analysis module may be data related to the measured reality. For example, the physical reality measurements 48085 may be raw data related to the position of the linear and/or rotary actuators of the surgical instrument. For example, this data may be in the form of voltage readings. In examples, an analog-to-digital converter (ADC) may be included and may transform the voltage readings into a bitstream, which may be used as the physical reality measurements 48085. This raw data may be sent to an analysis module located at the surgical hub or locally at the surgical instrument and the analysis module may output measured reality data 48075. Measured reality data 48075 may in a form better suited for comparison when compared to the raw physical reality measurements 48085.

The measured reality data 48075 may be compared to data associated with the model of expertise. For example, the model of expertise may include a data structure that holds model data. The model data may be referred to as with model reality data herein. This model reality data may be compared to measured reality data 48075 and a difference 48070 between the two may be generated. This difference 48070 between the model reality data and the measured reality data 48075 may be used as control feedback 48065 to adjust parameters that control the servomotors, such as the linear and rotary actuators described herein.

For example, the difference 48070 between the model reality data and the measured reality data may indicate that the speed at which the rotary actuators rotate is too high. The measure reality data 48075 associated with rotational speed may be greater than the model reality data associated with rotational speed for rotary actuator A positioned at a certain spot on the surgical instrument. Based on this indication, control feedback 48065 may be generated in which a parameter associated with the speed of the rotary actuator A may be decreased such that the speed of the rotary actuator is decreased.

The model of expertise 48055 may be used to generate the control feedback 48065 for the control of the actuators of the surgical instrument operating autonomously, e.g., as long as the difference 48070 between the modeled reality measurements data and the measured reality data 48075 falls within a threshold. If the difference 48070 between the two does not fall within a threshold 48115 (e.g., exceeds a threshold 48115), a failure mitigation model 48090 may be used (e.g., control loop grounded to zero 48110 and failure mitigation loop release from grounded zero 48105).

The failure mitigation model 48090 may generate parameters to be used by the surgical instrument operating autonomously. The parameters may be set to values associated with no risk. In examples, the failure mitigation model 48090 may turn the surgical instrument from autonomous to a manual setting. The surgical instrument using the failure mitigation parameters may control the linear and/or rotary actuators and other motor controls based on the failure mitigation feedback 48095.

Physical reality measurements 48085 may be generated to assess the performance of the failure mitigation control 48100. Similar to the model of expertise 48055, physical reality measurements 48085 may be in the form of raw data and may pass through an analysis module. The analysis module may generate measured reality data 48075, which may be used and compare against failure model data. The measured reality data 48075 that are associated with failure mitigation parameters may be compared to failure model reality data, for example, to assess the performance of the failure mitigation model 48090.

A notification of the switch from the model of expertise 48055 to the failure mitigation model 48090 may be sent via a message to a user of the surgical instrument. The message may include the parameters that are being set for the surgical instrument. In examples, when comparing the measured reality data 48075 to the model reality data, a heart rate of the patient may be compared. This data related to the heart rate may be checked against a threshold 48115. If the heart rate exceeds the threshold, the surgical instrument may switch from using a model of expertise script to a failure mitigation model script. The failure mitigation model 48090 may produce failure mitigation parameters. The parameters may be known to result in the surgical instrument being able to perform a surgical task with no risk involved.

FIG. 19 shows an example of performing an autonomous function with an ideal model 48120 and a failure model 48145, As shown in FIG. 19, an ideal model 48120 may include one or more surgical tasks. Ideal model 48120 and model of expertise may be used interchangeably herein. The ideal model 48120 may be generated by a surgical hub. For example, the ideal model 48120 may be generated by the surgical hub as described with respect to FIG. 18. The surgical tasks (e.g., each of the surgical tasks) of the ideal model, for example, surgical task one 48130, surgical task two, and surgical task K 48135, may be associated with one or more ideal metrics. The ideal metrics may be associated with ideal physiological and/or physical metrics related to the performance of the surgical task. For example, the surgical task may be performed autonomously. The ideal metrics may be related to physiological and/or physical expected conditions that the surgeon may expect to be present based on the surgical task. For example, the surgical task may be removing surrounding tissue from an organ, such as a colon. The ideal physical conditions and/or metrics for this surgical task may include the patient's heart rate being above a certain value and below a certain value (e.g., within an acceptable range). This may be considered the ideal heart rate range for mobilizing the colon. In examples, the surgical task may be resecting a tissue, which may involve the use of an endocutter. Resecting tissue may include one or more instrument capabilities being performed such as positioning the instrument body, controlling anvil jaw force, and managing reload alignment slot. These capabilities may be performed autonomously. For example, a first autonomous function may be associated with (e.g., may be a software module located within) the model of expertise as described herein. To perform the capabilities for the surgical task of tissue resection, the model of expertise may run the first autonomous function (e.g., the script of the autonomous function). Based on a failure mitigation threshold having been crossed, a second autonomous function may be called. The second autonomous function may be associated with (e.g., may be a software module located within) the failure mitigation model. To perform the capabilities for the surgical task of tissue resection, the model of expertise may switch from running the first autonomous function (e.g., the script of the first autonomous function) to running the second autonomous function (e.g., the script of the second autonomous function).

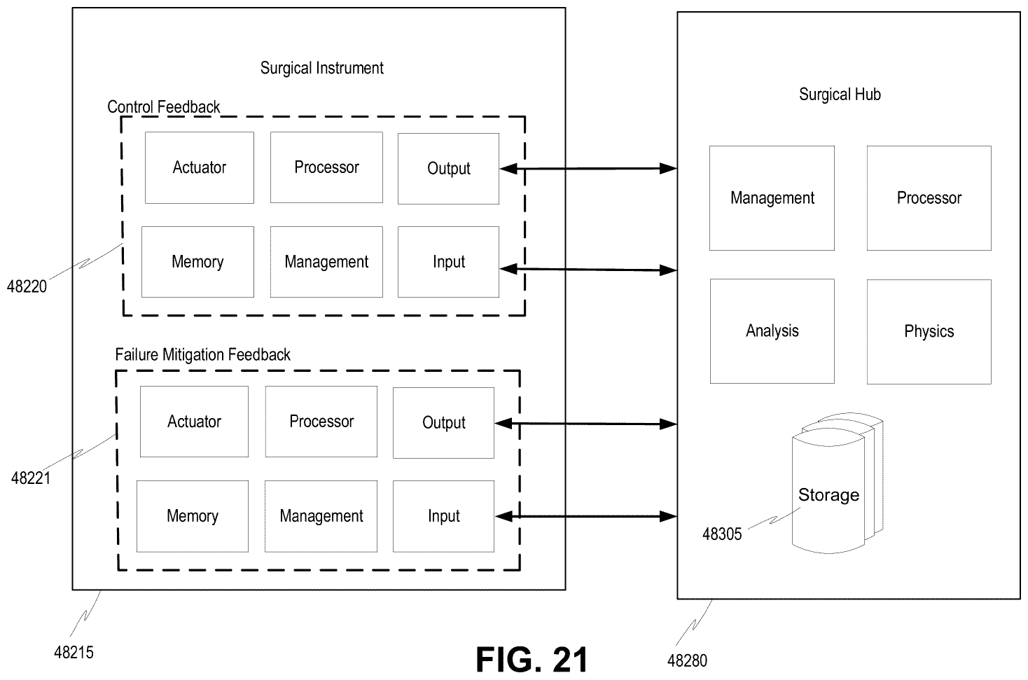

The physical and/or physiological ideal conditions may change based on the surgical task. For example, surgical task two may have different ideal physiological and/or physical conditions when compared to surgical task one. The physical metrics may include data generated from sensors from one or more actuators as described with respect to FIG. 18 and FIG. 21. In order for the surgical instrument to perform an autonomous function, the surgical instrument may perform surgical task one 48130, surgical task two, and/or surgical task K 48135. The ideal physical and/or physiological conditions may be compared to measured physiological and/or physical conditions, which, e.g., as shown with respect to the measured autonomous function 48160, the measured physiological and/or physical conditions may be generated 48155 from one or more of the following: sensors, actuators, data generated from robotic functions, data generated from the patient, such as physiological data, data generated from the surgeon, such as physiological surgeon data.

The data generated 48155 from the patient and/or surgeon may involve the use of wearable devices to measure biomarkers associated with the patient and/or surgeon. The wearables are described in greater detail under the heading “Monitoring Of Adjusting A Surgical Parameter Based On Biomarker Measurements” in U.S. patent application Ser. No. 17/156,28, filed Nov. 10, 2021, the disclosure of which is herein incorporated by reference in its entirety.

This data may be generated 48155 and used as an input to gather the physiological and/or physical metrics. The data associated with the ideal physical and/or physiological metrics may be used and compared to the data generated from the measured physical and/or physiological conditions (e.g., for each of the surgical tasks such as surgical task 1 48165, surgical task 2, and/or surgical task K 48170 associated with the measured autonomous function 48160 and a difference between the two may be generated and compared to a threshold 48180, which may take place at the surgical hub, or locally at the surgical instrument, as describe with respect to FIG. 18 and FIG. 21.

Determining the threshold 48180, e.g., at which the difference between the ideal physical and/or physiological conditions and the measured physical and/or physiological conditions is compared to, may be generated using a machine learning model 48185, which may include a neural network structure. The neural network structure may use one or more of the ideal physical and/or physiological conditions as parameters when determining the appropriate threshold level 48180. If the threshold level 48180, e.g., which may be referred to as the failure mitigation threshold level 48180, as described with respect to FIG. 21, has been exceeded based on the difference between the ideal physiological and physical conditions and the measured physical and physiological conditions, the surgical instrument may switch to using a failure model 48145.

The failure model 48145 may involve the surgical instrument being associated with failure mitigation feedback. The failure model 48145 may include physiological and/or physical failure metrics such as failure physical and/or physiological conditions. Similar to the ideal model, these conditions may be compared to measured physiological and physical conditions in the described herein. Determining both the ideal physical and physiological conditions and the failure physical and physiological conditions may involve the use of a machine learning model 48175 as described herein. For example, the machine learning model 48175 associated with the failure mitigation model 47145 may determine failure mitigation metrics that eliminate or minimize any risk associated with performing one or more surgical tasks (e.g., surgical task 1, 2, and/or K).

The machine learning model 48125 associated with the ideal model may determine one or more physical and/or physiological metrics associated with an optimized performance of the surgical task. The optimized performance may consider costs and/or benefits associated with the surgical task. For example, the surgical task one may involve the surgeon mobilizing a patient's colon. During the surgical task, a surgeon's heart rate may have an ideal physiological measurement. A patient's heart rate may have an ideal physiological measurement. The position of the instrument may have an ideal physical measurement, which may be determined by the position of one or more actuators. In examples, this data may be analyzed and a value may be generated. In examples, each of the ideal data collected from physiological and/or physical conditions may be compared to respective measured physiological and/or physical conditions. For example, the surgeon's ideal heart rate may be compared to the surgeon's measured heart rate as determined during surgical task one of the autonomous function. The patient's heart rate may be compared to the measured patient's heart rate during surgical task one 48130. In examples, if one of the differences between the ideal model physical condition and the measured model physical condition and/or physiological condition exceeds the failure mitigation threshold 48180, the surgical instrument may switch from operating on control feedback 48140 to operating on failure mitigation feedback 48141. The failure mitigation feedback 48141 may be described with respect to FIG. 18. In examples, when switching from surgical task one 48130 to surgical task two, a set of ideal physical and/or physiological metrics may be generated by the ideal model, which may be located on the surgical instrument and/or on a surgical hub.

In examples, the surgical instrument may pull data from a remote source to generate ideal metrics associated with surgical task two, which may be based on historical data associated with surgical task two. The historical data may be based on a facility's historical data or may be based on a general historical data associated with surgical task two. The physical and/or physiological conditions and/or metrics associated with the physical and/or physiological conditions may be manually inputted by a surgeon or another user of the surgical instrument.

FIG. 20 shows an example of performing an autonomous function with control feedback and failure mitigation feedback. An overview 48195 of performing an autonomous function with control feedback and failure mitigation feedback may be provided. Autonomous function and autonomous surgical task may be used interchangeably herein.

At 48200, a first autonomous function may be performed. The first autonomous function may be associated with the model of expertise as described with respect to FIG. 18. For example, the model of the expertise may output the parameter(s) to be used as the autonomous function is performed. The autonomous function may perform a surgical task associated with a surgical instrument. The controls of the surgical instrument such as the actuators described with respect to FIG. 18 and FIG. 21 may be controlled by control feedback. The control feedback may update the parameters based on the current performance of the autonomous function.

The control feedback may be generated when comparing modeled reality data associated with the model of expertise with measured reality data that is generated based on the physical and/or physiological measurements associated with performing the surgical task. For example, the physical measurements may include the position of the actuator(s) located on the surgical instrument performing the surgical task.

At 48205, the first autonomous function, which may be associated with the model of expertise, may be monitored. For example, one or more outputs may be generated based on the performance of the autonomous function.

The outputs may be the physical measurements and may be in the form of raw data. The outputs may be fed into an analysis module either at a surgical hub or locally at the surgical instrument. The outputs may include data generated based on situational awareness, for example, surgical context data may be sent to the surgical hub. Data generated based on situation awareness may include any data suitable for characterizing the current presentation of the patient in view of the ongoing surgical procedure. For example, such data may include procedural stage, current operating room (OR) arrangement, patient vitals, instruments, engaged, and/or the like. The surgical hub may use the surgical context data when generating measured reality data. The surgical context data, for example, may be sent to analysis module along with physical measurement data.

The model of expertise may generate parameters by using a machine learning model. The parameters may be used to perform the autonomous function. When generating the parameters to be used for the autonomous function, the machine learning model may consider one or more of the following: historical data; surgical context; the type of the first autonomous function, the type of the second autonomous function, historical data associated with the first autonomous function, number of outputs, or the degree of the one or more parameters that have crossed the failure threshold. Threshold and failure threshold may be used interchangeably herein. The one or more of the following described herein may apply when determining whether the difference between the measured reality data and the model reality data has crossed the failure threshold. The first autonomous function may be monitored (e.g., as described with respect to FIG. 18, the analysis module may output measured reality data which may be compared to model reality data and a difference between the two may be produced). The difference between the two may be compared to a failure threshold.

At 48210, if it is determined that the failure threshold has been exceeded, the surgical instrument may switch to performing a second autonomous function. The second autonomous function may be associated with the failure mitigation model. The failure mitigation model may produce failure mitigation feedback which may result in adjusting the parameters to a value which results in the surgical instrument being able to perform the surgical task with no risk involved. In examples, a difference between the measured reality data and the model reality data may be generated for each physical measurement obtained from the model of expertise. A physical measurement related to the speed of a rotary actuator may be generated. This data may be processed in an analysis module. Measured reality data related to the speed of the rotary actuator may be outputted from the analysis module.

Model data related to the speed of the rotary actuator may be contained in the model of expertise as a data structure such as in a list. The speed of the rotary actuator from the model data structure may be compared to the actual speed of the rotary actuator in the measured reality data and a respective output may be produced based on the difference between the two and compared to a respective failure threshold. This may be performed for multiple physical measurements which may produce respective measured reality data, which may be compared to respective model reality data. The differences may be compared to respective thresholds (e.g., failure thresholds).

In examples, if one of the respective differences between the measured reality data and the model reality data crosses the respective threshold, the surgical instrument may switch from the first autonomous function to the second autonomous function. The number of differences that have to cross the output before switching to the second autonomous function may be manually inputted by a user of the surgical instrument. For example, a surgeon may set three as a number of times the failure threshold must be exceeded by respected differences before switching to the second autonomous function.

The second autonomous function may be associated with the failure mitigation function as described herein. The difference between the measured reality data and the model reality data may be a single value. For example, the model of expertise may include an analysis module that may assess the difference between each measured reality data and each model reality data and may come up with a value based on the difference between the two. In such a case, the single difference from all of the respective measured reality data and the model reality data may be compared to a single failure threshold. When the single failure threshold is exceeded, the surgical instrument may switch from the first autonomous function to a second autonomous function to perform the surgical task.

A failure magnitude may be generated based on the difference. For example, a failure magnitude may be based on the degree at which the difference between the measured reality data and the model reality data crossed the failure threshold. For example, if the difference between the measured reality data and the model reality data exceeds the failure threshold by a greater amount, the failure magnitude may be higher than if the difference exceeds the failure threshold at a lesser amount. The failure magnitude may be used when generating the failure mitigation parameters. For example, the failure mitigation parameters may be dynamic and may operate within a range. A higher failure magnitude may result in the surgical instrument using parameters that result in less risk in terms of the of the completion of the surgical task. In examples, the failure magnitude may be used to switch the surgical instrument from autonomous to manual.

In examples, failure magnitude may be used to terminate all operations related to the surgical task. The failure magnitude may be generated based on one or more of the following: surgical context; the type of the first autonomous function; the type of the second autonomous function; historical data associated with the first autonomous function; the number of outputs that cross the failure threshold; or the degree at which one or more of the outputs crossed the failure threshold. Outputs may mean the difference between the measured reality data and the model reality data. In examples, instead of generating a difference between the measured reality data and the model reality data, the measured reality data may be compared to the threshold. From this comparison, it may be determined whether the threshold was crossed and, in such a case, the surgical instrument may switch from the first autonomous function to the second autonomous function.

Weights may be assigned to the outputs (e.g., each of the outputs) of the analysis module and/or to the measured reality data (e.g., each of the measured reality data). The speed of the actuators may be assigned a higher weight when compared to the position of the sensors (e.g., potentiometers). Assigning the weight may be performed by the analysis module either locally at the surgical instrument or on the surgical hub. Assigning the weight may take place on a remote service such as a cloud service. For example, the cloud service may be the Amazon Web Services lambda function. When comparing whether the difference between the model reality data and the measured reality data crossed the failure threshold, the weights may be considered. For example, if the difference between the measured reality data associated with the speed of the actuators crossed the failure threshold, it may be more likely that the surgical instrument switches to the second autonomous function. If the difference between the model reality data and the measured reality data associated with the position of the actuators crossed the failure threshold, the surgical instrument may be less likely to switch to the second autonomous function. Assigning the weights may be based on one or more of the following: historical data, surgical context, type of the first autonomous function, type of the second autonomous function, historical data associated with the first autonomous function, the number of the outputs that cross failure threshold (e.g., number of differences between the measure reality data and the model reality data that crossed the threshold); or a degree of the difference of the outputs crossing the failure threshold.

As described with respect to FIG. 18, a message may be sent to a user of the surgical instrument if the surgical instrument switches from the first autonomous function which is associated with the model of expertise and the second autonomous function, which is associated with the failure mitigation model. A set of recommendations may be included in the message to the user (e.g., surgeon). For example, the recommendations may include which measured reality data has exceeded the threshold and may suggest how the autonomous function may use failure mitigation feedback to prevent any complications. In examples, the surgical instrument may switch back to the first autonomous function which is the autonomous function associated with the model of expertise. These switches may occur multiple times during the performance of a surgical task. These switches may be based on the difference between the measured reality data and model reality data falling within or below a threshold. In examples, a risk assessment model may be included and used when determining whether to switch between autonomous functions.

Generating the recommendations may be based on the type of the first autonomous function, the type of the second autonomous function, historical data associated with the first autonomous function, the difference, the number outputs that cross the failure threshold, and/or the degree at which the outputs crossed the failure threshold. The model of expertise may initialize the parameters based on training data it received prior to performing the surgical task. The training data may be based on one or more of the following: surgical context, type of the first autonomous function, type of the second autonomous function, historical data associated with the first autonomous function a number of outputs that crossed the failure threshold or a degree of the one or more outputs crossing the threshold.

FIG. 21 shows an example of the overview of the surgical instrument 48215 with control feedback 48220 and the failure mitigation feedback 48221 and the surgical hub 48280. As shown with respect to FIG. 21, a surgical instrument 48215 may be in communication with a surgical hub 48280. In examples, the surgical hub 48280 may be a third-party service such as an edge service on a cloud platform.

The surgical instrument 48215 may include control feedback 48220 and failure mitigation feedback 48221. The control feedback 48220 may be used while performing an autonomous function associated with a surgical task. For example, if the surgical task is mobilizing a colon, the autonomous function may be freeing the colon from surrounding tissue, which may involve the use of an endocutter. The control feedback 48220 may set, e.g., via a message to the surgical instrument 48215, the parameters for the endocutter to perform the autonomous function, e.g. freeing the colon from surrounding tissues. The control feedback 48220 may adjust the one or more parameters (e.g., the autonomous parameters).

The parameters may be used as input for the actuators, which may include the one or more actuators described with respect to FIG. 18. The parameters may be adjusted based on feedback from a machine-learning model. For example, an error rate may be measured based on the performance of the autonomous function and if the error rate exceeds a threshold as assessed by a machine-learning model, feedback may be sent to the surgical instrument to adjust the one or more autonomous parameters.

The control feedback 48220 may include a memory. The memory may store the one or more current parameters. The memory may store the parameters via a database as described with respect to FIG. 18 and FIG. 19. Control feedback 48220 may include a management module. The management module may be responsible for sending an update message to the memory if the one or more autonomous parameters are updated. The control feedback may include a processor which may be responsible for running the instructions to perform the autonomous function.

The processor may be in communication with the memory. The memory may include an instruction memory which the processor may read or write to. The control feedback 48220 may be associated with the normal operation of the autonomous function to perform the associated surgical task (e.g., autonomously). The control feedback may have one or more autonomy levels. Switching autonomy levels may result in adjusting the one or more autonomous parameters. The autonomy level may be stored in the memory along with the one or more autonomous parameters associated with the autonomy level.

Control feedback 48220 within the surgical instrument may generate output. The output may be related to the performance of the autonomous function. The output may include the one or more parameters of the autonomous function. The output may be generated and may be sent via a message to the surgical hub 48280. The surgical hub 48280 may perform analysis on the output. The surgical hub 48280 may send a response message. In such a case, the surgical instrument 48215 may receive input data from the surgical hub 48280 via the response message. The input data may include an indication to update the one or more autonomous parameters associated with the autonomous function.

The machine-learning model, as described herein, may be located within the surgical hub 48280. For example, the machine-learning model may be a module within the surgical hub 48280. Storage 48305 may be included in the surgical hub. In examples, the storage 48305 may be off-disk storage. A management module of the surgical hub 48280 may be responsible for sending data associated with the output data of the control feedback 48220 to the storage 48305. The output data, as described herein, may be associated with a performance of the autonomous function and/or the parameters of the autonomous function. Such data may be stored in the storage 48305 along with a timestamp of when the output data was generated.

The management module of the surgical hub 48280 may send such data to the storage 48305. In examples, the analysis module may be included in the surgical hub 48280. The analysis module may compare the performance of the autonomous function as generated by the output module to a threshold (e.g., a failure mitigation threshold). The analysis module may determine whether the output data exceeds the failure mitigation threshold. In such a case, if such data does exceed the failure mitigation threshold, the surgical hub 48280 may send a message to the surgical instrument 48215 to switch from using control feedback 48220 to failure mitigation feedback 48221. The failure mitigation feedback 48221 may be the failure mitigation feedback as described with respect to FIGS. 18, 19 and 20.

The failure mitigation feedback 48221 may include one or more actuators. The one or more actuators may be the same actuators as described with respect to the control feedback 48220. The parameters used for the one or more actuators may be failure mitigation parameters. The failure mitigation parameters may be set to a value where no risk or minimum risk is expected based on the performance of the autonomous function. For example, the failure mitigation parameters may include values that result in 100% success rate of the autonomous function. The failure mitigation parameters may be stored in a memory associated with the failure mitigation feedback 48221. A processor of the surgical instrument may request those values when performing the autonomous function. Failure mitigation feedback may be data generated by the failure mitigation model (e.g., using the script associated with the second autonomous function) that is sent (e.g., signaled) to the hardware of the surgical instrument, for example, to control the surgical instrument autonomously as it performs the surgical task. The data generated may be set to values that ensure no risk, or at least reduced risk, is involved in the autonomous performance of the surgical task.

The failure mitigation parameters may be used as input for the actuators to perform the surgical task at hand. The failure mitigation parameters may be sent from the surgical hub 48280. The performance of the surgical instrument 48215 that is using the failure mitigation parameters may generate output data associated with the performance, which may be sent to surgical hub 48280 and analyzed. The analysis module may assure no risk is involved in the performance of the autonomous function.

The surgical hub 48280 may detect that risk is still present based on the failure mitigation parameters and/or the performance of the failure mitigation feedback output. The surgical hub 48280 may adjust the failure mitigation parameters, which may be sent via a message and received as input by the surgical instrument 48215. The failure mitigation parameters may be updated in memory. The management module associated with the failure mitigation feedback 48221 may be responsible for updating the memory. In order to perform the autonomous function with failure mitigation feedback 48221, the processor may write or read to the memory associated with the failure mitigation feedback 48221.

Pro-active monitoring and/or reactions may be based on encountered issues (e.g., automated issue resolution). Autonomous function oversight monitoring and/or failure mitigation may be provided, which may include monitoring of an autonomous function, magnitude and/or level for identification of failures of an autonomous operation (e.g., portion of an autonomous function). An identified failure may have a determination of importance of the failure based on risk, redundancy, timing of occurrence, magnitude of occurrence, or impact of the occurrence. The identified failure may be followed up with an indication of the failure to the user and/or to a system outside of the hub system. The indication may result in active correction of the issue. The indication may be to the user, the facility, maintenance, the manufacturer, and/or complaint database. The indication may result in a mitigation of the failure, and the result of the mitigation may be part of the indication to one or more systems and/or users. The mitigation may reduce system performance, for example, to enable the conclusion of the step or procedure. It may be shut-down the affected portion of the system. It may include a substitution of an alternative means of monitoring and/or controlling the medical hardware. It may allow for a pause and/or hold of instrument motions, for example, until the system failure is resolved.

Reporting may be provided, which may include complaint and/or issue automatic documentation. Complaint and/or issue automatic documentation may include one or more of the following: a user guided form and/or template identification and assistant may be used in completion; complaint categorization and/or routing (e.g., manufacturer directed issues and/or facility directed issues); machine learning aggregation and sorting for trends; or automated reporting and/or summarization. Complaint and/or issue automatic documentation may include the surgical hub being fully aware if a device within the operating room theater has had malfunction or error condition. This information may be autonomously pulled and pushed into the complaint database for review. The information may be categorized a number of ways. For example, a way may be to have the hub look into the device records and determine the severity (e.g., DFMEA severity) of the failure and use this as a category. A way may be to use the hospitals risk systems to base the categorization against.

Automated recording and/or reporting of failures, their potential causes, associated operation circumstances, resolutions or mitigation, and user interactions or experiences may be due to the failure. The system may record (e.g., automatically record) the log data (e.g., all log data). Data may be stored to a database associated with that system. In examples, the system may record (e.g., automatically record) the log data for a given period of time. For example, for rollover recording log, the system may log (e.g., automatically log) all data (e.g., regardless of other severity, criticality, etc.) for a given period of time. As the time period elapses, the system may automatically start to overwrite the oldest data first to preserve memory space. For period of accountability, the system may automatically log all data for a given period of time, for example, that it may be legally required and may automatically discard the data after that period of time has elapsed. The data that has been identified may be exempted from the automatic discard feature. For patient outcome, the log data may be held from the time of surgery, for example, until the outcome of the patient has been ascertained. If an adverse event for that patient is identified, the data may be held and not removed. If no adverse events are identified, after a certain period of time post surgery, the log data may be discarded.

Backup and/or auditing of the automation post operation may be used to review and/or document automated functions and their impact on the user, procedure step, and/or device outcome. User monitoring of reactions and response to an autonomous operation may be provided. Overriding of the primary response may be provided. For example, steps that the surgeon deviates from may be flagged for review. For example, steps within the procedure that the system believes are a risk and the surgeon deems it acceptable may be annotated as an override by the user and the number of these triggers may be used as a trigger for auditing (e.g., AI review, peer review, variations away from the IFU, etc.). A robotic system may perform intended procedure steps and a device failure may occur. It may be determined that the robotic system is not at fault, which may include fault logging and/or event logging. End of procedure auditing of autonomous operation may be logged based on procedure review, user reactions, and/or monitored outcomes to record success, failure, user choices, and/or impacts on subsequent steps. For example, automatic annotation of the recorded image with temporal overlay and device automation flags may be placed to provide context and situational awareness of the operation.

The control system (e.g., local facility and/or global main system) may be alerted of encountered issues. Intended use, indication of use and contraindications for device used during surgery may be provided. The medical devices may indicate within the IFU its intended use, indication of use and contraindications. During use of the devices (e.g., each device) the system may identify whether violations to the IFU were predicted to occur and may alert the user prior to the event. Provisions may be in place to override the system if surgeon deems it appropriate. For example, the control system may alert that the device is not to be used on ischemic tissue near the colon. The surgeon's determination may be that the area is the best place to proceed. The surgeon may override the system to continue. The event may be logged to be analyzed after the procedure. A number of firings for a device may be exceeded. It may be determined whether buttressing material was used for any of the firings which may impact total #of firings allowed. The procedure may have total procedure time constraints. For example, if the device has been set up for too long, the system may alert that elapsed time has been exceeded. The surgeon may override if decision to use is decided. Tissue may not be placed and oriented correctly in the jaws. Tissue placement and thickness may be determined. Insertion of a counterfeit cartridge may be determined. It may be ensured that the device is cleaned prior to cartridge reloading. The materials of the devices may be monitored to identify whether a patient has a hypersensitivity prior to use of the device and/or may alert the surgeon prior to the surgery. The issues may be aggregated into classifications. The classifications may include priority and/or criticality of failure, user confusion, and/or hardware failure (e.g., product inquiry). There may be escalation between classifications (e.g., frequency and/or severity). Low risk hardware issues may have redundant systems in place. If repeated use of redundant systems is initiated, it may use that as a trigger that the primary system is encountering issues. If the redundant system is only occasionally utilized, it may signify a necessary system or module reboot which may be considered standard operation

Self-service problem solving of the hub and attached systems may be provided Automation of image search features may be used within a procedure database for solution options. Images captured from the scope may be bounced off the database to identify healthy vs diseased tissue as confirmation prior to device activation of energy and/or stapling to minimize leaks and/or complications. In examples, it may be used to identify an optimal next step based on issue to alert the surgeon. For example, if the surgeon completed a staple line and perfusion occurred, it may alert the surgeon of the best approach to address the leak based on tissue type known historical procedures. For example, the hub may provide a recommendation (e.g., in the form of an SNS message) that that suturing may be the best technique to address leak or that an energy modality that minimizes time may provide the best technique. Images may be confirmed to a database for navigation confirmation. For example, as the scope is navigating to the intended site, it may have confirmation/verification checks of images from the scope compared to a known database to confirm the surgeon is going in the correct direction and alert when off course. Network connection resolution may be provided. The magnitude of the failure may be automatically defined and an appropriate response to an experienced issue may be used to handle the issue relative to functional operation and needs on the system. For example, this may be used for failures in operating room displays. If a current operating room display is deemed defective during the procedure, the system may transfer the contents of display to alternative displays. In examples, it may split-up information. The system may place an order for defective components. The maintenance may call set-up for repair of faulty equipment. The system may alert issues for tracking. Low-risk hardware issues may have redundant systems in place. Major faults and minor faults and the automated responses by the hub may be provided. For example, the hub may experience an issue with the video output. The power supply to the system may fail causing the total loss of video feeds. This may be classified as a major fault condition. Overlay system failures may include software errors. To ensure that the correct input is selected, the hub system may accommodate various forms of output from visualization systems. Control measures may ensure that the correct input (e.g., one that has video running) is selected for output. The display of the overlay may be stopped if the main fails (e.g., it cannot be trusted.) In such a case, it may be executed on a softcore or hardcore redundant processor. Overlay system failures may include hardware failures. Redundant hardware may be used to maintain full functionality of both systems and may include a voting system (e.g., similar to full-redundant FCS). Redundant hardware may provide full video function without providing overlay function. For a switch over hardware solution, the system may have redundant power supplies. The solution being difficult to achieve may result in it being an active solution (e.g., too many standards to switch over).

Overlay system failures may include power supply failures. In examples, a visualization system may be provided. The visualization system may fault. For example, the video may be frozen (e.g., but otherwise correct looking), which may be associated with the highest risk. A fault may be no video, fuzzy or interrupted video, monochrome video, color changed video (e.g., lowest risk), etc. Video mitigation may be provided. For example, a control measure may be to provide sufficient visualization to safely remove the instruments from the patient, after which a different visual platform may be found. This control measure may include a device that may process the raw MIPI sensor output from the visualization system and may render a Bayer pattern on the monitor. For example, white light Bayer pattern sensors may output luminance values for pixels (e.g., pixel), line-by-line and frame-by-frame, regardless of the color of the pixel. A series of red, green and blue filters on individual pixels may filter the incoming white light to provide intensity values for those colors. The image signal processing may use the Bayer pattern signal to calculate the missing color data for the pixel location (e.g., each pixel location). This process may be called demosaicing. By taking the raw sensor signal, placing it in memory, reading it from memory at the timing required for an HDMI display, and outputting it to a display, a rudimentary monochrome image may be produced to allow safe removal of the instruments from the patient. Quickboot or archiving of operational use conditions just prior to the failure or restart may be used to restore operation to the previous state. System resource management may be provided.

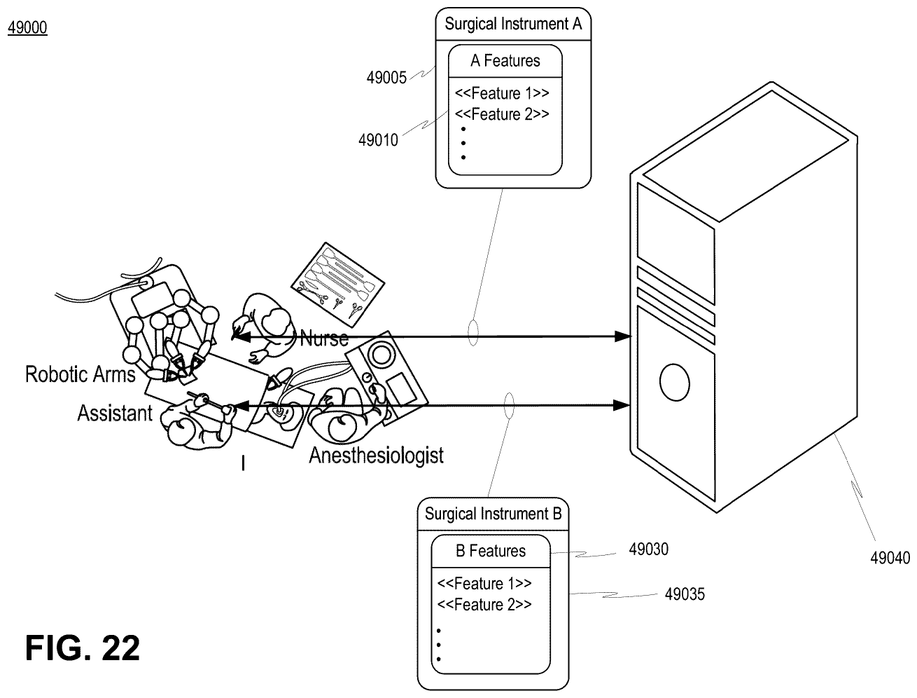

Referring to FIG. 22, an overview of the surgical autonomous system 49000 may be provided. Surgical instrument A 49005 and/or surgical instrument B 49035 may be used in a surgical procedure as part of the surgical system 49000. The surgical hub 4900 may be configured to coordinate information flow to a display of the surgical instrument. For example, the surgical hub may be described in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Example surgical instruments that are suitable for use with the surgical system 49000 are described under the heading “Surgical Instrument Hardware” and in U.S. Patent Application Publication No. US 2019-0200844 A1 (U.S. patent application Ser. No. 16/209,385), tided METHOD OF HUB COMMUNICATION, PROCESSING, STORAGE AND DISPLAY, filed Dec. 4, 2018, the disclosure of which is herein incorporated by reference in its entirety, for example.

FIG. 22 shows an example of a surgical autonomous system 49000. The system 49000 may be used to perform a surgical procedure on a patient who is lying down on an operating table in a surgical operating room. A robotic system may be used in the surgical procedure as a part of the surgical system. For example, the robotic system may be described in U.S. Patent Application Publication No. US 2019-0200844 A1 (U.S. patent application Ser. No. 16/209,385), tided METHOD OF HUB COMMUNICATION, PROCESSING, STORAGE AND DISPLAY, filed Dec. 4, 2018, the disclosure of which is herein incorporated by reference in its entirety. The robotic hub may be used to process the images of the surgical site for subsequent display to the surgeon through the surgeon's console.

Other types of robotic systems may be readily adapted for use with the surgical system 49000. Various examples of robotic systems and surgical tools that are suitable for use with the present disclosure are described in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

Various examples of cloud-based analytics that are performed by the cloud, and are suitable for use with the present disclosure, are described in U.S. Patent Application Publication No. US 2019-0206569 A1 (U.S. patent application Ser. No. 16/209,403), tided METHOD OF CLOUD BASED DATA ANALYTICS FOR USE WITH THE HUB, filed Dec. 4, 2018, the disclosure of which is herein incorporated by reference in its entirety.

In various aspects, an imaging device may be used in the surgical system and may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The one or more illumination sources may be configured to radiate electromagnetic energy in the visible spectrum as well as the invisible spectrum. The visible spectrum, sometimes referred to as the optical spectrum or luminous spectrum, is that portion of the electromagnetic spectrum that is visible to (e.g., can be detected by) the human eye and may be referred to as visible light or simply light. A typical human eye will respond to wavelengths in air that are from about 380 nm to about 750 nm.

The invisible spectrum (e.g., the non-luminous spectrum) is that portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device may be configured for use in a minimally invasive procedure. Examples of imaging devices suitable for use with the present disclosure include, but not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), tided METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” i.e., an operating or treatment room, necessitate the highest possible sterility of all medical devices and equipment. Part of that sterilization process is the need to sterilize anything that comes in contact with the patient or penetrates the sterile field, including the imaging device and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

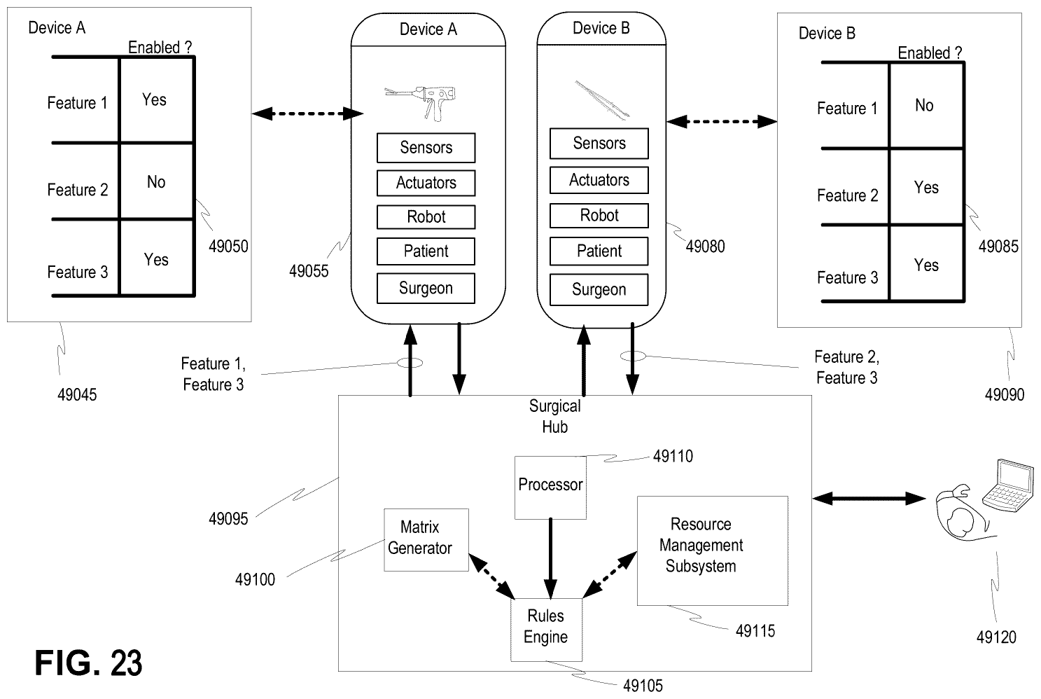

Surgical instrument A 49005 and/or surgical instrument B 49035 may comprise one or more capabilities. (e.g., surgical instrument A comprises capabilities B, Z, and D and surgical instrument comprises capabilities C, F, and E). The capabilities may be associated with features (e.g., A features 49010 associated with surgical instrument A 49005 and B features 49030 associated with surgical instrument B 49035) that the surgical instrument is capable of performing. Examples of the features may be described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example. For example, if the surgical instrument is an endocutter, one of the capabilities may be resecting tissue (e.g., tissue surrounding a colon when the surgeon in performing a colorectomy). The capabilities may comprise surgical tasks as described with respect to FIG. 23. For example, the capability of resecting tissue may comprise controlling an energy source, cutting, stapling, knob orientation, body orientation, body position, anvil jaw force, reload alignment slot management, and/or the like.

Data may be generated (e.g., by a monitoring module located at the surgical hub 49040 or locally by the surgical instrument) based on the performance of surgical instrument A 49005 and/or B 49035. The data may be relevant to how the surgical instrument is operating is doing in terms of performance. For example, the data may be associated with physical measurement physiological measurements, and/or the like. The measurements are described in greater detail under the heading “Monitoring Of Adjusting A Surgical Parameter Based On Biomarker Measurements” in U.S. patent application Ser. No. 17/156,28, filed Nov. 10, 2021, the disclosure of which is herein incorporated by reference in its entirety.

An indication of the data may be transmitted to the surgical hub 49040, for example, where it may be evaluated. In examples, the indication may be transmitted to a cloud service (e.g., amazon web services) as described herein. The data may be used as input in an analysis module.

FIG. 23 shows an example of an overview of an adapting interconnected surgical system. As shown in FIG. 23, one or more surgical instruments may be used to perform a surgical task. For example, device A 49045 may be associated with a surgical instrument. Device B 49090 may be associated with a second surgical instrument. The surgical instruments may include one or more features. For example, device A 49045 and/or B 49090 may be associated with one or more features (e.g., device A features 1, 2, and 3 49050 and device B features 1, 2, and 3 49085). The one or more features may be features that are to be performed autonomously (e.g., during the surgery). For example, a feature of device A 49045 may be the orientation of device A 49045. If this feature is enabled, as shown in FIG. 23, the orientation of device A 49045 may be determined (e.g., controlled) autonomously by the system.

If a feature is not enabled, the feature may be performed manually by a surgeon. A feature associated with device B 49090 may be clamping. If this feature is enabled, clamping for device B 49090 may be performed autonomously. If this feature is not enabled, clamping may be performed by a surgeon performing the surgical task. Enabling and disabling the one or more features associated with each of device A 49045 and device B 49090 may be dynamic. For example, while transitioning stages during the performance of a surgical task, the surgical hub 49095 may disable and/or enable one or more of the features associated with device A 49045 and/or device B 49090 based on an introduction or removal of surgical instruments into the surgical OR or based on an introduction of a new surgical system into the surgical OR.

Enabling and/or disabling may be determined based on metrics (e.g., device A metrics 49055 and device B metrics 49080) of the surgical instruments (e.g., how device A 49045 performed its feature, whether it is enabled and on autonomous mode or disabled and on manual mode). In such a case, the metrics may be generated based on one or more of the following sources, which may be located on the surgical instrument: sensors, actuators, robotic metrics, patient biomarkers, force, surgeon biomarkers, surgical context, number of staff, change in number of staff, etc. The patient and/or surgeon biomarkers may be associated with measurements taken from wearable devices in which the patient and/or surgeon may wear during the performance of the surgical task. The actuators may include one or more servomotors located on the surgical instrument. The metrics data associated with these categories may be transmitted to the surgical hub 49095 via a message (e.g., JSON message). The message may pass through a surgical application interface. The surgical hub 49095 may receive this data and may process the data via the surgical hub processor 49110.

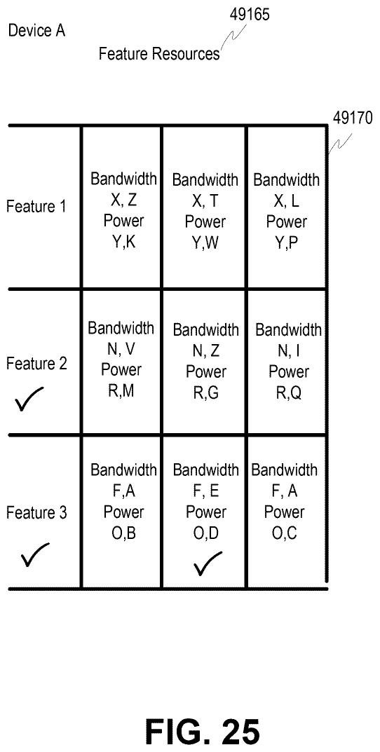

The one or more features may be enabled and/or based on available resources associated with the system. For example, the available resources may be stored in a resource management subsystem 49115 of the surgical hub. Device A 49045 and device B 49090 may use resources from the resource management subsystem 49115 to perform one or more of their features (e.g., autonomously). In examples, the surgical hub 49095 may determine which of the features to enable for the surgical instruments based on the resources available. The surgical hub 49095 may query the resource management subsystem 49115 in order to determine the resources available (e.g., total resources available), which may be described with respect to FIGS. 5 and 6. Determining which features to enable may involve the use of a rules engine 49105. The rules engine 49105 may be located within the surgical hub 49095. In examples, the rules engine 49105 may be located on a third party service. The features may be gathered by the surgical hub 49095 and used to generate a matrix (e.g., using a matrix generator 49100), as described with respect to FIG. 25.

For example, feature 1 of device A 49045 may be associated with (e.g., use) a certain amount of bandwidth and a certain amount of power. This information may be used by the surgical hub 49095 in order to generate a matrix, which may be, for example, a data structure that links the features of each surgical device to the resources (e.g., resource ranges) needed in order to perform the feature. The rules engine 49105 may be responsible for assessing different variations of allocating resources to features and may optimize which features to enable based on an evaluation at the surgical hub 49095. The features that are determined to be enabled may be sent to the surgical instruments via a message. In examples, the resources and/or features of the surgical instruments, may be manually inputted by medical staff member 49120.

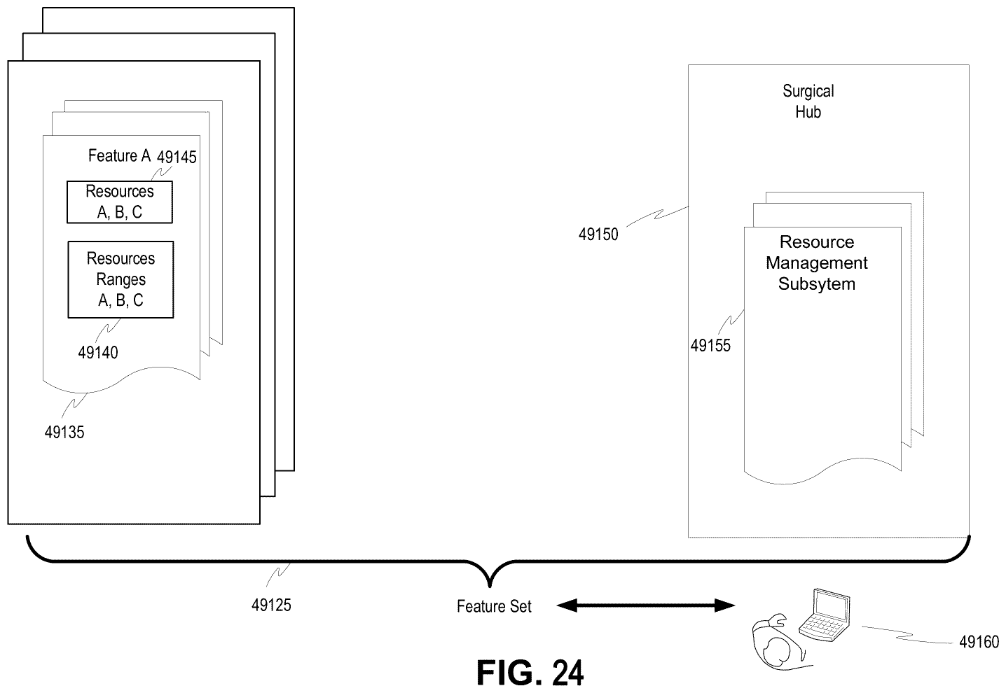

FIG. 24 shows an example of the relationship between feature sets 49125, surgical hub 49150, and resource management. A feature set 49125 may be included in the system. A feature set 49125 may be manually inputted by a user 49160, such as a surgeon or medical staff member. In examples, the feature set 49125 may be a learned set determined using a machine learning algorithm.

One more features may be associated with a surgical instrument. For example, as described with respect to FIG. 2, the one or more features may be functions that the surgical instrument may be able to perform autonomously. If the feature is enabled, this may suggest that the surgical instrument performs this function autonomously, for example, clamping.

The one or more features may be associated with one or more resources 49145. For example, feature A 49135 may be associated with resources 49145 A, B, and C. In examples, the features may be associated with resource ranges 49140. The resource range 49140 may be a range at which the surgical instrument is able to perform the function autonomously (e.g., the range may be include a value indicating an expected performance of the function, which may be based on historical data and surgical context). For example, there may be a minimum resource and maximum resource. In examples, there may be an optimal resource.

The one or more features which are linked to resources 49145 and/or resource ranges 49140 may be transmitted to a surgical hub 49150. The surgical hub 49150 may store and analyze such values in a resource management subsystem 49155, which may include one or more different data structures to hold and to query the features, the resources 49145, and the resource ranges 49140. The features which are enabled may be adjusted (e.g., by changing an autonomy level associated with the feature) based on introduction or removal of one or more surgical instruments or surgical systems. A higher level of autonomy may be associated with a more tasks of the feature being performed autonomously and a lower level of autonomy may be associated with less tasks of the feature being performed autonomously.

In examples, a second surgical hub may be introduced into the surgical OR. The second surgical hub may provide more allowable resources 49145 to be used by the surgical instruments, e.g., already present in the surgical OR. In such a case, more features may be enabled due to the increased allowable resources 49145 as determined by the rules engine of the surgical hub 49150.

Making the determination whether to enable and/or disable one or more features may be performed by the rules engine, as described with respect to FIG. 6. As the instrument (e.g., new instrument) or system (e.g., new system) is introduced, it may register with the surgical hub 49150. The surgical hub 49150 may configure the system or surgical instrument and may notify the other surgical instruments and/or systems already present in the surgical OR. The surgical instruments may have an identification associated with it, as well as the surgical systems, which may be assigned by the surgical hub 49150 at the time of registration.

A device (e.g., new device) and/or system may request (e.g., send a request message) to the surgical hub 49150 asking for permission to connect. The request message may indicate which resources 49145 a system (e.g., new system) has available to be used. A surgical instrument (e.g., new surgical instrument) may send a request message to the surgical hub 49150 asking to connect. The surgical instrument may indicate in the request message which features it is able to do autonomously along with the number of resources 49145 for the features.

The hub 49150 may determine, based on the current setup, whether to allow the surgical system and/or new surgical instrument to connect. After connected, the surgical hub 49150 may make a determination whether to adjust which features are enabled and/or disabled for the current system and may send such determination via a message to the surgical instruments. Based on receiving the message, surgical instruments may follow through and enable and/or disable the features indicated by the surgical hub 49150.

Enabling and/or disabling features may take into account performance metrics, such as latency. The surgical hub 49150 may include an optimization module, which may run cost analysis by enabling and/or disabling a variation of the features in determining an optimal feature set to use for the surgical task at hand.

FIG. 25 shows an example of a matrix generator 49170 and feature determination. As shown in FIG. 25, feature resources 49165 may be associated with a surgical instrument, for example, device A. Feature resources 49165 may be associated with the matrix generated with respect to FIG. 23. For example, feature resources 49165 may show the matrix that was generated, which shows the link between features of device A and resources, as shown in FIG. 25 (e.g., feature one of device A). As the device is connected to the surgical hub, the device may send the features along with the resources associated with the device to the surgical hub as described with respect to FIG. 24. The resources may be the resources used in order to perform the feature of the surgical instrument autonomously. As shown in FIG. 25, feature one of device A may be associated with bandwidth X. The matrix generator 49170 may combine the resources of a secondary device introduced into the surgical hub and run an optimization technique to figure out which features should be enabled in order to best perform the surgical task at hand.

For example, device A feature one may be associated with bandwidth resource (e.g., associated with bandwidth, power, payload effort, size of data packet, transmit frequency, etc.) X and the matrix generator 49170 may input device B's bandwidth of Z and the rules engine (e.g., which obtains this combination from the matrix generator) may determine a cost of enabling feature one with this combination. The matrix generator 49170, in a second variation, may use feature one's bandwidth of X with another device with bandwidth of T and the rules engine may determine a cost associated with this combination. In examples, the matrix generator 49170 may use feature one's bandwidth of X to be combined with a new device introduced into OR which has a bandwidth of L and the rules engine may determine a cost of this combination. After assessing these different variations, the surgical hub may determine (e.g., via the rules engine) whether to enable feature one with a particular tested combination of resources based on a cost analysis, which may be included in an analysis module. If a surgical system is introduced into the OR, feature one's bandwidth of X may be used in a matrix generator 49170 along with a bandwidth (e.g., total bandwidth) of the surgical system, which may be used to determine whether feature one may be enabled.

As shown in FIG. 25, another resource that may be used to determine whether features one, two, or three are enabled may be power. Feature one of device A may be held constant while introduced devices associated with power resources may be used in the matrix generator 49170 and a cost associated with each combination may be determined and used to determine whether feature one should be enabled. This technique may be used for features two and three. For example, in order to perform feature two autonomously for device A, it may use a bandwidth of N. The matrix generator 49170 may hold the bandwidth constant while combining different variations of other devices' bandwidth to determine whether feature two should be enabled. After the features are determined by the surgical hub or a third-party service, the surgical hub may send a message to each of the surgical instruments on which features to enable and may send a notification to a user (e.g., surgeon). In examples, the surgical hub may send a message to a user telling them to disable one or more features manually based on the introduction or removal of a surgical instrument or a surgical system.

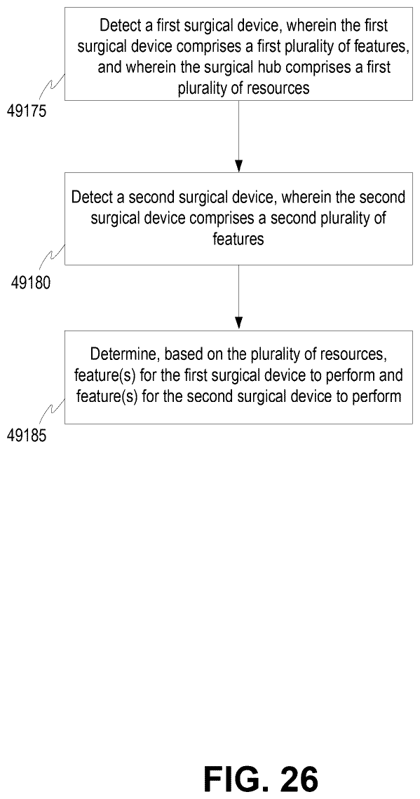

The cost analysis performed by the rules engine may include a measure of the performance of the surgical task. For example, the surgical hub may run a simulation (e.g., locally or remotely via a cloud service) with each of the resource combinations generated by the matrix. Simulation framework may be described in “Method for Surgical Simulation” in U.S. patent application Ser. No. 17/332,593, filed May 27, 2021, the disclosure of which is herein incorporated by reference in its entirety. The performance metrics may be gathered based on the simulation. For example, a CPI may be determined as well as a latency and throughput. An overall score may be generated for each of the combinations and the surgical hub may determine the maximum score and use the combination of features that resulted in the maximum score for the surgical task simulation. In examples, determining which combination should be used may involve a rules engine assigning weights to performance metrics and other considerations associated with the surgical task. Determining the rules may be based on one more of the following: surgical context, type of features, historical data, or types of resources, as described with respect to FIG. 26. For example, situational awareness may provide the system with additional context not privy to a device (e.g., each device) which may allow the system to optimally adapt. Having awareness of surgical context may provide advantage for the system to adjust appropriately. Historical data may show procedure risk is higher for a certain patient. Historical data may include global and/or patient specific information.

FIG. 26 shows a flow chart of an adapted interconnected surgical system. A surgical hub may be used for autonomous determination of surgical features in a surgical environment. At 49175, the surgical hub may (e.g., include a processor configured to) detect a first surgical device in the surgical environment. The first surgical device may include a first plurality of features. The surgical hub may include a plurality of resources.

At 49180, the surgical hub may detect a second surgical device in the surgical environment. The second surgical device may include a second plurality of features.

At 49185, one or more features may be determined from the first plurality of features for the first surgical device to perform based on the plurality resources. One or more features may be determined from the second plurality of features for the second surgical device to perform based on the plurality of resources.

In examples, the surgical hub may include a plurality of ports. The first surgical device may link to the surgical hub via a first port from the plurality of ports. The second surgical device may link to the surgical hub via a second port form the plurality of ports.

Determining the one or more features from the first plurality of features for the first surgical device to perform and the one or more features from the second plurality of features for the second surgical device to perform may involve using optimization (e.g., Newtonian method) associated with one or more rules. The optimization associated with one or more rules may be based on one or more of the following: surgical context, type of the first plurality of features, type of second plurality of features, historical data, or the plurality of resources.

In example, the surgical hub may determine a surgical context associated with the surgical environment. The surgical hub may determine the one or more features from the first plurality of features for the first surgical device to perform and the one or more features from the second plurality of features for the second surgical device to perform based on the plurality of resources and the surgical context.

The surgical hub may send an indication of the determined one or more features to a user of the first surgical device and a user of the second surgical device. The surgical hub may detect a third surgical device in the surgical environment. The third surgical device may include a third plurality of features. The surgical hub may adjust the one or more features from the first plurality of features for the first surgical device to perform and the one or more features from the second plurality of features for the second surgical device to perform based on the plurality of resources and the third plurality of features. One or more feature from the third plurality of features for the third surgical device to performed may be determined based on the plurality of resources.

The surgical hub may determine one or more optional features from the first plurality of features for the first surgical device to perform and one or more optional features from the second plurality of features for the second surgical device to perform based on the plurality of resources. The optional features may be selected by a user of the first surgical instrument and a user of the second surgical instrument. The surgical hub may determine one or resources from the plurality of resources for the first surgical device and one or resources from the plurality of resources for the second surgical device. The determined one or more resources may be used to perform the determine one or more features.

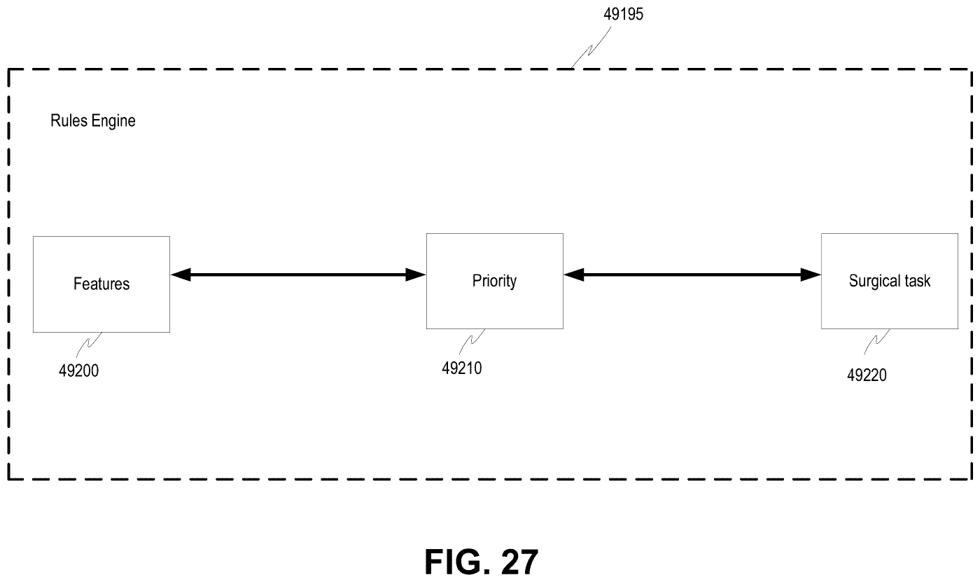

FIG. 27 shows an example of an overview of a rules engine 49195. In examples, a rules engine 49195 be included in a third-party system, which the surgical hub may have access to (e.g., using a security key). The rules engine 49195 may be linked to the matrix generator as described with respect to FIG. 23. The rules engine may assign weights based on the importance of the features 49200 of the surgical instrument that may be performed autonomously, as well as the resources associated with the surgical instruments. For example, feature one as described with respect to FIG. 25 may be given a higher weight when compared to feature two. Feature two as described with respect to FIG. 25 of device A may be given a higher weight than feature three. The weights may be determined based on surgical context and/or historical data. The weights may be determined based on results gathered from surgical simulations.

Weight may be assigned to performance metrics. Weights may be assigned to resources for each of the features 49205. For example, for feature one, as described with respect to FIG. 25, bandwidth may be assigned a higher weight when compared to power. As described with respect to FIG. 25, power for feature two of device A may be assigned a higher weight than bandwidth. This may depend on the type of feature 49200 that is being performed autonomously. For example, power may be assigned a higher weight for performing orientation autonomously when compared to the power used to perform clamping autonomously.

Based on the weights, a priority 49210 may be assigned to the resources associated with each of the features 49200. A high and/or low priority 49210 may be determined. A feature 49200 associated with a high priority 49210 may be chosen to be enabled over a feature 49200 associated with a low priority 49210. As the surgical task 49220 is performed and is executed, the weights may be dynamic based on surgical context. The rules engine 49195 may determine update weights for each of the features 49200 of the surgical instruments. The weights may be updated based on a new surgical instrument or new system being introduced into the surgical OR.

Autonomous system interactions may be provided. Adaptation of the autonomous options and/or processes may be based on the automatic identification and/or connection in between the system and secondary systems. Adaptation of automated functional options may be based on the interconnections of the systems. For example, the surgical hub may connect to one or more secondary systems and in establishing connections may adjust the operations that may be conducted autonomously. The adjustments may include setup, updating operational systems, designating communication priorities or processor load priorities, and/or establishing communication between secondary systems (e.g., two secondary systems) directly.

Autonomous communication establishment and/or registration may be provided. The surgical hub and/or instrument may be associated with a setup and/or a configuration at startup or access to a smart network. The surgical hub and/or or instrument setup and/or configuration at startup or access to a smart network may be described herein. In examples, if a device requests and/or establishes a connection to the hub defined network, the hub may define for the device a type of communication, frequency, system security level (e.g., causing the device to adjust its security level), flagging technique, revisions of software, etc. During this defining of communication exchange, the hub, based on the procedure, network backlog/latency, capacity level, and/or procedure, may adjust the anticipated intercommunication levels to the situation it is monitoring. The hub may define that multiple systems within its network communicate directly (e.g., using network bandwidth) and/or communicate through the hub (e.g., allowing it to record and adapt communication based on additional data), which may depend on the capabilities and capacities of the hub at the time and for the specific procedure. For example, the hub may determine the mode (e.g., best mode) of communications between devices (e.g., two devices) and may tell the devices how to communicate with each other. The hub may send flagging information to the devices to check back in with the Hub, for example, based on a certain change. The hub may request that the device adjusts the frequencies it operates on, the magnitude the system communicates at (e.g., in dB), and/or the protocols it uses based on the amount of noise and/or data detected within the OR by the hub. The hub may adjust numerous systems communication instructions based on the introduction of a device, the priority of its data or risk in its function relative to other systems within the network, to provide the highest priority or highest risk the data that uses the most capacity for interaction. It may define the communication interactions between systems (e.g., two systems) within its system to tune their communication. For example, the hub may detect a device's signal is decreasing or that the next step in the procedure uses a higher bandwidth to communicate the appropriate amount of data. The hub may enable an antenna (e.g., additional antenna) to try and increase the signal strength or it may tell the device to increase the signal output strength.

Connecting to an available network and/or requesting registration as a participant in the hub control matrix may be provided. Automated identification (ID) and/or registration may be based on detected aspects of the network and/or device. Unlinked and connectable surgical devices may be aware of the networks surrounding it. The awareness may allow for a selective interrogation of the possible connection options. The options may be compared to a pre-existing table or hierarchal order of networks it should have a preference for connecting to. The device may announce its intentions to connect and the network may provide information relevant to which of the networks are anticipating its arrival and/or of the ones that do not want it to interfere or try to connect to. This may be based on the procedure step, procedure progress, instruments in communication (e.g., currently in communications), arrival of duplicate devices or incompatible devices, and/or another situational use. Automated adaptation of a system function may be based on a parameter of the establishment of communication between systems. The parameter may be a step in the procedure. For example, on establishing the connection, the device may adapt the system if (e.g., only if) it occurs within a certain step in the procedure.

Automatically changing and/or updating software and/or functional testing the assembled configuration (e.g., after first assembly) may be used to confirm adaptation. The software may update the configuration settings. A validation of the update may be used. Functional check of the configuration may include one or more of the following. It may include verification of transducer function. It may include verification of torque. It may include articulation angle maximums. It may include re-establishing the device zero position and/or home. Shipping issues may cause the device to fall out of calibrations. For example, an energy device may be announced and/or detected by the hub. The hub may know the next step and/or use case (e.g., based on factory set configurations, surgeon selected/modified/machine, or clinical database learning) for the announced device in the procedure. The hub may automatically send configuration settings to the device for its activation.

Selective automatic establishment of communication between systems within the same operating room (OR) network may be provided, which may include distributed analysis, communication and data verification by co-networked OR devices. For example, interactive communication of operating parameters between a device and a hub controlling the procedure plan and/or flow of the surgery may include one or more of the following. A surgical stapling device may be preset at the factory to 15 mm/sec cutting speed by default. The stapler may be energized within the OR and may establish communication with the hub. The hub may recognize the device. The hub may analyze a database and determine that the optimal speed for this procedure is 5 mm/sec and a 20 second pre-compression delay. The hub may communicate to the device and send updates of the cutting speed and compression times. The device may use the parameters and check that the values are within the operation boundaries of the device system. The device may communicate back to hub that 5 mm/sec is on the lower bound of the systems motor performance, which may potentially result an insufficient sealing staple line delivery and/or device performance degradation. The hub may take the updated information and determine a risk profile at higher cutting speeds. An acceptable increase may be determined and passed back to the device. The device may verify the parameters and acknowledge to the HUB that it is ready. One or more of the techniques described herein may done autonomously. If an acceptable solution cannot be achieved, the HUB may alert the OR personal. Based on the risk levels of the patient, device and procedure, the system may set an overall procedure risk level. Depending on the calculated risk level, the system may complete the task autonomously or if the risk is deemed too high, the system may wait to be acknowledged by the surgeon before completing the procedure step. A mobile flexible endoscopic robotic may be rolled into the OR for a portion of trans-bronchial imaging and dissections and/or mobilization. If the system becomes aware it is operating in close proximity with the patient of other smart or digital eco-system device (e.g., lap retractors, staples, energy devices, etc.), it may establish communication with the devices when it controls the system close to the other systems to help avoid collisions or inadvertent tissue tension situations.

Autonomous identification of trackable and/or identifiable elements may be within system range. Automatic identification of wearable systems may include one or more of the following. It may include automatic determination of an active user of the wearable, which may involve using previous datasets to determine the user and/or automated verification and/or confirmation of identify (e.g., use of fingerprint of previous biomarker to verify the user). It may include categorization of user's characteristics such as job (e.g., skillset, competency level, equipment trained on, etc.), determination of use of monitored data, and/or prioritization of monitored data relative to other data sources simultaneously processed by the system. Automatic defining of reporting frequency, registration, and/or data collection parameters. Instrument, tool, medical stock identification may be provided, which may include detection of make, model, and/or serial number. Auto-determination of system limits or reach may include which systems the hub is allowed to control, acceptable reach for communication establishment, and/or physical and/or functional boundaries to track ability.

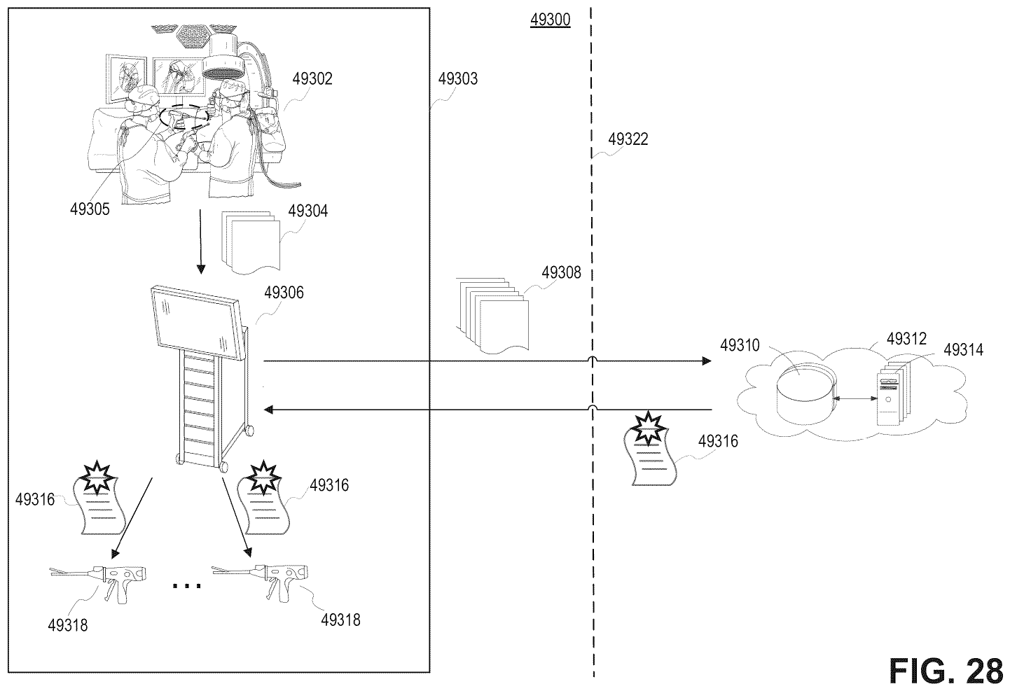

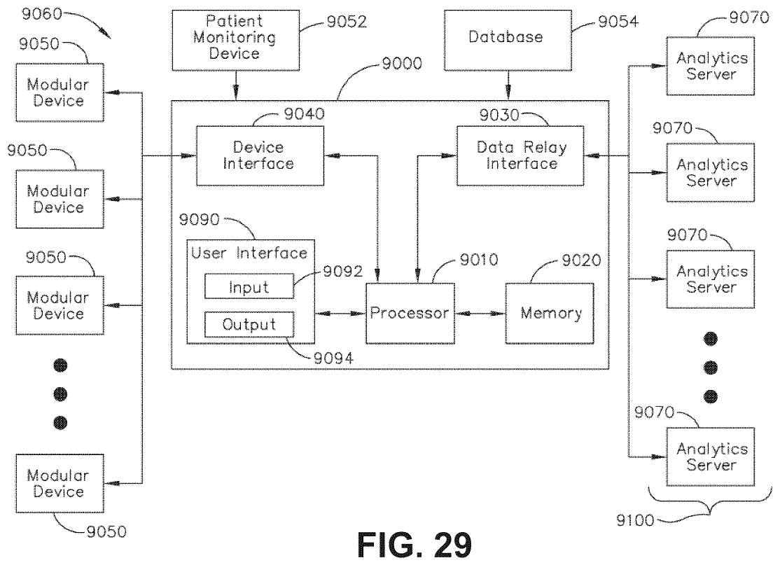

FIG. 28 illustrates an example of autonomous update of surgical device control algorithm(s) 49300. Control algorithm(s) may be pre-installed on a surgical device as the surgical device is manufactured. Each pre-installed control algorithm may be a baseline-version and my be ready to be executed when the surgical device is operated as part of a surgical procedure. Each pre-installed control algorithm may be specific to a surgical device type associated with the surgical device. As shown, a surgical device 49305 (e.g., a modular device 9050 as described in FIG. 29) may be used in a surgical procedure 49302 in operating room 49303. For example, surgical device 49305 may be a surgical stapler that is preinstalled with a baseline control algorithm associated with controlling force-to-close (FTC) and/or a baseline control algorithm associated with controlling force-to-fire (FTF), or a baseline control algorithm associated with controlling FTC and FTF. Surgical device 49305 may be any of the modular devices, as described herein.

When surgical device 49305 is activated in operating room 49303, surgical device 49305 may communicate (e.g., pair or link) with surgical hub 49306 (e.g., before surgical device 19305 is operated as part of the surgical procedure 49302). It may be determined whether the activated surgical device's 49305 preinstalled baseline control algorithm(s) is the latest version, e.g., in response to surgical device's 49305 communicating with the surgical hub 49306. In some examples, the surgical hub 49306 may push a latest version (e.g., an up-to-date version) of the control algorithm to surgical device 49305. Surgical device 49305, in response, may determine whether the preinstalled baseline control algorithm is a same version as the last version of the control algorithm. If it is determined that the preinstalled baseline control algorithm is an older version, surgical device 49305 may replace it with the latest version from surgical hub 49306; otherwise, surgical device 49305 discards the latest version from surgical hub 49006. In some examples, the surgical device 49305 may communicate with surgical hub 49306 to determine whether its preinstalled baseline control algorithm(s) is the latest version. The surgical device 49305 may request version information of the control algorithm that is available on surgical hub 49306. If the version information indicates that the preinstalled baseline control algorithm is not the latest version, the surgical device 49305 may retrieve from surgical hub the latest version of the control algorithm.

Surgical device 49305 may be operated (e.g., by a surgeon) in the surgical procedure 49302, which may be a lung segmentectomy. Perioperative data, such as operation data associated with the surgical device 49305, may be sensed by surgical device 49305. For example, the operation data may include the wait time before a firing is initiated. The operation data may include the FTC over time. The operation data include the FTF over time. The operation data may be sent to surgical hub 49306. Perioperative data, such as outcome data of surgical procedure 49302, may be sent to surgical hub 449306. For example, the outcome data may include data indicating whether there was air or fluid leakage at the surgical site, whether the staples of a particular staple line were formed properly, and/or whether there was bleeding at the surgical site. The operation data and outcome data may be paired as paired data 49304. The paired data 49304 may further include control algorithm information that is associated with the operation data. For example, the control algorithm information may include a unique identifier and a version number of the control algorithm that controls FTC. Perioperative data are described in greater detail in FIG. 194's detailed description in U.S. Patent Application Publication No. US 20190206562 A1 (U.S. patent application Ser. No. 16/209,416), tided Method of hub communication, processing, display, and cloud analytics, filed Dec. 4, 2018, the disclosure of which is herein incorporated by reference in its entirety.

Surgical hub 49306 may send paired data 49304 associated with surgical procedure 49302 to a remote system 49312. The remote system may include remote server 49314 (e.g., an analytics server 9070 described in FIG. 29) coupled to a storage device 49310. The remote system may be a cloud computing system, e.g., 9100 as described in FIGS. 15 and 17.

Surgical hub 49306 may send to remote system 49312 paired data 49308 associated with a plurality of surgical procedures 49302 (e.g., after having accumulated paired data 49304 from different surgical procedures over a period of time). The plurality of surgical procedures may be of one surgical procedure type or more than one surgical procedure type. Surgical hub 49306 may send to remote system 49312 other perioperative data associated with the plurality of surgical procedures 49302, such as preoperative data that includes patient-specific information (e.g., age, employer, body mass index (BMI), or any data that can be used to ascertain the identity of a patient).

In an example, the surgical hub 49306 may be located within a data protection boundary 49322 associated with a medical facility (e.g., a hospital), such as a Health Insurance Portability and Accountability Act (HIPAA) boundary. Surgical hub 49306 may redact paired data 49308 before sending it to remote system 49312 if paired data 49304 includes patient private information, such as age, employer, body mass index (BMI), or any data that can be used to ascertain the identity of a patient. The redaction process is described in greater detail under the heading of “Data Management and Collection” in U.S. Patent Application Publication No. US 20190206562 A1 (U.S. patent application Ser. No. 16/209,385), tided Method of hub communication, processing, storage and display, filed Dec. 4, 2018, the disclosure of which is herein incorporated by reference in its entirety.

Remote system 49312 may receive paired data 49308. The remote system 49312 (e.g., in response) may aggregate and/or analyze the received paired data 49308 to determine whether there are correlation(s) between operation data and outcome data. Remote system 49312 may determine that an update is needed for a control algorithm associated with a surgical device type (e.g., a surgical stapler) based on a determination that there is a correlation between the operation data and the outcome data. For example, if remote system 19312 determines there is a correlation between an aspect of a control algorithm and a negative outcome, remote system 49312 may determine an updated control algorithm 49316 of a surgical device type is needed and may generate the updated control algorithm 19316.

The remote system 49312 (e.g., in response to generating the updated control algorithm 49316) may send the updated control algorithm 49316 to surgical hub 49306. In response to receiving the updated control algorithm 49316, surgical hub 49306 may push it to paired surgical device(s) 49318 that of the surgical device type that is associated with the updated control algorithm 49316 (e.g., if a corresponding control algorithm installed on the paired surgical device(s) 19318 is an older version of the control algorithm 49316). For example, when a new surgical device communicates (e.g., pairs) with surgical hub 49306, the surgical hub 49306 may push the updated control algorithm 49316 to the newly added surgical device. In an example, the surgical hub 49306 may push the updated control algorithm 49316 to the newly added surgical device and not to the surgical devices that are in communication with the surgical hub, and have already received the updated control algorithm 49316. In some examples, surgical hub 49306 may push the updated control algorithm to the paired surgical devices that are in communication with the surgical hub 49306. The paired surgical devices may determine whether to update the corresponding algorithm installed on them, e.g., as described herein.

FIG. 29 illustrates a block diagram of a computer-implemented adaptive surgical system 9060 that is configured to adaptively generate control program updates for modular devices 9050, in accordance with at least one aspect of the present disclosure.

Modular devices include the modules (as described in connection with FIGS. 3 and 29, for example) that are receivable within a surgical hub and the surgical devices or instruments that can be connected to the various modules. The modular devices include, for example, intelligent surgical instruments, medical imaging devices, suction/irrigation devices, smoke evacuators, energy generators, ventilators, and insufflators. Various operations of the modular devices described herein can be controlled by one or more control algorithms. The control algorithms can be executed on the modular device itself, on the surgical hub to which the particular modular device is paired, or on both the modular device and the surgical hub (e.g., via a distributed computing architecture). In some exemplifications, the modular devices' control algorithms may control the devices based on data sensed by the modular device itself (i.e., by sensors in, on, or connected to the modular device). This data can be related to the patient being operated on (e.g., tissue properties or insufflation pressure) or the modular device itself (e.g., the rate at which a knife is being advanced, motor current, or energy levels). For example, a control algorithm for a surgical stapling and cutting instrument can control the rate at which the instrument's motor drives its knife through tissue according to resistance encountered by the knife as it advances.

Although an “intelligent” device including control algorithms that respond to sensed data can be an improvement over a “dumb” device that operates without accounting for sensed data, if the device's control program does not adapt or update over time in response to collected data, then the devices may continue to repeat errors or otherwise perform suboptimally. In an example, operational data collected by the modular devices may be combined with the outcomes of each procedure (or step thereof). The combination may be transmitted to an analytics system. In one exemplification, the procedural outcomes can be inferred by a situational awareness system of a surgical hub to which the modular devices are paired, as described in U.S. patent application Ser. No. 15/940,654, tided SURGICAL HUB SITUATIONAL AWARENESS, which is herein incorporated by reference in its entirety. The analytics system can analyze the data aggregated from a set of modular devices or a particular type of modular device to determine under what conditions the control programs of the analyzed modular devices are controlling the modular devices suboptimally (e.g., if there are repeated faults or errors in the control program or if an alternative algorithm performs in a superior manner) or under what conditions medical personnel are utilizing the modular devices suboptimally The analytics system can then generate an update to fix or improve the modular devices' control programs. Different types of modular devices can be controlled by different control programs; therefore, the control program updates can be specific to the type of modular device that the analytics system determines is performing suboptimally. The analytics system can then push the update to the appropriate modular devices connected to the analytics system through the surgical hubs.

In one exemplification, the surgical system includes a surgical hub 9000, multiple modular devices 9050 communicably coupled to the surgical hub 9000, and an analytics system 9100 communicably coupled to the surgical hub 9000. Although a single surgical hub 9000 is depicted, it should be noted that the surgical system 9060 can include any number of surgical hubs 9000, which can be connected to form a network of surgical hubs 9000 that are communicably coupled to the analytics system 9010. In one exemplification, the surgical hub 9000 includes a processor 9010 coupled to a memory 9020 for executing instructions stored thereon and a data relay interface 9030 through which data is transmitted to the analytics system 9100. In one exemplification, the surgical hub 9000 further includes a user interface 9090 having an input device 9092 (e.g., a capacitive touchscreen or a keyboard) for receiving inputs from a user and an output device 9094 (e.g., a display screen) for providing outputs to a user. Outputs can include data from a query input by the user, suggestions for products or mixes of products to use in a given procedure, and/or instructions for actions to be carried out before, during, or after surgical procedures. The surgical hub 9000 further includes an interface 9040 for communicably coupling the modular devices 9050 to the surgical hub 9000. In one aspect, the interface 9040 includes a transceiver that is communicably connectable to the modular device 9050 via a wireless communication protocol. The modular devices 9050 can include, for example, surgical stapling and cutting instruments, electrosurgical instruments, ultrasonic instruments, insufflators, respirators, and display screens. In one exemplification, the surgical hub 9000 can further be communicably coupled to one or more patient monitoring devices 9052, such as EKG monitors or BP monitors. In another exemplification, the surgical hub 9000 can further be communicably coupled to one or more databases 9054 or external computer systems, such as an EMR database of the medical facility at which the surgical hub 9000 is located.

When the modular devices 9050 are connected to the surgical hub 9000, the surgical hub 9000 can sense or receive perioperative data from the modular devices 9050 and then associate the received perioperative data with surgical procedural outcome data. The perioperative data indicates how the modular devices 9050 were controlled during the course of a surgical procedure. The procedural outcome data includes data associated with a result from the surgical procedure (or a step thereof), which can include whether the surgical procedure (or a step thereof) had a positive or negative outcome. For example, the outcome data could include whether a patient suffered from postoperative complications from a particular procedure or whether there was leakage (e.g., bleeding or air leakage) at a particular staple or incision line. The surgical hub 9000 can obtain the surgical procedural outcome data by receiving the data from an external source (e.g., from an EMR database 9054), by directly detecting the outcome (e.g., via one of the connected modular devices 9050), or inferring the occurrence of the outcomes through a situational awareness system. For example, data regarding postoperative complications could be retrieved from an EMR database 9054 and data regarding staple or incision line leakages could be directly detected or inferred by a situational awareness system. The surgical procedural outcome data can be inferred by a situational awareness system from data received from a variety of data sources, including the modular devices 9050 themselves, the patient monitoring device 9052, and the databases 9054 to which the surgical hub 9000 is connected.

The surgical hub 9000 can transmit the associated modular device 9050 data and outcome data to the analytics system 9100 for processing thereon. By transmitting both the perioperative data indicating how the modular devices 9050 are controlled and the procedural outcome data, the analytics system 9100 can correlate the different manners of controlling the modular devices 9050 with surgical outcomes for the particular procedure type. In one exemplification, the analytics system 9100 includes a network of analytics servers 9070 that are configured to receive data from the surgical hubs 9000. Each of the analytics servers 9070 can include a memory and a processor coupled to the memory that is executing instructions stored thereon to analyze the received data. In some exemplifications, the analytics servers 9070 are connected in a distributed computing architecture and/or utilize a cloud computing architecture. Based on this paired data, the analytics system 9100 can then learn optimal or preferred operating parameters for the various types of modular devices 9050, generate adjustments to the control programs of the modular devices 9050 in the field, and then transmit (or “push”) updates to the modular devices' 9050 control programs.

Additional detail regarding the computer-implemented interactive surgical system 9060, including the surgical hub 9000 and various modular devices 9050 connectable thereto, are described herein.

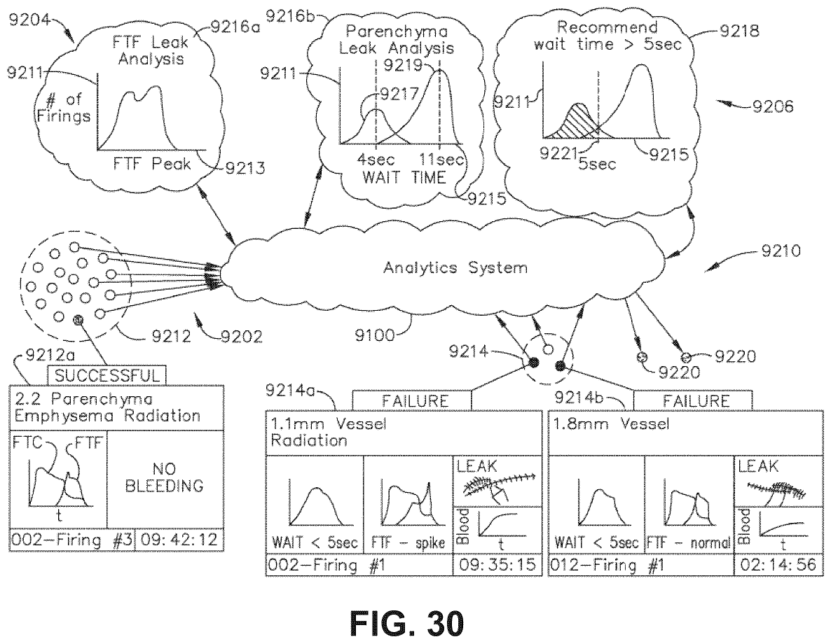

In order to assist in the understanding of the process 9100 illustrated in FIG. 29 and the other concepts discussed above, FIG. 30 illustrates a diagram of an illustrative analytics system 9100 updating a surgical instrument control program, in accordance with at least one aspect of the present disclosure. In one exemplification, a surgical hub 9000 or network of surgical hubs 9000 is communicably coupled to an analytics system 9100, as illustrated above in FIG. 29. The analytics system 9100 is configured to filter and analyze modular device 9050 data associated with surgical procedural outcome data to determine whether adjustments need to be made to the control programs of the modular devices 9050. The analytics system 9100 can then push updates to the modular devices 9050 through the surgical hubs 9000, as necessary. In the depicted exemplification, the analytics system 9100 comprises a cloud computing architecture. The modular device 9050 perioperative data received by the surgical 9000 hubs from their paired modular devices 9050 can include, for example, force to fire (i.e., the force required to advance a cutting member of a surgical stapling instrument through a tissue), force to close (i.e., the force required to clamp the jaws of a surgical stapling instrument on a tissue), the power algorithm (i.e., change in power over time of electrosurgical or ultrasonic instruments in response to the internal states of the instrument and/or tissue conditions), tissue properties (e.g., impedance, thickness, stiffness, etc.), tissue gap (i.e., the thickness of the tissue), and closure rate (i.e., the rate at which the jaws of the instrument clamped shut). It should be noted that the modular device 9050 data that is transmitted to the analytics system 9100 is not limited to a single type of data and can include multiple different data types paired with procedural outcome data. The procedural outcome data for a surgical procedure (or step thereof) can include, for example, whether there was bleeding at the surgical site, whether there was air or fluid leakage at the surgical site, and whether the staples of a particular staple line were formed properly. The procedural outcome data can further include or be associated with a positive or negative outcome, as determined by the surgical hub 9000 or the analytics system 9100, for example. The modular device 9050 data and the procedural outcome data corresponding to the modular device 9050 perioperative data can be paired together or otherwise associated with each other when they are uploaded to the analytics system 9100 so that the analytics system 9100 is able to recognize trends in procedural outcomes based on the underlying data of the modular devices 9050 that produced each particular outcome. In other words, the analytics system 9100 can aggregate the modular device 9050 data and the procedural outcome data to search for trends or patterns in the underlying device modular data 9050 that can indicate adjustments that can be made to the modular devices' 9050 control programs.

In the depicted exemplification, the analytics system 9100 executing the process 9200 described in connection with FIG. 29 is receiving 9202 modular device 9050 data and procedural outcome data. When transmitted to the analytics system 9100, the procedural outcome data can be associated or paired with the modular device 9050 data corresponding to the operation of the modular device 9050 that caused the particular procedural outcome. The modular device 9050 perioperative data and corresponding procedural outcome data can be referred to as a data pair. The data is depicted as including a first group 9212 of data associated with successful procedural outcomes and a second group 9214 of data associated with negative procedural outcomes. For this particular exemplification, a subset of the data 9212, 9214 received 9202 by the analytics system 9100 is highlighted to further elucidate the concepts discussed herein.

For a first data pair 9212a, the modular device 9050 data includes the force to close (FTC) over time, the force to fire (FTF) over time, the tissue type (parenchyma), the tissue conditions (the tissue is from a patient suffering from emphysema and had been subject to radiation), what number firing this was for the instrument (third), an anonymized time stamp (to protect patient confidentiality while still allowing the analytics system to calculate elapsed time between firings and other such metrics), and an anonymized patient identifier (002). The procedural outcome data includes data indicating that there was no bleeding, which corresponds to a successful outcome (i.e., a successful firing of the surgical stapling instrument). For a second data pair 9212b, the modular device 9050 data includes the wait time prior the instrument being fired (which corresponds to the first firing of the instrument), the FTC over time, the FTF over time (which indicates that there was a force spike near the end of the firing stroke), the tissue type (1.1 mm vessel), the tissue conditions (the tissue had been subject to radiation), what number firing this was for the instrument (first), an anonymized time stamp, and an anonymized patient identifier (002). The procedural outcome data includes data indicating that there was a leak, which corresponds to a negative outcome (i.e., a failed firing of the surgical stapling instrument). For a third data pair 9212c, the modular device 9050 data includes the wait time prior the instrument being fired (which corresponds to the first firing of the instrument), the FTC over time, the FTF over time, the tissue type (1.8 mm vessel), the tissue conditions (no notable conditions), what number firing this was for the instrument (first), an anonymized time stamp, and an anonymized patient identifier (012). The procedural outcome data includes data indicating that there was a leak, which corresponds to a negative outcome (i.e., a failed firing of the surgical stapling instrument). It should be noted again that this data is intended solely for illustrative purposes to assist in the understanding of the concepts discussed herein and should not be interpreted to limit the data that is received and/or analyzed by the analytics system 9100 to generate control program updates.

When the analytics system 9100 receives 9202 perioperative data from the communicably connected surgical hubs 9000, the analytics system 9100 proceeds to aggregate and/or store the data according to the procedure type (or a step thereof) associated with the data, the type of the modular device 9050 that generated the data, and other such categories. By collating the data accordingly, the analytics system 9100 can analyze the data set to identify correlations between particular ways of controlling each particular type of modular device 9050 and positive or negative procedural outcomes. Based upon whether a particular manner of controlling a modular device 9050 correlates to positive or negative procedural outcomes, the analytics system 9100 can determine 9204 whether the control program for the type of modular device 9050 should be updated.

For this particular exemplification, the analytics system 9100 performs a first analysis 9216 of the data set by analyzing the peak FTF 9213 (i.e., the maximum FTF for each particular firing of a surgical stapling instrument) relative to the number of firings 9211 for each peak FTF value. In this exemplary case, the analytics system 9100 can determine that there is no particular correlation between the peak FTF 9213 and the occurrence of positive or negative outcomes for the particular data set. In other words, there are not distinct distributions for the peak FTF 9213 for positive and negative outcomes. As there is no particular correlation between peak FTF 9213 and positive or negative outcomes, the analytics system 9100 would thus determine that a control program update to address this variable is not necessary. Further, the analytics system 9100 performs a second analysis 9216 b of the data set by analyzing the wait time 9215 prior to the instrument being fired relative to the number of firings 9211. For this particular analysis 9216 b, the analytics system 9100 can determine that there is a distinct negative outcome distribution 9217 and a positive outcome distribution 9219. In this exemplary case, the negative outcome distribution 9217 has a mean of 4 seconds and the positive outcome distribution has a mean of 11 seconds. Thus, the analytics system 9100 can determine that there is a correlation between the wait time 9215 and the type of outcome for this surgical procedure step. Namely, the negative outcome distribution 9217 indicates that there is a relatively large rate of negative outcomes for wait times of 4 seconds or less. Based on this analysis 9216 b demonstrating that there is a large divergence between the negative outcome distribution 9217 and the positive outcome distribution 9219, the analytics system 9100 can then determine 9204 that a control program update should be generated 9208.

Once the analytics system 9100 analyzes the data set and determines 9204 that an adjustment to the control program of the particular module device 9050 that is the subject of the data set would improve the performance of the modular device 9050, the analytics system 9100 then generates 9208 a control program update accordingly. In this exemplary case, the analytics system 9100 can determine based on the analysis 9216 b of the data set that a control program update 9218 recommending a wait time of more than 5 seconds would prevent 90% of the distribution of the negative outcomes with a 95% confidence interval. Alternatively, the analytics system 9100 can determine based on the analysis 9216 b of the data set that a control program update 9218 recommending a wait time of more than 5 seconds would result in the rate of positive outcomes being greater than the rate of negative outcomes. The analytics system 9100 could thus determine that the particular type of surgical instrument should wait more than 5 seconds before being fired under the particular tissue conditions so that negative outcomes are less common than positive outcomes. Based on either or both of these constraints for generating 9208 a control program update that the analytics system 9100 determines are satisfied by the analysis 9216 b, the analytics system 9100 can generate 9208 a control program update 9218 for the surgical instrument that causes the surgical instrument, under the given circumstances, to either impose a 5 second or longer wait time before the particular surgical instrument can be fired or causes the surgical instrument to display a warning or recommendation to the user that indicates to the user that the user should wait at least 5 seconds before firing the instrument. Various other constraints can be utilized by the analytics system 9100 in determining whether to generate 9208 a control program update, such as whether a control program update would reduce the rate of negative outcomes by a certain percentage or whether a control program update maximizes the rate of positive outcomes.

After the control program update 9218 is generated 9208, the analytics system 9100 then transmits 9210 the control program update 9218 for the appropriate type of modular devices 9050 to the surgical hubs 9000. In one exemplification, when a modular device 9050 that corresponds to the control program update 9218 is next connected to a surgical hub 9000 that has downloaded the control program update 9218, the modular device 9050 then automatically downloads the update 9218. In another exemplification, the surgical hub 9000 controls the modular device 9050 according to the control program update 9218, rather than the control program update 9218 being transmitted directly to the modular device 9050 itself.

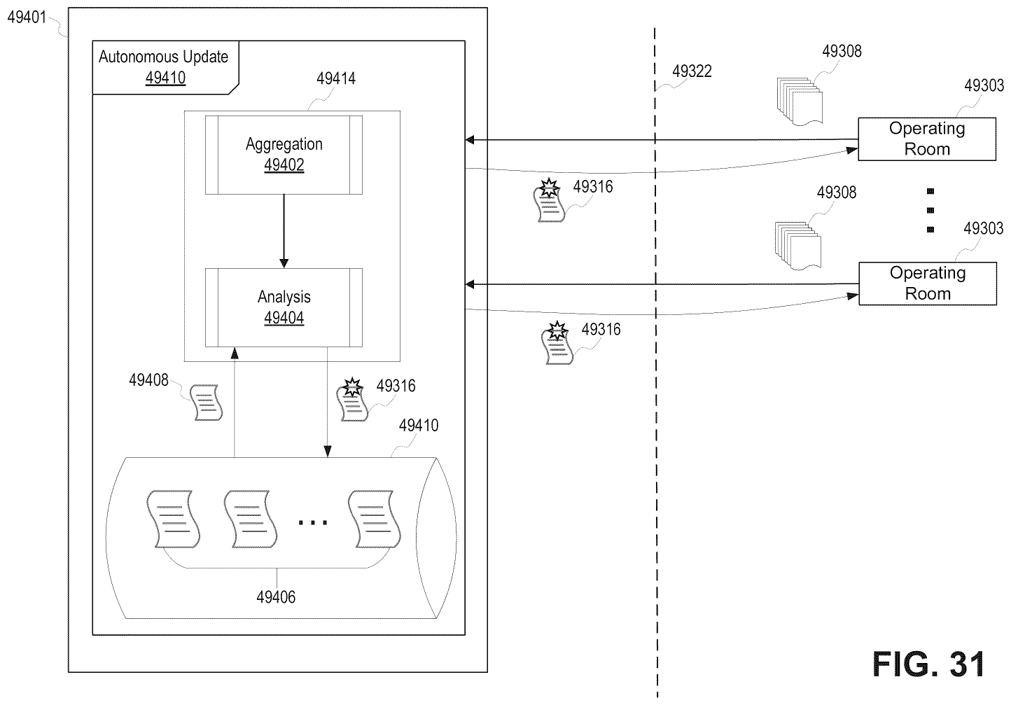

FIG. 31 illustrates an example process of autonomous update of surgical device control algorithm. As shown, there may be a plurality of operating rooms 49303 in a medical facility (e.g., a hospital). The plurality of operation room 49303 may be within data protection boundary 49322 associated with the medical facility (e.g., as described herein). The plurality of operating rooms 49303 (e.g., via surgical hubs 49306) may send paired data 49308 to a remote system 49401 (e.g., beyond the data protection boundary 49322 after redacting patient private information in paired data 49308 as described herein).

Remote system 49401 (e.g., remote system 49312) may include an autonomous update subsystem 49410. Autonomous update subsystem 49410 may include an analytics server 49414 (e.g., 49314 described in FIG. 28) and a datastore 49410 (e.g., 49310 described in FIG. 28). Analytics server 49414 may include an aggregation process 49402 and an analysis process 49404.

Datastore 49410 may store a control algorithm collection 49406. Control algorithm collection 49406 may include latest versions of control algorithms. For example, each surgical device type may have one or more control algorithms associated its operation. Such control algorithms of each surgical device types may be stored in datastore 49410. A unique identifier and a version number of each control algorithm may be stored in datastore 49410.

After remote system 49401 receives paired data 49308, autonomous update subsystem 49410 may determine whether paired data 49308 is to be discarded or used for generation of control algorithm updates. In examples, autonomous update subsystem 49410 may determine whether the control algorithms that are included in paired data 49308 (e.g., control algorithm information associated with the operation data part of paired data 49308) are up to date. If the version numbers associated with the control algorithms indicate they are not the latest version numbers, autonomous update subsystem 49410 may discard them (e.g., perform no further processing on them). If the version numbers associated with the control algorithms indicate they are the latest version numbers, autonomous update subsystem 49410 may perform further processing on the associated paired data/operation data.

Aggregation process 49402 may aggregate paired data 49308 based on a surgical device type. Aggregation process 49402 may aggregate paired data 49308 based on a surgical procedure type. Aggregation process 49402 may aggregate paired data 49308 based on a surgical device type and a surgical procedure type. Aggregation process 49402 may aggregate paired data 49308 based on any other such categorie(s).

Based on the aggregated paired data, analysis process 49404 may identify correlation(s) between aspect(s) of operation data and outcome data that are associated with a surgical device. For example, analysis process 49404 may perform an analysis (e.g., analysis 9216 b in FIG. 30) of the data set by analyzing the wait time 9215 prior to a surgical stapler being fired relative to the number of firings 9211. Analysis process 49404 may determine that there is a distinct negative outcome distribution (e.g., 9217 in analysis 9216b as shown in FIG. 30) and a positive outcome distribution (e.g., 9219 in analysis 9216b as shown in FIG. 30). In an example, the negative outcome distribution may have a mean of 4 seconds and the positive outcome distribution may have a mean of 11 seconds. Analysis process 49404 may determine that there is a correlation between the wait time (e.g., 9215 in analysis 9216b as shown in FIG. 30) and the type of outcome for this surgical procedure step. That is, the negative outcome distribution may indicate that there is a relatively large rate of negative outcomes for wait times of 4 seconds or less.

Based on this analysis, analysis process 49404 may determine that an update to the control algorithm associated with controlling wait time (e.g., an FTC control algorithm, such as 49408) should be generated for surgical staplers. An updated control algorithm 49316 may be generated and the updated control algorithm 49316 may include a constraint that the wait time before firing must be at least 5 seconds. The updated control algorithm 49316 may be sent to datastore 49406 to update or replace the existing control algorithm 49408. The updated control algorithm 49316 may be sent to the plurality of operating rooms 49303 (e.g., the surgical hubs 49306 in the operating rooms 49303).

Autonomous update subsystem 49410 may autonomously update control algorithms as described herein as remote system 49401 continues receiving paired data 49308 from the plurality of operating rooms 49303. Such paired data 49308 may be received from operating rooms 49303 in a medical facility, medical facilities in a geographic location, medical facilities in a geographic region, or medical facilities in various geographic regions.

In an example, paired data 49308 may be received from a geographic region that did not previously send paired data 49308 to remote system 49401. In such case, paired data 49308 from the geographic region may present different data patterns, e.g., because surgeons/medical professionals may be trained to operate surgical device differently. For example, based on the aggregated paired data (including paired data 49308 from the new geographic region), analysis process 49404 (e.g., in analysis 9216b as shown in FIG. 30) may identify in the case of a surgical stapler a new correlation between wait time and outcome data. A negative outcome distribution may emerge as having a mean of 16 seconds. Based on this analysis, analysis process 49404 may determine 9204 that another update to the control algorithm associated with controlling wait time (e.g., an FTC control algorithm, such as 49408) should be generated for surgical staplers. An updated control algorithm 49316 may be generated and the updated control algorithm 49316 may include a constraint that the wait time before firing must be at least 5 seconds and less than 16 seconds. The updated control algorithm 49316 may be sent to datastore 49406 to update or replace the existing control algorithm 49408. The updated control algorithm 49316 may be sent to the plurality of operating rooms 49303 (e.g., the surgical hubs 49306 in the operating rooms 49303).

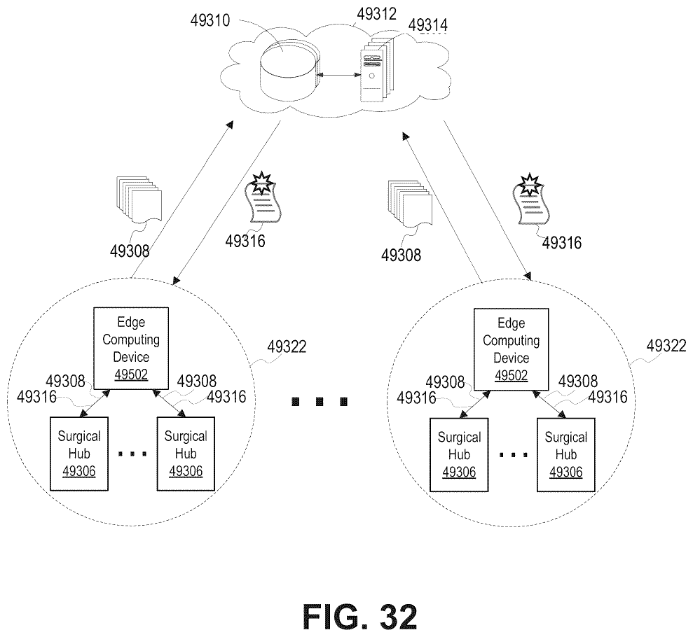

FIG. 32 illustrates an example process to autonomous update of surgical device control algorithm. As shown, there may be a plurality of data protection boundaries 49322 associated with the corresponding medical facilities (e as described herein). Surgical hubs 49306 within each 49322 may send paired data 49308 to remote system 49312 (e.g. as described in FIGS. 14 and 17) In response, surgical hubs 49306 may receive updated control algorithms 49316 from remote system 49312 (e.g., as described in FIGS. 14 and 17).

Alternatively, surgical hubs 49306 within each 49322 may send paired data 49308 to an edge computing device 49502 and edge computing device 49502, in response, may aggregate and/or analyze paired data 49308 to determine whether there are correlation(s) between operation data and outcome data. The edge computing device/system is described in greater detail in U.S. patent application Ser. No. 17/384,151, tided MULTI-LEVEL SURGICAL DATA ANALYSIS SYSTEM, filed Jul. 23, 2021, the disclosure of which is herein incorporated by reference in its entirety.

Like remote system 49312 (or remote system 49401), edge computing device 49502 may include autonomous update subsystem 49410, as described in FIG. 31. Edge computing device 49502 may determine that an update is needed for a control algorithm associated with a surgical device type (e.g., a surgical stapler) based on a determination that there is a correlation between the operation data and the outcome data. For example, if edge computing device 49502 determines there is a correlation between an aspect of a control algorithm and a negative outcome, remote system 49312 may determine an updated control algorithm 49316 of a surgical device type is needed and may generate the updated control algorithm 49316. In response to generating the updated control algorithm 49316, edge computing device 49502 may send the updated control algorithm 49316 to surgical hubs 49306.

Unlike remote system 49312, edge computing device 49502 is located within data protection boundary 49322 and, consequently, surgical hubs 49306 may send paired data 49308 in it unredacted form to edge computing device 49502. In such case, aggregation process 49402 of edge computing device 49502 may aggregate unredacted paired data 49308 further based on patient private information and analysis process 49404 may identify correlation(s) between aspect(s) of operation data and outcome data that are associated with a surgical device further based on patient private information. For example, aggregation process 49402 of edge computing device 49502 may aggregated unredacted paired data 49308 by surgical device type and by patient age group. In an example, analysis process 49404 may perform an analysis (e.g., analysis 9216 b in FIG. 30) and may identify determine a negative outcome distribution and a positive outcome distribution for each patient age group. In such manner, the updated control algorithm for a surgical stapler (e.g., an updated FTC control algorithm) may include different constraints for different age groups, such as wait time must be at least 5 seconds for patients that are 20 or younger and wait time must be at least 7 seconds for patients that are between 20 and 50 and wait time must be at least 10 seconds for patients that are older than 50.

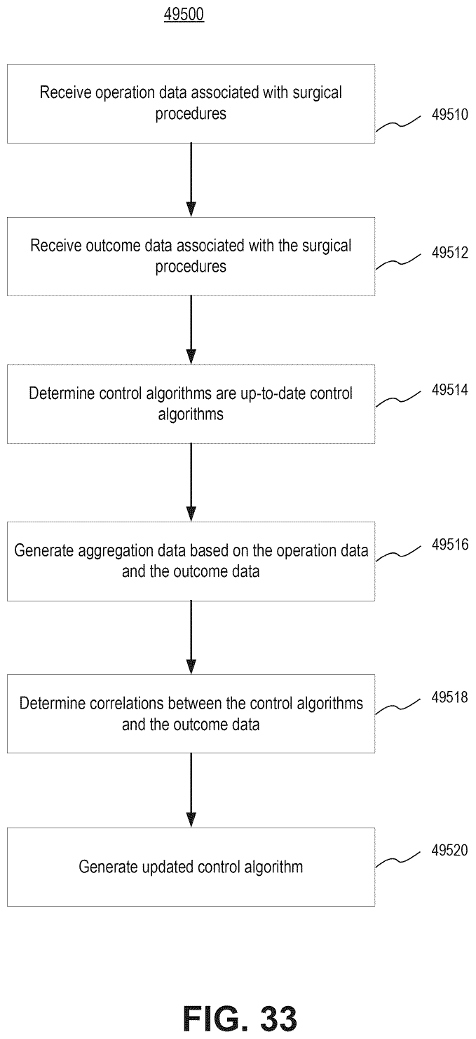

FIG. 33 is a flow chart of an example process of autonomous update of surgical device control algorithm, 49500. The process may be performed by remote system 49312 or edge computing device 49502 described in FIG. 32.

At 49510, operation data associated with surgical procedures are received. For example, first operation data associated with a first surgical procedure and second operation data associated with a second surgical procedure may be received. The first operation data may be associated with a first aspect of a control algorithm of a first surgical device. The second operation data may be associated with a first aspect of a control algorithm of a second surgical device. The first surgical device and second surgical device may be of a first surgical device type. For example, the first operation data may be received from the first surgical device and the second operation data may be received from the second surgical device. For example, the first operation data and the second operation data may be received from a surgical hub or two different surgical hubs.

At 49512, outcome data associated with the surgical procedures may be received. For example, first outcome data associated with the first surgical procedure may be received. Second outcome data associated with the second surgical procedure may be received. For example, the first outcome data associated with the first surgical procedure may be received from a surgical hub or a surgical visualization device.

At 49514, control algorithms are determined to be up-to-date control algorithms. For example, each of the control algorithm of the first surgical device and the control algorithm of the second surgical device may be determined to be an up-to-date control algorithm associated with the first surgical device type.

At 49516, aggregation data may be generated based on the operation data and the outcome data. For example, the aggregation data may be generated based on at least the first operation data, the second operation data, the first outcome data, and the second outcome data. For example, the outcome data may comprise the first outcome data and the second outcome data.

At 49518, correlations between the control algorithms and the outcome data may be determined. For example, based on at least the first aggregation data, a correlation may be determined between the first aspect of the up-to-date control algorithm and outcome data.

For example, the determined correlation may comprise a correlation between the first aspect of the up-to-date control algorithm and a negative surgical outcome and a correlation between the first aspect of the up-to-date control algorithm and a positive surgical outcome.

At 49520, updated control algorithms may be generated. For example, based on the determined correlation, an updated up-to-date control algorithm may be generated. For example, the updated up-to-date control algorithm may be published for a surgical device or a surgical hub to download via an interface. For example, the updated up-to-date control algorithm may be sent to a plurality of surgical hubs.

Autonomous adjustments to control algorithms and/or approaches of a scope, view instruments associated with the scope, and/or a displaying system associated with the scope may be used to configure the devices capture, device operation and/or device display aspects, e.g., to maximize communication data and user control of the displayed data.

Displayed data associated with an instrument may be autonomously adapted based on recorded usage. Autonomous adjustments of a video stream and/or interpretation of a portion of the video stream may be used to adapt system notifications and warnings. Based on detected movement of a tool (e.g., via a camera or a scope), system parameters may be automatically adjusted. In examples, if a device moves closer and in position to start a use sequence (such as cutting with an energy tool), the system may automatically start to get the device ready to perform an associated use task (such as heating the blade, performing safety and/or functional checks, etc.). In examples, if a device moves away from an intended use site (such as near a critical structure) and/or the device is outside the field of view, the device may be placed into a safe mode or may be prevented from continued use.

Adjustments to approaches to tissue(s) may be made based on detected device location and/or detected device use. The method of approach to tissue that the device is using may be video captured. Based on this approach, the user may be informed of different approaches. The system may be automatically attempt to compensate for the poor angle of attack (e.g., associated with the device).

Calibration of control algorithms may be implemented. Autonomous control algorithm calibrations may be implemented so that different devices may operate to a same practical outcome. In an example, linear staplers may measure their corresponding speed (e.g., cutting member advancing speed or staple firing speed) as part of factory data collection. Such speed data may be aggregated against speed data associated other linear staplers. For example, the aggregation may be performed in a remote system, such as remote system 49312, or an edge computing system, such as edge computing device 49502. The updated speed algorithm(s) may be generated and may be pushed to linear staplers for use in a future operation. When used in a surgical procedure, the linear staplers may be able to perform a calibrated approach (e.g., using calibrated speed algorithm(s)) for optimized outcomes. In examples, different linear staplers may run at different firing speeds within some manufacturing limit. This manufacturing difference, for example, via calibration of firing algorithm(s) using firing speed data, may be used as an input for data aggregation/data analysis (e.g., 49402 and 49404 in FIG. 31) to determine optimized algorithmic output of firing speed. Other calibrations (e.g., required calibrations based on factory settings) may be skipped of nor performed (e.g., after optimized algorithms have been determined and other calibrations are no longer needed).

Calibration(s) of control algorithms associated with surgical device(s) may be performed intraoperatively. A surgical device may start a firing. The surgical device, at least based on outcomes during a firing, may adjust (e.g., may be able to) adjust associated control algorithm(s) during a firing and/or between firings. In examples, the surgical device may (e.g., may be able to) detect that it has stalled. The stalled surgical device based on at least on the detection that it has stalled may be able to automatically adjust its force gains (e.g., in an area where it shouldn't have stalled). In examples, an articulation mechanism may degrade over time and may result in less repeatable outcomes. Articulation mechanism may include a mechanism of articulation associated with an end effector, which may include articulation force, articulation angle, articulation direction, and adjustment thereof in the articulation process. Articulation mechanism are described in greater detail in in PCT Patent Application Publication No. WO2018142277A1, tided Robotic Surgical System and Methods for Articulation Calibration, filed Jan. 30, 2018, the disclosure of which is herein incorporated by reference in its entirety. Such a degrading of articulation mechanism may cause the zero position to shift. The surgical device may (e.g., in response) automatically adjust accordingly. For example, the surgical device may automatically adjust based on a visual indication of angle or forces that bias the articulation system during trocar extraction.

Calibration may be performed at regular time intervals. The frequency of Calibration may be determined based on a last known calibration of the system (e.g., time associated with the last known calibration of the system). In an example, a system may (e.g., intentionally) introduce a degree of uncertainty into automated optimization. In examples, the system may be outside of its optimal calibration time window and may still be within acceptable limits for use. As a result, an uncertainty offset may continue to increment over time. A system may make recommendation and/or optimizations based on an associated uncertainty limit. For example, when the system makes recommendations and/or optimizations, it may not be allowed to optimize parameters or settings beyond its associated uncertainty limit(s). The system may perform checks, for example, periodical checks. In an example, the system may perform a calibration check(s) when the system is idle or not operating. In an example, the system may perform calibration check(s) that may be performed automatically at the beginning of surgery and/or if a surgical device is coupled with a surgical system (e.g., robotic surgical system), the surgical device is initialized, or the surgical device is reset.

A system may perform calibration error check(s). For example, the system may perform one or more checks on different areas to confirm if calibration was performed correctly. By assessing data from different areas, the system may determine if the calibration was in fact valid, or if there was another issue within the system, which may have led to an invalid calibration. Different areas may refer to a set of subsystem components. For example, a lighting or scoping system may execute a sequence with expected outputs to determine if a calibration error is active. In an example, a different subsystem may be utilized to move motors and to check if calibration was performed correctly.

Intra-operative adjustments to surgical procedures may be implemented. Based on surgical decisions that are within a surgeon's control, a system may make adjustment(s) to control algorithms and/or surgical device operation. In examples, a surgeon may clamp a surgical device for a given amount of time. The amount of time may be a portion of a recommended clamping time. The amount of time may be chosen to achieve optimal performance of the surgical device. Based on the available dataset, the surgical device may adjust the speed of firing to optimize the outcome of the stapling and cutting operation accordingly.

The bounds of adjustments to algorithms or adjustments to the device operation may be created to limit surgical operation and may be dynamically changed based on the progression of the surgery. In examples, the surgeon may be firing a linear stapler across a combination of healthy and diseased tissues. Based on tissue measurements, the linear stapler may be loaded with a specific profile. As the stapler knife and sled transition from the healthy tissue into the diseased tissue, the surgical device limits and parameters may automatically be adjusted to a new profile. In examples, the surgeon may be performing a sleeve gastrectomy on a patient and may have performed the initial firing with a blue cartridge. The stapler, in response to measuring compression time or tissue thickness, may make adjustments to the firing algorithm based on the dataset for blue cartridges. For the next firing, the surgeon may swap to a different cartridge, and as a result, the system may automatically load a new dataset for limits or other parameters within the algorithm or the surgical device.

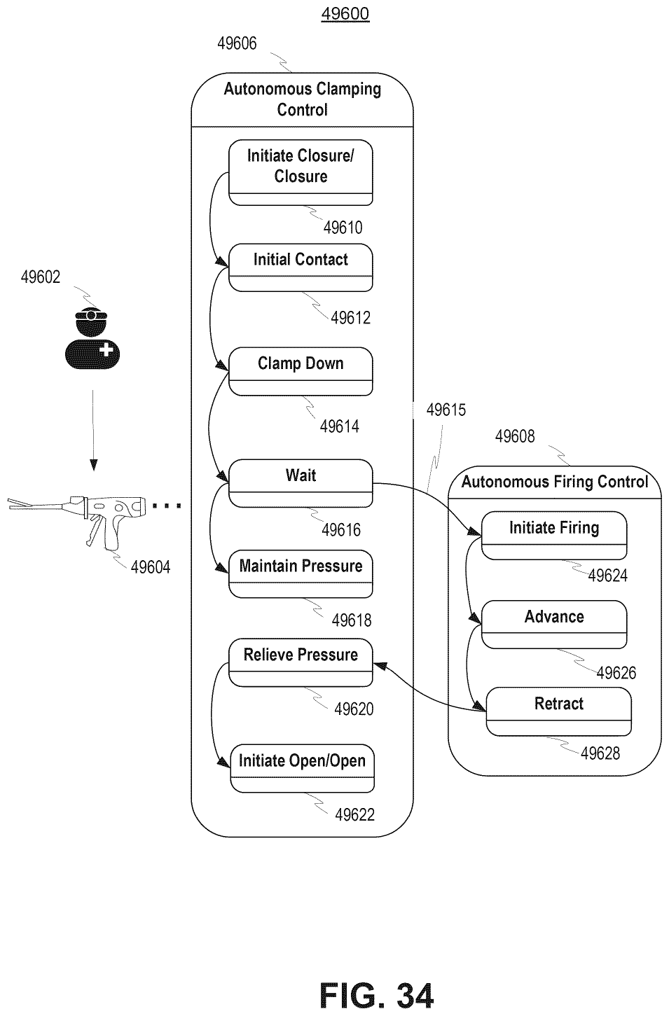

FIG. 34 illustrates an example autonomous operation of a surgical instrument, 49600. The surgical instrument may be a smart surgical stapler. Additional details about operation of a smart stapler are disclosed in U.S. patent application Ser. No. 16/209,423, titled METHOD OF COMPRESSING TISSUE WITHIN A STAPLING DEVICE AND SIMULTANEOUSLY DISPLAYING THE LOCATION OF THE TISSUE WITHIN THE JAWS, filed Dec. 4, 2018, the disclosure of which is herein incorporated by reference in its entirety.

A smart surgical stapler's operation may include clamping control and firing control. Clamping control may include control associated with one or more the following steps: initiating closure/closure of a clamping jaw, initial contact with a tissue, clamping down (e.g., to a pre-determined pressure), wait (e.g., for a pre-determined period during which tissue creep occurs), maintaining pressure (e.g., during firing), relieving pressure (e.g., after firing is complete), initiate opening a clamping jaw/opening a clamping jaw. Firing control may include control associated with one or more the following steps: initiating firing (e.g., after wait), advancing a cutting member (e.g., a knife), firing staples, or retract the cutting member.

The smart surgical stapler's operation may include closure control. The closure control may include control associated with closing the clamping jaw (e.g., until the clamping jaw makes the initial contact with the tissue). A smart surgical instrument's operation may include opening control. The opening control may include control associated with opening the clamping jaw, for example, after the cutting member is retracted to the starting position.

A smart surgical stapler's operation may be controlled autonomously. The autonomous operation may be at different levels. One level of autonomous operation may include autonomously performing clamping down a clamping jaw to a pre-determined level (e.g., a pre-determined pressure or a pre-determined closure rate (e.g., 50% vs. fully closed)), wait (e.g., for a pre-determined period), initiating firing (e.g., after a pre-determined period of wait elapses), retracting a cutting member (e.g., after advancing of the cutting member is complete and staples are fired). One level of autonomous operation may include autonomously performing pre-defined clamping control between initial contact with a tissue and firing is ready to be initiated (e.g., when the wait period completes). One level of autonomous operation may include autonomously performing initiating firing and firing the cutting member to end-of-distal stroke and wait until manually indicated (e.g., by a surgeon) to retract the cutting member. One level of autonomous operation may include autonomously performing initiating firing, firing the cutting member to end-of-distal stroke, and retracting the cutting member to a starting position. One level of autonomous operation may include autonomously performing clamping down a clamping jaw to a pre-determined level, wait, initiating firing, retracting the cutting member, relieving pressure on the tissue (e.g., not including releasing the tissue/opening the clamping jaw).

The level of autonomous operation of a smart surgical stapler may be indicated by a healthcare professional (e.g., a surgeon). A healthcare professional may actuate a clamping control trigger and at the end of the autonomous clamping control hold (e.g., keep pressed) the clamping control trigger. In such case, the clamping control operation may be autonomous, and the healthcare professional may take control back and perform manual firing control. A healthcare professional may actuate the clamping control trigger momentarily and may then release it. In such case, the clamping control operation and the firing control operation may be autonomous. A healthcare professional may actuate the firing control trigger and then may hold the trigger. In such case, the firing control operation may be manual. A healthcare professional may actuate the firing control trigger and may then release it (e.g., after actuating the clamping control trigger first and then at the end of the autonomous clamping trigger hold the clamping control trigger, as described herein). In such case, the firing control operation may be autonomous (e.g., regardless of whether the clamping control operation is autonomous or manual). A healthcare professional may actuate and hold the clamping control trigger and then may actuate and hold the firing control trigger. In such case, the clamping control operation and the firing control operation may both be manual. A healthcare professional may release a hold on an actuation control trigger (e.g., the clamping control trigger or the firing control trigger). In such case, the clamping control operation or the firing control operation may transition from manual to autonomous mode. A healthcare professional may reactivate a hold on an actuation control trigger (e.g., the clamping control trigger or the firing control trigger) when the clamping control operation or the firing control operation is during its autonomous operation. In such case, the clamping control operation or the firing control operation may transition from autonomous to manual. Further in such case, the healthcare professional may subsequently release the control trigger. In this manner, the clamping control operation or the firing control operation may transition from manual to autonomous. A healthcare professional may actuate the opening control and release the control. In such case, the opening control operation may be autonomous. A healthcare professional may actuate the opening control and hold the control. In such case, the opening control operation may be manual.

As illustrated in FIG. 8, a smart surgical stapler 49604 may include a processor that is configured with one or more control algorithms for its autonomous operation. The smart surgical stapler 49604 may include a control system, for example, as described in FIG. 5. The smart surgical stapler 49604 may be configured with a control algorithm associated with autonomous clamping control operation 49606 (“autonomous clamping control algorithm”). The smart surgical stapler 49604 may be configured with a control algorithm associated with autonomous firing control operation 49608 (“autonomous firing control algorithm”). The smart surgical stapler 49604 may be configured with a control algorithm associated with the autonomous clamping control operation and the autonomous firing control operation. The autonomous clamping control algorithm 49606 may include one or more of the following: initiating closure 49610 of a clamping jaw, initial contact 49612 with a tissue, clamping down 49614 (e.g., to a pre-determined pressure), wait 49616 (e.g., before initiating firing 49624), maintaining pressure 49618 (e.g., during firing), relieving pressure 49620 (e.g., after firing is complete), or opening a clamping jaw 49622. The autonomous firing control algorithm 49608 may include one or more of the following: initiating firing 49624, advancing a cutting member 49626 (and associated staple firing), or retracting the cutting member 49628.

The smart surgical stapler 49604 may receive a first discrete signal associated with clamping control operation (e.g., via a control circuit). The first discrete signal may be initiated (e.g., via the control circuit) by healthcare professional's actuation of a clamping control trigger. In response to the first discrete signal, a first continuous signal may be generated (e.g., via the control circuit) to cause a continuous application of force (e.g., on a clamping jaw) based on autonomous clamping control algorithm 49606.

Based on autonomous clamping control algorithm 49606, the continuous application of force may cause a clamping jaw to clamp down 49614 to reach a predefined tissue compression pressure and/or to reach within a predefined range of tissue compression pressures (e.g., when fully closed). A clamping jaw may be caused to clamp down 49614 in a controlled manner. In an example, between the initial contact 49612 with the tissue and 50% closure of the clamping jaw, a first pre-defined rate of closure may be used. Between the 50% closure of the clamping jaw and when a predefined tissue compression pressure is reached, a second pre-defined rate of closure may be used. In an example, between the initial contact 49612 with the tissue and 50% closure of the clamping jaw, a first pre-defined rate of increase in tissue compression pressure may be used. Between the 50% closure of the clamping jaw and when a predefined tissue compression pressure is reached, a second pre-defined rate of increase in tissue compression pressure may be used.

Based on autonomous clamping control algorithm 49606, the first continuous signal may be adjusted autonomously. For example, the continuous signal may be adjusted based on one or more measurements. The continuous application of force may be adjusted to cause a clamping jaw to adjust its closure rate when clamping down 49614, e.g., based on a pre-defined tissue compression pressure limit (e.g., a tissue load limit). Tissue compression pressure limits (e.g., tissue load limits) may be based on safety characteristics, such as risk of tissue damage or other concerns such as excessive tissue movement. If the tissue compression pressure (e.g., sensed by the clamping jaw) is measurement to be about to exceed (e.g., have exceeded) a pre-defined tissue compression pressure limit, the closure rate may be reduced to a lower rate (e.g., a pre-defined lower rate) or the closure may be paused (e.g., paused completely). The closure may be paused for a pre-defined period, such as 1 or 2 seconds. Such reduction of closure rate or pause of closure may allow the tissue to viscoelastically relax. When the tissue compression pressure measurement falls under an acceptable threshold (e.g., a pre-defined threshold), the closure rate may be increased back to the previous closure rate or may be resumed to the previous closure rate, accordingly.

The continuous application of force may be adjusted to cause a clamping jaw to restrict clamping down 49614, e.g., based on tissue property measurement(s) (e.g., tissue impedance measurement(s), which may indicate presence of rigid object(s) if the measurements are higher than expected measurements associated with a tissue. Visual detection of rigid object(s) may be used to supplement tissue property measurements to detect presence of the rigid object(s). In response to detection of rigid object(s), the continuous application of force may pause to cause the clamping jaw to stop during clamping down 49614. In such case, a healthcare professional 49602 may be provided with an opportunity to address the detection (e.g., opening the clamping jaw and removing the detected rigid object(s) manually).

After the pre-defined tissue compression pressure is reached for a fully closed clamping jaw (e.g., after clamping down 49614 completes), the continuous application of force may cause the clamping jaw to hold the tissue for a pre-defined period of time (also known as wait time 49616/tissue creep) before firing(s) is/are initiated 49624 (e.g., based on autonomous firing control algorithm 49608, as described herein).

The continuous application of force may cause the clamping jaw to maintain pressure/grip 49618 on the tissue during the firing sequence (e.g., when firing(s) is/are initiated 49624, during the period the cutting member is advancing 49626, and during the period the cutting member is retracting 49628, e.g., based on autonomous firing control algorithm 49608, as described herein). In an example, when the cutting member is advancing 49626 and hence pushing the tissue and increasing the load on the tissue, additional clamping force may be applied to the clamping jaw. The additional clamping force may be proportional to the increased load on the tissue. The additional clamping force may be utilized to maintain pressure/grip 49618 (e.g., constrain the tissue) and minimize tissue movement.

The continuous application of force may cause the clamping jaw to maintain pressure/grip 49618 on the tissue, which may include applying additional clamping force on the tissue during the firing sequence (e.g., based on autonomous clamping control algorithm 49606 as described herein). For example, the firing sequence may include multiple firing phases and the cutting member may pause (e.g., briefly) at the end of each firing phase. In such case, additional clamping force is applied to expel fluid from the cut tissue (e.g., for a pre-defined period) during the pause and the clamping jaw resumes advancing the clamping jaw after the pause.

The continuous application of force may cause the clamping jaw to maintain pressure/grip 49618 on the tissue, which may include progressively closing further during the firing sequence (e.g., based on autonomous clamping control algorithm 49606, as described herein). For example, when the cutting member is advancing and hence pushing the tissue and an increasing firing load is required to cut the tissue, the clamping jaw may be further closed progressively. The progressive closure of the clamping jaw may be proportional to the increased firing load. The progressive closure may help stabilize the tissue and hence reduce the firing load required to cut the tissue.

The smart surgical stapler 49604 may receive a second discrete signal associated with firing control operation (e.g., via a control circuit, as described herein). In an example, the second discrete signal may be initiated (e.g., via the control circuit) by healthcare professional's actuation of a firing control trigger. In an example, the second discrete signal may be actuated autonomously by autonomous clamping control algorithm 49606. The e.g., autonomous actuation may be in response to the completion of step Wait 49616. In response to the second discrete signal, a second continuous signal may be generated (e.g., via the control circuit) to cause a deployment operation (e.g., advancing 49626 a cutting member and retracting 49628 the cutting member to a starting position) based on autonomous firing control algorithm 49608.

Based on autonomous firing control algorithm 49608, the second continuous signal may cause a cutting member to advance a controlled manner. For example, advancement may accelerate to a pre-defined speed and maintain at that speed until it is sensed that there is no more tissue ahead of the cutting line and subsequently deaccelerate to a stop. Firing control algorithm(s)/control program(s) are described in greater detail in U.S. patent application Ser. No. 16/209,416, tided METHOD OF HUB COMMUNICATION, PROCESSING, DISPLAY, AND CLOUD ANALYTICS, filed Dec. 4, 2018 and in U.S. patent application Ser. No. 16/209,423, titled METHOD OF COMPRESSING TISSUE WITHIN A STAPLING DEVICE AND SIMULTANEOUSLY DISPLAYING THE LOCATION OF THE TISSUE WITHIN THE JAWS, filed Dec. 4, 2018, the disclosure of which is herein incorporated by reference in its entirety.

Based on autonomous firing control algorithm, advancement of the cutting member may be adjusted autonomously. For example, advancement of the cutting member may be adjusted autonomously based on a measurement. In an example, control algorithm may cause the advancement of the cutting member to pause if a sensed firing load exceeds a pre-defined threshold. In some cases, the pause may last a pre-defined amount of time, before advancement is resumed. In some cases, the pause may last until a tissue load (e.g., viscoelastical load properties due to the tissue) measurement falls below an acceptable threshold, before advancement is resumed. In some cases, there is a max number of times advancement may be attempted to be resumed after a pause and if a max number of attempts is reached, advancement may be completely stopped and manual intervention of a healthcare professional 49602 may be required.

In an example, control algorithm may cause the advancement speed of the cutting member to adjust based on force(s) (or load) sensed on clamping jaws. If a sensed firing load increases at rate that's above a predefined threshold, the advancement speed may be reduced for a predefined amount of time. After the predefined amount time elapses, the advancement speed may be increased to the previous advancement speed.

In an example, control algorithm may cause the advancement to completely stop if a maximum firing load (e.g., a maximum advancement force) or the maximum firing load is sensed more than a pre-defined maximum number of times. In an example, control algorithm may cause the retraction of the cutting member to completely stop if a maximum firing load (e.g., a maximum advancement force) or the maximum firing load is sensed more than a pre-defined maximum number of times.

Predefined settings may be used to control automation or discrete motions. The setup configuration of the surgical instrument may have a setting that instructs the surgical instrument to run fully autonomous to complete, run partially to a predefined step, or remain in discrete mode with limited or no autonomous action. A tiered system-based autonomy architecture may be implemented. A surgical instrument may come from factory set up for a full manual mode. The surgical instrument may enter an operating room (OR) and a surgical hub may establish a communication pathway to the surgical instrument. The pathway may be interrogated for speed and accuracy. If communication path is adequate, the surgical hub may instruct the surgical instrument about the level of autonomy that may be used in a surgical procedure. Specific break points may be established in an autonomous operation and the surgical instrument may pause and hold until a healthcare professional (e.g., a surgeon) or an OR personnel acknowledges the break and command the surgical instrument to continue. Breakpoints may be established based on individual healthcare professional 's preferences and/or may use the A1 collective data for common break points. Previous uses by a specific healthcare professional may be used to learn the healthcare professional's preferences and may allow the surgical hub to instruct the surgical instrument to set an autonomy level to a pre-defined setting. Detected issues with the device, previous procedure steps, patient biomarkers, or healthcare professional biomarkers, or surgical hub communications with the device may set the level of automated action the surgical instrument is capable of using.

An automated operation may be locked out based on detection of an incorrect situation. For example, adaptive close typically allow automatic action of the closure tube while the firing system is operating. If the closure system is near it limits or if it detects significantly early contact with tissue, the adjustment during firing may be disabled. The same triggers that control prevention of automation may adjust another system's automation.

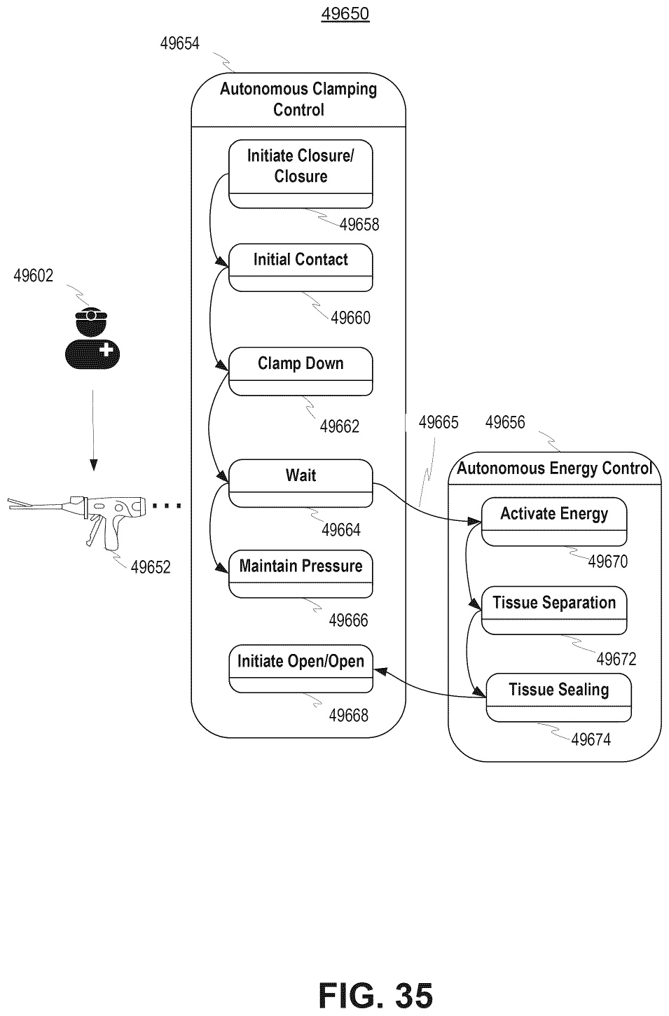

FIG. 35 illustrates an example autonomous operation of a surgical instrument, 49650. The surgical instrument may be a smart energy device. Additional details about operation of a smart energy device are disclosed in U.S. patent application Ser. No. 16/209,453, tided METHOD FOR CONTROLLING SMART ENERGY DEVICES, filed Dec. 4, 2018, the disclosure of which is herein incorporated by reference in its entirety.

A smart energy device may be a harmonic device (e.g., an ultrasonic scalpel). An ultrasonic scalpel includes an upper blade (which may include or may be a tissue pad) is an inactive one, which helps in grasping tissue(s) and prevents the vibration energy from spreading further while a lower active jaw vibrates and denatures protein in the tissue(s) to form a sticky coagulum. The mechanical vibrations may be produced by the piezoelectric transducers embedded in the device (e.g., in the upper blade and/or the lower active jaw) which convert the applied (e.g., produced) electrical energy to mechanical vibrations which are then transferred to the active blades for cutting or coagulation. The ultrasonic scalpel operates at a frequency of 55.5 kHz and has five power levels. Increasing a power level may increase cutting speed and decrease coagulation. Less power may decrease cutting speed and increase coagulation.

A smart energy device's (e.g., an ultrasonic energy device, such as an ultrasonic scalpel) operation may include clamping control and energy control. Clamping control may include control associated with one or more the following steps: initiating closure of a clamping arm/closure of a clamping arm, initial contact with a tissue, clamping down (e.g., to a pre-determined pressure), wait (e.g., for a pre-determined period during which tissue creep occurs), maintaining pressure (e.g., during energy generation), or initiating opening a clamping arm/opening a clamping arm. Energy control may include control associated with one or more the following steps: activate energy generation (e.g., by an energy blade), tissue separation (e.g., by an energy blade), or tissue sealing (e.g., by an energy blade).

A smart energy device 49652 (e.g., an ultrasonic energy device) may include a processor that is configured with one or more control algorithms for its autonomous operation, for example, as described in FIG. 5 herein. The smart energy device 49652 may be configured with a control algorithm associated with autonomous clamping control operation 49654. The smart energy device 49652 may be configured with a control algorithm associated with autonomous energy control operation (e.g., autonomous energy control algorithm 49656). The smart energy device 49652 may be configured with a control algorithm associated with the autonomous clamping control operation and the autonomous energy control operation. The autonomous clamping control algorithm 49654 may be associated with one or more steps, such as initiate closure/closure 49658 of a clamping arm, initial contact 49660 with a tissue, clamping down 49662 (e.g., to a pre-determined pressure), wait 49664 (e.g., before activating energy 49624), maintaining pressure 49666 (e.g., during energy generation), initiating opening a clamping arm/opening a clamping arm 49668 (e.g., after tissue sealing 49674 is complete). The autonomous energy control algorithm 49656 may be associated with one or more steps, such as activating energy 49670, energy generation for tissue separation 49672, energy generation for tissue sealing 49674.

The smart energy device 49652 (e.g., an ultrasonic energy device) may receive a first discrete signal associated with clamping control operation (e.g., via a control circuit). The first discrete signal may be initiated (e.g., via the control circuit) by healthcare professional's actuation of a clamping control trigger. In response to the first discrete signal, a first continuous signal may be generated (e.g., via the control circuit) to cause a continuous application of force (e.g., on a clamping arm) based on autonomous clamping control algorithm 49654.

Based on autonomous clamping control algorithm 49654, the continuous application of force may cause a clamping arm to initiate closure/closure 49658, make initial contact 49660 with a tissue, clamp down 49662 (e.g., to a pre-determined pressure), wait 49664 (e.g., before activating energy 49624), maintain pressure 49666 (e.g., during energy generation), initiate opening a clamping arm/open a clamping arm 49668 (e.g., after tissue sealing 49674 is complete).

The smart energy device 49652 may receive a second discrete signal associated with energy control operation (e.g., via a control circuit). The second discrete signal may be initiated (e.g., via the control circuit) by healthcare professional's actuation of an energy control trigger (or autonomous actuation by autonomous clamping control algorithm 49654, e.g., in response to the completion 49665 of step wait 49664). In response to the second discrete signal, a second continuous signal may be generated (e.g., via the control circuit) to cause a deployment operation (e.g., energy generation for tissue separation 49672 and energy generation for tissue sealing 49674) based on autonomous energy control algorithm 49656.

Based on autonomous energy control algorithm 49656, the second continuous signal may cause the energy blade to generate energy to separate the tissue. For example, the energy blade may generate energy of a first predefined power level (e.g., a higher level) that is sufficient to separate/cut issue.

During the tissue separation, the tissue content may be monitored/measured to determine whether to adjust the energy generation to seal the tissue. For example, ratio of collagen to elastin of the tissue may be measured (e.g., continuously during tissue separation). If collagen is measured to have been denatured below a predefined threshold, the energy blade may generate energy of a second pre-defined power level (e.g., a lower level) that is sufficient to seal the tissue.

During the issue sealing, the tissue content may be monitored/measured to determine whether to stop the energy generation (e.g., after tissue sealing is complete), e.g., to avoid damaging the upper clamping arm. For example, amount of the tissue may be measured (e.g., continuously during tissue sealing). If it is detected that no tissue is between the clamping arm and the energy blade, energy generation is stopped.

The clamping pressure between the active blade (e.g., the energy blade) and inactive blade (e.g., the clamping arm) may impact the tissue separation/tissue sealing (e.g., transection of tissue/vessel sealing) and/or may alter the frequency and/or impedance and/or cause blade fatigue. The clamping pressure may controlled (e.g., manually controlled) by the healthcare professional and may (e.g., drastically) alter the intended results. As described herein, allowing the smart energy device to autonomously control the clamping pressure and the amount/level of energy applied based on the tissue/vessel or intended action may improve consistency to therapeutic treatment and/or minimize trauma to unintended areas.

Based on autonomous clamping control algorithm 49654, the continuous application of force may cause a clamping arm to autonomously control grasping and tissue manipulation. In the case of tissue manipulation, pressure between jaws may be controlled to not damage tissue. Clamping pressure may be increased or decreased as healthcare professional grasps and moves tissue. When the healthcare professional moves the tissue, additional loads can be placed on the tissue as it moves/stretches, and/or loads on the tissue may reduce and the tissue fall out of the jaws. In such case, damage to the tissue may be caused, distractions and/or delays in procedure may be caused. The type of tissue the smart energy device is about to grasp (e.g., via sensor(s) of the smart energy device and/or visual detection data of the tissue from an image system/scope, as described herein) may be identified. When a scope/smart energy device detect the movement, direction, and load imparted on the tissue, the clamping pressure may be increased/decreased based on autonomous clamping control algorithm 49654.

The use of the visual feedback through a scope in conjunction of a smart energy device's control algorithm for monitoring impedance (e.g., autonomous energy control algorithm 49656) may be used to alter the clamping pressure and energy levels delivered to the smart energy device. The scope and the smart energy device 49652 (e.g., via the response of both systems) may control the therapeutic treatment (e.g., as opposed to the control by the healthcare professional). In an example, a bi-polar energy device may rely on clamping pressure and heat for therapeutic treatment and the approach described herein for use of visual feedback and control algorithm for monitoring impedance may be utilized for maintain the clamp pressure based on tissue type and/or vessel size, e.g., to optimize sealing.

Autonomous operation of a system actuation based on the detection of the maximum combined forces applied to the tissue may be implemented to minimize inadvertent tissue trauma during interaction. The clamping force may be limited by the tangential pull force and the clamping combined to limit tearing. The loading on tissue may be autonomously calculate based on the cumulative loading of multiple devices and this information may be used to influence the activation of an additional device. In an example, two graspers may be used to hold tissue in position for an energy transection (e.g., tissue transection with an energy device). Tissue load may be calculated based on the loading between these graspers. Tissue load during energy activation (or energy generation) may influence the quality of the seal. The energy activation level (or energy generation level) may be adjusted based on the calculated tissue load. Subtle autonomous adjustments to the grasper positions may be made to alter the tissue loading, e.g., reduce loading for improved seal quality.

Haptic feedback may be provided to a healthcare professional (e.g., a surgeon) when autonomous operation deviates from planned procedure steps and locations. For example, ortho style geo-fencing around tumor resection in solid organ may be implemented. Creation of liver resection plane may be implemented with respect to lobe/zone of tumor being removed.

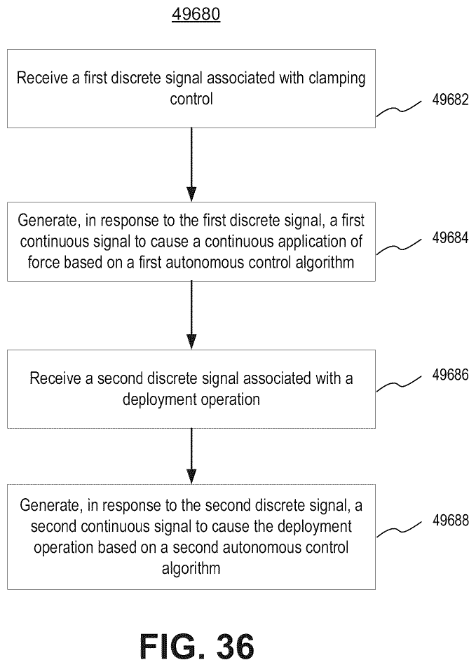

FIG. 36 is a flow chart of an example autonomous operation of a surgical instrument (e.g., a smart surgical device), 49680. At 49682, a first discrete signal associated with clamping control was received. For example, the smart surgical device may be a smart surgical cutting device or a smart surgical energy device. The first discrete signal may be associated with initiating closure of a clamping jaw. The first discrete signal may be triggered by a healthcare professional.

At 49684, a first continuous signal to cause a continuous application of force based on a first autonomous control algorithm, in response to the first discrete signal, is generated. For example, the continuous application of force may be adjusted autonomously based on at least a first measurement.

For example, the smart surgical device may be a smart surgical cutting device. The continuous application of force may be applied during one or more of the following steps: initial contact, clamping down, wait, maintaining pressure, or relieving pressure. The first measurement may be one of the following: a load on a clamping jaw at a first contact with a tissue, a load on the tissue when clamping down, and a tissue measurement that indicates presence of a rigid object.

For example, the smart surgical device may be a smart surgical energy device. The continuous application of force on a tissue may be applied during one or more of the following the clamping control: initial contact, clamping down, wait, or maintaining pressure. The first measurement may be a position of a tissue between a clamping arm and an energy blade.

At 19686, a second discrete signal associated with a deployment operation is received. For example, the smart surgical device may be a smart surgical cutting device. The deployment operation may be advancing of a cutting member and retracting of the cutting member.

For example, the smart surgical device may be a smart surgical energy device. The second discrete signal may be associated with initiating a firing sequence. The second discrete signal may be triggered by a healthcare professional or autonomously. The deployment operation may be generation of energy.

At 49688, a second continuous signal to cause the deployment operation based on a second autonomous control algorithm, in response to the second discrete signal, is generated. For example, the deployment operation may be adjusted autonomously based on at least a second measurement. The second measurement may be a ratio of collagen to elastin in the tissue.

Scope function may be autonomously controlled, e.g., based on tissue parameter(s) and/or healthcare professional-defined parameter(s). In the case of focusing/zooming function, a focal point may be adapted autonomously, e.g., based on monitoring end-effectors' current primary action and/or current interaction location. In the case of repositioning to control field of view, repositioning may be controlled to follow actions of a healthcare professional's instrument(s). Repositioning may be controlled to be based on a next step associated with a surgical procedure plan or an indication of a next step by the healthcare professional. Repositioning may be controlled to balance between two separate imaging sources to maximize field of view. In the case of adjustment of imaging configuration based on situational awareness, adjustment may be made to change from visual light to a multi-spectral wavelength or may be made to change back (e.g., based on the job(s), outcome(s), constraint(s) (JOC) at hand). In the case of monitoring of imaging outside of the displayed field of view and correlations of objects that are detected to identify potential interactions, displayed field of view may be digitally limited to a level smaller than the CMOS arrays are capable of detecting. A mesh of detectors may be used to look for object(s) and determine their location(s) and potential for collisions/interactions. In the case of signaling undesirable outcome not currently visible on the main screen, a popup (e.g., in a corner of main monitor) may be used to show a detected leak currently not visible (e.g., leak currently not visible due to off screen or leak currently not visible in current visual spectrum). For example, pancreatic leaks may be clear and very difficult to perceive, and alternate visualization technique(s) may be used to detect these and the healthcare professional may be alerted accordingly.

Surgical device movement controls (e.g., articulation) may be modified autonomously based on orientation of the surgical device on a healthcare professional monitor. Video analysis may be performed to determine a position of an end effector relative to the healthcare professional monitor. The healthcare professional controls may be adjusted based on the screen orientation of the end effector. For example, if the healthcare professional thinks in terms of left vs right, she/he may think in terms of left and right relative to the monitor screen. In such case, the method of movement control modification described herein may mitigate any confusion that may exist when handling of end effectors is for awkward positions. The method of movement control modification as described herein may be relative to the monitor being viewed by the healthcare professional (e.g., the left button on the monitor may move the end effector left). The method of movement control modification may be irrespective of the orientation of the surgical device's healthcare professional interface. In an example, a healthcare professional may place an endocutter in a patient and may handle the device in unknown position relative to the device's shaft. Real time video analysis may identify the end effector with its anvil facing up or down. The analysis may determine the end effector's position relative to the healthcare professional monitor (e.g., on the left or right of the monitor). A surgical hub (e.g., a control tower) may communicate to endocutter to adjust the controls of the endocutter (e.g., for a consistent healthcare professional experience). In such manner, the healthcare professional may operate articulation with minimal confusion (e.g., relative to the healthcare professional's view, such as left articulation button corresponding to going left and right articulation button corresponding to going right).

A detection mechanism may be implemented to monitor where the surgical device may be located. Choices may be provided regarding notifying information about a surgical device. The notified information may indicate how to handle the surgical device. For example, the information may include instructions regarding how to disconnect the surgical device and/or how to dispose the surgical device. Disposing off substances of very high concern may be regulated by one or more of a regional authority, a national authority, or a local authority. For example, The Batteries Directive of the European Union regulates the manufacturing and disposal of batteries and accumulators in the European Union to protect human health and the environment from hazardous substances such as mercury and cadmium. Similarly, Waste from Electrical and Electronic Equipment (WEEE) is another directive that focuses on waste electrical and electronic equipment or e-waste. WEEE directive's focus is on preventing the creation of WEEE, contributing to the efficient use of resources and the retrieval of secondary raw materials through re-use, recycling, and other forms of recovery, and improving the environmental performance of everyone involved in the life cycle of EEE.

Choices of how to notify to a healthcare professional about handling a surgical device that may include substances of very high concern, and how to disconnect and dispose such surgical devices may help adhere to the sustainability of directives for a manufacturer (e.g., the manufacturer of the surgical devices). A surgical system may be adaptable to additions based on, for example, the geographic location or a country. The surgical system may be updated and/or directives may be provided as they become available.

A surgical hub may provide instructions for disposing off batters, electronics, and/or substances of very high concern (SVHC) based on location-specific ordinances. In an example, a surgical hub may adapt to a predetermine disposal system. The disposal system may be country-specific and/or locality-specific wherein the surgical device or the medical facility (e.g., a hospital) may be located. In an example, the surgical hub may determine its geolocation. Based on the geolocation, the surgical hub may determine the country-specific, the province-specific or state-specific, and/or the locality-specific ordinances for compliance.

In an example, batteries may be separated and placed into a battery waste stream. For example, a surgical hub may direct the operating room personal about the relevant stream for example, depending on the device and the battery chemistry.

In an example, a surgical hub may have access to a surgical device's bill of materials (BOM). Based on the surgical device's BOM, the surgical hub may perform a check (e.g., a periodical check) for any updates to the safety data sheets (e.g., material safety data sheet (MSDS) or pathogen safety data sheet (PSDS)) for any updated disposal instructions. The surgical hub may perform check over the internet. The surgical hub may scan a manufacturers' database to determine the latest directives about the materials associated with a surgical device. A surgical hub may provide instructions a health care provider (e.g., an OR associate) about how to being the recapture of components for reclamation.

A surgical hub may determine geolocation and use the determined geolocation to determine the proper disposal instructions associated with a surgical device. The surgical hub may use the geolocation for determining region-specific and/or country-specific cleaning and sterilization protocols. In an example, the surgical hub may use a surgical device's device code to determine its location. In another example, the surgical hub may use global positioning system (GPS), network, hospital identifier, a manufacturer's cloud-based system to determine the location of the surgical device. In another example, the surgical hub may use the internet protocol (IP) address and/or software license to determine the location of the surgical device. In an example, the surgical hub may use the airport codes to determine the location of the device. The mechanism by which the location may be determined may be healthcare professional selectable. In an example, the surgical device (e.g., the surgical EEPROM may be coded with region-specific and/or country-specific information. In an example, a surgical device may determine location as a part of initialization check by checking settings (e.g., field service settings).

In an example, disposal bins with smart scanning of surgical devices or components used in the surgical device may be provided for proper disposal. Specific disposal bins may be provided for disposing off various categories of disposable surgical devices and/or components in the surgical devices. A surgical device and/or the component may be matched with a disposal bin for disposing off a surgical device or a component into proper disposal streams. In case a mismatch between a surgical device or a component and a disposal bin is found, a healthcare provider (e.g., and OR associate) may be notified of the mismatch. In an example, the disposal bins may have near-field communication (NFC) or radio frequency identifier (RFID) readers to track various types or surgical devices and/or components being dropped into those disposal bins. In an example, surgical devices with NFC or RFID type chips may be checked, for example, when the devices are dropped into a disposal bin to ensure proper placement in a disposal stream.

Systems and/or devices for smart disposal may be provided for cooperative interaction between one or more healthcare professionals, one or more disposal bins, and/or one or more surgical hubs. The disposal bins may be wired or wirelessly in communication with the one or more surgical hubs. In an example, disposal bins may communicate with a healthcare professional regarding the type of disposal that may occur or may be expected to occur between a disposal bin and a surgical device or a component to be disposed. The communication may be direct or via the surgical hub or an application. In an example, mini display on a disposal bin may be provided to communicate disposal instructions to a healthcare professional.

In an example, information and/or signals associated with a smart disposal bin may match with the information and/or signals associated with a surgical hub healthcare professional interface. In an example, an indication (e.g., in the form of LED lights or other means of communication) may be provided to indicate a match or a no match between the signals on a smart disposal bin and signals on hub UI. In an example, bins may be provided for disposal, reclamation and/or reuse. Each of the bins may have green, blue, and/or an orange color codes. A surgical hub display may communicate the type of disposal for each surgical device or component a healthcare professional may be handling. The surgical hub display may indicate a green light for a device to be disposed, and the light on the green bin may begin to flash or light up. In an example, a disposal bin may wait for a surgical device to be disposed. When the surgical device is dropped into the disposal bin, and the RFID scanner detects the correct device, the light may stop flashing and the surgical hub may indicate a confirmation message that disposal of the surgical device was successful. If the healthcare professional drops the surgical device in a wrong disposal bin, the disposal bin may flash red, or warning light and the surgical hub display may indicate an error indicating that the disposal of the surgical device was not successful.

In example, a surgical hub healthcare professional interface may be aware (e.g., actively aware) of status of disposal bins. The surgical hub may cross-check (e.g., actively cross-check) instructions with components in the disposal bins. In an example, a surgical hub may provide instructions (e.g., real-time instructions) for disassembly of surgical devices. For example, the surgical hub may provide the instructions we it may detect surgical devices and/or components being placed in the disposal bins. In an example, the surgical hub may keep track of a checklist of what is to be disposed of in a disposal bin against what is placed in the disposal bin.

A surgical hub may account for lost or missing devices during disposal of a surgical device and/or a component. In an example, a surgical hub may be aware of surgical devices and/or components that may be used during a surgical procedure, and smart disposal bins may cross check that each of the surgical devices used in a surgical procedure is accounted for during disposal to confirm lost or missing devices.

Disposal bins and/or one or more surgical hubs may communicate the disposal information with other hospital systems. In an example, the disposal bins and/or one or more surgical hubs may communicate with a healthcare facility inventory management system. In an example, cross checks may be made between the expected surgical devices that may be used in a surgical procedure with the actual surgical devices that were used and disposed of. Such comparison information may be communicated with the healthcare facility inventory management system.

In an example, disposal bins and/or one or more surgical hubs may communicate the disposal information with the cleaning staff of the healthcare facility. The disposal information may include information about cleaning and/or disposal unit within the healthcare facility, so they know what to expect when disposed devices are received. In an example, the disposal information may communicate with the cleaning staff of the healthcare facility that one or more disposal bins are full.

Smart disposal bins, for example, with device ID mechanisms, may collect surgical device data as the surgical devices and/or components are dropped into the disposal bins. For example, the data collected or some of the data collected may be stored on the surgical devices without communicating it with the surgical hub. In an example, smart disposal bins may scan devices and extract device data as they are dropped into the bins. The smart disposal bins may communicate the collected data with a surgical hub or a manufacturer's cloud system. In an example, smart disposal bins may connect to the surgical devices via RFID, NFC, etc. In an example, smart disposal bins and/or the surgical devices may interact with a manufacturer's cloud system via a gateway device.

In an example, an application on a mobile device (e.g., a phone application or a tablet application) may be used with device ID mechanisms and may be used to gain access to a surgical hub or a manufacturer's cloud-based data systems.

In an example, cleaning and/or sterilization staff may utilize a mobile device (e.g., a phone or a tablet) application with ability to scan and ID devices. The application may be integrated with the surgical hub networks for full interconnectivity, or when the surgical hub is not available, connect to a manufacturer's cloud site to gain access to device cleaning and sterilization protocols. Protocols may be communicated to the healthcare professional through the mobile device. Device ID, by way of NFC, RFID, BLE, etc. may be used to automatically extract device data and upload it to the manufacturer's cloud system.

In an example, an application (e.g., a portable application) may be provided on a mobile device with download and upload ability to accesses cleaning and/or sterilization protocols. The mobile device may utilize one or more of the following device identifying mechanisms: a QR code, a BLE connection, an NFC, or an RFID.

In an example, the application may be directly connected to the manufacturer's cloud system. Such an arrangement may be utilized in case of healthcare facilities that may not have access to a surgical hub. An access to surgical device cleaning and/or sterilization protocols may be provided. Step by step instructions about the cleaning and/or sterilization may be provided to healthcare professionals.

In an example, an application may utilize location information to provide country or region-specific cleaning and/or sterilization protocols and methods. The application may provide location services. The location may be automatically detected by using mechanisms as described herein or specified while setting up an account associated with the use of the application. The application may auto-connect to a manufacturer's customer service or a call center for help.

In an example, the application (e.g., alternatively or additionally) may be connected to a surgical hub system. The application may have special permissions (e.g., limited permissions) with surgical hub connectivity. For example, cleaning and/or sterilization staff may have limited access or no access to a portion of surgical hub system. The access may be limited to the cleaning and/or sterilization related information. The application may communicate with interconnected hospital systems, for example, for OR system and/or inventory tracking, etc.

The application may communicate with one or more sterilization groups to inform them about the upcoming tasks they may need to perform. For example, cleaning staff personnel in OR may scan surgical devices while disassembling or disposing them. In an example, when a device intended to be sterilized is scanned, the sterilization group (e.g., in the same healthcare facility location or in a different healthcare facility location) may be notified of the incoming devices.

The application may be utilized to identify or confirm lost and/or missing surgical devices. A surgical hub may track surgical devices used during surgical procedures. The application may scan each of the surgical devices during cleaning procedures and may confirm that each of the surgical devices have been accounted for. The application may confirm disposal (e.g., proper disposal) when application is in communication with a smart disposal system. The application may notify maintenance when equipment is ready for service

Data associated with a surgical device may be autonomously uploaded to a cloud system. The data upload may be initiated during device cleaning and/or sterilization. One or more surgical devices may store the surgical data associated with surgical devices throughout a surgical procedure. In an example, the surgical data may be stored on the device, for example, when connectivity with a surgical hub is not available. The surgical data may include device motor data, failures, error codes, etc.

In an example, surgical devices may have limited or no connectivity with a surgical hub. In an example, the surgical devices may not have surgical hub to collect data associated with surgical devices.

Surgical device data may be extracted from the surgical devices when they are scanned in for cleaning. The data extraction may be performed using BLE, RFID, NFC or other communication protocols.

Surgical device data processing may occur autonomously. Such processing may not be accessible to cleaning and/or sterilization staff. Surgical device data processing may occur autonomously once a surgical device is connected.

Surgical device data may be sent from an application (e.g., an application on a mobile device) to a surgical hub system or directly to a manufacturer's cloud system. In case of manufacturer's cloud, a gateway device may be used between surgical devices and a manufacturer's could system.

Intra-operative autonomous device evaluation, adjustment or refurbishment may be provided. Surgical devices used over a period of time in surgical procedures may degrade in performance or be damaged in a way that they do not perform optimally but may still be usable. Such surgical devices may be autonomously refurbished within a surgical procedure.

In an example, harmonic teflon pads may be made available and replaceable intraoperively. A teflon pad replacement cartridge/tool may be provided for longer than usual surgical procedures (e.g., extremely long surgical procedures), or in situations in which teflon pads are most prone to damage. The harmonic device may exit the patient, and autonomously be inserted into this tool where the damaged pad is removed from the device and a replacement pad is positioned into place.

In an example, as the teflon pads are damaged, if they cannot be replaced, a mechanical clamp arm adjustment may be made to raise or lower the pivot of the clamp arm and optimize the gap setting.

A surgical device's operational output of outcomes may be mapped back to the functional degradation of a part of a surgical device. The outputs may be used as an input to a system to indicate to the healthcare professional that the current remaining or expected performance and its degradation relative to original. The automated monitoring and comparison may be used to trigger updates to control programs and/or replacement or swapping out of parts or aspects of the device to revert the device's performance to its original level. In an example, an RF bipolar surgical device electrode conductivity or contamination may be used to trigger or indicate when the jaws of the surgical devices are to be cleaned. Cleaning of the jaws may be performed autonomously and/or intraoperatively. If the cleaning of the jaws does not result in a desired improvement or the degradation of the functionality is not reverted to a desired level, a selectively replaceable portion may be exchanged or replaced autonomously and/or intraoperatively. Such replacement may be performed, for example, when the system measures inappropriate resistance in an intentional short activation when the device is inserted or removed from the trocar. Data collected over a period of time from the continuous and autonomous monitoring and/or checking of the surgical device and the automatic comparison may be utilized for providing a better understanding of the surgical device.

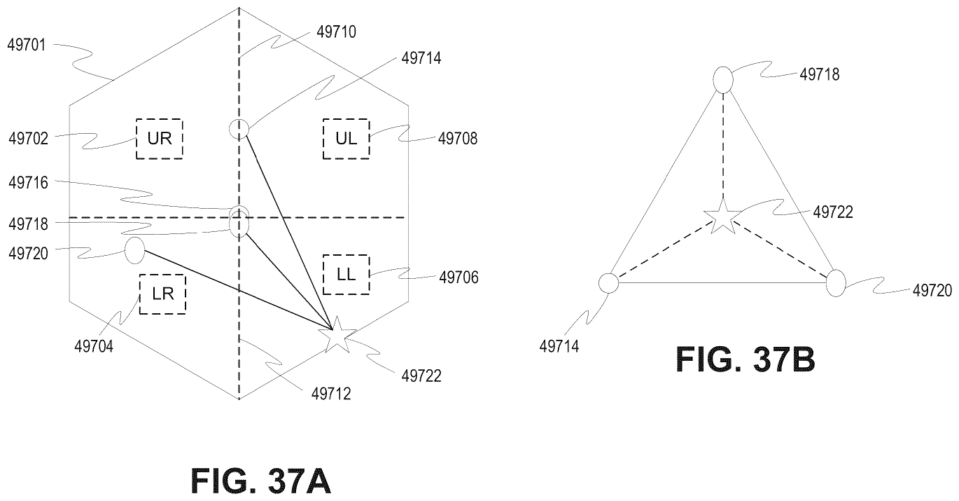

In laparoscopic surgery, trocars may be utilized to seal the skin openings, while permitting entry and removal of surgical instruments needed for a surgical procedure. FIG. 37A and FIG. 37B illustrate example trocar placements during a surgical procedure. As illustrated in FIG. 37A, a shape 49701 represents the abdomen of a patient's front. The shape 49701 is divided into upper right quadrant (UR) 49702, upper left quadrant (UL) 49708, lower right quadrant (LR) 49704, and lower left quadrant (LL) 49706, with umbilicus 49716 in the center. A midline consisting of an upper midline 49710 and a lower midline 49712 divides the shape 49701 into equal left and right halves. An oval shape 49718 overlapping with umbilicus 49716 represents a location of an incision for a laparoscope's trocar port. An oval shape 49720 in the LR area represents a location of an incision for a harmonic energy device's trocar port. A circle shape 49714 on the upper midline 49710 represents a location of an incision for a grasper's trocar port. A star shape 49722 represents a location of a target anatomy (e.g., sigmoid colon in a laparoscopic sigmoid colectomy). 49718, 49720, 49714 represent surgical choices of incision location for an incision for a trocar port. Solid lines between 49720 and 49722, between 49718 and 49722, between 49714 and 49722 represent the spatially relationships among the laparoscope, the harmonic device, the grasper when the three of them are all pointed at the target anatomy 49722 during a surgical procedure. Such spatial relationships represent a spatial arrangement of laparoscope and two surgical instruments that provide sufficient visibility of the surgical instruments as the three of them are working on the target anatomy. Such arrangement may be referred to as triangulation.

FIG. 37B shows a field-of-view perspective of the spatial relationships among the laparoscope, the harmonic device, and the grasper when the three of them are all pointed at the target anatomy 49722 during a surgical procedure. As such, the sight of the harmonic device and the sight of the grasper are maximized when the three of them are both pointed at and working on the target anatomy 49722 during a surgical procedure.

Instrument (e.g., surgical instrument) may perform autonomous action(s) during reloading, repositioning, and/or cleaning for completion of an action (e.g., a surgical action). For example, autonomous repositioning of an energy device may be performed during cleaning. Keeping the jaws and/or the blade clean and/or free of debris throughout a surgical procedure may prevent tissue and/or debris build-up, which may lead to unintended generator error(s) that may require troubleshooting (e.g., additional troubleshooting). A system (e.g., within the energy device or with a surgical hub linked to the energy device) may autonomously monitor for when the jaws require cleaning and may retract the energy device from a surgical site to be cleaned. After the jaws are cleaned, the energy device may autonomously return to the position (e.g., the exact position) the device was at prior to being cleaned.

A system (e.g., a system within a surgical instrument or within a surgical hub linked with the surgical instrument) may use contextual information (e.g., contextual information gathered from additional surgical hub inputs) to determine a time (e.g., a most appropriate time) during operation to remove (e.g., from a surgical site) and clean. For example, a surgical instrument may be automatically removed from a surgical site and cleaned during a monotonous mesentery or omentum separation. If a healthcare professional (e.g., a surgeon) is dissecting (e.g., carefully) a critical structure, a risk (e.g., a risk of tissue/debris build-up) may be alerted to the healthcare professional and the healthcare professional may be allowed to continue using the surgical instrument.

A number of remaining steps (e.g., surgical steps) and/or a remaining distance of dissection may be used to determine when to clean a surgical instrument. The determination may be based on the surgical instrument performance and/or impact (e.g., anticipated impact) of interruption of progress (e.g., momentum of surgical progress)

A stapling device may be autonomously repositioned (e.g., after reloading of cartridge). A stapling device may require reloading during a surgical procedure (e.g., based on a length of area that is stapled). After the stapling device is fired it may be detected that another reload is required and the stapling device may autonomously retract from a surgical site, e.g., to a position that was easily accessible for the reload. After the reload, the stapling device may return to a position (e.g., an exact position where the stapling device was prior to the reload). The stapling device may identify a reload (e.g., a required reload) and may confirm the reload (e.g., confirming the required reload is correct).

An endocutter reload tray may be positioned such that a system (e.g., a system within the endocutter or a surgical hub) may autonomously remove the endocutter from a patient and may autonomously reload the device. Proper positioning of the reloads may ensure that arms (e.g., robotic arms holding the endocutter) may be moved without interfering with other arms (e.g., robotic arms) or obstructions. An optimal reload tray position may be determined, e.g., based on a surgical procedure type, devices installed, a healthcare professional's (e.g., a surgeon's) preferred arm positions, etc. A system (e.g., a system associated with a surgical hub) may position the reload tray, and/or may direct assistant(s) for proper positioning. The reload tray may be attached to an unused robotic arm and the tray may move to a position (e.g., a most appropriate position) when needed. The in-room (e.g., operating room) monitoring of a surgical hub may be used to determine an exact location of a holding structure relative to a robotic arm trocar holding location. In such manner, the robotic arm may be positioned in a location (e.g., a best location) for reload automatically. If robot arm(s) that can only retract the instrument to a point where it is still within the trocar. In such case, a healthcare professional (e.g., a surgeon) may be required to remove the instrument from an instrument driver (e.g., a tool driver) and may manually remove one cartridge and load another. Once a newly loaded instrument (e.g., tool) is re-attached to the instrument driver (e.g., the tool driver), the robotic arm may (e.g., then) finish the automation of motion for repositioning. In such example, the automatic motions may be retraction and/or re-positioning and loading a new cartridge may be automated or may be performed manually.

Registration to marker(s)/instrument(s) may be for tracking/repositioning. Positioning of instrument/tools may be difficult to perform because it may require tracking of an anatomy (e.g., an underlying anatomy) and/or may require maintaining registration to the underlying anatomy. Maintaining registration to the underlying anatomy may allow the instrument/tool to register its position in a space (e.g., a body cavity space) prior to retracting. By identifying markers/anatomical structures, the instrument/tool may reposition itself back into its previous position. For example, the instrument/tool may account for any patient movement/anatomy movement and may reposition itself to its previous point (e.g., adjusting to compensate for the movement). The instrument/tool may use other instruments/devices/tools as markers for registration in a space (e.g., a body cavity space) during repositioning when the instrument/tool is being cleaned or reloaded.

Virtual access boundaries for large motion automatic reposition(s) may be implemented. A surgical instrument may be controlled to autonomously reposition obstructions during navigation to a treatment site. A surgical procedure may require manipulation and dissection of a tissue, organ(s) and/or repositioning obstruction(s) to gain access to the treatment site. For example, a vision system, an imaging system, and an A1 control may be used to identify a targeted area, obstructions and may autonomously control the graspers/retractors to move the obstructions, e.g., to gain the most access. In such case, the required access needed may be visible or determined based on the healthcare professional's (e.g., the surgeon) knowledge of the volume of space required for an instrument to pass and/or gain access and/or size of resection that the healthcare professional was planning to remove. In some examples, pre-operative imaging (e.g., imaging associated with previous surgical procedures, such as gold standard procedures), patient biometrics, and/or the scope may be used to determine optimal control of organ/tissue/obstruction repositioning, such as determining how much/far organs/tissue/obstructions may be moved to minimize trauma.

A surgical device's range of motion may be autonomously controlled. Virtual boundaries may be created for a surgical device's range of motion (e.g., to constrain the range of motion/articulation). For example, virtual boundary/structure may be created for each surgical instrument/device autonomously. Such virtual boundary/structure may constraint a healthcare professional's movement(s) to a certain volume (e.g., to serve as a guide to the healthcare professional, which may identify if she/he needed to reposition and may protect the patient from the instrument/device making unattended contact, which may damage unintended areas). Such virtual boundary/structure may be shown on a monitor (e.g., a healthcare professional's monitor) and/or may restrict the movement(s) of the instrument/device in areas outside of the intended treatment area.

A fire-and-forget system operation (e.g., of a surgical instrument) may be implemented. The fore-and-forget system operation may be sequential. The fore-and-forget system operation may be based on healthcare professional selected input and/or autonomous instrument control. For example, autonomous fire-and-forget system operation of monopolar devices may be implemented. Monopolar devices may use an electrosurgical generator, which may have two primary functions, such as cut and coagulate settings. The cut function may use unmodulated continuous waveform. The unmodulated continuous waveform may result in a flow of low energy electron and may generate minimal smoke production during tissue cutting. The coagulation function may use modulated interrupted waveform. The modulated interrupted waveform may be associated with a high energy electron flow and may generate more smoke production with high temperature but better hemostasis. A monopolar device may have an ability to use a continuous and/or mix/blend current to dissect tissue to achieve hemostasis. Autonomous selection of cut, coagulate and/or a blended energy may be applied, e.g., based on detection of an imaging system and/or identification of tissue type(s). An amount of energy and a direction of energy may be autonomously controlled, e.g., based on tissue and/or surrounding structures.

For example, autonomous fire-and-forget system operation of a bi-polar and/or an ultrasonic device may be implemented. Bi-polar/ultrasonic devices may be used for dissection and/or tissue-sealing and hemostasis. Automation of the firing may be performed to control a speed to close the jaws, to control clamp pressure required (e.g., based on a tissue/vessel type), to control wait time of compression, and/or to control the energy level(s) that are applied. Monitoring through a scope device may be used to verify/confirm and/or may be used to modulate or adjust.

For example, autonomous fire-and-forget system operation of combo energy devices may be implemented. Combo energy device may autonomously control a type of energy that is applied based on the tissue type and/or vicinity to surrounding structure(s), e.g., to minimize unintended tissue damage. Visual detection through a scope device and identification of surrounding structures may be used to control the energy the device is configured to (e.g., is able to) activate.

For example, autonomous fire-and-forget system operation of stapling devices. Automation of the firing may be performed to control a speed to close the jaws, to control clamp pressure required (e.g., based on a tissue/vessel type), to control wait time of compression, and/or to control a speed of the motor to drive the firing mechanism.

Autonomous fire-and-forget tissue tension monitoring may be implemented. Sealing and/or transecting vessels/tissue/organs when using an energy device and/or a stapling device may be altered, e.g., based on tension under a targeted area/zone that is being fired on. Automatically monitoring the tension under a targeted area/zone may be used prior to activating the firing of the device. In the case of harmonic devices, bench top testing (e.g., bench top testing used to submit for regulatory approval) may indicate that sealing of vessels is performed under an axial tension of 50 g on the vessel. To optimizes a system (an autonomous fire-and-forget system), the jaws may apply a 50 g axial load on the tissue for sealing.

Tissue tension may be manipulated as a variable in sealing. Subtle device movement(s) may be used to increase/decrease tissue tension, e.g., based on sealing/transecting/speed prioritization. For example, a tissue may be detected as mesentery. The energy device/instrument may prioritize a faster speed and may increase tension during energy activation. In the case of increasing speed of transection, the energy device/instrument may apply a movement (e.g., a subtle movement) to lift an end effector (e.g., orthogonally in relation to jaw clamping). Such lift may be performed during the entire energy activation or for a portion of the energy activation, e.g., based on the speed required. Such movement may be subtle (e.g., imperceptible to the healthcare professional).

In an example, a tissue may be detected as a critical vessel. The energy device/instrument may prioritize seal quality and may decrease tension during energy activation. In the case of prioritizing seal quality, tissue tension may be reduced (e.g., minimized). Reduction of tissue tension may be performed by monitoring end effector joint loads and/or tissue characteristics through visualization and moving (e.g., subtly) the end effector away from direction(s) of higher load. Tissue tension may change through the sealing cycle and subtle movement(s) may be performed throughout sealing, e.g., to ensure minimal tension.

One or more triggers may be used to assess tissue tension. In examples, visual analysis of a tissue may be performed and changes in tissue coloration adjacent to jaws may be a trigger. Visual analysis of width of a tissue may be performed and high tension may be indicated by a narrow tissue, which may be a trigger. Perfusion analysis may be performed, and decreased perfusion may be a trigger/indicator that excess tension is being applied. Device loading may be monitored, and shaft loads and/or jaw loads may be detected as a trigger. Tissue impedance and the like may be monitored and changes in tissue impedance (e.g., relative to clamp load) in combination with jaw gap and/or tissue position may indicate changes in tissue tension. Tissue position in jaws may be monitored and used to detect a trigger for assessing tissue tension. In the case of a tissue with no tension, under a given clamp load may have a certain area that is taken up in the jaws. If tissue tension is increased, the tissue may narrow within the jaws, which may indicate tension. Secondary devices, such as an ultrasound probe or other means, may be used to monitor tissue properties and detect a trigger for assessing tissue tension.

Semi-autonomous robotic arm repositioning may be implemented. Autonomous arm/stand repositioning may be implemented to minimize motion and/or interaction with object(s). For example, manual arm positioning in admittance mode may be based on geofencing (e.g., according to anatomic scans), e.g., for optimized control and reach (e.g., after a surgical instrument/device is docked on the arm/stand). Arm pre-positioning may be implemented for introduction of new instruments in a surgical procedure (e.g., an endocutter). existing in-use arms and/or positions (e.g., potential positions) outside a patient body may be evaluated (e.g., considered) to minimize interaction between a new arm and existing arm(s) for access to a surgical site. A virtual instrument/tool for simulation of location with end-effectors may be used (e.g., in the evaluation described herein).

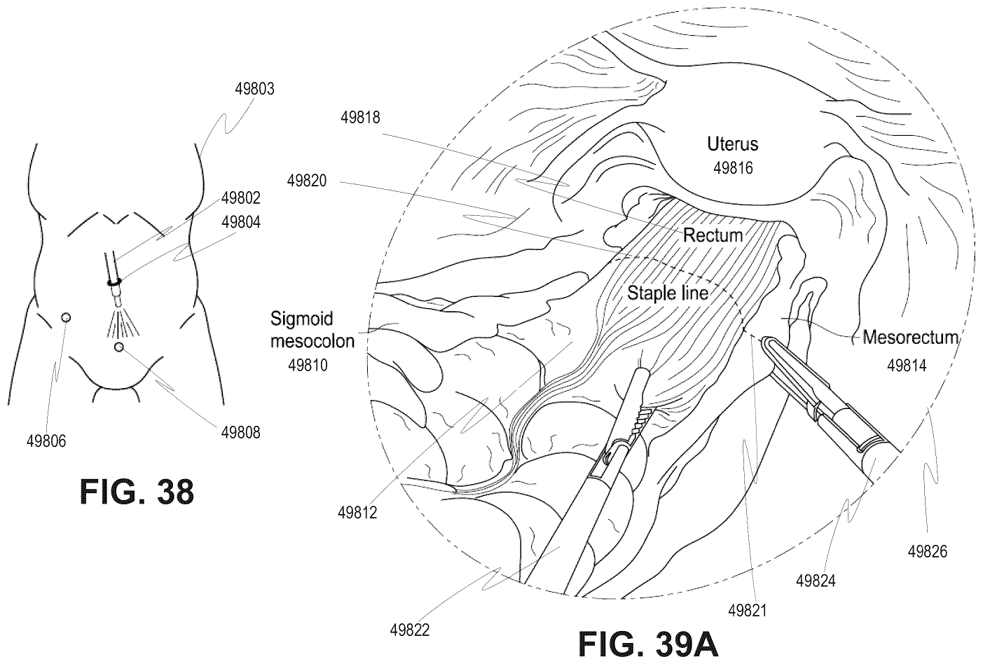

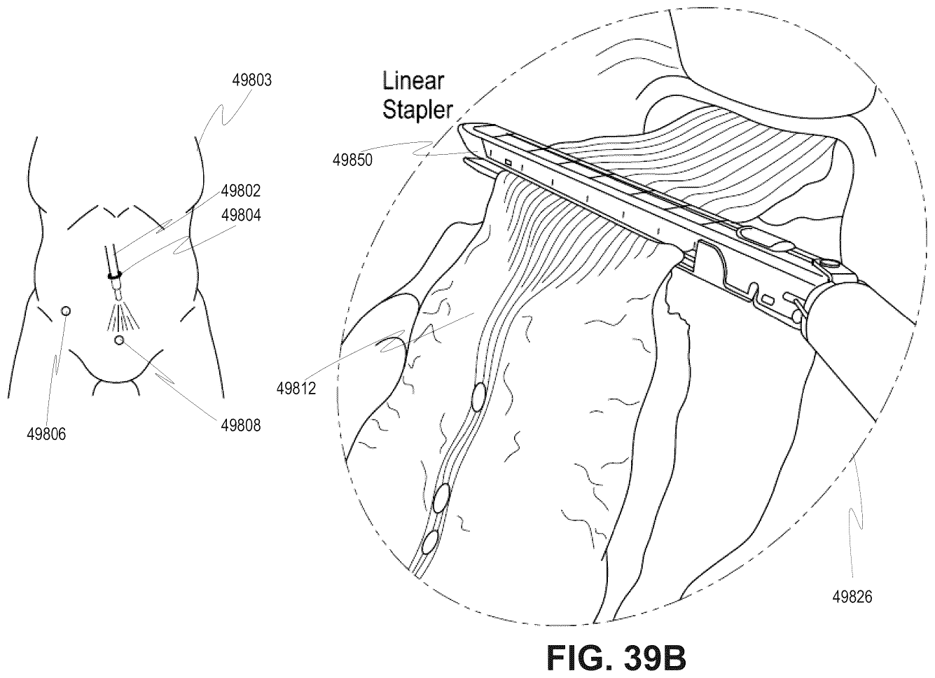

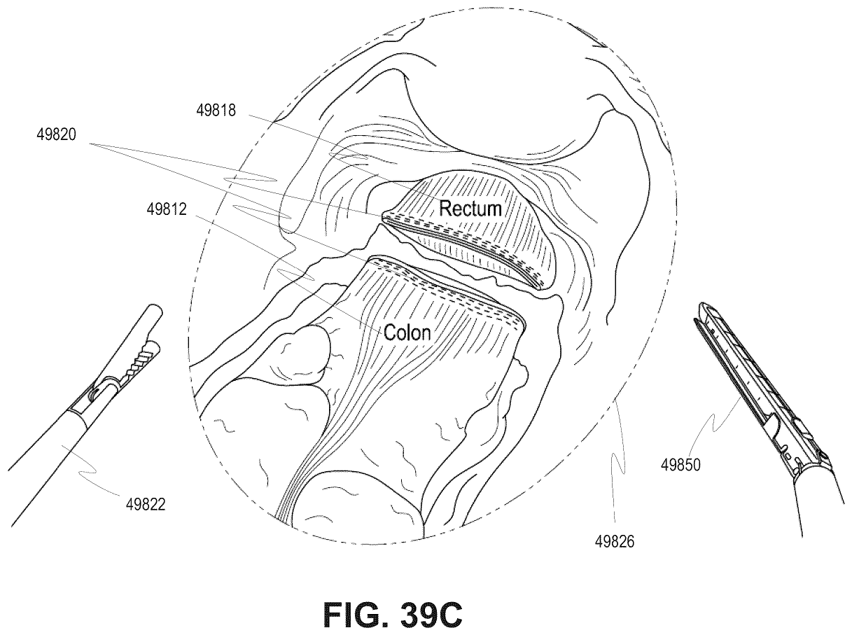

FIG. 38 illustrates example trocar placements in a laparoscopic surgical procedure. A first trocar port 49804 (e.g., 49718 as described in FIG. 37A through FIG. 37B) may be a port near the umbilicus (e.g., 49716 as described in FIG. 37A). A scope device (e.g., a laparoscope) 49802 may be inserted into trocar port 49804 to create a field of view (e.g., 49826 described in FIG. 39A through FIG. 39B). The field of view may be presented (e.g., via live stream) to a display device (e.g., for a healthcare professional to view). A second trocar port 49806 and a third trocar port 49808 may be two other trocar ports. Port 49806 may be a port for a grasper and port 49808 may be a port for an energy device or a linear stapler, or vice versa. The three trocar ports 49804, 49806, and 49808 form a triangulation, as described herein (e.g., in FIG. 37A through FIG. 37B).

FIG. 39A illustrates an example surgical step autonomously controlled by a computing device. The computing device may be a robotic computing device that controls one or more surgical devices/instruments. As illustrated in FIG. 39A, field of view 49826 may be associated with a surgical procedure, such as a laparoscopic sigmoid colectomy procedure. A laparoscopic sigmoid colectomy procedure may include the following surgical steps: initiate, access, mobilize colon, resect sigmoid, perform anastomosis, and conclude. A surgical step may include surgical tasks. For example, the surgical step access may include the following surgical tasks: dissect adhesions, dissect mesentery, and identify ureter.

As illustrated, field of view 49826 shows the computing device performing autonomous operation associated with surgical task dissect mesentery. Field of view 49826 shows a surgical site's anatomy, which includes sigmoid mesocolon 49810, sigmoid colon 49812, mesorectum 49814, rectum 49818, and uterus 49816. Field of view 49826 shows surgical instruments for the dissect mesentery surgical task, such as a grasper 49822 and an energy device 49824.

The computing device may include a processor. The computing device (e.g., the processor included in the computing device) may be configured to control a surgical device to operate autonomously within a predefined boundary. Based on a condition being satisfied, the computing device may be configured to determine a safety adjustment to the operation. The computing device may be configured to control the surgical device to operate based on the safety adjustment.

In an example, the computing device may be configured to control grasper 49822 to operate autonomously within a predefined boundary to perform surgical task dissect mesentery (e.g., as shown in FIG. 39A). Graspers (e.g., grasper 49822) may be used to mobilize, hold and/or place under tension a tissue (e.g., sigmoid colon 49812). The predefined boundary may be a virtual movement boundary associated with surgical task dissect mesentery.

A virtual movement boundary may be regions or adjustable geo-fencing defined by a healthcare professional (e.g., a surgeon), e.g., to safeguard against autonomous action outside of a pre-defined area. Such pre-defined area may use healthcare professional-defined operational parameters of the autonomous operation. A surgical instrument may be allowed to operate within the operational parameters in an autonomous fashion. In an example, surgical instruments/devices outside of the current scope field of view may be prohibited from autonomous operation. For example, a healthcare professional may draw a virtual line showing the location of the line or path to track with the surgical instrument. In such manner, the healthcare professional may set where to cut/staple and may (e.g., then) monitor the surgical instrument as the surgical instrument completes a surgical task. Variables and feedback may be processed by the computing device (e.g., in or near real time), which may enable the surgical instrument to make adjustments to the operation based on detected properties and/or behaviors. In examples, the tissue thickness may be determined during closing/closure. Wait time may be pre-programmed based on tissue type(s) and thickness in clamping jaws. Cutting speed(s) (e.g., advancing speed(s) of the cutting member, such as a knife) may be set, for example, pre-configured and/or based on the previously used parameters. In such manner, control of individual aspects of surgical instrument operation by the healthcare professional may be limited and the healthcare professional may be enabled to place additional concentration on procedural step(s) (e.g., more than on each individual firing).

As illustrated in FIG. 39A, staple line 49820 may be a virtual movement boundary defined by the healthcare professional for performing surgical step of resect sigmoid as described herein. Staple line 49820 may be marked and superimposed on the anatomy in field of view 49826 (e.g., using 3D model/augmented reality). Staple line 49820 may mark the line of resection for the surgical step of resect sigmoid. Staple line 49820 may extend to the line of dissection 49821 in mesorectum 49814 for the surgical task of dissect mesentery. In the case of energy device 49824, surgical task of dissect mesentery may be controlled to perform autonomously by a computing device, e.g., by following the dissection line 49821.

FIG. 39B illustrates an example autonomous operation of a surgical instrument. As illustrated, field of view 49826 may be associated with a laparoscopic sigmoid colectomy procedure. Field of view 49826 shows a surgical site's anatomy (e.g., as illustrated in FIG. 39A). As illustrated, operation of surgical step resect sigmoid may be autonomously controlled. As illustrated, linear stapler 49850 is controlled by the computing device to autonomously resect sigmoid colon 49812 by following staple line 49820.

FIG. 39C illustrates an example operation of a surgical instrument. As illustrated, field of view 49826 may be associated with a laparoscopic sigmoid colectomy procedure. Field of view 49826 shows a surgical site's anatomy (e.g., as illustrated in FIG. 39A through FIG. 39B). As illustrated, autonomous operation of surgical step resect sigmoid is limited. Surgical step resect sigmoid has been completed, grasper 49822 and linear stapler 49850 are being retracted from the surgical site. In such case, autonomous operation of the jaws in grasper 49822 and/or linear stapler 49850 are locked (e.g., prohibited). Autonomous operation of the jaws in the grasper 49822 and/or the linear stapler 49850 are locked, for example, to avoid unintended damage to the tissue(s) during the retraction.

Referring to FIG. 39A, grasper 49822 may be autonomously controlled to mobilize, hold and/or place under tension a tissue (e.g., sigmoid colon 49812). The computing device may perform tissue tension measurement(s) to ensure safety related to the tissue. Strain measurements (e.g., measurement of strain applied to the tissue) may be performed. In an example, an imaging system may be used and marks (e.g., dots) may be placed on the tissue to allow relative force to be calculated on the tissue (e.g., using a 3D model of the anatomy and augmented reality). In an example, a stretchable flex circuit may be used as a temporary implantable device, for example, to provide measurements of a strain gauge and to provide information about the condition of the tissue. In an example, measurement(s) of the strain applied to grasper 49822 may be performed. The measurement(s) of the strain applied to grasper 49822 may be performed using a strain gauge in grasper 49822. Acceleration measurements (e.g., acceleration of motion of grasper 49822) may be performed.

Sharp changes in measurements may be monitored. For example, sharp change(s) in acceleration may be detected. If acceleration starts to slow down when a constant force is applied, it may be an indication that the tissue is resistive (e.g., more resistive than average). It may be detected that there is a sudden increase in force (e.g., strain). It may be detected that there is a sudden decrease in force (e.g., strain). It may be detected that there is a transition from a steady state in force to a state where acceleration starts to increase. In such cases, it may be an indication that grasper 49822 may have lost hold of the tissue and/or grasper 49822 may have started to tear the tissue. In response, the computing device may send a control signal to grasper 49822 to cause reduction of gasping force/strain.

Measurements of strain/acceleration may be based absolute value(s). Strain/acceleration may be measured to higher than an acceptable threshold (e.g., a maximum strain/force/tension). An acceptable threshold may be based on direction of force applied to issue/grasper 49822. An acceptable threshold may be based on the force grasper 49822 may tolerate (e.g., safely). An acceptable threshold may be a speed threshold by which grasper 49822 may move, which may limit grasper's 49822 acceleration. An acceptable threshold may be movement limits that are based on the cavity of the patient being operated on. In examples, the movement limits may be (e.g., dynamically) based on the cavity size available. The movement limits may be more constrained as grasper 49822 moves closer to different parts in the body cavity.

Referring to FIG. 39B, linear stapler 49850 may be autonomously controlled to cut/staple a tissue (e.g., sigmoid colon 49812). In an example, linear stapler 49850 may be stopped of its cutting cycle before linear stapler 49850 completes the cycle and in such case linear stapler 49850 may alert that other devices/system it has stopped cutting prematurely.

In an example, in the event that a fault condition (e.g., a condition internal to the linear stapler 49850) is detected very early in the firing sequence. An inrush current for the firing sub-system of linear stapler 49850 may be monitored during the initial moments of the firing sequence. For example, 100 ms into the firing sequence, the current may be detected to be too low, and the computing device may stop linear stapler 49850 before any significant amount of firing is performed.

In an example, a fault or concern may be detected in the tissue seal quality (e.g., indicating the tissue seal is compromised), the cutting cycle may be stopped. For example, a staple may be detected to be deformed, or energy device may have failed to produce a quality seal. In such case, a subsequent cutting action may not be performed.

In an example, an instrument condition may be detected and firing type may be changed accordingly. For example, such condition may be a low battery condition or may be motor overheating condition. Under such condition(s), linear stapler 49850 may automatically change its firing mode (e.g., such as to pulsed), e.g., to be more energy/thermally efficient and to ensure the firing cycle is able to be successfully completed.

In an example, no change in firing type may be made, which may be the nominal or default state of linear stapler 49850. For example, in such case, it is assumed that everything is working correctly and there is no reason for the system to not behave in this fashion.

Linear stapler 49850 may be autonomously controlled to cut/staple a tissue (e.g., sigmoid colon 49812), e.g., based on healthcare professional selection(s). Healthcare professional selection(s) may include selection of precision or cutting cycle completion rate. In an example, a healthcare professional may select a 60 mm load for a 50 mm cut and such selection may be programmed via the computing device. In such case, the cutting cycle may stop 10 mm early and may retract at the end of the 50 mm cut. In an example, a healthcare professional may select the entire available length of the cartridge for the cutting cycle. In such case, the cutting cycle may stop at the end of default cut cycle (e.g., a 50 mm cut with a 50 mm load).

Referring to FIG. 38, as illustrated trocar locations 49804, 49806, 49808 may be determined. Such trocar locations may be determined manually (e.g., by a human, such as a surgeon). In an example, a healthcare professional may enter a position of a trocar on a computer screen (e.g., in an OR), e.g., with assistance of laser imaging and positioning and/or an external sensor (e.g., such as a camera used for patient positioning). In such manner, absolute positioning of the trocar in space may be determined, such as position of the trocar on the patient and/or the angle at which the trocar is inserted into the patient's body.

Such trocar locations may be determined automatically (e.g., using automated Trocar insertion and final placement optimization). The computing device may suggest an optimal port placement, e.g., by causing to project laser points on a patient's body to indicate an optimal (e.g., best optimal) placement position. A healthcare professional may accept or reject such suggestion. The computing device may cause to have complete control over pressure being applied to insert the trocar. After the tip of the trocar has pierced the outer tissue, the trocar may determine that it has detected internals and in such case the knife associated with the trocar may be retracted to avoid cutting any internal structures. During the insertion, the trocar may detect obstruction and may stop the insertion. Different such trocars may be discriminated (e.g., by the computing device). Surgical instrument/device/tool may be inserted into such trocars. Such insertion may be automatic or manually performed by a healthcare professional (e.g., by a surgeon). Limits may be associated with the speed and/or forces a trocar/device may perceive. The computing device (e.g., using camera(s) and sensor(s)) may monitor the manual process and may alert the healthcare professional, if an error is detected.

Location and/or orientation of a surgical instrument/device/tool may be determined, e.g., based on associated trocar locations. In an example, when a surgical instrument/device is inserted into a trocar, the surgical instrument/device may be synchronized with the trocar automatically. In such manner, the location/orientation of the surgical instrument/device (e.g., in a body cavity) may be determined based on the location/angle of the trocar insertion. Triangulation (e.g., as shown in FIG. 37A through FIG. 37B) of the surgical instrument(s)/device(s) in the body cavity may be controlled based on the location/angle of the trocar insertion. Based on the port placement, the computing device may adjust movement of surgical instrument/device/tool to minimize movement issues. For example, the computing device adjust movement of one surgical instrument/device/tool to reflect a trocar port placement, e.g., based on linear distance(s) and/or angular distance(s).

A patient's body location/orientation may be determined. In an example, a smart hospital bed may use pressure sensors to detect where the body is and may send that information to the computing device. In an example, smart bands around key extremities (e.g., wrist(s), ankle(s), etc.) may be used to detect a patient's motion and the motion information may be used build a model of the body. Such model may guide the positioning of surgical instrument(s)/device(s) in the body cavity. In an example, a camera may capture the position of the patient and may use captured information to build a machine learning/AI model. Such model may guide the positioning of surgical instrument(s)/device(s) in the body cavity. The models described herein may help determine absolute positioning of surgical instrument(s)/device(s) in space (e.g., body cavity).

The computing device may determine movement of a surgical instrument/device and associated safety limits based on the relationship between the trocars, the patient body, and surgical instruments/devices. For example, as illustrated in FIGS. 38 and 39A, location/orientation of energy device 49824 and grasper 49822 may be determined based on location/angles of trocars 49806 and 49808, respectively, e.g., after energy device 49824 and trocar 49806 are synchronized (e.g., post-device insertion) and grasper 49822 and trocar 49808 are synchronized (e.g., post-device insertion). Patient body's 49803 location/orientation may be determined (e.g., based on a smart bed (not illustrated) the patient is lying on). In an example, the computing device may determine that a distance between energy device 49824 and grasper 49822 is below a safety threshold and may determine to cause energy device 49824 to retract by a predefined distance (e.g., 1 mm), e.g., to avoid potential collision between energy device 49824 and grasper 49822.

Confirmation, authorization, and/or initiation of intended activation of an automated step may be directed by a healthcare professional (e.g., a surgeon). The automation may be monitored, for example, via one or more authorizations of steps (e.g., a sequence of authorizations by a surgeon). In such manner, greater control over a surgical procedure may be maintained and uncertainty of the surgical procedure and/or patient-related variability may be mitigated.

A robotic system (e.g., the computing device described herein) may be trained to sequence though steps and may learn from the steps. A healthcare professional (e.g., a surgeon) may manually move surgical instruments/devices required position(s) and may acknowledge the position(s) are correct position(s) (e.g., a correction location with x, y, z coordinates). The computing device may recognize step(s) in addition to the step(s) the healthcare professional performed previously and may see authorization form the healthcare professional to add the additional step(s) to a surgical procedure map (e.g., a surgical procedure plan that includes all the steps to perform the procedure). A conditional robotic breakpoint may be used based on sensor or environmental conditions associated with the robotic system.

The computing device (e.g., a robotic system) may automatically define breakpoints based on the obvious differences in steps (e.g., that were performed by the healthcare professional) and may allow the healthcare professional to confirm the breakpoints (e.g., to provide more authorization of the breakpoints). Breakpoints may be defined based on complexity of operation, the tool, the healthcare professional training, risk, etc. In an example, the computing device may submit (e.g., place) breakpoints for further assessment (e.g., by a surgeon) before proceeding with a surgical procedure. Such obvious differences may be detected, e.g., based on monitored parameters like video stream data, tissue impendence data, force data, etc. In the case of a sleeve gastrectomy step, the staple operation may encounter mid-cycle a staple that causes it to exceed a high force threshold, which may cause a pause to allow additional creep and lower the force/trauma on the tissue. In such case, the staple operation may start back up and may automatically continue. At the end of the stroke, a clear breakpoint may occur (e.g., because a healthcare professional can clearly see the staple operation is at the end of its motion) and the staple operation may wait for the healthcare professional to allow activation of retraction after the breakpoint.

The obvious breakpoints described herein may be clear delineation(s) of a sequential set of automated steps, where a healthcare professional (e.g., a surgeon) may be verifying the operation to ensure the automation is performing the steps. In some examples, such similar operation may be part of a closed loop control.

In some examples, repeated steps may be desired, and a healthcare professional may have a means to indicate to repeat a previous set of automated steps. A healthcare professional may be provided with the ability to modify and/or add additional steps if the healthcare professional feels the need to replicate step(s). In the case that a surgical device indicates a tissue is positioned incorrectly, the healthcare professional may request to open and reposition the surgical device. The healthcare professional may determine that more tissue needs to be removed, e.g., to ensure a good margin. The surgical device may provide the ability to adapt the sequence of steps, e.g., based on unique tissue conditions. In examples, unique tissue may be automatically recognized by the surgical device. Recognition may be based on physician knowledge or prior knowledge, such as a patient is prone to bleeding, has low blood pressure reading, etc.

During an unforeseen event, emergency, a healthcare professional may take full control of the automated steps (e.g., regardless of if it deviates from defined automated procedure steps). In an example, robotic arms may be reverted safely back to safe position(s). A system (e.g., a robotic system) may pause and may await direct command(s) from the healthcare professional.

Verification of the automation step operation or the healthcare professional initiation of the automated step may be performed by a healthcare professional (e.g., a surgeon). Verification of an out of sequence step may be performed to avoid triggering of any accidental request of the automated step. For example, the healthcare professional may partially clamp on a tissue and may accidentally initiate articulation or firing. The system may verify (e.g., with the healthcare professional) that the requested operation is intended before an automated set of steps is started. In an example, a display may not be available or part of the system. In such a case, the system may first provide haptic feedback to the healthcare professional to confirm that the healthcare professional intends to perform the detected function and subsequent reactivation may be then allowed to initiate the automation with no feedback. The healthcare professional (e.g., the surgeon) may input predefined breakpoints in the automated steps, for example, to ensure the verification and completion of an automated step.

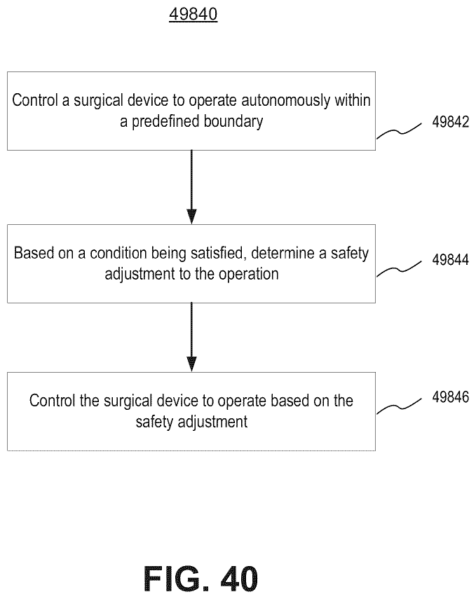

FIG. 40 is a flow chart of an example autonomous operation associated with a surgical device, 49840. At 49842, a surgical device may be controlled to operate autonomously within a predefined boundary. For example, the surgical device may be a smart grasper, a smart surgical stapler, or a smart energy device. The predefined boundary may be a virtual movement boundary associated with a surgical task. The predefined boundary may be a field of view defined by a scope device.

At 49844, based on a condition being satisfied, a safety adjustment to the operation may be determined. In the case that the surgical device is a smart grasper, the condition may be a tissue tension measurement associated with the smart grasper being equal to or greater than a maximum tissue tension and the safety adjustment may be a reduction of grasping force.

In the case that the surgical device is a smart surgical stapler, the condition may be an inrush current measurement being below a lowest threshold and the safety adjustment may be stopping a firing sequence.

In the case that the surgical device is a smart energy device, first placement data associated with a first trocar and second placement data associated a second trocar may be received. The first trocar may be associated with a smart grasper and the second trocar is associated with the smart energy device. First location data associated with the smart grasper may be determined based on the first placement data. Second location data associated with the smart energy device may be determined based on the second placement data. Third location data associated with a patient body and first orientation data associated with the patient body may be received. The condition may be that a distance between the smart energy device and the smart grasper is below a threshold and the safety adjustment may be a movement adjustment of the smart energy device based on the first location data, the second location data, the third location data, and the first orientation data.

At 49846, the surgical device may be controlled to operate based on the safety adjustment. In the case that the surgical device is a smart grasper, the condition may be a tissue tension measurement associated with the smart grasper being equal to or greater than a maximum tissue tension and the safety adjustment may be a reduction of grasping force. The controlling the surgical device to operate based on the safety adjustment may comprise sending a control signal to the surgical device to cause the reduction of grasping force.

In the case that the surgical device is a smart surgical stapler, the condition may be an inrush current measurement being below a lowest threshold and the safety adjustment may be stopping a firing sequence. The controlling the surgical device to operate based on the safety adjustment may comprise stopping sending a control signal to the surgical device to cause the firing sequence to stop.

Enormous amounts of surgical data are generated during surgical procedures. Surgical tasks performed during and/or after the surgical procedure are continually completed using the generated surgical data. Surgical systems may request specific task-related data for a surgical task before performing the task or the surgical systems may receive the wholesale surgical data and may parse through the complete dataset to find the specific task-related data needed for the task. This may create a delay or use unnecessary resources in performing the surgical task. Parsing through wholesale surgical procedure data to find task-specific data for a task is inefficient and poses a bandwidth problem as wholesale data is communicated to a surgical system that only needs a portion of the data.

Systems, methods, and instrumentalities are disclosed for automatic compilation, annotation, and dissemination of surgical data to surgical systems and/or devices to anticipate (e.g., in advance of, before, in preparation for) related automated operations. A surgical computing system may be configured to obtain data (e.g., surgical procedure data) associated with, for example, a patient being treated in the operating room, healthcare professionals (HCPs) participating in the surgical procedure, the surgical devices and/or equipment used in the surgical procedure, surgical sensors, a surveillance system in the operating room, a surgical hub, and/or the like. The surgical procedure data may be compiled, for example, by a surgical computing system. The surgical computing system may be configured to annotate the surgical procedure data. The annotations may indicate a surgical context and/or a surgical procedure step. The surgical computing system may identify surgical systems, for example, that may use the annotated data for related surgical tasks. The surgical computing system may determine data needs (e.g., data to be used for a task) for an identified surgical system (e.g., target surgical system). The computing system may generate selectively discriminated data (e.g., a data package, a data stream) for the target surgical system, for example, by decompiling the annotated surgical procedure data. The selectively discriminated data may include at least a portion of the annotated surgical procedure data (e.g., complete annotated surgical procedure data). The surgical computing system may send the selectively discriminated data to the target surgical system, for example, for the target surgical system to use in a subsequent task.

The surgical computing system may determine the data needs for the target surgical system based on the surgical procedure data, for example, using situational awareness (e.g., as described herein with respect to FIG. 7). The surgical computing system may determine a current surgical context and/or a current surgical procedure step in the surgical procedure, for example, based on the surgical procedure data. Based on the current surgical context and/or current surgical procedure step, the surgical computing system may determine a subsequent surgical context and/or subsequent surgical procedure step (e.g., the next surgical context and/or surgical procedure step to occur after the current surgical context and/or surgical procedure step). The surgical computing system may determine tasks associated with the subsequent surgical context and/or subsequent surgical procedure step to be performed by surgical systems. The surgical computing system may determine the data needs based on the determined tasks associated with the subsequent surgical context and/or subsequent surgical procedure step. For example, the surgical computing system may anticipate the data to be used for the tasks in the subsequent surgical context and/or subsequent surgical procedure step.

The surgical computing system may perform selective redaction on the surgical procedure data, annotated surgical procedure data, and/or data package. The surgical computing system may perform redaction, for example, on pre-identified data and/or conditional aspects of the data. For example, the surgical computing system may perform redaction on confidential aspects of the surgical procedure data (e.g., in accordance with the Health Insurance Portability and Accountability Act (HIPAA)). Redaction may include removing confidential aspects of the surgical procedure data, for example, by removing the confidential data, replacing the data (e.g., with generic and/or default values), scramble, encrypt, and/or the like to render the confidential aspects unreadable. The surgical computing system may redact unusual and/or unexpected data. The redaction may be temporally and/or geo-fence controlled.

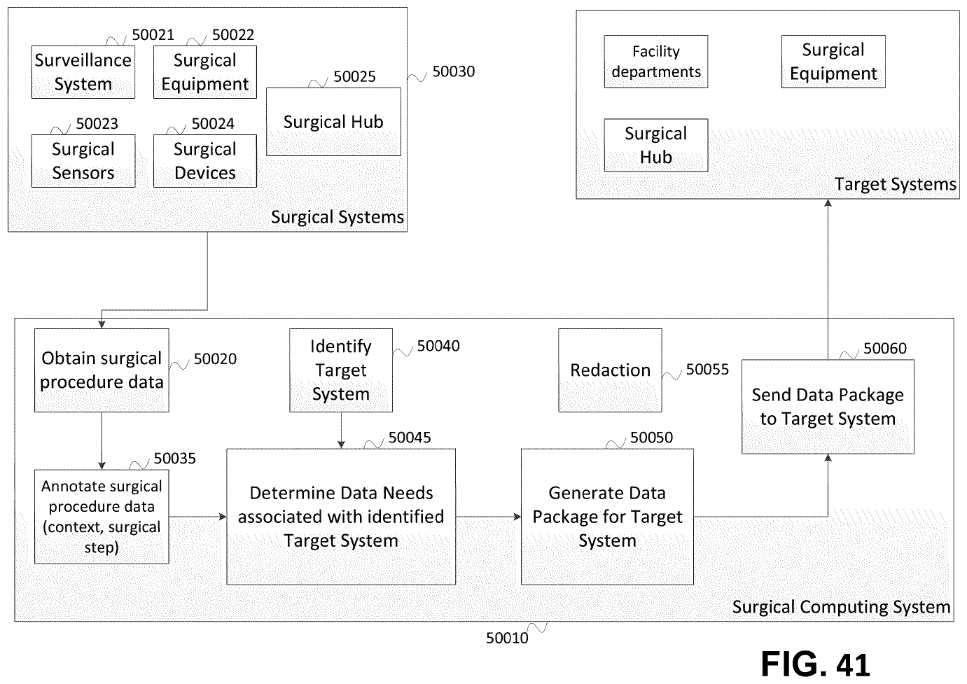

FIG. 41 illustrates an example flow diagram of a surgical computing system automatically performing selective dissemination of annotated surgical procedure data to surgical systems. As shown in FIG. 41 at 50020, a surgical computing system 50010 may obtain surgical procedure data. The surgical procedure data may be obtained from the surgical systems 50030, for example. At 50035, the surgical computing system 50010 may annotate the surgical procedure data. As shown at 50040 in FIG. 41, the surgical computing system 50010 may identify/determine a target system (e.g., target surgical system). At 50045, the surgical computing system 50010 may determine data need(s) associated with the target system. The data needs may be associated with a task, for example, to be performed by the target system (e.g., in a subsequent/future/next surgical step). The data needs may be associated with the data used (e.g., required) in performing the task. As shown at 50050, the surgical computing system 50010 may generate a data package (e.g., a data stream), for example, for the target system. The data package may be generated based on the annotated surgical procedure data and/or the data needs associated with the target system. As shown at 50055, the surgical computing system 50010 may be configured to perform redaction. The redaction may be performed on the obtained surgical procedure data, the annotated surgical procedure data, the data package, and/or the like. At 50060, the surgical computing system 50010 may send the data package to the target system. The target system may be for example a surgical hub, a surgical instrument, surgical equipment, systems associated with a facility department, a billing system, and/or the like.

The surgical computing system 50010 may obtain surgical procedure data, for example, from systems/devices in the surgical systems 50030. For example, the surgical computing system 50010 may obtain surgical procedure data from one or more of a surveillance system 50021, surgical equipment 50022, surgical sensors 50023, surgical devices 50024, surgical hub(s) 50025, and/or the like.

The surgical procedure data may be data associated with a surveillance system, a surgical sensor (e.g., biomarker sensor, HCP sensor, etc.) surgical equipment, surgical devices, surgical tools, staffing for a surgical procedure, OR setup/layout, surgical procedure steps, consumables used for a surgical procedure, electronic medical records, imaging scans and/or results, surgical outcomes, and/or the like. For example, the surgical procedure data may include raw data from the surgical systems the data is obtained from. The surgical procedure data may be processed data (e.g., from the surgical hub 50025). For example, the surgical hub 50025 may process obtained surgical sensor data. The processed raw data may indicate surgical events, surgical procedure steps, timing, surgical outcomes, device utilization, and/or the like. The surgical computing system 50010 may obtain the processed surgical procedure data.

The surgical computing system 50010 may compile the obtained surgical procedure data. In examples, the surgical computing system 50010 may obtain compiled surgical procedure data. For a surgical procedure, the surgical hub 50025 may obtain data from the surgical systems (e.g., operating room (OR)) that the surgical hub is associated with. The surgical hub 50025 may compile the obtained surgical procedure data and send the compiled surgical procedure data to the surgical computing system 50010. The surgical computing system 50010 may use the obtained compiled surgical procedure data for annotation and/or dissemination to target systems.

The surgical computing system 50010 may annotate the surgical procedure data (e.g., compiled surgical procedure data). For example, the surgical computing system 50010 may annotate the surgical procedure data based on situational awareness (e.g., as described herein with reference to FIG. 7). The surgical computing system 50010 may determine surgical context data, which may indicate a surgical event, a time, a surgical procedure step, and/or the like, for example, based on the surgical procedure data. The annotations may be associated with the determined surgical context, surgical event, time, surgical procedure step, and/or the like. The annotations may be used to give context to the surgical procedure data, for example, if the surgical procedure data is decompiled.

As shown at 50040, the target system(s) may be identified. For example, the target systems may include surgical systems (e.g., in the OR and/or in the facility), facility department systems, surgical equipment, surgical hubs, a billing system and/or the like. The target systems may be performing tasks (e.g., surgical tasks) associated with the surgical procedure. For example, target systems (e.g., facility department systems) may be configured to schedule replacement, repair, and/or cleanup of an OR and consumables (e.g., materials used during the surgical procedure).

The target systems may use surgical procedure data to perform the tasks. For example, a surgical task in a subsequent surgical procedure step may be performed autonomously, based on surgical procedure data associated with earlier surgical tasks and/or surgical procedure steps. For example, a surgical procedure data associated with a first surgical procedure step may be used by a target system to perform a surgical task in a second surgical procedure step. For example, surgical procedure data may include patient surgical sensor data associated with a patient biomarker. The patient biomarker information may be used by a target system to perform a surgical task in a subsequent surgical procedure step/phase. For example, the target system may alter parameters associated with the task based on the patient biomarker information.

At 50045, the surgical computing system 50010 may determine data need(s) associated with the identified target system. The data needs may be a set of data used (e.g., by the target system) to perform a surgical task. The surgical computing system 50010 may determine the data needs based on the surgical task the surgical system is configured to perform (e.g., configured to perform at a later time or subsequent surgical step). For example, the data needs may be anticipated by the surgical computing system 50010.

The surgical computing system 50010 may determine (e.g., anticipate) the task (e.g., to be performed by the target system) based on situational awareness, for example, using the surgical procedure data and/or annotated surgical procedure data. For example, the surgical computing system 50010 may determine a current surgical context and/or surgical procedure step based on the surgical procedure data. Using the current surgical context and/or surgical procedure step, a subsequent surgical context and/or surgical procedure step may be determined. The subsequent surgical context and/or surgical procedure step may be determined (e.g., anticipated) using a surgical procedure plan and the current surgical context and/or surgical procedure step.

For example, the surgical computing system may determine (e.g., based on the surgical procedure data), that the surgical procedure is currently in a first surgical procedure step. The surgical computing system may identify a second surgical procedure step as the surgical procedure step that follows the first surgical procedure step in the surgical procedure plan. The surgical computing system may determine surgical tasks associated with the second surgical procedure step. With the knowledge of the surgical tasks, data needs associated with the surgical tasks may be determined. The surgical computing system may anticipate the data needs before the second surgical procedure step occurs. The surgical computing system may determine the data needs associated with the target surgical system, for example, without the target system sending a request indicating the data needs.

Various examples of surgical steps and corresponding data needs, and are suitable for use with the present disclosure, are described in U.S. patent application Ser. No. 17/156,287, titled METHOD OF ADJUSTING A SURGICAL PARAMETER BASED ON BIOMARKER MEASUREMENTS, filed on Jan. 22, 2021, the disclosure of which is herein incorporated by reference in its entirety. For example, a thoracic surgery may be performed, such as a lung lobectomy. A surgical computing system may determine that the lung lobectomy is in a surgical step associated with managing major vessels. The surgical computing system may determine that the surgical procedure is in the major vessel management step, for example, based on surgical procedure data (e.g., determining a current surgical task associated with the major vessel management step is being performed). For example, the surgical computing system may determine that the current surgical task is ligating a patient's pulmonary artery (e.g., based on the surgical procedure data). The surgical computing system may determine that the lobe removal surgical step is a subsequent surgical step (e.g., the next surgical step) in the lung lobectomy. The surgical computing system may determine that the lobe removal surgical step follows the major vessel management step. The surgical computing system may determine that the lobe removal step includes using a surgical stapler (e.g., linear stapler) for a surgical task. For example, the surgical stapler may be used in a surgical task associated with transecting a fissure. The surgical stapler may be adaptively controlled, for example, using dynamic parameters for operating the surgical stapler based on the surgical procedure data. Surgical procedure data from previous surgical procedure steps and/or tasks may be used to determine the parameters used for operating the surgical stapler. The surgical computing system may determine the data needs of the surgical stapler used in the fissure transection. The surgical computing system may selectively discriminate the data that may be used for the fissure transection from the surgical procedure data. The surgical computing system may send the selectively discriminated data to surgical stapler, for example, in anticipation of the task (e.g., before the surgical step involving the surgical stapler). The selectively discriminated data may be sent to the surgical stapler, for example, before a request for the specific data is received.

A data package/stream (e.g., comprising selectively discriminated data) may be generated, for example, for the target system. The surgical computing system 50010 may generate the data package/stream for the target system using the annotated surgical procedure data based on the determined data needs associated with the target system. The data package/stream may be generated, for example, using automated selective discrimination (e.g., selecting portions of the annotated surgical procedure data set for dissemination to the appropriate systems). For example, the data package/stream may include data relevant to the data needs and exclude data not used by the target system. Automated selective discrimination may enable sending discrete chunks of data (e.g., rather than wholesale communication of all the compiled surgical procedure data) to communicate with target systems. Automated selective discrimination may minimize bandwidth issues and/or storage issues.

In examples, the surgical computing system may select a portion of the annotated surgical procedure data to generate the data package/stream. The surgical computing system may decompile the associated data from the complete set of annotated surgical procedure data. The decompiled data may be tagged (e.g., time tagged) using situational awareness and/or the surgical context. The portion of the annotated surgical procedure data may be a subset of complete set of annotated surgical procedure data. For example, the data package/stream may include a sub-set of procedure steps, a subset of resources used in the surgical procedure (e.g., consumables used, costs, time logged, etc.), redacted information, and/or the like.

The surgical computing system 50010 may perform redaction on the surgical procedure data. For example, the surgical computing system 50010 may perform redaction on the surgical procedure data (e.g., the obtained surgical procedure data), the annotated surgical procedure data (e.g., based on the annotation), the data package/stream, and/or the like.

At 50060, the data package/stream may be sent to the target system. The target system may include surgical systems, facility systems, and/or the like. For example, the target system may include a device maintenance scheduling system (e.g., for determining when to service/repair a surgical tool/device). The target system may include a surgical hub that is performing a surgical procedure. The surgical hub may obtain the data package/stream and use the data package/stream for subsequent surgical procedure steps.

In examples, the computing system may perform automated selective discrimination of data sets (e.g., automatically annotated datasets) and may send (e.g., disseminate) specific data packages to different systems. The computing system may send data packages containing data associated with the data needs of the target systems. The computing system may decompile obtained surgical procedure data and annotate the decompiled data (e.g., with a surgical context and/or time tags, for example, using situational awareness). The computing system may send the decompiled data (e.g., discrete chunks of data) to a target system. The computing system may refrain from sending the compiled surgical procedure data (e.g., entire set of surgical procedure data) by sending (e.g., only sending) the decompiled data. Sending the decompiled data may minimize bandwidth issues and/or storage issues.

In examples, the decompiled data (e.g., select packets of data) may be separated. The decompiled data packets may be separated based on a characterization, a risk level, a prioritization, a magnitude of change from the expected (e.g., outlier data), hierarchical segmentation, system utilization, and/or the like.

In examples, the surgical procedure data may be used during the automated selective discrimination (e.g., as described herein). For example, if a portion of surgical procedure data is different (e.g., significantly different and/or different beyond a threshold value) than as expected, the portion of surgical procedure data that is different (e.g., outlier data, unexpected data) may be included in the data package. For example, the surgical computing system may obtain statistical data associated with the performed surgical procedure. The statistical data may include expected and/or average data. The statistical data may include expected deviations (e.g., thresholds) associated with the expected and/or average data. The surgical computing system may determine that surgical procedure data deviates from the expected and/or average data, for example, beyond a threshold. The surgical computing system may flag the surgical procedure data, and the flag may indicate the deviation. In examples, the portion of surgical procedure data that is different (e.g., deviant from expected values) may be tagged with an indication (e.g., indicating that it is different from expected). A computing system may include in the data package surgical procedure data that makes up a threshold amount of data (e.g., disproportionate amount of the data). The surgical procedure data that makes up a threshold amount of data may be tagged with an indication (e.g., indicating that the data makes up the threshold amount of data).

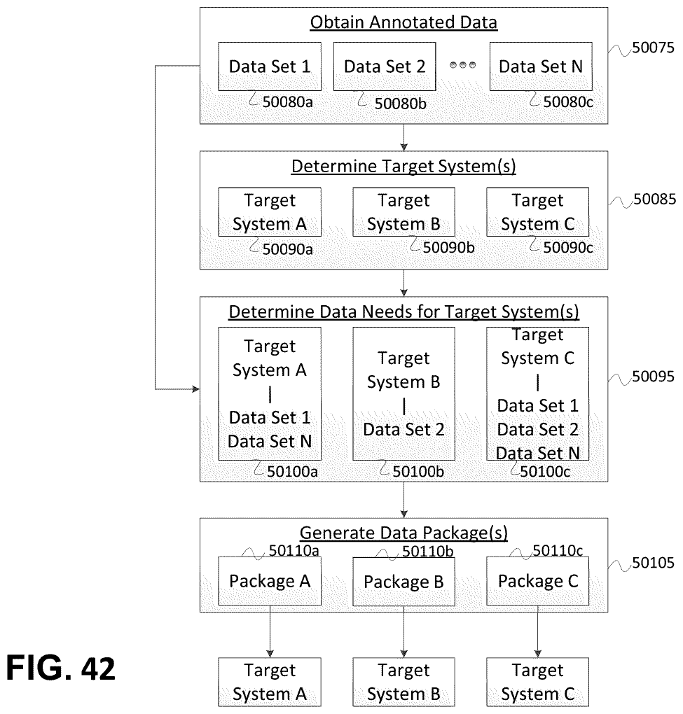

FIG. 42 illustrates an example of generating and sending data packages to target systems. Surgical procedure data (e.g., compiled surgical procedure data) may be obtained. The surgical procedure data may include the data (e.g., all the data) associated with the surgical procedure. Target systems may use portions of the surgical procedure data, for example, to perform surgical tasks. The target systems may not need or require the entire compiled surgical procedure data. Sending the compiled surgical procedure data (e.g., entire set of surgical procedure data) may cause unnecessary bandwidth and/or storage issues.

As shown in FIG. 42 at 50075, surgical procedure data may be obtained. The obtained surgical procedure data may be annotated, and or annotation may be performed on the obtained surgical procedure data. The annotated surgical procedure data may include datasets (e.g., multiple datasets), such as, for example, Data Set 1 50080a, Data Set 2 50080b, Data Set N 50080c, etc.

As shown at 50085, the target system(s) may be determined/identified. The target system(s) may include systems that receive surgical procedure data to perform tasks, such as, for example, surgical tasks, data storage, facility management, and/or the like. Target system(s), such as Target System A 50090a, Target System B 50090b, and/or Target System C 50090c may be determined/identified by the surgical computing system. For example, the target system(s) may be identified based on the current surgical context (e.g., current surgical procedure step). The target system(s) may be identified as performing tasks, for example, in the current surgical context and/or a subsequent surgical context.

As shown at 50095, data needs may be determined for the target system(s). Different target systems may be associated with different data needs. For example, a first data needs may be associated with Target System A 50090a. As shown at 50100a, the first data needs may be associated with Data Set 1 50080a and/or Data Set N 50080c. A second data needs may be associated with Target System B 50090b. As shown at 50100b, the second data needs may be associated with Data Set 2 50080b. A third data needs may be associated with Target System C 50090c. As shown at 50100c, the third data needs may be associated with Data Set 1 50080a, Data Set 2 50080b, and/or Data Set N 50080c. The data needs may be determined, for example, based on the annotated surgical procedure data (e.g., as described herein).

As shown at 50105, the data package(s) may be generated. The data package(s) may be generated for the target system(s). The data package(s) may include data associated with the data needs (e.g., only data associated with the data needs) for the target system(s). The target system(s) may receive a portion of the surgical procedure data (e.g., a portion of the complete version of the annotated surgical procedure data). For example, the data package may include redacted information, a sub-set of the procedure steps, and/or a subset of data collected (e.g., consumables used, costs, time logged, etc.).

As shown in FIG. 42, a generated Package A 50110a may include data associated with the data needs associated with Target System A 50090a. For example, Package A 50110a may include Data Set 1 50080a and/or Data Set N 50080c. A generated Package B 50110b may include data associated with the data needs associated with Target System B 50090b. For example, Package B 50110b may include Data Set 2 50080b. A generated Package C 50110c may include data associated with the data needs associated with Target System C 50090c. For example, Package C 50110c may include Data Set 1 50080a, Data Set 2 50080b, and/or Data Set N 50080c. The data packages may be sent to the target system(s).

Redaction may be performed on surgical procedure data. The redaction may be performed on the compiled surgical procedure data, annotated surgical procedure data, data packages, individual data sets, and/or the like. Redaction may be performed more than once. Selective redaction may be automated. For example, selective redaction may be automated based on pre-identified and/or conditional aspects of the data. For example, surgical contexts, events, images, datasets, etc. may be used (e.g., as a filter) to select portions of the data for removal. Portions of the data may be selected for removal and may be redacted from generated data packages.

For example, pre-defined portions of surgical procedure data may follow a set of rules for inclusion and/or exclusion in data packages. Data may be redacted based on satisfying a condition of exclusion from the data set. A portion of data within a dataset may be redacted (e.g., only a portion of the dataset may be redacted). Data may be redacted, for example, if it is associated with confidential information (e.g., HIPAA information). Data may be redacted, for example, if the data is unusual and/or unexpected (e.g., different from an expected value beyond a threshold value). The data may be flagged as unusual and/or unexpected (e.g., but not redacted). Data may be redacted, for example, based on temporal conditions and/or location conditions (e.g., geo-fenced controlled). For example, conditions for redacting data may be associated with one or more of the following: whether an amount of time has passed; when the data leaves the network/system; when the surgical procedure is completed; if the data is transferred to a protected archive; and/or the like. For example, the data may be redacted after (e.g., only after) a predetermined amount of time has passed.

Redacted data may be flagged, for example, such as tagged with an indication indicating that the data is redacted. The redacted data may be flagged such that it indicates that the information is to be removed (e.g., automatically removed). The redacted data may be flagged such that it indicates the condition(s) associated with the redaction.

For example, redaction may be performed on data based on a classification. For example, a classification may be determined for surgical procedure data (e.g., a portion of surgical procedure data). For example, surgical procedure data may include private and/or confidential information (e.g., according to HIPAA guidelines). The surgical computing system may determine a private classification for surgical procedure data that includes private and/or confidential information. Redaction may be performed on surgical procedure data determined to have a private classification. The surgical computing system may refrain from performing redaction on surgical procedure data that does not include private and/or confidential information.

For example, redaction may be performed on data based on a target system (e.g., to which the data is to be sent). Surgical procedure data may be redacted based on a classification associated with the surgical procedure data and the target system. For example, a target system may be a surgical system within or outside of a patient privacy protection boundary. The target system may be a surgical system at a geographic location, a network-based location, or organization-based location, which may indicate whether the system is inside or outside of the patient privacy protection boundary. The geographic location may be outside the HIPAA boundary. The surgical computing system may determine to redact surgical procedure data classified as private information based on the target system's geographic location (e.g., because it is outside the HIPAA boundary). Confidential information (e.g., with respect to HIPAA) may not be permitted to be sent outside the HIPAA boundary. The target system may be a surgical system at a geographical location within the HIPAA boundary. The surgical computing system may determine to refrain from redacting surgical procedure data classified as private information. For example, a data storage may be located in the cloud network (e.g., outside the HIPAA boundary). The surgical computing system may redact confidential information before sending data to the cloud network data storage. For example, a patient's electronic medical record storage may be located in a facility (e.g., within the HIPAA boundary). The surgical computing system may refrain from performing redaction before sending the data to the patient's electronic medical record storage.

In examples, a computing system may annotate (e.g., automatically annotate) surgical procedure data (e.g., video feeds) during a surgical procedure. The annotations may indicate surgical step(s) and/or time scales associated with the surgical procedure. The computing system may flag aspects of the annotation (e.g., and/or the video feed) in different coding aspects. For example, a first coding aspect may be associated with the computing system maintaining the integrated surgical procedure data (e.g., video feed) as whole. A second coding aspect may be associated with the computing system redacting data after a secure storage (e.g., primary secure storage) of the surgical procedure is completed (e.g., after the surgical procedure ends). A third coding aspect may be associated with the computing system redacting portions of data (e.g., aspects of the data) before transmission of the data packages to the target systems (e.g., after the data packages leave the primary secure storage).

In examples, the secure storage may be configured with an amount of time. The secure storage may eliminate the stored data, for example, after the configured amount of time. The secure storage may perform an automatic deletion of the data. The automatic deletion may be associated with conditions. The automatic deletion may be modified (e.g., the amount of time before deletion may be extended/shorted and/or the automatic deletion may be cancelled) based on the conditions. The conditions may be associated with events, for example, that may occur after storage, such as, patient complications, readmission, hospital acquired infections, legal/billing issues, and/or the like.

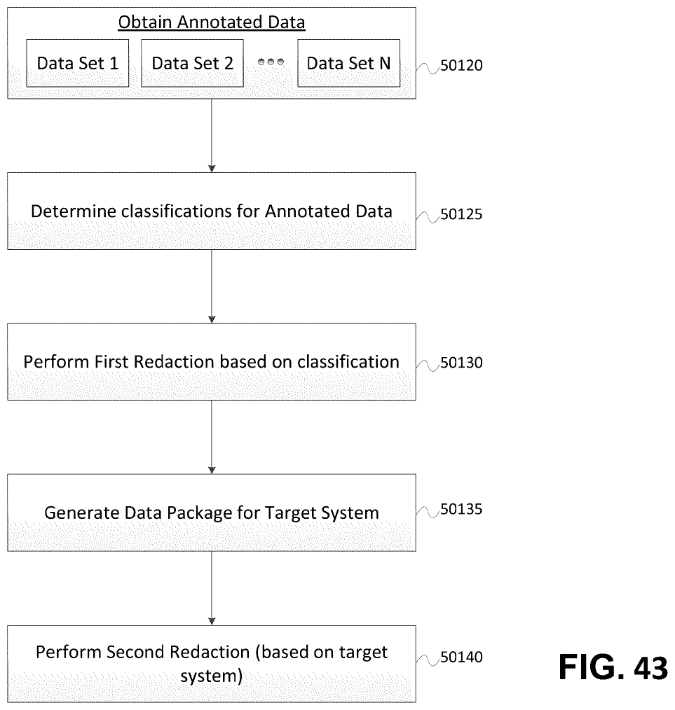

FIG. 43 illustrates an example of selectively redacting of data in a data set. As shown at 50120, surgical procedure data (e.g., annotated surgical procedure data) may be obtained. The surgical procedure data may include multiple dataset(s). At 50125, a classification may be determined for the surgical procedure data. The surgical procedure data, dataset(s) within the surgical procedure data, portions of surgical procedure data, subsets of surgical procedure data, etc. may be associated with a classification.

For example, a subset of the surgical data may be classified as confidential information (e.g., in terms of HIPAA). For example, a subset of surgical data may be classified as inaccurate, erroneous, an outlier, and/or the like.

At 50130, a first redaction may be performed on the surgical procedure data, for example, based on the determined classification. Confidential information may be redacted, for example, before sending to a different system (e.g., target device). The subset of the surgical data may be redacted (e.g., required to be redacted) before sending the data, for example, based on the classification of being confidential information (e.g., in terms of HIPAA). The subset of the surgical data may be redated, for example, based on the subset of surgical data being classified as inaccurate. The computing system may determine to refrain from redacting the inaccurate surgical data, for example, to notify HCPs about the inaccurate data. For example, if the HCP is aware of the inaccurate data, the HCP may alter surgical procedure steps or use different surgical equipment.

At 50135, the data package(s) for the associated target system(s) may be generated. The data package(s) may include the redacted data.

At 50140, a second redaction may be performed, for example, on at least a portion of the data in the data packages. For example, the second redaction may be performed based on the target system associated with the data package. For example, the target system may be outside HIPAA boundaries and subject to confidentiality rules. Confidential information (e.g., in terms of HIPAA) may be redacted before sending the data to the target system outside HIPAA boundaries. A target system may within HIPAA boundaries, where the same confidential information may not be redacted (e.g., may not be required to be redacted).

Surgical procedure data determined to be redacted may be stored, for example, before performing the redaction. For example, surgical procedure data determined to be confidential information (e.g., with respect to HIPAA) may be redacted before being sent outside the HIPAA boundary. The surgical computing system may store the surgical procedure data to be redacted in a local storage, for example, to preserve the surgical procedure data. The surgical procedure data to be redacted may be stored, for example, as a backup and/or if the surgical procedure data to be redacted is needed at a later time.

Automated data packages may be generated for facility systems. The automated data packages may be sent to facility systems, for example, to schedule device/tool replacement, repair and/or cleanup of an operating room, replenishing consumables in a facility room, and/or the like.

For example, a data package may be sent to a facility product re-ordering system. The data package may include data associated with products used in a surgical procedure. The data associated with the products used during the surgical procedure may indicate the consumable resources that were used in a previous surgical procedure and are missing in the operating room for a planned surgical procedure. The data package may indicate that the product to be replenished is not in stock in the facility, for example, based on facility information. The data package may indicate a request to the facility product re-ordering system to indicate when the earliest the planned surgical procedure can be conducted (e.g., because of the delay in restocking the missing consumable) and/or indicate alternative instruments/consumables that may be used to conduct the planned surgical procedure (e.g., to make the procedure occur earlier).

A data package may be sent to a facility system associated with cleaning and maintenance. For example, the data package may include data associated with the planned surgical procedure, the tools/devices/equipment used in the planned surgical procedure, the tools/devices/equipment currently stocked in the operating room, and/or the like. The data package may include data verifying that the surgical tools/devices/equipment are present and ready for use for the planned surgical procedure. The data package may include data indicating that the operating room is not ready for the planned surgical procedure and/or indicate a time that the operating room will be ready.

A data package may be sent to a facility system associated with sterilization (e.g., of surgical equipment and/or tools in an operating room). For example, the data package may include data that indicates verification of the instruments/devices/equipment that will be used in the planned surgical procedure. The data package may include data that indicates that surgical equipment for the planned surgical procedure is sterile and/or ready to use for the planned surgical procedure. The data package may include data that indicates that the surgical equipment is not cleaned. The data package may include data that indicates that the surgical equipment is in the process of being cleaned. The data package may include data that indicates a prioritization of a specific surgical device/tool/equipment for sterilization (e.g., that is currently not clean or is unavailable).

A data package may be sent to a facility system associated with staffing and surgical procedure timing. For example, the data package may include data indicating the planned surgical procedure timing. The data package may include data associated with the HCPs scheduled for the surgical procedure. The data package may include identification information associated with the staff. For example, specific surgical tasks may be performed by specific HCP roles in the surgical procedure. The data package may indicate staff that are suitable for the surgical task. The data packages may indicate staff that are available for the surgical procedure. The data packages may indicate staff based on a classification, such as surgeon preference, OR setup, and/or the like.

The data package(s) may be sent to the target system(s), for example, for surgical procedure documentation. The data package(s) may be sent for automated population of the surgical procedure documentation. The data package(s) may be sent to system databases (e.g., linked system databases in the facility). The data package(s) may be associated with updating, annotating, and/or transcribing information associated with the surgical procedure.

The data package(s) may be sent to the target system(s), for example, for billing population, annotation, and/or classification. The data package(s) may include information associated with surgical procedure steps performed in the surgical procedure. The information associated with the surgical procedure steps performed in the surgical procedure may be linked to billing codes (e.g., diagnosis related group billing codes). The data package(s) may include data used by the facility billing systems to track the billing information. For example, the computing system may determine the data to include in the data package for the billing systems. The computing system may further annotate the data in the data package (e.g., tag with metadata), for example, to classify the data with associated billing codes. The annotated data may be used to update the data sets to be used by the target system(s). The data package(s) may be sent to the target system(s), for example, for billing tasks.

The data package(s) may be sent to the target system(s), for example, for stock maintenance (e.g., consumable maintenance, surgical tool maintenance, surgical device maintenance, and/or the like). For example, the data package(s) may include information indicating products used (e.g., pulled) during the surgical procedure. The data package(s) may include information indicating the serial number associated with the products used. The data package(s) may include information indicating the number of uses for a surgical tool, surgical device, and/or surgical equipment during the surgical procedure. The data package(s) may include information indicating that the surgical tool, surgical device, and/or surgical equipment are due for maintenance, replacement, repair, disposal, returning, sterilization, cleaning, and/or the like. The data package(s) may include information indicating any issues and/or notes associated with the product use during the surgical procedure (e.g., if there were issues using the product and/or the product malfunctioned).

The data package(s) may be sent to the target system(s), for example, for electronic medical record (EMR) database population. For example, the data package(s) may include information associated with the patient and the surgical procedure. The data package(s) may include information associated with the details, annotations, recordings, procedure notes, and/or the like from the surgical procedure. The data package(s) may be used to update (e.g., automatically update) the EMR database for the patient's record. The data package(s) may include information associated with the surgical procedure aspect, surgical instruments used, surgical tasks performed, alternative treatments that were performed, and/or the like. The data package(s) may include the surgical video(s) (e.g., annotated surgical video(s)) associated with the surgical procedure. The surgical video(s) may be used as a record of the surgical procedure and a record for the surgical steps and/or surgical tasks conducted during the surgical procedure.

The dissemination of information/data (e.g., automated dissemination of information/data) may be documented/annotated. For example, the automated steps performed associated with one or more of obtaining surgical procedure data, annotating surgical procedure data, determining the target system(s), determining the data needs associated with the target system(s), generating the data packages for the target system(s), redaction of a portion of the data, sending the data package to the target system(s), etc. may be documented (e.g., annotated). For example, the computing system may document and/or record the autonomous operation associated with the dissemination of the surgical procedure data. The surgical computing system may document and/or record user responses to the autonomous operation (e.g., overrides, verifications, confirmations, and/or the like).

The computing system (e.g., if performing automated dissemination of data to target systems) may document responses to the data sets and actions taken with respect to the data sets. Machine learning data may be generated, for example, based on the computing system's responses to datasets. The machine learning data may be used to train an artificial intelligence (AI) model. The AI model may be trained and/or used for performing subsequent automation tasks. The machine learning data may be associated with one or more of the following: a failure of a surgical device/tool, degraded performance (e.g., associated with a surgical device/tool, user technique actions, and/or the like), data relating to user biomarkers, surgical procedure steps, staff interactions, user behavior (e.g., related to an unexpected event), and/or the like.

The computing system may generate machine learning data associated with a failure of a surgical device during a surgical procedure. For example, the computing system may determine that a surgical device failed to perform properly and/or generated inaccurate data. The computing system may determine the surgical device failure, for example, based on other surgical procedure data obtained during the surgical procedure. The computing system may determine to adjust the magnitude of inaccurate data associated with a surgical device, for example, based on the other surgical procedure data obtained during the surgical procedure. The computing system may generate machine data associated with the recalibration (e.g., adjusting the magnitude of the inaccurate data). The computing system may determine a type of failure associated with the surgical device. The type of failure may be used, for example, to escalate the magnitude of associated data (e.g., surrounding data generated by associated devices) that may be attached to the failure data. The machine learning data may indicate devices associated with the surgical device failure (e.g., that provide to and/or use data associated with the surgical device failure). The machine learning data may indicate that the associated devices may need to be augmented (e.g., adjusted) and/or recorded, for example to limit (e.g., minimize) the propagation of failure and/or improve device to device reliance.

The computing system may generate machine learning data associated with degraded performance, for example, associated with user technique actions, surgical instruments, and/or the like. For example, machine learning data may be generated that indicates that performance has fallen below a threshold. The performance may have degraded, for example, based on the user technique actions and/or surgical instrument. The machine learning data may be generated based on tracking of the user control interaction coupled with a surgical instrument. The machine learning data may include recorded data indicating a repeated user-controlled action, for example, that may result in degraded function. The machine learning data may be applied to an AI model, for example, to prevent performing the user-controlled action coupled with the surgical instrument that resulted in degraded function. The AI model may improve to prevent subsequent surgical procedures from using the operations that are associated with degraded performance and/or function.

The computing system may generate machine learning data associated with improved performance (e.g., unexpected improved performance). For example, the computing system may determine that user techniques associated with a surgical instrument is performing above a threshold. The computing system may record/document surgical procedure data (e.g., from the surgical hub), for example, to investigate the performance (e.g., to determine the cause of the improved performance). The recorded surgical procedure data may include information associated with the events occurring in the OR during the surgical procedure during the improved performance. The computing system may obtain diagnostic data (e.g., request device diagnostic data) for systems (e.g., internal systems), for example, to determine whether the systems (e.g., internal systems) have been affected. The computing system may enable the surgical hub to request associated devices (e.g., devices associated with the improved performance) to perform internal diagnostic checks. The machine learning data associated with the improved performance may include an indication that indicates (e.g., to a user) to provide additional information (e.g., contextual information) on tasks/techniques that may have been performed differently (e.g., which may have caused the improved performance). The computing system may determine (e.g., verify) whether the system associated with the improved performance actually experienced an improved performance. For example, the computing system may indicate (e.g., to a user) to verify that the result was unanticipated. The computing system may verify that the algorithm associated with determined whether there is improved performance is accurately rating performance.

The computing system may generate machine learning data associated with metadata relating to use biomarkers, procedure steps, staff interactions, user behavior, and/or other surgical events/interactions (e.g., related to an unexpected event). For example, the machine learning data may include documentation of medicines and/or patient biomarkers that may be associated with the unexpected event. The machine learning data may include staffing information, for example, such as the presence of staff in the OR at the time of the unexpected event. The machine learning data may include the surgical procedure step and/or sequential action variances to the surgical procedure plan. For example, variances to the surgical procedure plan may include deviations of action from the initial procedure plan, such as, changes of approach to a surgical site (e.g., internally and/or trocar placement), that may have contributed to the unexpected event. Variances to the surgical procedure plan may include unanticipated complications (e.g., excessive adhesions encountered during mobilization). The machine learning data may include biomarkers of the staff, for example, that may indicate that the biomarkers are elevated and/or depressed irregularly from a steady state (e.g., a threshold state). The machine learning data may include a listing of available surgical instruments that the user chose not to use.

For example, the computing system may document (e.g., perform automated documentation) user responses, which may include creating data associated with a user's reaction to the computing system's performed operations (e.g., autonomous operations, such as annotating datasets and/or generating data packages for target systems using the annotated datasets). For example, the computing system may determine that a user performed an override action on an autonomous operation (e.g., operation associated with selective dissemination of data/information to the target system(s)). The computing system may create machine learning data associated with the override action, for example, such that the AI model learns that the performed autonomous action is associated with an override action by the user. The machine learning data may include data associated with the events and/or the risk of events leading up to the user override. The AI model may be used to perform subsequent autonomous actions. The computing system may use the AI model to avoid performing the autonomous action associated with the override action.

The computing system may determine retention conditions for surgical procedure data. For example, the computing system may determine to keep data in storage for longer/shorter periods based on one or more of the following: storage space availability, communication ability, system utilization, level of the data, facility rules, retention procedures, and/or the like. For example, data retention for data may be determined based on the storage space or communication constriction (e.g., low storage space). For example, data may be retained for a shorter amount of time based on the storage space and/or communication constriction. Less data may be stored based on the storage space and/or communication constriction (e.g., as compared with data storage if the storage space is not limited and/or communication is not constricted). The computing system may indicate to scale down storage and/or retention, for example, based on successful results and/or lack of events. Storage overload may trigger alternative operations for storage and/or archival of data.

A hierarchy of data retention may be used. For example, a level of data may be adjusted based on a retention period. Metadata may be released and deleted at a differing retention period. A hierarchy of data retention may be determined, for example, based on a depth, measure, and/or relationship to the patient. For example, patient biomarker data may be associated with the longest retention. The patient biomarker may be input to electronic medical records (e.g., for long retention). Annotated video data may have a longer retention period than secondary instrument data. The source and/or integrity of the data may affect the data retention. For example, calculated and/or derived data may be associated with a shorter retention period than directly measured data. Video and/or timeline-based data may be associated with a long retention period as compared to annotations and/or overlaid data on the video. Product inquiry data may be associated with different levels of data retention. For example, product inquiry may have a different retention period than other instrument operation parameters. A user request may be used to adjust the product inquiry retention period of data. In examples, patient recovery events may be used to release data from storage.

The computing system may generate machine learning data associated with the outcomes resulting from automated operations. For example, the machine learning data may include data associated with expected outcomes and actual outcomes associated with the planned automated operations. The machine learning data may flag operations involving automation. The machine learning data may flag successful performance of an automated operation, for example, relative to the expected outcome (e.g., planned response). The machine learning data may flag automated operations that were modified and/or overridden. The machine learning data may include characterizations associated with the automated steps, for example, to enable user oversight and improved trust that the automated operation was successful (e.g., completed the task successfully.

For example, automated operations may be associated with a pre-procedure CT scan, an MRI, and/or an active lap imaging. The scans and/or imaging may be used in an automated operation, for example, to identify and/or highlight the margins of the tumor (e.g., based on linking together landmarks and linking points). The user may see the real-time imaging from the scans and/or images. The user may determine the margins based on the real-time imagine. The user may determine that the margins (e.g., margins generated by the automated operation and/or margins determined by the user) need to be adjusted based on the automated operation. Verification that the margin creation steps were performed accurately may be performed. Identification of additional information determined at real-time (e.g., that caused an adjustment to the margins) may be performed.

Storage of surgical procedure data may be automated. The target systems (e.g., the computing system may send data packages to) may be storage systems. For example, the storage systems may be automatically determined. The location and/or duration of surgical procedure data storage may be determined (e.g., automatically determined). Recall parameters associated with the store surgical procedure data may be determined. Recall parameters may be associated with how the stored surgical procedure data may be recalled, when the stored surgical procedure data may be recalled, and/or how the surgical procedure data may be recalled. For example, the stored surgical procedure data may be recalled based on monitored data (e.g., current surgical procedure data).

Storage identification and/or pruning may be automated. For example, identification and/or segmentation of the data for transport (e.g., data packages) may be automated. The data packages may be generated (e.g., as described herein) and sent to the target system(s) that may use (e.g., require) the information in the data packages. The computing system may determine a retention period associated with the sent data package, an archival method for the data package, and/or a storage location for the data package. The computing system may determine redaction and/or a protected configuration of the data within the data package, for example, for the target system the data package is sent to. The data package may include segregated data. The data in the data package may be segregated based on a surgical job/task, an outcome, a constraint, a technique, a user, a procedural step, and/or the like. The data package may be organized based on the segregations in the data package. For example, data for linked and/or similar procedure steps may be segmented (e.g., for review and/or export). For example, data associated with a particular surgical instrument (e.g., an Enseal device) may be pooled together for review together, which may allow the users, facility, and/or manufacturer to review the operation together. A storage location may be determined for erroneous and/or irregular surgical procedure data. The computing system may use an alternative process for storage and/or review of the data, for example, if the data is determined to be erroneous and/or irregular. Data resulting in an incident and/or complication may be stored differently.

Surgical procedures may use patient specific procedure plans in planning and executing a surgery. Creating a patient-specific procedure plan for an operation may be performed manually by healthcare professionals and takes a lot of time and effort. Healthcare professionals may parse through large amounts of pre-surgical data and patient specific data in planning surgeries for a patient. The time spent devising the patient specific procedure plan may include time spent not performing other duties. However, surgeons cannot just use a procedure plan template for each surgery as each surgery is tailored to a patient's specific needs. There are many variables to take into account when determining a patient specific procedure plan.

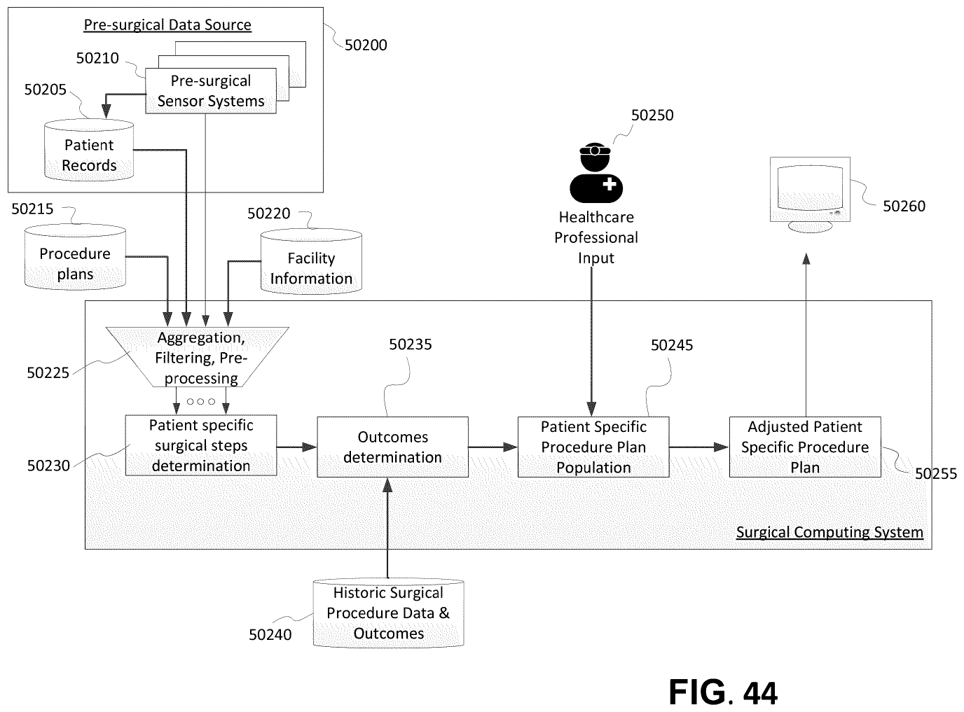

FIG. 44 illustrates an example aggregation of pre-surgical data and generation of a patient specific procedure plan. As shown in FIG. 44, a surgical computing system may obtain surgical procedure data (e.g., pre-surgical procedure data). The surgical procedure data may be used to generate a patient specific procedure plan. The surgical procedure data may be associated with pre-surgical data sources 50200 (e.g., scans, images, electronic medical records, etc.), procedure plans 50215 (e.g., procedure plan templates), facility information 50220 (e.g., staffing, room availabilities, etc.), and/or the like. The pre-surgical data sources may include patient records 50205 and/or pre-surgical sensor systems 50210. The surgical procedure data may be patient-specific surgical procedure data. Patient-specific surgical procedure data may include data associated with a patient and/or a patient's scheduled surgical procedure. For example, patient-specific surgical procedure data may include pre-surgical tests and/or images obtained for a planned surgical procedure.

The surgical procedure data may be automatically collected. For example, the surgical computing system may automatically obtain surgical procedure data associated with a patient specific surgical procedure. The surgical computing system may send requests for and may receive information associated with the patient and/or patient specific surgical procedure. Scans, images, and/or tests (e.g., preoperative scans, images and/or tests) performed may be automatically collected for the surgical procedure. Surgical procedure data may be collected from patient biomarker systems, patient records, and/or other pre-surgical sensor systems.

As shown at 50225 in FIG. 44, the surgical procedure data may be processed. For example, the surgical procedure data may be aggregated and/or compiled. The surgical procedure data may be synchronized, for example, during the aggregation. The aggregation may include summarizing the surgical procedure data. The aggregation may include alignment of the surgical procedure data, for example, such as aligning preoperative images. For example, the surgical procedure data may include different presurgical patient images. The aggregation may include aligning the pre-surgical patient images and/or overlaying the images, for example, to provide context for the patient specific surgical procedure. The aligned pre-surgical patient images may be used to identify areas for the surgical procedure (e.g., tumors, surgical sites, organs, etc.) and/or blind spots in the images.

The surgical procedure data may be filtered. Filtering may be performed to determine inaccurate and/or missing surgical procedure data. Filtering may be performed, for example, so the surgical procedure data may be interpreted. Pre-processing may be performed on the surgical data, for example, to condition the data for analysis.

For example, the surgical procedure data may be processed for aggregation and/or compiling into a baseline procedure plan starting point. Surgical procedure data may be used to determine subsurface and/or volumetric information associated with a patient's anatomy. For example, medical imaging (e.g., X-ray, fluoroscopy, MRI, CT, and/or ultrasound scans) may provide information associated with the patient's anatomy. The surgical procedure data may be used (e.g., multiple images may be used and/or aggregated) to plan and/or intraoperatively guide a surgical procedure. Target surgical sites and/or supplementary landmarks may be identified, for example, using the surgical procedure data. The surgical procedure data may be used in automation of image processing and/or pattern recognition algorithms, for example, to determine a location associated with features (e.g., unique features) that may be used as a reference point in the surgical procedure. Image sources (e.g., multiple image sources) may enable an artificial intelligence system to process images (e.g., instantly) and/or make decisions based on the target sites and landmarks in navigating to the site. Image sources may enable spatial awareness, for example, for a guidance system. The surgical procedure data (e.g., preoperative images) may register with the patient on the operating table, for example, so the system may identify a path (e.g., optimal path) for the surgeon to follow and/or continue autonomously. The computing system may identify insertion points (e.g., alternative insertion points) and/or indicate the insertion points to the surgeon, for example, for additional instruments and/or trocar placement. Medical images may be used as feedback (e.g., real-time feedback) for a surgical procedure. The medical images used as feedback may improve surgical precision, improve accuracy, reduce margins, avoid sensitive tissues and nerves, improve consistency of treatments, and/or the like. Fiducial markers (e.g., additional fiducial markers) may be implanted (e.g., which may create landmark during the imaging, for example, for the processing of the image(s) and/or use of reregistering when the patient was on the operating table.

Patient pre-surgical data may be aggregated. Aggregated pre-surgical data may include the patient's biometrics, medical records, diagnostic imaging, disease state and/or progression, previous treatments, and/or the like. The aggregated pre-surgical data may be used to create a baseline procedure plan. The baseline procedure plan may include variables for the surgical procedure, such as, for example, access, patient positioning, preferred instrument mix, and/or the like. The surgeon may simulate the created baseline procedure plan, adjust the plan, add to the plan, and/or modify variables in the plan.

Relationships and/or interactions between data in the patient pre-surgical data may be identified. For example, data in the patient pre-surgical data may be conflicting. Data in the patient—pre-surgical data may be related and/or amplify each other. For example, interactive biomarkers may be highlighted (e.g., automatically highlighted) based on a determination that the biomarkers are related.

Thresholds of combined and/or interrelated effects of the patient pre-surgical data may be indicated. For example, patient pre-surgical data may interrelate above a threshold amount, which may affect how the pre-surgical data should be analyzed. For example, a patient may be taking a blood thinner (e.g., Warfrin and/or Heparin) to minimize blood clot complications. A blood test may indicate low platelet counts. A relationship (e.g., interaction) between the blood thinner and the low platelet count may be determined. The cumulative impact of bleeding may be greater than what the dosage of the blood thinner would account for. A higher probability of bleeding in the surgical procedure may be determined and indicated (e.g., in the baseline procedure plan). The surgeon may determine to modify the baseline procedure plan, for example, based on the indication of the increased bleeding risk. In an example, the computing system may modify the baseline procedure plan, for example, based on the determination of the increased bleeding risk. An alternative surgical step (e.g., different energy device, different surgical approach, different mobilization path for freeing up resected tissue, and/or use secondary hemostat adjunct), for example, may be used to account for the co-morbidity interaction.

Biomarkers, treatments, and/or pre-operative steps (e.g., pre-operative targets) may be determined to be conflicting and/or beyond a threshold within a baseline procedure plan. The conflicting biomarkers, treatments, and/or pre-operative steps may be identified. The conflicts may be highlighted, for example, in the baseline procedure plan. For example, heart rate and/or blood pressure biomarkers may be high in the pre-operative assessment or monitoring. The patient may be taking medication for the heart rate and/or blood pressure. A conflict may be determined based on that the biomarkers should be in lower range based on the use of the drug. The discrepancy (e.g., irregularity) may be highlighted and/or indicated to the surgeon. The surgeon may use the discrepancy to determine a course of action (e.g., treatment plan and/or modification to the baseline procedure plan).

Images (e.g., pre-operative images) and/or tests (e.g., pre-operative tests) may be summarized, aggregated, and/or aligned (e.g., automatically aligned). The summary, aggregation, and/or alignment may be included in the baseline procedure plan. Proposed adaptions for a baseline procedure plan, for example, may be determined based on the pre-operative data. For example, pre-operative scans may be imported and/or overlaid onto the baseline procedure plan. The overlay may indicate tumor location. The tumor location may be aligned between the scans, which may allow the user (e.g., surgeon) to visualize the location and/or orientation of the tumor. Different angles from different images may provide a better context for tumor location. The overlaid images may be used to adjust baseline margins to align with the tumor scan integrations.

Overlaid images may be used to indicate blind spots and/or lack of visualization. Blind spots may lead to an issue during the surgical procedure. The indication of blinds spots and/or lack of visualization may indicate that data is questionable and/or absent. If data is questionable and/or absent, alternative surgical tasks and/or methods may be determined. Other data may be substituted in for the questionable and/or absent data, for example, from a different imaging source (e.g., live imaging source) and/or a different pre-operative scan.

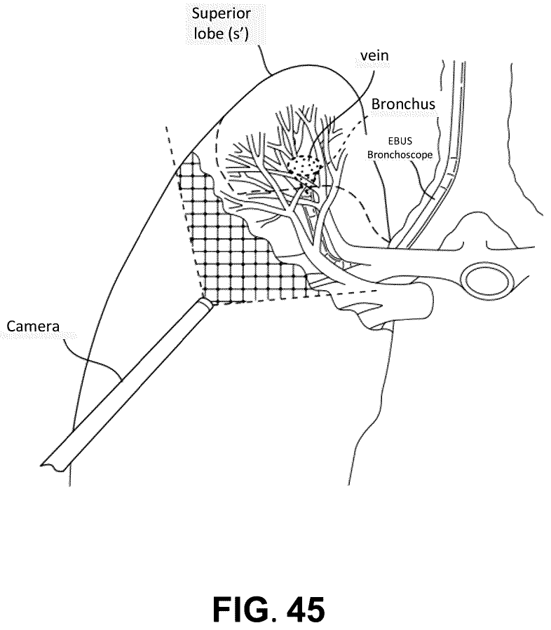

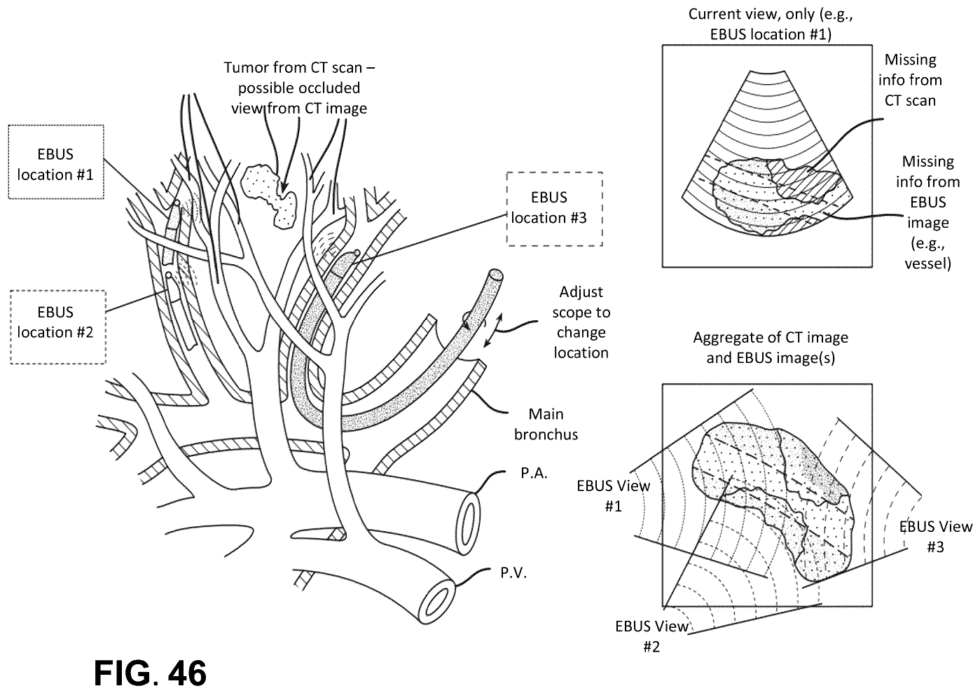

FIG. 45 illustrates an example of an image of a lung generated from multiple sources. A computerize tomography (CT) scan of a lung may be obtained. In examples, portions of an image may be occluded from the CT view of the tumor. The tumor may be occluded, for example, in a CT scan. FIG. 46 illustrates an example image sourced by a laparoscopic camera or endobronchial ultrasound bronchoscopy (EBUS) to fill in missing portions of a full 3D view. As shown in FIG. 46, a laparoscopic camera or EBUS may be used to fill in visualizations missing from other scans. The scans may be aggregated to get a full picture of the lungs. Each scan may not be able to produce a complete image, but aggregating the images may provide a clearer picture to fill in missing portions. Different cameras and/or camera angles may be used to provide the complete lung image. The aggregated image may be provided to the user to convey necessary information. The aggregated image may indicate that information may be needed to be input to complete any missing information and/or indicate that the scans may need to be reperformed. The information missing from the occluded view from the CT scan may be supplemented by aggregating different images from different sources, for example, to provide a complete picture.

Patient-specific surgical steps may be determined, for example, as shown at 50230. The patient-specific surgical procedure may include patient-specific surgical steps. The patient-specific surgical steps may be determined based on the surgical procedure data (e.g., processed surgical procedure data). For example, the patient specific-surgical steps may be determined based on the pre-surgical data sources and a procedure plan template associated with a planned surgical procedure. For example, a thoracic surgical procedure may be planned for a patient. The patient specific surgical procedure may use a thoracic surgical procedure plan template to plan the procedure. The pre-surgical data sources (e.g., with the thoracic surgical procedure plan template) may be used to determine the patient-specific surgical procedure steps.

The patient-specific surgical steps may be associated with surgical tasks. Surgical procedures may be performed using one or more alternative surgical tasks. Surgical tasks may be performed using one or more alternative surgical instruments. For example, a surgical task may involve using a surgical stapler. The surgical task may be completed by the surgical stapler using one or more energy levels, such as, for example, a first energy level or a second energy level. Using the first energy level with the surgical stapler may lead to a first outcome and using the second energy level with the surgical stapler may lead to a second outcome. The first outcome may be improved over the second outcome. The surgical task and/or surgical steps associated with the better outcomes (e.g., as compared with alternative tasks and/or steps) may be preferred for a surgical procedure. Performing a surgical task in a surgical procedure step may affect subsequent surgical tasks and/or subsequent surgical procedure steps. For example, performing a surgical task may affect the rest of the surgical tasks to be performed during the surgical procedure.

The patient specific surgical tasks may be associated with better outcomes, for example, as compared with generic surgical tasks in a surgical procedure plan template. Accommodating a patient's needs and/or the facility's resources may enable a more efficient and/or successful outcome for the surgical procedure. The patient specific surgical tasks may be determined, for example, based on patient specific information, the facility information, staffing information (e.g., HCP availabilities, HCP roles, HCP experience, HCP specialties, etc.), and/or the like.

Characterizations may be determined for the patient specific surgical steps. The patient specific surgical steps may be characterized, for example, based on outcome success, efficiency, risk, efficacy, and/or the like. For example, a patient specific surgical step may be associated with a range associated with outcome success. Patient specific surgical steps may be associated with different surgical outcomes. Characterizations may be determined, for example, based on the patient-specific surgical data and/or historic data associated with the surgical procedure.

In examples, an outcome success for a surgical task option may be characterized. Based on the patient-specific surgical data, an outcome success for a surgical task option may be determined. For example, a first surgical task option may involve using a surgical stapler with a first set of parameters and a second surgical task option may involve using a surgical stapler with a second set of parameters. The first set of parameters and the second set of parameters may result in different outcomes based on the patient's anatomy and/or patient-specific surgical data. For example, if a patient is prone to bleeding, a set of parameters that may increase bleeding may be associated with a lower outcome success. The outcome successes may be characterized, for example, to help select which surgical task option to use in the surgical procedure.

In examples, risk for a surgical task option may be characterized. Based on the patient-specific surgical data, a risk level (e.g., critical risk, moderate risk, low risk, no risk, etc.) may be determined for a surgical task option. For example, a first surgical task option using a first surgical device may create more risk for a patient as compared to a second surgical task option using a second surgical device. The first surgical task option may create more risk, for example, because the first surgical device may negatively interact with the patient based on the patient's anatomy and/or patient-specific surgical data. The first surgical task option may pose a critical risk for the patient, whereas the second surgical task option may pose a moderate risk. The characterized risk levels may be used, for example, to select a surgical task option to use in the surgical procedure.

In examples, efficiency for a surgical task option may be characterized. Based on the patient-specific surgical data, efficiency associated with performing a surgical task may be determined. A first surgical task option may be completed more efficiently than a second surgical task option, for example, based on the surgical device used. For example, a surgical device with a stronger energy generation may hasten the surgical procedure, as compared to a surgical device with a weaker energy generation. Efficiency of a surgical task option may be affected by patient anatomy and/or patient-specific surgical data. For example, a patient's anatomy may lend itself to a certain surgical device over another, which may increase the efficiency of the surgical task option.

Outcomes may be determined (e.g., predicted) for surgical tasks and/or surgical procedure steps, for example, as shown at 50235. Outcomes may be associated with surgical outcomes, complications, efficiency, and/or the like. Outcomes may be determined, for example, based on patient risk, patient survivability, procedure time, and/or the like (e.g., which may be determined based on the obtained data). For example, a first outcome may be determined (e.g., predicted) for a first (e.g., primary) surgical task and a second outcome may be determined (e.g., predicted) for a second (e.g., alternative) surgical task. The first outcome may be associated with a higher success rate as compared with the second outcome. The first outcome may have a higher success rate, for example, based on the patient specific data. For example, if the first surgical task uses a first surgical tool and the second surgical task uses a second surgical tool, the first surgical tool may be more appropriate given the surgical procedure and/or the patient. Therefore, using the first surgical tool may result in a better chance of success.

Outcomes may be determined for surgical tasks, for example, even when there is incomplete and/or conflicting data (e.g., that is used to determine the outcome). For example, data may be conflicting and/or incomplete which may lead to an incorrect outcome prediction. The outcome prediction may take into account the conflicting and/or incomplete data, for example, when determining the outcome. An indication may be sent, for example, that may indicate that the determined outcome is determined based on incomplete and/or conflicting information. The indication may indicate an outcome prediction certainty associated with a typical procedure (e.g., without missing information). The indication may indicate that the determined outcome may be refined, for example, by verifying and/or providing the correct information (e.g., used in the outcome determination). For example, an outcome may be determined for a first surgical task. The outcome may be determined based on incomplete information. The outcome may indicate an outcome prediction certainty range (e.g., 70-90%). The outcome may indicate to the HCP to provide information to refine the outcome prediction certainty range. An HCP may provide the missing information. Using the provided missing information, the outcome prediction certainty range may be refined (e.g., to be 85%-90%).

Outcomes may be determined based on previous surgical procedure data (e.g., from previous procedures) and/or previous surgical procedure outcomes. As shown at 50240, historic surgical procedure data and/or outcomes may be obtained (e.g., from a cloud storage). The historic surgical procedure data and/or outcomes may be used to determine outcomes associated with a current surgical procedure and/or surgical task. The predicted outcome may be stored in the historic procedure data and/or outcomes. The actual outcome of the surgical task/step/procedure may be stored in the historic surgical procedure data and/or outcomes, for example, after the surgical task/step/procedure is collected. The historic data may be used to calculate outcome predictions in subsequent surgical procedures.

The historic surgical procedure data and/or outcomes may be used, for example, to provide context to the patient specific surgical steps/tasks. For example, a cloud aggregation of the outcomes from historic results may be used to provide context associated with the patient specific surgical steps/tasks (e.g., highlight the implication on the patient specific surgical tasks/steps and/or baseline plan). The historic surgical procedure data and/or outcomes may be used to adapt the patient specific surgical steps/tasks (e.g., the baseline procedure plan), for example, based on changes in practices (e.g., best practices), clinical trends, and/or the like. The historic surgical procedure data and/or outcomes may be overlaid on the patient specific surgical step/task and/or modified by the patient data, for example, to identify surgical decision points (e.g., for the user to consider in planning the surgical procedure).

A patient specific procedure plan may be populated, for example, as shown at 50245. The patient specific procedure plan may be populated with surgical steps and/or patient specific surgical tasks. The surgical tasks may be the surgical tasks determined based on the patient specific presurgical data (e.g., attributed with a predicted outcome). The patient specific procedure plan may include a recommended set of surgical steps/tasks (e.g., for each surgical procedure step/task). The patient specific procedure plan may include alternative surgical steps for the recommended surgical tasks. The patient specific procedure plan may provide alternative options for HCPs to select and/or give feedback on. Options that are not relevant to the patient and/or options that may lead to unsuccessful outcomes, may be determined and excluded from the patient specific procedure plan.

Recommended surgical tasks/steps may be determined for the patient specific procedure plan. The recommended surgical tasks/steps may be determined, for example, based on the determined outcomes (e.g., predicted outcomes) associated with the patient specific surgical steps/tasks. The recommended surgical tasks/steps may be the surgical tasks/steps that are associated with a higher predicted outcome success. For example, the populated patient specific procedure plan may include the surgical tasks associated with the highest predicted successful outcome. The recommended surgical task/steps may be the surgical tasks/steps associated with the facility information (e.g., staffing information, tool availability, OR availability, etc.). The recommended surgical task/steps may be the surgical tasks/steps associated with surgeon preferences.

A user (e.g., HCP) may provide input to the patient specific procedure plan, for example, as shown at 50250. For example, an HCP may select an alternative option for a surgical task (e.g., instead of the recommended surgical task). The tasks following the selected alternative option may be affected by the selection, for example, if choosing the alternative task changes how the procedure (e.g., rest of the procedure) can be performed. For example, if the recommended surgical task uses a first surgical tool and the HCP selects the alternative surgical task using a second surgical tool, subsequent surgical tasks using the first surgical tool may be altered (e.g., to consider the use of the second tool). The subsequent surgical tasks may not necessarily need to adapt to the selection of the alternative surgical task option. As shown at 50255, the patient specific procedure plan may be adjusted. The patient specific procedure plan may be adjusted, for example, based on the user input and/or in accommodation of a previously revised surgical task/step.

The HCP input may act as an override to the automated population of the patient specific procedure plan. HCPs may still control how the planned surgical procedure can be performed. For example, the patient specific procedure plan may be used as a baseline (e.g., so the HCP can see the potential strategies and/or options for the surgical procedure). The automated population of the patient specific procedure plan may reduce the workload of the HCPs. The HCPs may focus on selecting the preferred surgical tasks/steps rather than parse through the presurgical data and/or facility information to determine a surgical procedure plan.

The patient-specific procedure plan and/or adjusted patient specific procedure plan may be generated, for example, for the surgical procedure. As shown at 50260, the generated patient specific procedure plan may be displayed. The patient-specific procedure plan may be displayed, for example, as a viewable report (e.g., single viewable report). The patient-specific procedure plan may be sent to a surgical control system. The surgical control system may instruct surgical instruments and/or surgical systems, for example to carry out surgical tasks autonomously (e.g., based on the patient-specific procedure plan). For example, the surgical control system may determine parameters for a surgical instrument to use during a surgical procedure based on the patient-specific procedure plan.

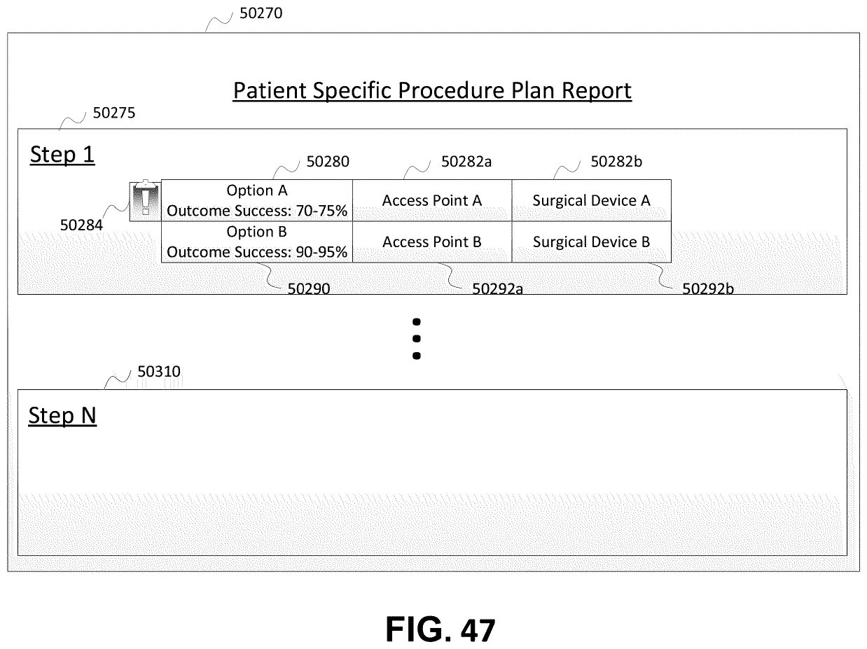

FIG. 47 illustrates an example of a patient specific procedure plan report. The patient specific procedure plan report 50270 may include the surgical procedure steps for a patient specific surgical procedure. The surgical procedure steps may include Step 1 50275 through Step N 50310, for example. The surgical procedure steps may include surgical tasks. The surgical tasks may include a recommended surgical task and/or one or more alternative surgical tasks.

For example, Step 1 50275 may include multiple surgical task options. Step 1 50275 may include Option A 50280 and/or Option B 50290. Option A 50280 may be associated with a first outcome success (e.g., predicted outcome success), which may be the range between 70-75%. Option B 50290 may be associated with a second outcome success, which may be the range between 90-95%. Option A 50280 may use Access Point A 50282a and/or Surgical Device A 50282b. Option B 50290 may use Access Point B 50292a and/or Surgical Device B 50292b. Option A 50280 may be flagged, for example, if the surgical task is associated with incomplete and/or conflicting information/data. For example, Option A may have been determined using data/information that may be incomplete and/or conflicting. As shown at 50284, Option A 50280 may be flagged indicating the HCP to review the data/information. For example, the HCP may provide the missing data/information and/or verify the conflicting data/information. The outcome success associated with Option A may be revised based on the HCP input.

The patient specific procedure plan report may be interacted with (e.g., by an HCP). For example, an HCP may select one or more surgical tasks for a surgical step. The selection may affect the subsequent surgical tasks/steps in the surgical procedure. For example, a selection of a surgical task in Step 1 50275 may affect surgical task options in subsequent steps, such as Step N 50310, for example.

The patient specific procedure plan may include the recommended starting point, surgical tasks, and/or alternative surgical tasks for the surgical procedure. The patient specific procedure plan may include (e.g., an aggregation of) potential complications, an identification of auxiliary information, and/or the like. The patient specific procedure plan may include an identification of a relationship (e.g., an interaction) between procedure steps and the aggregated patient data. The patient specific procedure plan may include an initial access port location identification (e.g., to improve surgical site access), for example, which may be determined based on the procedure step, instrument selection, and/or patient data.

For example, the patient specific procedure plan may include an aggregation of the potential complications and/or identification of auxiliary information. For example, the patient specific procedure plan may include highlights and/or notations indicating identified co-morbidities that may interact, for example, during the surgical procedure. Complications may be determined, for example, such as identifying that co-morbidities may amplify the effects of each other and/or affect treatments that may be selected. Interactive disease states may be calculated and/or indicated, for example, that may increase the probability of complications.

The patient specific procedure plan may include an indication associated with a marginal biomarker. The indication associated with the marginal biomarker may be related to an identified disease state.

The patient specific procedure plan may include a notification associated with procedural steps that may be impacted and/or may be altered. The procedural step may be determined to be impacted and/or indicated to be altered, for example, based on one or more of the following: the determined disease, the state of the disease's advance, situational awareness of the HCP, facility, and/or staff, and/or the like.

The patient specific procedure plan may include an indication (e.g., highlight) associated with an identification of a metallic object. For example, a metallic object (e.g., clip, staple, buttress, etc.) may be detected. The patient specific procedure plan may include an indication (e.g., on an image and/or scan) that may indicate the location (e.g., relative location) of the metallic object. The indication may indicate areas where a surgical tool/device (e.g., ultrasonic device and/or radio frequency bipolar instrument) may interact poorly (e.g., harm the patient and/or not function properly), for example, due to the metallic object. The patient specific procedure plan may include a suggestion of a procedural step option to avoid the metal. The patient specific procedure plan may include a suggestion of alternative instrument(s) to use in the high metal areas. The patient specific procedure plan may include an adaptability of an energy algorithm and/or an integrity test (e.g., to improve detection) before adverse events occur and/or to verify integrity after passing through an area.

The patient specific procedure plan may include an indication indicating a relationship (e.g., interaction) between procedure steps and the patient specific surgical data. For example, the relationship (e.g., interaction) between procedure steps and the patient specific surgical data may be identified. A patient may be scheduled for a surgical procedure, such as a colorectal sigmoid resection (e.g., for Crohn's disease, which may refer to a chronic condition associated with inflammation). Chron's disease may affect and/or be related to (e.g., interact with) patient biomarkers and/or physiologic aspects. The co-morbidities may be affected. The co-morbidities of the patient data and/or therapies (e.g., blood pressure, blood sugar, blood thinners, pain reliever, etc.) may have physiologic effects that may (e.g., have the potential to) impact (e.g., interact with) device choices, device setups, or procedure steps. The procedure plan may be adapted based on the relationship (e.g., interaction). For example, the patient specific procedure plan may indicate a recommendation to adapt the procedure plan based on the relationship (e.g., interaction).

The patient specific procedure plan may include an access port location (e.g., initial access port location). An access port location (e.g., for a surgical procedure) may be identified, for example, based on the patient specific surgical data, instrument selection, surgical procedure step, and/or the like. The surgical site access may be improved, for example, based on the access port location. The access port location may be associated with a location on the patient and/or an angle of a surgical device being used at the surgical access site.

The patient specific procedure plan may indicate an amount of access, for example, for one or more pre-selected access ports (e.g., the amount of access each pre-select access port may provide). The patient specific procedure plan may indicate an interaction overlap for instruments used in an area, for example, based on trocar locations. The patient specific procedure plan may include an overlay of the patient specific data and/or images on the populated procedure plan. The patient specific procedure plan may indicate the trocar location(s). The patient specific procedure plan may indicate access capabilities, for example, based on the combination of the patient specific surgical data.

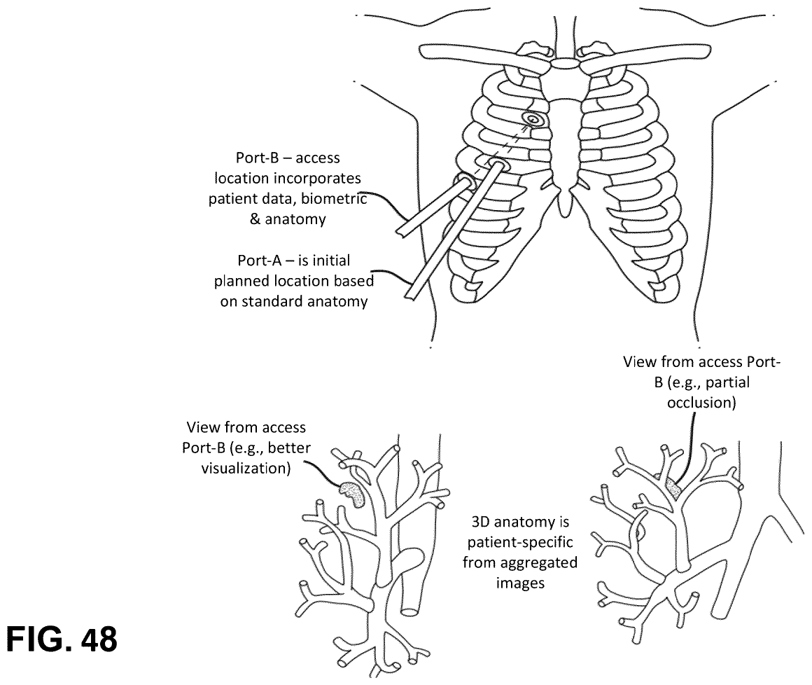

FIG. 48 illustrates an initial access port location identification. For example, access ports may be identified. As shown in FIG. 48, a first access port (e.g., Port A) and a second access port (e.g., Port B) may be identified. The access ports may be determined based on a standard human anatomy. The access ports may be determined based on the standard human anatomy and/or the patient specific surgical data (e.g., biometric data and/or pre-surgical imaging of the patient's anatomy). The patient specific procedure plan may indicate a view of the patient, the surgical site, and/or access ports. The patient specific procedure plan may indicate occlusion (e.g., partial occlusion), for example, based on the patient specific surgical data/images. The patient specific procedure plan may include an anatomy image (e.g., 3D anatomy), which may be patient specific from aggregated images. The patient specific procedure plan may indicate the planned access port, for example, through the rib cage for instruments and/or a camera. The patient specific procedure plan may include an image with an overlay that may show an alternative access port approach, for example, that may provide additional access and/or visualization (e.g., to see occluded areas and/or better access of a tumor).

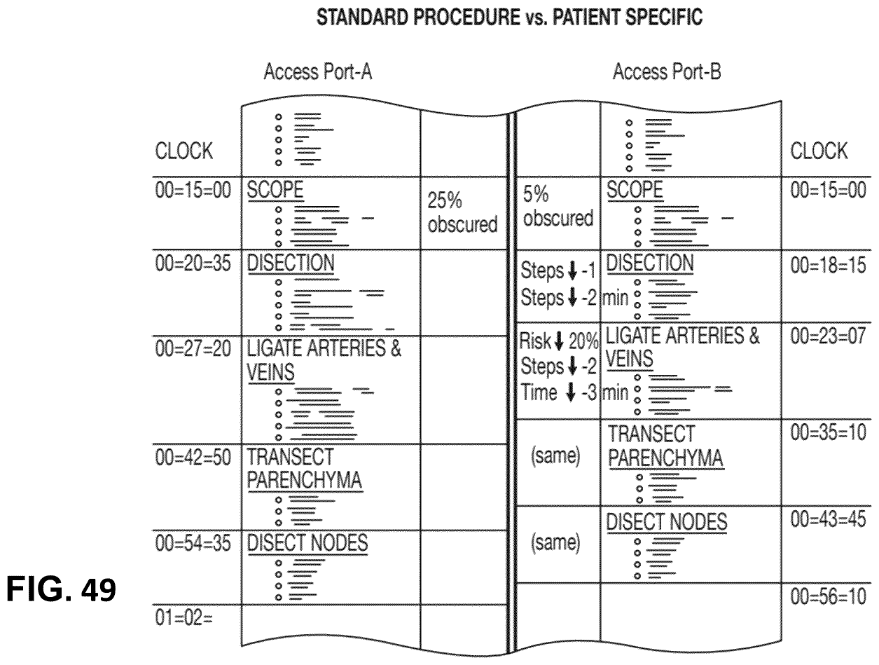

FIG. 49 illustrates an example overlay of patient data and imaging on a procedure plan. As shown in FIG. 49, the patient specific procedure plan and/or the overlay may indicate surgical procedure steps that may be affected. The surgical procedure steps may be affected based on the selected access port. For example, using different access ports may change the surgical procedure plan. The overlay may be used to indicate surgical procedure steps that are affected by poor access ports.

Instrument positioning and/or access envelopes may be determined. The instrument positioning and/or access envelopes may be determined, for example, based on the patient specific surgical data and/or surgical procedure templates. The instrument movement envelope inside (e.g., end-effector, shaft) and/or outside (e.g., shafts, shrouds, robotic arm) the patient wall may be forecasted. The instrument positioning and/or access envelopes may be displayed. For example, the instrument positioning and/or access envelopes may be included in the patient specific procedure plan.

Identification of robotic aspects (e.g., outside the patient) may be automated. The interaction of the robotic aspects may be determined (e.g., automatically). For example, the identification may be performed using the patient specific surgical data, surgical procedure steps, surgical procedure baseline/template, and/or surgical device/tool selection. Conflicts (e.g., potential conflicts) and/or collision may be determined (e.g., as a baseline). Alternative access port locations may be indicated, for example, based on the potential conflicts and/or collisions. Alternative access port locations, patient positions, instrument mixes, and/or the like may be determined (e.g., and highlighted), for example, to minimize determined complications.

Optimal instrument positioning and/or access envelopes may be determined, for example, such that improper interactions (e.g., sword fighting) of shafts inside the patient's body (e.g., as the HCP interacts with the surgical site) is minimized. The determined instrument positioning and/or access envelopes may be determined, for example, to prevent handle, arm and/or shroud collisions (e.g., based on the interaction of the access port location, procedural step, and/or patient specific surgical data).

The patient specific procedure plan may be determined (e.g., automatically determined), for example, based on facility information. Facility information may include surgical tool availability, surgical device availability, inventory stock, surgical equipment availability, staffing, facility room availability, and/or the like. The facility information may be used to adjust the patient specific procedure plan. For example, the patient specific procedure plan may indicate to use a first surgical tool during the surgical procedure, but the first surgical tool may not be available (e.g., not sterilized, down for repair/maintenance). The patient specific procedure plan may be adjusted to use a second surgical tool (e.g., alternative surgical tool) in place of the first surgical tool. The patient specific procedure plan may be adjusted (e.g., subsequent tasks/steps in the procedure plan may be adjusted) based on the adjusted use of the second surgical tool.

The computing system may perform verification (e.g., automatic verification) with the planned surgical procedure. For example, an automated verification may be performed for the instrument needs, the procedure date, the facility usage, the shipments being received associated with surgical tools/consumables used for the surgical procedure, and/or the like. The computing system may determine (e.g., automatically determine) issues associated with the planned surgical instruments to be used in the surgical procedure.

A scheduling time for the surgical procedure may be determined, for example, based on the patient specific procedure plan. The computing system may determine a scheduling time based on the instruments selected in the patient specific procedure plan, staffing availability, facility availability, and/or the like. For example, the scheduled time may be a time when the selected instruments are available (e.g., mostly available, all available).

Devices/consumables may be ordered, for example, based on the inventory information and the devices/consumables that are selected to be used in the surgical procedure. For example, the computing system may determine that surgical consumables are to be used in the surgical procedure and may determine that the inventory is running low. The computing system may order replacement/extra consumables for the surgical procedure, for example, in the case that the replacement/extra consumables are used (e.g., needed) to complete the procedure.

The patient specific procedure plan may include an indication associated with facility inventory and surgical devices selected for the surgical procedure. The indication may indicate inventory alternatives (e.g., inventory that may be used instead of the selected inventory in the surgical procedure plan). The indication may highlight surgical devices that may be missing/unavailable (e.g., in the facility) that were selected for the surgical procedure.

The computing system may determine the patient specific procedure plan based on facility information and/or other surgical procedure being planned. Medical facilities perform many surgical procedures, and the surgical procedures may occur simultaneously and/or overlap in time. Surgical devices may be used in more than one surgical procedure. A surgical device and/or consumable may have already been selected for a surgical procedure, and therefore may not be used in a different surgical procedure occurring at the same time. The computing system may consider (e.g., in determining the patient specific procedure plan) other surgical procedures being planned. The computing system may interact with other users planning procedures that may use the same staffing/personnel and/or instruments. The computing system may prioritize surgical procedures. For example, a first surgical procedure may be prioritized over a second surgical procedure. The first surgical procedure may have priority in device selection and/or consumable selection. The prioritization may be performed based on a requirement associated with the surgical procedure, a risk level associated with the surgical procedure, and/or the like.

In examples, a surgeon may develop a baseline surgical procedure plan/map. The baseline plan may include the surgical devices to be used in the procedure. The computing system may determine (e.g., based on facility information and the baseline plan), that a selected surgical instrument (e.g., an Echelon 60) is not available in stock. The computing system may indicate that the surgical instrument is not available for the procedure. The computing system may indicate an alternative surgical instrument (e.g., Echelon 45), for example, that may be available. The computing system may indicate to the surgeon that the selected surgical instrument is not available and recommend the alternative surgical instrument. The surgeon may accept or deny the recommendation. The computing system may update the baseline procedure plan, for example, if the surgeon accepts the recommendation to use the alternative surgical instrument. The baseline procedure plan may be updated to accommodate the use of the alternative surgical instrument (e.g., instead of the selected surgical instrument). For example, the baseline procedure plan may be adjusted such that the procedure step may include the possibility of extra firings from the alternative stapler (e.g., to accommodate the differences in the two surgical devices). The computing system may indicate that the adjusted baseline procedure plan has been adjusted to increase a number of cartridges (e.g., to compensate for the difference with the alternative surgical instrument). The computing system may indicate to the surgeon a cartridge color selection for the extra firings. The computing system may update the baseline procedure plan (e.g., if the surgeon accepts the modifications), and may order the additional supplies based on the changed procedure plan. The computing system may verify that the additional approach (e.g., extra firings using the alternative surgical tool) is acceptable from a risk perspective.

The patient specific procedure plan may be determined, for example, based on room, patient, and/or staff availabilities. The patient specific procedure plan may overlay room, patient, and/or staff availabilities on a baseline procedure plan. The patient specific procedure plan may include follow-ups based on the room, patient, and/or staff availabilities. For example, automatic patient scheduling for the procedure and/or follow-ups may be performed.

For example, the computing system may perform scheduling correlation (e.g., automated scheduling correlation) for the facility rooms, equipment, personnel, and/or surgeon availability. The computing system may determine a time (e.g., optimal time) for the planned procedure, for example, based on the surgical procedure (e.g., acute critical need of the procedure). For example, the computing system may consider whether a planned surgical procedure is urgent. The computing system may perform escalation associated with a surgical procedure timing and/or scheduling, for example, based on the patient needs and/or other planned procedures. The computing system may aggregate the patient's scheduled treatments, for example, to determine a comorbidity implication associated with the planned surgical procedure. The computing system may consider pre-operative patient progress to predefined goals (e.g., weight loss), for example, as a trigger to change procedure scheduling. The computing system may update patient biomarkers and/or patient examination of patient health from monitoring HCPs (e.g., surgeon, primary care physician, pharmacist, physical therapist, and/or the like). The computing system may perform a comparison (e.g., automatic comparison) between patients (e.g., patients of other scheduled procedures), that may impact and/or create a conflict with the current scheduling. The computing system may proposed changes to scheduling of the surgical procedures, for example, to resolve the determined conflicts.

The computing system may perform procedure scheduling based on personnel availability and/or scheduling. The computing system may consider the availabilities of the HCPs, for example, including the anesthesiologist, assistant physician, back table nurse specialist, oncology/imagine specialist, a technician, a urologist, and/or the like.

Systems, methods, and instrumentalities are disclosed for identification of image shapes based on situational awareness of a surgical image and annotation of shapes or pixels. A surgical video comprising video frames may be obtained. Surgical context data for a surgical procedure may be obtained. Elements in the video frames may be identified based on the surgical context data using image processing. Annotation data may be generated for the video frame, for example, based on the surgical context data and the identified element(s).

Elements within a surgical video may be identified, for example, by a surgical computing system. For example, image shapes may be identified in the surgical video, and the image shapes may be linked to surgical elements. For example, the image shapes may be identified as structures, organs, features, and/or other elements in a surgical procedure. For example, an image element may be identified as a lung, tumor, blood, artery and/or other anatomical elements. Features associated with the elements may be identified. The identified features may include, for example, one or more of the following: status, condition, type, tissue type (e.g., fat, fat and vessel, duct, organ, fat and organ, connective tissue, etc.), tissue condition (e.g., inflamed, friable, calcified, edematous, bleeding, charred, etc.), and/or the like.

The identification of such elements and/or features may be performed, for example, using image processing on the surgical video (e.g., a surgical video frame) and using situational awareness associated with the surgical image. Situational awareness may be accomplished using surgical context data and/or surgical procedure data obtained via a situational awareness system (e.g., as described herein with respect to FIG. 7). Annotation data may be determined based on the identified elements. The annotation data may be inserted into the surgical video.

A surgical video or surgical video frame may be parsed into grouped elements and/or groups of pixels. Throughout a surgical video, the elements may exhibit movement, behavior, and/or outcomes (e.g., surgical events, such as bleeding). The elements may be tracked between surgical video frames (e.g., consecutive frames) in the surgical video. Tracking data (e.g., behavior, movement, and/or outcome data) may be determined, for example, using the surgical context data and the identified elements. The tracking data may be included in the annotation data and inserted into the surgical video. The tracking data may be used for verification of anticipated movements, behaviors, and/or outcomes.

The surgical computing system may perform annotation of pixels within an image. The annotation of pixels may be used for machine learning, object identification, object tracking, self-identification, and/or the like. The annotation may include situational identification and annotation of individual pixels and/or groups of pixels in the surgical video.

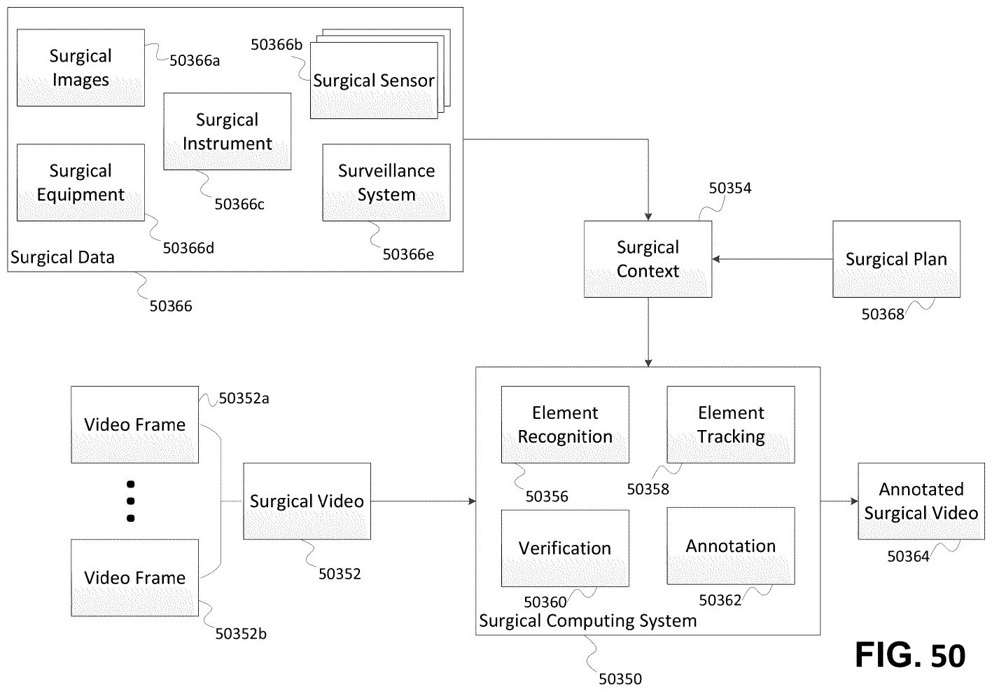

FIG. 50 illustrates an example of annotating surgical video based on situational awareness of the surgical image. As shown in FIG. 50, a surgical computing system 50350, which may be the surgical hub 20006 as shown in FIG. 1, may obtain a surgical video 50352 and a surgical context 50354 (e.g., surgical context data).

The surgical context 50354 may indicate a surgical procedure, a surgical event, a surgical step, a surgical phase, a surgical task, a complication, and/or the like. The surgical context 50354 may be determined. For example, the surgical context 50354 may be determined using situational awareness (e.g., as described herein with respect to FIG. 7).

As shown, the surgical context 50354 associated with the surgical video and/or surgical procedure may be determined, for example based on surgical data 50366 (e.g., surgical procedure data). The surgical data 50366 may be surgical data generated before, during, and/or after a surgical procedure. For example, the surgical data 50366 may be the surgical procedure data 50020 as shown in FIG. 41. The surgical data 50366 may include surgical data generated from surgical systems, such as, for example, surgical images 50366a, surgical sensor(s) 50366b (e.g., wearables), surgical instrument(s) 50355c, surgical equipment 50366d, surveillance system(s) 50366e, and/or the like.

The surgical context 50354 may be determined, for example, based on the surgical data 50366 and a surgical plan 50368 (e.g., surgical procedure plan). The surgical plan 50368 may include information associated with a surgical procedure, such as, for example, the surgical procedure steps, surgical procedure tasks, surgical instruments used, surgical equipment used, and/or the like. The surgical plan 50368 may be a patient-specific surgical procedure plan (e.g., as described herein with respect to FIGS. 11, 14, and 16). The surgical context 50354 may be a patient-specific surgical context for the surgical procedure. The surgical context 50354 may be determined, for example, based on the patient's specific anatomy and/or medical records.

The surgical context 50354 may indicate a surgical procedure step, for example. The surgical procedure step may be associated with performing an incision on a portion of a lung. The surgical context 50354 may include information associated with the surgical instruments used, surgical equipment used, the organs in the surgical site, features in the surgical site, and/or the like. The surgical context 50354 may be used to guide the surgical procedure.

As shown in FIG. 50, the surgical computing system 50350 may obtain a surgical video 50352. The surgical video may be composed of video frames (e.g., surgical video frames), for example, such as video frame 50352a and video frame 50352b. The surgical video may be a video of the surgical procedure.

The surgical computing system 50350, which may be the surgical hub 20006 as described in FIG. 1, may perform element recognition and/or element identification, as shown at 50356. The surgical video may be associated with an endoscopic surgery (e.g., inside the body video). For example, the surgical video may capture video inside a patient's body during the surgical procedure. The surgical video may capture video showing elements inside the patient's body, such as, organs, structures, features, anatomy, surgical instruments (e.g., a portion of a surgical instrument, for example, such as a tip, jaw, knife, end effector, etc.), tissue, tumors, arteries, veins, bronchus, and/or the like. The elements inside the patient's body may be identified.

Element identification and/or element recognition may be performed based on the surgical context 50354 and situational awareness (e.g., as described herein), for example, using image processing (e.g., as described herein). As described herein, the surgical context 50354 may provide context for the surgical procedure and/or elements identified in a surgical video during the surgical procedure. For example, a surgical context 50354 may provide information associated with a lung surgical procedure. The surgical context 50354 may be used to identify elements in a surgical video, such as a lung. The surgical context 50354 may be used to distinguish elements in a surgical video from other similar elements. For example, organs may share a similar shape in a surgical video, but with the surgical context, the proper organ may be identified. For example, during a lung surgical procedure, the surgical context may be used to identify the element as a lung rather than the element being a different organ with a similar shape as lungs. For example, the surgical context 50354 may indicate that a particular surgical device is being used, which may be used to identify the surgical device or a portion of the surgical device in a surgical video frame(s).

The surgical computing system 50350 may identify elements in the surgical video 50352. The surgical computing system 50350 may perform element identification and/or element recognition on the surgical video 50352, for example, by using the video frames. The surgical computing system 50350 may perform element identification and/or element recognition on each video frame, for example, such as a first video frame (e.g., the video frame 50352a) and a second (e.g., subsequent or previous) video frame (e.g., video frame 50352b). The video frames may be processed to identify elements within each video frame.

The surgical video 50352 and/or video frames may be composed of pixels. The elements in the video frames may be composed of groups of pixels, for example. A first group of pixels may be clustered and identified as making up a first element in the video frame. The surgical computing system 50350 may determine the first group of pixels associated with the first element. The identified element(s) may be comprised of a respective group of pixels. The identified element(s) may be separated into sub-elements that comprise the respective element. The sub-elements may be comprised of sub-groups of pixels associated with each sub-element.

The surgical computing system may use one or more selection methodologies, for example, to identify elements in a surgical video. The selection methodologies may be associated with using one or more of the following: a bounding box; a polygon; a point (e.g., keypoint); a cuboid; semantic segmentation; etc.

For example, the surgical computing system may use a bounding box selection methodology to identify elements in a surgical video. A bounding box selection methodology may be associated with using a rectangular structure to match the element. For example, the rectangular structure may relate to x(min)/y(min) and x(max)/y(max). The bounding box may enable detection and/or recognition of objects of differing classifications. The boxes may be anatomically located, for example, corresponding to the expected location organs, structures, and/or tissues (e.g., based on the identification of key landmarks, such as, for example, other organs, structures, and/or tissues). For example, differentiation between elements of similar shapes may be enabled (e.g., differentiation of the stomach from the liver), for example, regardless of the shape and/or color similarity. The boxing (e.g., automatic boxing) may enable the surgical computing system to define the differences (e.g., between elements of similar shape and/or color), for example, using limited processing in a gross approach.

For example, the surgical computing system may use a polygon selection methodology to identify elements in a surgical video. A polygon selection methodology may be associated with using a series of selected points that when used together may create a poly structure that may enable classification differentiation. For example, the points may be a perimeter of an element. The points may be a perimeter based on one or more of a color, texture, a morph of multi-spectral imaging with visual imaging, etc. The number of points defining the polygon may increase processing and/or selection timing.

For example, the surgical computing system may use a cuboid selection methodology to identify elements in a surgical video. A cuboid selection methodology may be associated with using a box (e.g., three-dimensional box) selection that enables for multi-dimensional viewing.

For example, the surgical computing system may use a semantic segmentation selection methodology to identify elements in a surgical video. The semantic segmentation selection methodology may be associated with pixel by pixel tissue semantic annotation of the tissue (e.g., all the tissue). An overlay of where the elements (e.g., organs and/or tissue) may be generated. How to anticipate the elements may be determined. In examples, each pixel and/or group of pixels may be annotated and/or labeled as to what element the pixel is associated with.

The surgical computing system may identify (e.g., automatically identify) that the selection methodology used for the element identification is inappropriate (e.g., inaccurate). For example, the surgical computing system may use a baseline approach that is predefined. For example, if a threshold amount of irregularities and/or discrepancies are identified using a selection methodology, the surgical computing system may adjust (e.g., automatically adjust) the selection methodology. The surgical computing system may compare the results (e.g., between the different selection methodologies) and select the selection methodology that results in the least amount of resources used and/or least amount of false positives.

The surgical computing system may perform selection delineation. Selection delineation may be associated with instance segmentation and/or bordering. For example, the surgical computing system may perform instance segmentation associated with the identified elements. Instance segmentation may include taking an element and separating it into subsets of multiple instances. The elements in the surgical video 50352 and/or video frames may comprise sub-elements. For example, an element may be an organ (e.g., lungs) and the organ may have differentiable sections.

The identified element(s) may be separated into sub-elements that comprise the respective element. The sub-elements may be comprised of sub-groups of pixels associated with each sub-element. For example, the surgical computing system may identify an element to be the small intestines or jejunum. The jejunum may be separated into multiple anatomic sections (e.g., four anatomic quadrants). The surgical computing system may identify the overall structure of the jejunum. The surgical computing system may perform bunching of a quadrant group in the jejunum to define the four portions of the jejunum. The surgical computing system may use the differentiable portions of the jejunum to determine information associated with the video frame. For example, the surgical computing system may use the information associated with the differentiable portions to identify where the surgical system is viewing.

For example, the group of pixels associated with an element may be further divided into sub-groups of pixels. The sub-groups of pixels may compose the sub-elements. For example, a first sub-group of pixels may include the pixels that compose the left lung, and a second sub-group of pixels may include the pixels that compose the right lung. A first sub-group of pixels may include the pixels that compose a first lobe in the left lung, and a second sub-group of pixels may include the pixels that compose a second lobe in the left lung.

For example, lungs may be composed of a left lung and a right lung. The left lung may be differentiable from the right lung (e.g., the left lung may be smaller than the right lung and/or may be composed of a notch, for example, to give room for the heart). The lungs may be composed of (e.g., divided into) lobes. The lungs may be composed of five lobes. For example, the left lung may comprise two lobes, and the right lung may comprise three lobes. The lobes may be differentiable from each other. The surgical computing system may identify the sub-elements (e.g., lobes, left lung, and/or right lung) within an element, based on the surgical context data 50354.

The surgical computing system may use surrounding elements and/or features in the video frame, for example, to identify the lungs and/or sub-elements of the lungs (e.g., lobes). The surgical computing system may use the shape of the lungs, the ribcage, and/or other hard landmarks to orient itself, for example, and separate the different segments of the lungs. The user may section and/or review the portions of interest, and/or note the effects on the other portions.

The surgical computing system may perform bordering, for example, in the video frame, based on the surgical context data 50354. The surgical computing system may differentiate the tissues and/or organs, for example, from connective tissue background. The differentiation may be performed, for example, once a structure (e.g., element) is identified and/or selection and divided into instances. A border around the sub-elements (e.g., instances) may be defined. The object may be defined (e.g., self-defined), for example, based on the border. If the border is overlapped with a border of another segment, the shared border may be aligned between the organs and/or tissues, for example, to eliminate the overlap.

The surgical computing system may highlight and/or delineate the elements in the surgical video and/or video frame(s). Once the structures are identified and/or segmented, the surgical computing system may change the contrast and/or brightness of the video frame, for example, to accentuate elements and/or features in the frame. Objects of interest may be selected to be accentuated. The objects of interest may have the contrast and/or brightness changed, for example, to allow a user to differentiate between the objects of interest and other elements in the surgical video. Instances and/or sub-elements may be accentuated, for example, based on the surgical context data 50354.

The surgical computing system may determine characteristics and/or features associated with the identified elements. For example, the surgical computing system may determine the element identification. The surgical computing system may determine characteristics and/or features, such as, for example, one or more of the following: status, condition, type, tissue type (e.g., fat, fat and vessel, duct, organ, fat and organ, connective tissue, etc.), tissue condition (e.g., inflamed, friable, calcified, edematous, bleeding, charred, etc.) and/or the like.

As shown in FIG. 50, the surgical computing system 50350 may perform element tracking 50358. The surgical computing system may perform element tracking (e.g., object tracking) for example, for elements in the surgical video. Element tracking may include determining tracking information for elements in the video frames. For example, an element in a first video frame may be identified in a second video frame. A behavior, movement, and/or outcome may be determined for the element using the first video frame and second video frame. For example, the second video frame may show the element in a different location than the element was in the first video frame. Information about the element across video frames may be determined.

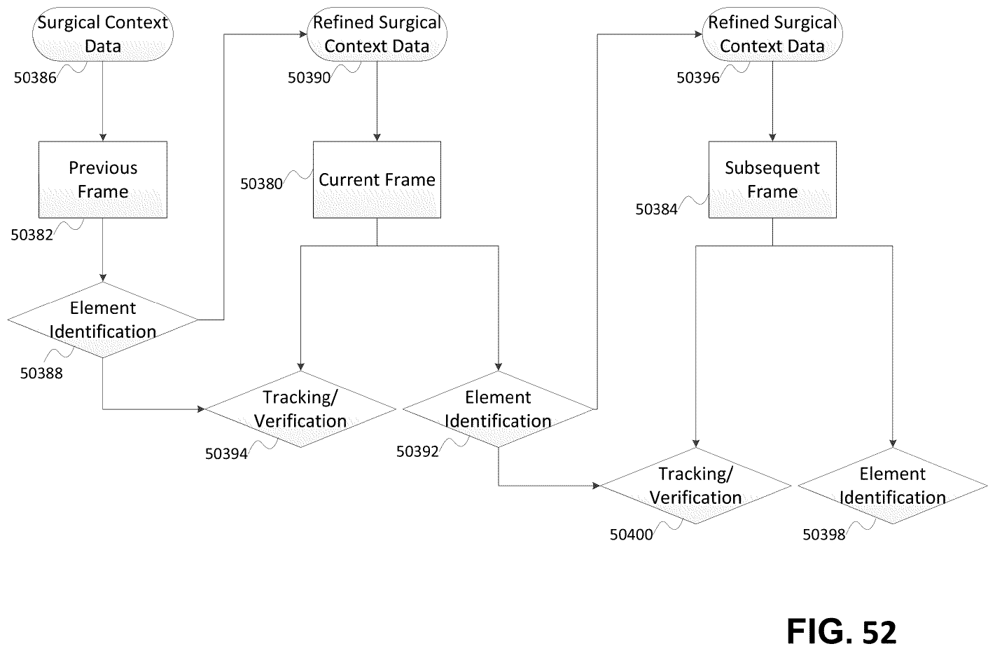

The surgical computing system 50350 may perform verification, for example, as shown at 50360. The verification may include verifying the identified elements and/or tracking. Element tracking and verification will be described in more detail with reference to FIG. 52.

As shown in FIG. 50, the surgical computing system 50350 may generate annotation data, for example, based on the performed element recognition, element tracking, and/or verification (e.g., as shown at 50362). The surgical computing system may determine annotation data for the surgical video and/or video frames. The computing system may determine annotation data for the surgical video and/or video frames, for example, using the identified elements in the video frames and the surgical context data. For example, the annotation data may include element identification information and/or element tracking information. The annotation data may include sub-element identification within the elements. The annotation data may include general information associated with the video frame, such as, for example, the surgical procedure step, surgical task, surgical event, and/or the like, associated with the video frame. The annotation data may include one or more of the following: element identification information, element grouping information, element subgrouping information, element type information, element description information, element condition information, surgical step information, surgical task information, surgical event information, surgical instrument information, surgical equipment information, and/or the like.

The annotation data may be generated based on a surgical event indicated in the surgical context. Event directed annotation (e.g., automatic annotation) may be performed. For example, annotation may be automated using the surgical computing system. Data flagging may be performed, for example by the surgical computing system or a sensor. For example, event directed annotation may be performed for a coupled surgical task, a coupled instrument, or instrument setup.

Event directed annotation may be performed which may be associated with micro-outcomes of a previous surgical step. For example, a previous surgical step may indicate bleeding. The event directed annotation may include determining annotation data to include the magnitude of the bleeding, a timing of the bleeding (e.g., how many frames the bleeding occurs for), a time associated with releasing from the primary surgical instrument, a secondary follow-up interventional surgical tool use, and/or the like.

Event directed annotation may be associated with determining out of body images. For example, the surgical computing system may be configured to redact (e.g., blank out) images that are detected to be out of body.

Event directed annotation may include inserting annotation data including local and/or global metadata. Local metadata may include lead up information, for example, information associated with neighboring video frames. Local metadata may include continuous variables and/or discrete variables. Global metadata may include information associated with the patient, instrument, procedure, surgeon, and/or the like. The metadata may be inserted into the surgical video.

Event directed annotation may include inserting annotation data associated with a response to an identified event. For example, a video frame may be associated with a surgical event. A response to the surgical event may be determined. An algorithm (e.g., querying algorithm) may be used to detect a resulting event. For example, charring may be detected. The annotation data may indicate to focus on the generator data, for example, to provide information on the detected event.

Annotation may be performed during a live surgical procedure and/or after a surgical procedure. Annotation data associated with complicated improvements to the surgical procedure may be performed after the surgical procedure. Live annotation may include simpler annotation and/or processing tasks. For example, live annotation may be performed to minimize the need for secure storage and/or processing of the live surgical procedure data.

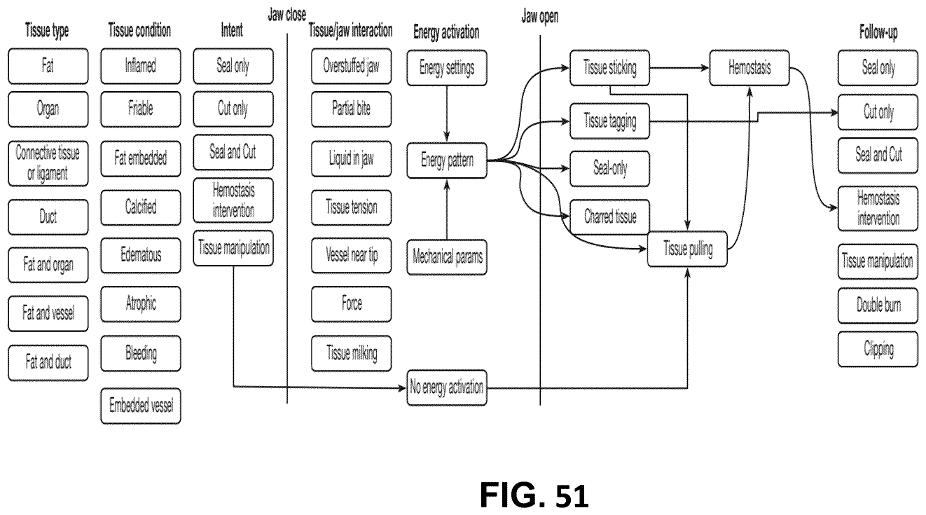

Annotation may be guided by a surgical procedure map. FIG. 51 illustrates example annotations associated with a surgical procedure map. The surgical procedure map may be included in a surgical procedure plan. The surgical procedure map may indicate potential surgical steps and/or surgical outcomes associated with a surgical procedure. For example, the surgical procedure map may indicate potential characteristics associated with elements (e.g., organs, structures, and/or features) in the surgical procedure. For example, the surgical procedure map may indicate potential tissue types, tissue conditions, and/or procedure intents associated with a surgical procedure. The surgical procedure map may guide a surgical procedure. The surgical procedure map may be used as a dictionary, for example, to perform annotation of surgical video and/or video frames associated with a surgical procedure.

The surgical procedure map may indicate a surgical workflow. For example, the surgical procedure map may indicate potential outcomes associated with the surgical workflow.

In some examples, the annotation data may be inserted into the surgical video. The annotation data may be information associated with the frame itself and/or metadata associated with the surgical procedure and/or events. An annotated surgical video 50364 may be generated and output, for example, to surgical systems, storage, and/or the like. The annotation data may be inserted into the surgical video, for example, on the video frame level. The annotation data may be attached to the video frame. The annotation data may be inserted into the surgical video, for example, on a pixel level (e.g., pixel group level). For example, each pixel in a video frame may have respective annotation data inserted into the pixel. The annotation data for each pixel may include information identifying the element the pixel is associated with.

In examples, a group of pixels may be associated with lungs. The surgical computing system may identify the lungs and determine annotation data associated with the lungs. The surgical computing system may insert lung annotation data in the group of pixels associated with the lungs. The annotation data inserted into the group of pixels may include identification information, lung condition information, and/or the like. The surgical computing system may identify instances of the lungs (e.g., lobes of the lungs). The instances of the lungs may be composed of sub-groups of pixels. The surgical computing system may insert sub-element annotation data associated with the respective instances of the lungs in the associated sub-groups of pixels. For example, the surgical computing system may insert respective sub-element annotation data in the sub-group of pixels associated with a first lobe in the left lung. The sub-element annotation data inserted in the sub-group of pixels associated with the first lobe in the left lungs may include identification information that may indicate the sub-element.

In some examples, the annotation data may be used to generate control signal (e.g., indicating the determined control parameters), for example, to a surgical control system. In some examples, the annotation data may be stored and/or sent separately from the surgical video (e.g., to a surgical control system, to a surgical analysis system, etc.).

FIG. 52 illustrates an example flow of determining element tracking information and verification for elements in a surgical video. The surgical video may include video frames. For a selected video frame, element tracking information and verification may be determined, for example, using a previous video frame(s). Element tracking information may be used for element tracking information and verification determination for subsequent video frame(s). As shown in FIG. 52, a current frame 50380 of the surgical video may be the frame that is temporally after a previous frame 50382 and comes before a subsequent frame 50384. The surgical computing system may perform element identification on the previous frame 50382 (e.g., as shown at 50388). The element identification on the previous frame may be performed based on surgical context data 50386 (e.g., as described herein).

The surgical context data 50386 may be refined and/or updated (e.g., as shown at 50390 in FIG. 52). For example, the surgical context data may be refined and/or updated based on the identified element(s) in a first video frame. Element(s) in a second video frame may be identified, for example, using the refined and/or updated surgical context data (e.g., using image processing). Annotation data for the second video frame may be determined using the refined surgical context data and the element(s) identified in the second video frame.

For example, the surgical context data 50386 may be refined and/or updated using the identified elements in the previous frame 50382. For example, the identified elements in the previous frame 50382 may provide additional information about the surgical context of the surgical procedure. The additional information may be used to verify the surgical context data. If there are discrepancies between the surgical context data and the identified elements, the surgical context data may be updated. For example, the surgical context data may indicate that a first surgical instrument is being used at a time associated with the previous frame 50382. The element identification information may indicate that a second surgical instrument is being used in the previous frame 50382 (e.g., instead of the first surgical instrument). The surgical context data may be refined and/or updated to be used in subsequent frames. The element identification information may indicate that the first surgical instrument is being used in the previous frame 50382 (e.g., same as the surgical context data). The surgical context data may be verified, for example, based on the confirming identification information.

The refined surgical context data 50930 may be used for element identification, element tracking, and/or verification for the current frame 50380. Element identification may be performed for the current frame 50380 (e.g., as shown at 50392), for example, using the refined surgical context data 50390 (e.g., as described herein). Element tracking and/or verification may be performed on the surgical video, for example, using the previous frame 50382 and the current frame 50380. Although not shown in FIG. 52, those skilled in the art would appreciate that element tracking information derived from a current video frame may be used to refine and/or update the element identification of previous video frame(s).

As shown at 50394, the element tracking and/or verification may be performed using the identified elements from the previous frame 50382 and the identified elements from the current frame 50380. Tracking information associated with a surgical video may be determined, for example, using multiple video frames. Elements identified in a first video frame and elements identified in a second video frame may be used, for example, to determine tracking data. The tracking data may be associated with element behavior, element movement, and/or outcome information (e.g., associated with the elements). The determined tracking data may be included in the annotation data.

For example, movement, behavior, and/or outcome information may be determined for the elements across the previous frame 50382 and the current frame 50380. An element in the previous frame 50382 may be identified as in a different location in the current frame 50380. Tracking data may be determined based on the movement of the element.

Differences in an element between the previous frame 50382 and the current frame 50380 may indicate a behavior of the element. For example, the element may be determined to be pulsing and/or exhibiting a repetitive movement across video frames. Repetitive movement of an element may be confirmed, for example, by tracking the element across video frames. Element identification may also be performed using the repetitive movement identified for an element. For example, an element may behave and/or move in a way that is indicative of a specific organ and/or feature. Tracking data of an anticipated rhythmic movement of an element may indicate the organ and/or feature. The determined movement may also be used to improve a perimeter bounding of the element (e.g., as described herein with respect to selection methodology), for example, relative to the background of the video frame. The tracking data may be used to confirm and/or refute the type and/or classification of an element.

Differences in an element between the previous frame 50382 and the current frame 50380 may indicate an outcome associated with the element. For example, an element in the previous frame 50382 and the current frame 50380 may be identified as an organ. The condition of the organ in the previous frame 50382 may be identified as not bleeding, for example. The condition of the organ in the current frame 50380 may be identified as bleeding. The change in conditions from not bleeding to bleeding may indicate a surgical event. The bleeding event may be determined and may be included in the annotation data.

The element identification of the current frame 50392 may be used to further refine and/or update the surgical context data (e.g., as shown at 50396). The further refined and/or updated surgical context data 50396 may be used for element identification, element tracking, and/or verification for a subsequent frame (e.g., such as subsequent frame 50384). For example, the element identification of the subsequent frame may be performed, as shown at 50398. Element tracking and/or verification may be performed (e.g., as shown at 50400) based on the current frame 50380 (e.g., elements from the current frame 50392) and the subsequent frame 50384 (e.g., elements from the subsequent frame 50398).

The tracking data and/or information may be verified, for example, using anticipated tracking information. The anticipated tracking information may be obtained, for example, based on the surgical context data and/or a surgical procedure plan. The annotation data may include an indication that indicates that the elements and/or tracking data is verified. The annotation data may include an indication that indicates that the elements and/or tracking data may need further verification.

Verification may be performed for the identified elements in the video frame(s), for example, based on anticipated tracking information. The anticipated tracking information may be associated with a surgical context (e.g., as indicated by the surgical context data). The anticipated tracking information may be determined, for example, based on the surgical context data, such as by using the surgical procedure plan (e.g., patient-specific surgical procedure plan, as described herein with reference to FIG. 44). For example, a surgical procedure plan may indicate that a surgical instrument is to be used during a surgical step. An element may be verified as a specific surgical instrument, for example, based on the surgical computing system identifying the element as being the specific surgical instrument and the surgical context data indicating that the specific surgical instrument data is proper for the surgical procedure step. The verification may be included in the annotation data. For example, the annotation data may indicate that the identified elements are verified.

Jobs, outcomes, and/or constraints may be determined, for example, for a surgical video. The jobs, outcomes, and/or constraints may be determined based on the surgical context data. Surgical task information associated with a first video frame may be determined using the surgical context data and identified elements in the first video frame. Complication information may be determined associated with the determined surgical task, for example, using the surgical context data, the surgical task information, and the identified elements in the first surgical video frame. Outcome information associated with the surgical task may be determined, for example, based on the surgical context data, the surgical task information, and the complication information.

Jobs, outcomes, and/or constraints may be compiled, for example, for a surgical procedure and/or surgical steps. For example, the annotation data may be used to compile the jobs, outcomes, and/or constraints. The compiled jobs, outcomes, and/or constraints may be used (e.g., with machine learning), for example, to identify issues, change control algorithms and/or parameters, recommend procedural changes for subsequent steps and/or procedures, and/or the like.

Surgical task information may be determined for a video frame in a surgical video. For example, surgical task information may be determined using surgical context data and the identified elements in a video frame. For example, the surgical task information may indicate information associated with the performed surgical task in the video frame. The video frame may be associated with removing a portion of the lungs using a surgical instrument. The surgical task information may include information associated with the surgical instrument used (e.g., type of surgical instrument, surgical instrument parameters, etc.), the time of the removal of the portion of the lungs, and/or the like.

Complication information may be determined for a video frame in the surgical video. For example, the complication information may be determined based on the surgical context data, the surgical task information, and the identified elements in the video frame. The complication information may include information indicating complications detected in the video frame. The complication information may include information indicated possible complications that may occur based on the analysis of the video frame. For example, a video frame may be annotated with information indicating a risk of a complication in subsequent surgical steps in the surgical procedure. The annotation data may indicate the potential complication.

Outcome information may be determined for a video frame in the surgical video. The outcome information may be associated with the surgical task in the video frame. The outcome information may be determined based on the surgical context data, the surgical task information, and the complication information. The outcome information may indicate a likelihood of success for a surgical procedure step. The outcome information may indicate a likely result of the surgical task being performed in the video frame. The outcome information may be used to alter subsequent surgical tasks and/or steps in the surgical procedure.

Change and/or control parameters may be determined for a surgical procedure by analyzing the surgical video frame(s). Change and/or control parameters may be determined, for example, based on the surgical task information, complication information, and/or the outcome information. A determination to change parameters associated with a surgical procedure may be performed. Parameter may include parameters associated with operating a surgical instrument, surgical equipment, and/or the like. Based on a determination to change parameters associated with a surgical procedure, change and/or control parameters may be determined. A control signal may be generated. The control signal may include an indication that indicates the determined control parameters. The control signal may be sent to a surgical control system, for example, that send parameter information to surgical systems (e.g., surgical instruments, surgical equipment, and/or the like). The change and/or control parameters may be used to perform the surgical procedure.

Change parameters for a surgical procedure may be determined based on the annotation data. For example, change parameters may be determined based on the surgical task information, complication information, and/or outcome information. For example, the outcome information may indicate a potential complication for the surgical procedure, for example, if the current parameters were to be used for subsequent surgical tasks and/or steps. The surgical computing system may determine different parameters may be used that are associated with a better surgical outcome (e.g., more efficient procedure, lower risk procedure, and/or better outcome).

The surgical computing system may determine to change parameters associated with performing the surgical procedure. The surgical computing system may determine the change parameters to perform the surgical procedure. The surgical computing system may send a control signal (e.g., indicating the determined control parameters), for example, to a surgical control system. The surgical control system may control the surgical instruments, surgical equipment, and/or the like associated with performing the surgical procedures. The parameters may be adjusted (e.g., automatically adjusted) to use the change parameters indicated in the control signal, for example, by the surgical control system.

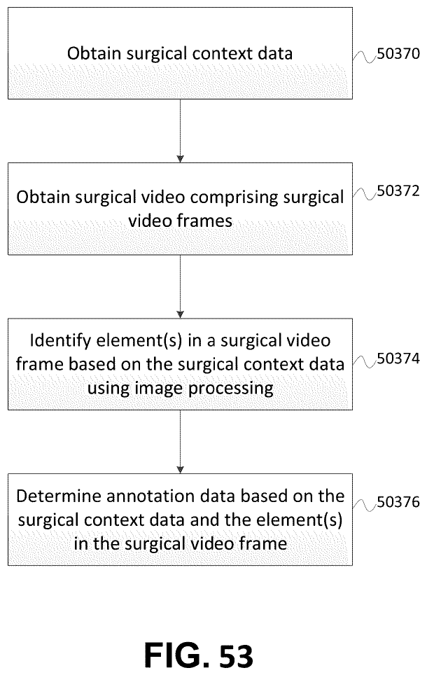

FIG. 53 illustrates an example flow diagram of generating annotation data associated with a surgical video using surgical context data and situational awareness. As shown, at 50370, surgical context data may be obtained. The surgical context data may be obtained, for example, using surgical procedure data, a surgical procedure plan, and/or the like. As shown at 50372, a surgical video comprising surgical video frames may be obtained. As shown at 50374, element(s) may be identified in a surgical video frame. The element(s) may be identified, for example, based on surgical context data. The element(s) may be identified, for example, using image processing (e.g., as described herein).

As shown at 50376, annotation data may be generated, for example, based on the surgical context data and the element(s) identified in the surgical video frame. For example, the annotation data may include one or more of the following: element identification information, element grouping information, element subgrouping information, element type information, element description information, element condition information, surgical step information, surgical task information, surgical event information, surgical instrument information, surgical equipment information, and/or the like. In some examples, the annotation data may be inserted into the surgical video. The annotation data may be inserted into the video frame. For example, the annotation data may be inserted on a pixel level (e.g., respective annotation data is inserted into each pixel and/or group of pixels). In some examples, the annotation data may be used to generate control signal (e.g., indicating the determined control parameters), for example, to a surgical control system. In some examples, the annotation data may be sent separately from the surgical video (e.g., to a surgical control system, to a surgical analysis system, etc.).

In a surgical procedure, parameters may be used to determine between primary control and confirmation control for a process and/or an operation. For example, a parameter may be monitored in a surgical procedure. An uncertainty associated with the parameter may be determined. The uncertainty associated with the monitored parameter may be used (e.g., as a means) to determine between primary control and confirmation control, for example, of a process and/or operation.

The reliability and/or uncertainty of data from surgical procedure data (e.g., from surgical sensor(s)) may be used, for example, to determine how to perform an automated task. For example, the reliability and/or uncertainty of data from a surgical sensor(s) (e.g., wearables) may be used to determine which of the sensors to use to drive and/or perform an automated task.

The detected reliability and/or uncertainty of the monitored data and/or the magnitude of the uncertainty of a first sensor or detection array and a second detection array may be used, for example, to determine prioritization. For example, the prioritization may be associated with a prioritization of each array in a hierarchy of control of a system or an automated task.

For example, vital organ proximity detection system(s) may analyze the body cavity. An automated task may use a device (e.g., surgical instrument) to move within the body cavity. Sensors may be used, for example, to capture data associated with analyzing the body cavity. Based on the multiple sensors, an autonomous system (e.g., surgical computing system, which may be the surgical hub 20006 as described in FIG. 1) may determine (e.g., using an algorithm) to determine which sensor has the most accurate (e.g., best) proximity measurement (e.g., as compared with the other sensors) for locating the vital organs (e.g., for determining a safe path for the movement, for example, of the surgical instrument in the body cavity). A camera sensor may have a blocked view, but the camera sensor may have tracked the organs and energy device to estimate (e.g., guess) at the locations (e.g., of the vital organs). The device (e.g., surgical instrument in the body cavity) may have an ultrasonic sensor that may locate close objects. The autonomous system may cross reference both sensors (e.g., the camera sensor and the ultrasonic sensor) and determine the safest movement based on the reliability of the data received from the sensors. The organ risk may be considered depending on the device being used to navigate the body cavity. For example, activating an energy device close to the heart is riskier than activating close to the lung.

A surgical job or surgical step may be used to explain (e.g., provide context for) the choices the surgical system may have for controlling the operation, for example, based on multiple (e.g., two) related but differing data streams and a means for determining the reliability of each and how the choices may affect automated control of the process.

A target of uncertainty data may include impendence and/or reflectivity, for example, that may be used to characterize tissue for compression and/or mechanical properties. The impendence and/or reflectivity data may include an unstable measure or a conflicting data element (e.g., less impendence may be related to ore compression). Then, if the data uncertainty exceeds a threshold level, another measure (e.g., reflectivity and/or ultrasonic imaging) may be used as a supplement and/or may replace the use of impendence.

Reliability and/or risk level of an aspect of the surgical procedure may be used, for example, to determine the control reliance on a specific monitored data stream. Automated risk analysis may be performed, for example, to determine a control mechanism reliance on a specific data stream.

Automatic event recording and storage may be performed. For example, device controls may use aggregated intraoperative information from other devices in use in the same surgical procedure.

For example, surgical procedure data may be autonomously collected (e.g., from multiple surgical devices). The surgical procedure data may be aggregated from multiple devices throughout the surgical procedure.

The surgical procedure data may be aggregated throughout the procedure, for example, to predict high level patient and/or tissue factors (e.g., that may influence outcomes associated with other devices used in the surgical procedure). For example, if a surgical procedure data indicates that a patient bleeds more than average (e.g., risk factor), the risk factor may be indicated to other surgical devices to be used in the other surgical devices' algorithms and/or operations. For example, stapling parameter and/or controls (e.g., clamp loads, precomp times, firing speeds, etc.) may be adjusted, for example, based on autonomously collected and interrogated tissue and/or patient information from energy device activations throughout the procedure.

For example, surgical procedure data indicating that energy activations throughout the procedure may have taken longer than typical to ensure an adequate seal. The outcome may be associated with a patient-specific factor, for example, where the patient is more prone to bleed (e.g., as compared to others) and/or does not clot as quickly as normal anatomies. The information may be included and/or used in a stapling algorithm and/or operation. The surgical system may make adjustments to the surgical instrument parameters (e.g., clamp load, precompression time, firing speeds, etc.) to reduce staple line bleeding, for example, if the patient has a higher propensity for bleeding. The surgical hub may recommend a tighter staple than the surgeon may typically use for the procedure.

Surgical device data may be mapped to a location in the patient where the data is being generated. For example, video analysis and/or other markers may be used to track the locations of device usage throughout the procedure, for example, to map the collected device data for future device usage. In examples, during mobilization in a lower anterior resection (LAR), an energy transection near the rectum may provide data that indicates lower relative tissue perfusion and/or lower relative likelihood for the staple line to bleed in that area. In examples, in the same procedure, an energy transection up the colon may be more perfuse and may be more likely to ooze. To limit intra-operative bleeding, staple controls and reload selection may be different at each location. The surgical system may track the energy activations by location and may track the position of endocutter firings. If the endocutter is in close proximity to where a given energy activation was completed, the endocutter controls and/or parameters may be adjusted accordingly.

The surgical system may track device data. For example, visible bleeding from video analysis may be determined. Energy device tissue sealing data (e.g., time to seal and/or complete a transection) may be obtained and/or determined. Perfusion measures may be determined. Tissue load curves from various device clamping may be used to determine density and/or perform viscoelasticity analysis. Tissue fluid content may be determined, for example, based on impendence (e.g., from energy devices).

A surgeon may actively request tissue information. An energy device tissue interrogation mode may be used to detect tissue thickness and/or condition for use in stapling. Feedback may be given to the surgeon and support cartridge selection and/or directly apply information to compression and/or firing algorithms.

For example, a surgeon may activate the tissue interrogation mode on a bipolar device prior to transecting with a stapler. The bipolar device may go into a non-therapeutic energy delivery mode. The surgeon may clamp and release on the intended staple line transection are, for example, while the non-therapeutic energy passes between the jaws. The impendence and/or other energy data may be used to estimate tissue thickness. The surgical system may communicate the estimated tissue thickness and recommend staple reload parameters to the surgeon. The data recorded from the tissue interrogation may be used a part of the stapling compression and/or firing algorithm, for example, if the surgeon uses the stapler. Bipolar devices may use impedance as a predictor. Harmonic devices may use ultra-low energy mode. Tissue resistance, density, vibration response, and/or the like may be used as a predictor (e.g., by harmonic devices). Stapling algorithms may be adjusted based on energy device feedback to determine tissue type, thickness, density, condition, and/or the like. Energy device feedback may be used to confirm tumor margins prior to stapling.

Event and/or situational relevance may be determined for a surgical procedure, for example, to control automatic storage of an event and timing. Relevance, risk, and/or criticality of a task may be used to determined, for example, if, when, where, and/or how often the aspects of the situation and/or event are stored.

Motion sensing may be used, for example, instead of frame to frame comparison, for example for automatic event recording and/or storage. Event based cameras may be used. Identification and re-identification of objects in combination with a memory of key and/or common object previous placement may be performed to allow for the object location to be stored. The stored coordinated system may be adjusted, for example, as the object is moved, for example, to free up inter-frame comparison of pixels and images for systems that have a stationary, fixed, and/or predefined location. The amount of image processing on portions of the system view that are less consequential (e.g., but object location still matters), may be limited.

1. A method, comprising:
determining an autonomous operation parameter; and
generating a control signal for an autonomous operation based on the autonomous operation parameter.

2. The method of claim 1, further comprising:
sending the control signal for the autonomous operation to a smart surgical device.

3. The method of claim 1, further comprising:
obtaining surgical data, wherein the autonomous operation parameter is determined based on the surgical data.

4. The method of claim 1, further comprising:
obtaining surgical context data, wherein the autonomous operation parameter is determined based on the surgical context data.

5. A surgical computing device comprising a processor configured to:
determine an autonomous operation parameter; and
generate a control signal for an autonomous operation based on the autonomous operation parameter.

6. The surgical computing device of claim 5, wherein the processor is further configured to:
send the control signal for the autonomous operation to a smart surgical device.

7. The surgical computing device of claim 5, wherein the processor is further configured to:
obtain surgical data, wherein the autonomous operation parameter is determined based on the surgical data.

8. The surgical computing device of claim 5, wherein the processor is further configured to:
obtain surgical context data, wherein the autonomous operation parameter is determined based on the surgical context data.

9. The surgical computing device of claim 5, wherein the processor is further configured to:
monitor the autonomous operation by tracking an output associated with a control feedback; and
in response to detecting the output crossing a threshold, switch to a second autonomous operation.

10. The surgical computing device of claim 5, wherein the processor is further configured to:
detect a first surgical device in the surgical environment, wherein the first surgical device comprises a first plurality of features,
detect a second surgical device in the surgical environment, wherein the second surgical device comprises a second plurality of features; and
determine one or more features from the first plurality of features for the first surgical device to perform and one or more features from the second plurality of features for the second surgical device to perform.

11. The surgical computing device of claim 5, wherein the processor is further configured to:
receive first operation data associated with a first surgical procedure and second operation data associated with a second surgical procedure, wherein the first operation data is associated with a first aspect of a control algorithm of a first surgical device, wherein the second operation data is associated with a first aspect of a control algorithm of a second surgical device, and wherein the first surgical device and second surgical device are of a first surgical device type;
receive first outcome data associated with the first surgical procedure and second outcome data associated with the second surgical procedure;
determine that each of the control algorithm of the first surgical device and the control algorithm of the second surgical device is an up-to-date control algorithm associated with the first surgical device type;
generate first aggregation data based on at least the first operation data, the second operation data, the first outcome data, and the second outcome data;
based on at least the first aggregation data, determine a correlation between the first aspect of the up-to-date control algorithm and outcome data; and
based on the determined correlation, generate an updated up-to-date control algorithm.

12. The surgical computing device of claim 5, wherein the processor is further configured to:
receive a first discrete signal associated with clamping control;
generate, in response to the first discrete signal, a first continuous signal to cause a continuous application of force based on a first autonomous control algorithm, wherein the continuous application of force is adjusted autonomously based on at least a first measurement;
receive a second discrete signal associated with a deployment operation; and
generate, in response to the second discrete signal, a second continuous signal to cause the deployment operation based on a second autonomous control algorithm, wherein the deployment operation is adjusted autonomously based on at least a second measurement.

13. The surgical computing device of claim 5, wherein the processor is further configured to:
obtain surgical procedure data from a plurality of surgical systems;
annotate the surgical procedure data with surgical context data;
determine, based on at least in part the annotated surgical procedure data, a data needs associated with a subsequent target system task associated with a target system;
generate a data package associated with the data need, the data package comprising a subset of the annotated surgical procedure data; and
send the data package to the target system.

14. The surgical computing device of claim 5, wherein the processor is further configured to:
obtain patient specific pre-surgical data for a patient specific surgical procedure;
obtain a surgical procedure template comprising surgical procedure steps;
determine one or more surgical task options for a first surgical procedure step and a second surgical procedure step based at least in part on the patient specific pre-surgical data;
determine a respective characterization for the one or more surgical task options based on the patient specific pre-surgical data, wherein the respective characterizations are associated with a respective predicted outcome; and
generate a populated patient specific procedure plan comprising the one or more surgical task options for the first surgical procedure step and the second surgical procedure step and the respective characterizations associated with the one or more surgical task options.

15. The surgical computing device of claim 5, wherein the processor is further configured to:
obtain surgical context data;
obtain a surgical video comprising one or more surgical video frames;
identify one or more elements in a first surgical video frame based on the obtained surgical context data using image processing, wherein the one or more elements comprise a respective group of pixels; and
generate annotation data for the first surgical video frame based on the surgical context data and the one or more elements in the first surgical video frame, wherein the annotation data comprises respective element annotation data associated with the identified one or more elements.

16. A method for automating a surgical task, the method comprising:
operating at a first level of automation of a plurality of levels of automation associated with the surgical task;
obtaining an indication to switch to a second level of automation of the plurality of levels of automation associated with the surgical task; wherein the indication is based on detecting a trigger; and
operating at the second level of automation of the plurality of levels of automation associated with the surgical task based on the indication.

17. The method of claim 16, wherein the indication is based on monitoring a performance of the first level of automation.

18. The method of claim 16, wherein the performance of the first level of automation is based on real-time surgical data.

19. The method of claim 16, further comprising:
receiving an updated control algorithm;
determining the updated control algorithm is more up-to-date than an installed control algorithm;
replacing the installed control algorithm with the updated control algorithm; and
operating using the updated control algorithm.

20. The method of claim 19, wherein replacing the installed control algorithm with the updated control algorithm comprises replacing coefficients associated with the installed control algorithm with updated coefficients associated with the updated control algorithm.
